(19) United States
(12) Patent Application Publication
(10) Pub. No.: US 2009/0143285 A1
(43) Pub. Date: Jun. 4, 2009

(54) TARGET PROTEIN AND TARGET GENE FOR DRUG DISCOVERY AND SCREENING METHOD

(75) Inventors: Hideaki Sueoka, Osaka (JP); Tadakazu Yamauchi, Shizuoka (JP); Kouichi Tsuchiya, Tokyo (JP); Katsuhisa Murayama, Osaka (JP); Morikazu Kito, Kanagawa (JP); Takeshi Tsutsumi, Osaka (JP); Yuko Isono, Kanagawa (JP); Kazuo Komiya, Hyogo (JP); Noriyuki Inomata, Kanagawa (JP); Yorimasa Suwa, Tokyo (JP); Ken Horiuchi, Tokyo (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: REVERSE PROTEOMICS RESEARCH INSTITUTE CO., LTD., Tokyo (JP)

(21) Appl. No.: 11/909,634

(22) PCT Filed: Mar. 24, 2006
(86) PCT No.: PCT/JP2006/306778

§ 371 Date: Dec. 17, 2007

(30) Foreign Application Priority Data
Mar. 25, 2005 (JP) 2005-089609

(51) Int. Cl. A61K 38/16; C12Q 1/68; G01N 33/566

(52) U.S. Cl. 514/12; 435/6; 435/7.8

(57) ABSTRACT

The present invention provides target proteins and target genes for bioactive substances such as drugs, and means that enable the development of novel bioactive substances using the same. More specifically, the present invention provides target proteins and target genes for bioactive substances; screening methods for substances capable of regulating bioactivities; bioactivity regulators; a bioactive substance derivative production method; a complex comprising a bioactive substance and a target protein, and a method of producing the complex; and kits comprising a bioactive substance or a salt thereof; determination methods for the onset or risk of onset of a specified disease or condition, determination methods for susceptibility to a bioactive substance, and determination kits used for the determination methods, and the like.

### TECHNICAL FIELD

The present invention relates to target proteins and target genes that are useful for the development of bioactive substances, for example, drug discovery; a screening method for a bioactive substance and the substance obtained by the screening method; a bioactivity regulator; a bioactive substance derivative and a method of producing the derivative; and a complex comprising a bioactive substance and a target protein therefor and a method of producing the complex, and the like.

### BACKGROUND ART

Traditionally, the success rate of new drug research and development is quite low, with only one or two of about 100 research projects ending successfully with the launch of a new drug (D. Brown and G. Superti-Furga, Drug Discovery Today, December, 2003). This is mostly because of premature termination of the development due to a problem with the economy, safety or efficacy of the new drug candidate compound (Dimasi, Clin. Pharmacol. Ther., 69, 297-307, 2001).

Pharmaceutical companies are spending 10 to 20% of their sales on R&D activities; it is of paramount importance to efficiently spend R&D budgets for pharmaceutical companies to be highly competitive. Furthermore, because about 80% of R&D expenditures are spent for costly clinical studies in the developmental stage, it is critical to select appropriate candidate compounds in the initial stage prior to progress to the developmental stage.

In recent years, on the other, the genome sequences of a variety of organisms have been elucidated and analyzed at the global level. For the human genome, in particular, a worldwide cooperative research project was implemented, and completion of analysis of all sequences thereof was announced in April 2003. As a result, it is becoming possible to analyze complex biological phenomena in the context of the functions and control of all genes, or networks of gene-gene, protein-protein, cell-cell, and individual-individual interactions. The genome information thus obtained has been significantly revolutionizing a number of industries, including drug development, as well as in academic sectors.

For example, it has been reported that there are about 480 kinds of target proteins for drugs having been in common use to date, and that these target proteins are limited to membrane receptors, enzymes, ion channels, or nuclear receptors and the like (J. Drews, Science, 297, 1960-1964, 2000). Meanwhile, target protein search based on genome information has discovered an extremely large number of target proteins, including novel proteins not covered in the conventional range of target proteins one after another, which are estimated to total about 1,500 kinds (A. L. Hopkins & C. R. Groom, Nature Reviews; Drug Discovery, 1, 727-730, 2002).

However, despite the fact that the research and development expenditures spent by pharmaceutical companies are increasing due to rises in infrastructuring costs for coping with vast amounts of data like genome information and clinical developmental costs, the number of new drugs approved per year is tending to decrease on the contrary (Nature Reviews; Drug Discovery, February 2003). This shows that the above-described genome information is actually not efficiently utilized.

As a means for overcoming these circumstances, Nagashima et al. invented “Method, System, Apparatus, and Device for Discovering and Preparing Chemical for Medical and Other Uses” and filed a patent application for that invention (National Publication of Translated Version No. 2004-509406).

Disclosed in that patent application are methods, systems, databases, user interfaces, software, media, and services that are useful for the evaluation of compound-protein interactions, and are also useful for the utilization of the information resulting from such an evaluation intended to discover compounds in medical and other areas. Furthermore, it is intended to produce a very large pool of novel target proteins for drug discovery, novel methods for designing novel drugs, and a pool of small substances for therapeutic purposes that are virtually synthesized as having been inconceivable in the past.

Specifically, disclosed in that patent application was a method of identifying a protein or partial protein that is appropriate as a novel drug discovery target, which comprises the following steps:

(i) a step for selecting a plurality of proteins or partial proteins showing desired affinity and specificity for a selected target compound;
(ii) a step for identifying the structure and function of the protein or the partial protein; and
(iii) a step for selecting a single protein or single partial protein having a desired function, and a method of discovering a drug, which comprises the following steps:
(i) a step for investigating the chemical structure of the target compound selected using the above-described method; and
(ii) a step for chemically modifying the structure of the selected target compound to optimize the affinity and specificity of the modified compound for the protein or the partial protein, which is appropriate as a novel drug target.

Furthermore, another feature of the method disclosed in that patent application resides in that the selected target compound is a compound approved for medical use.

Conventional drugs that have been used to date include many drugs for which target proteins are unknown, or for which target proteins are known but not all of whose pharmacological effects and adverse effects can be explained by mechanisms mediated by the proteins.

Typically, aspirin, one of the drugs that have longest been used, may be mentioned. When aspirin was launched in the market for the first time more than 100 years ago, the mechanism for its anti-inflammatory action was unclear. About 70 years later, aspirin was found to have cyclooxygenase (COX) inhibitory action. Still 20 years later, it was demonstrated that COX occurred in two subtypes: COX-1 and COX-2, that the primary pharmacological effect of aspirin was based on COX-2 inhibition, and that COX-1 inhibitory action was the cause of adverse effects such as gastrointestinal disorders. However, not all the target proteins for aspirin have been elucidated. In recent years, aspirin has been shown to exhibit anticancer action and antidementic action in clinical settings, but these pharmacological effects cannot be explained by COX inhibition. On the other, recent years have seen many papers reporting that aspirin acts on transcription factors such as IKKβ and on nuclear receptors such as PPAR-γ, but the association of these and the various pharmacological effects of aspirin remains unclear.

For these reasons, elucidating target proteins for traditionally used drugs can be said to be a very effective approach to discovering novel drug discovery target proteins.

Hirayama, one of the inventors of the above-described published patent, and others generated a database integrating the structural and physical property data on about 1,500 kinds of drugs commercially available in Japan, and found that existing pharmaceutical compounds share structural features (Chem-Bio Informatics Journal, 1, 18-22, 2001). Drugs that have been commonly used to date can be described as excellent in that they have cleared the issues of localization in the body and safety in their developmental processes. Searching novel target proteins with these existing drugs as probes, and selecting novel drug candidate compounds on the basis of their structures is thought to be a highly reasonable and efficient approach.

A second problem arises concerning how to make use of the genome information during the search for novel target proteins. Solely determining the genome sequence is not sufficient to ensure the elucidation of the functions of all genes and the discovery of drug discovery target proteins. It is estimated that in humans, about 30,000 to 40,000 kinds of genes are present; taking into consideration variants from alternative splicing, there are reportedly more than 100,000 kinds of mRNA. It is important, therefore, that out of the vast amount of new genes revealed from the genome sequence, those having useful functions in industrial applications, including drug development, should be efficiently selected and identified.

In many cases, in the genome sequences of eukaryotic organisms, each gene is divided into a plurality of exons by introns; therefore, it is impossible to accurately predict the structure of the protein encoded by the gene solely from the sequence information on the gene. In contrast, for a cDNA prepared from intron-excluded mRNA, information on the amino acid sequence of protein is obtained as information on a single continuous sequence, enabling easy determination of the primary structure thereof.

In particular, analyzing a full-length cDNA enables the identification of the mRNA transcription initiation point on the genome sequence based on the 5′-terminal sequence of the cDNA, and also enables analysis of the stability of mRNA contained in the sequence and of factors involved in expression control in the translation stage. Also, because the ATG codon, which serves as the translation initiation point, is present on the 5′ side, translation into protein in the right frame can be achieved. Therefore, by using an appropriate gene expression system, it is also possible to mass-produce the protein encoded by the cDNA, and to express the protein and analyze the biological activity thereof. Hence, it is considered that by performing an analysis using a protein expressed from full-length cDNA, important information that could not be obtained solely by genome sequence analysis is obtained, and that it is possible to discover novel target proteins that do not lie in the conventional category of drug discovery target proteins.

### DISCLOSURE OF THE INVENTION

The objects of the present invention are to provide target proteins and target genes for the development of bioactive substances (e.g., drug discovery), and various means that enable the development of novel bioactive substances using the same and the like.

The present inventors diligently investigated new drug discovery target proteins that can be useful for the development of new drugs, by analyzing interactions between human proteins and compounds that have been used as drugs by the SEC/MS method, and found novel target proteins and novel target genes that are useful for the development of bioactive substances, for example, drug discovery. The present inventors conducted further investigations based on this finding, conceived that substances that regulate the expression or function of these genes are capable of regulating various bioactivities, and that substances capable of regulating various bioactivities are developed by screening substances that regulate the expression or function of these genes, and by derivatizing these bioactive substances so that the expression or function of the target genes therefor can be regulated, and the like, and completed the present invention.

Accordingly, the present invention is as follows:

[1] A method for screening a substance capable of regulating an action associated with bioactive substance X, which comprises determining whether or not a test substance is capable of regulating the expression or function of target protein Y or a gene that encodes the protein, wherein the combination of bioactive substance X and target protein Y is any of the following (a1) to (a110) (referred to as “combinations A” as required):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3,

SEQ ID NO:14 or SEQ ID NO:19 or a protein homologous thereto or a variant thereof;

(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.
[2] The method according to [1] above, which comprises the following steps (a) to (c):
(a) a step for bringing the test substance into contact with target protein Y;
(b) a step for measuring the functional level of the protein in the presence of the test substance, and comparing said functional level with the functional level of the protein in the absence of the test substance;
(c) a step for selecting a test substance that alters the functional level of the protein on the basis of the result of the comparison in (b) above.
[3] The method according to [1] above, which comprises the following steps (a) to (c):
(a) a step for bringing the test substance and cells allowing a measurement of the expression of target protein Y or a gene that encodes the protein into contact with each other;
(b) a step for measuring the expression level of the gene in the cells in contact with the test substance, and comparing said expression level with the expression level of the gene in control cells not in contact with the test substance;
(c) a step for selecting a test substance that regulates the expression level of the gene on the basis of the result of the comparison in (b) above.
[4] The method according to [1] above, which comprises the following steps (a) to (c):
(a) a step for bringing the test substance into contact with target protein Y;
(b) a step for measuring the ability of the test substance to bind to the protein;
(c) a step for selecting a test substance capable of binding to the protein on the basis of the result from (b) above.
[5] The method according to [1] above, which comprises the following steps (a) to (c):
(a) a step for bringing the test substance and a target protein Y-binding substance into contact with target protein Y;
(b) a step for measuring the ability of the target protein Y-binding substance to bind to the protein in the presence of the test substance, and comparing said ability with the ability of the target protein Y-binding substance to bind to the protein in the absence of the test substance;
(c) a step for selecting a test substance that alters the ability of the target protein Y-binding substance to bind to the protein on the basis of the result of the comparison in (b) above.
[6] A method for screening a substance capable of regulating a function associated with target protein Y, which comprises comparing the ability of a test substance to bind to target protein Y or the action associated with the test compound, with the ability of bioactive substance X to bind to target protein Y or the action associated with the bioactive substance, wherein the combination of target protein Y and bioactive substance X is any of the following (b1) to (b23) (referred to as “combinations B” as required):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.
[7] A substance obtained by the method according to any one of [1] to [6] above.
[8] An agent of regulating a bioactivity, which comprises a substance obtained by the method according to any one of [1] to [6] above.
[9] An agent of regulating an action associated with bioactive substance X, which comprises a substance that regulates the expression or function of target protein Y or a gene that encodes the protein, wherein the combination of bioactive substance X and target protein Y is any combination of combinations A.
[10] The agent according to [9] above, wherein the substance that regulates the expression or function of target protein Y or a gene that encodes the protein is a substance that suppresses the expression or function of the gene.
[11] The agent according to [10] above, wherein the substance that suppresses the expression or function of target protein Y or a gene that encodes the protein is an antisense nucleic acid, ribozyme, decoy nucleic acid, siRNA, antibody or a dominant negative mutant, or an expression vector thereof.
[12] The agent according to [9] above, which comprises target protein Y, or an expression vector comprising a nucleic acid that encodes the protein.
[13] An agent of regulating a function associated with target protein Y, which comprises bioactive substance X, wherein the combination of target protein Y and bioactive substance X is any combination of combinations B.
[14] A method of producing a derivative of bioactive substance X, which comprises derivatizing bioactive substance X so as to be able to regulate the expression or function of target protein Y or a gene that encodes the protein, wherein the combination of bioactive substance X and target protein Y is any combination of combinations A.
[15] A method of producing a derivative of a substance capable of regulating a function associated with target protein Y, which comprises derivatizing bioactive substance X so as to be able to regulate the ability of bioactive substance X to bind to target protein Y, wherein the combination of target protein Y and bioactive substance X is any combination of combinations B.
[16] A bioactive substance derivative obtained by the method according to [14] or [15] above.
[17] An agent of regulating a bioactivity, which comprises a bioactive substance derivative obtained by the method according to [14] or [15] above.
[18] A complex comprising bioactive substance X and target protein Y thereof, wherein the combination of bioactive substance X and target protein Y is any combination of combinations A or combinations B.
[19] A method of producing the complex according to [18] above, which comprises bringing the bioactive substance and the target protein therefor into contact with each other.
[20] A kit comprising the following (i) and (ii):
(i) bioactive substance X or a salt thereof;
(ii) target protein Y, a nucleic acid that encodes the protein, an expression vector comprising the nucleic acid, cells that enable a measurement of the expression of target protein Y or a gene that encodes the protein, or an expression vector comprising the transcription regulatory region of a gene that encodes target protein Y and a reporter gene functionally linked thereto;

wherein the combination of bioactive substance X and target protein Y is any combination of combinations A or combinations B.

[21] A method for determining the onset or risk of onset of a disease or condition associated with an action of bioactive substance X, which comprises the following steps (a) and (b):
(a) a step for measuring the expression level and/or polymorphism of target protein Y or a gene that encodes the protein in a biological sample collected from an animal;
(b) a step for evaluating the onset or likelihood of onset of the disease or condition on the basis of the measured expression level and/or polymorphism;

wherein the combination of bioactive substance X and target protein Y is any combination of combinations A.

[22] A method for determining the onset or risk of onset of a disease or condition associated with a function of target protein Y, which comprises the following steps (a) and (b):
(a) a step for identifying the polymorphism of the gene that encodes target protein Y in a biological sample collected from an animal;
(b) a step for evaluating the onset or likelihood of onset of the disease or condition on the basis of the presence or absence of a particular type of polymorphism;

wherein the particular type of polymorphism alters the ability of target protein Y to bind with bioactive substance X,

wherein the combination of target protein Y and bioactive substance X is any combination of combinations B.

[23] A kit for determining the onset or risk of onset of a disease or condition associated with an action of bioactive substance X, which comprises the following (i) and (ii):
(i) a means capable of measuring the expression level and/or polymorphism of target protein Y or a gene that encodes the protein;
(ii) a medium recording the relationship between the disease or condition and the expression level and/or polymorphism of the gene;

wherein the combination of bioactive substance X and target protein Y is any combination of combinations A.

[24] A kit for determining the onset or risk of onset of a disease or condition associated with a function of target protein Y, which comprises the following steps (i) and (ii):
(i) a means capable of identifying the polymorphism of a gene that encodes target protein Y;
(ii) a medium recording the relationship between the disease or condition and the polymorphism of the gene;

wherein the particular type of polymorphism alters the ability of target protein Y to bind with bioactive substance X,

wherein the combination of target protein Y and bioactive substance X is any combination of combinations B.

[25] A method for determining susceptibility to bioactive substance X in a disease or condition associated with an action of bioactive substance X, which comprises the following steps (a) and (b):
(a) a step for measuring the expression level and/or polymorphism of target protein Y or a gene that encodes the protein in a biological sample collected from an animal;
(b) a step for predicting the effect of the bioactive substance on the basis of the measured expression level and/or polymorphism;

wherein the combination of bioactive substance X and target protein Y is any combination of combinations A.

[26] A method for determining susceptibility to bioactive substance X in a disease or condition associated with a function of target protein Y, which comprises the following steps (a) and (b):
(a) a step for determining the type of the polymorphism of the gene that encodes target protein Y in a biological sample collected from an animal;
(b) a step for predicting the effect of bioactive substance X in the disease or condition on the basis of the presence or absence of a particular type of polymorphism;

wherein the particular type of polymorphism alters the ability of target protein Y to bind with bioactive substance X,

wherein the combination of target protein Y and bioactive substance X is any combination of combinations B.

[27] A kit for determining susceptibility to bioactive substance X in a disease or condition associated with an action of bioactive substance X, which comprises the following (i) and (ii):
(i) a means capable of measuring the expression level and/or polymorphism of a gene that encodes target protein Y;
(ii) a medium recording the relationship between the effect of bioactive substance X and the expression level and/or polymorphism of the gene;

wherein the combination of bioactive substance X and target protein Y is any combination of combinations A.

[28] A kit for determining susceptibility to bioactive substance X in a disease or condition associated with a function of target protein Y, which comprises the following (i) and (ii):
(i) a means capable of identifying the polymorphism of a gene that encodes target protein Y;
(ii) a medium recording the relationship between the effect of bioactive substance X and a particular type of the polymorphism of the gene;

wherein the particular type of polymorphism alters the ability of target protein Y to bind with bioactive substance X,

wherein the combination of target protein Y and bioactive substance X is any combination of combinations B.

### BRIEF DESCRIPTION OF THE DRAWINGS

FIG. 1 is a schematic diagram showing a SEC interaction screening system using a micro-LC column.

FIG. 2 is a schematic diagram showing a SEC interaction analysis using a micro-LC column.

FIG. 3 is a schematic diagram showing a SEC interaction screening system using a spin column.

FIG. 4 is a schematic diagram showing a SEC interaction analysis using a spin column.

### BEST MODE FOR CARRYING OUT THE INVENTION

### 1. Target Proteins and Target Genes for Bioactive Substances

The present invention provides target proteins and target genes for the development of bioactive substances.

A bioactive substance means any substance that has an action on the body. The bioactive substance can be an exogenous substance such as a drug, vitamin, herbal medicine ingredient, or food ingredient, and can be an endogenous substance such as a cytokine, growth factor, or hormone. When a given bioactive substance is intended, it is expressed as bioactive substance X as required.

Bioactive substance X includes the bioactive substances capable of regulating the expression or function of target protein Y or a gene that encodes the protein, described below, for example, bioactive substances capable of binding to target protein Y. Specifically, bioactive substance X can be picotamide, methoxsalen, terfenadine, cyclosporin A, pancuronium bromide, hydroxocobalamin, amphotericin B, protriptyline, rifampicin, solanine α, amethopterin, benztropine, sulfasalazine, nalidixic acid, astemizole, chlorprothixene, loperamide, fluphenazine, mefloquine, perphenazine, perhexyline, raloxifene, simvastatin, benoxinate, pioglitazone, thioproperazine, quinacrine, GBR12909, benzethonium, albendazole, acetopromazine amikacin, amiodarone, apigenin, buprenorphine, celestine blue, chlorambucil, chlorhexidine, chlorpromazine, cinchonine, clofazimine, clomiphene, cyproheptadine, deferoxamine, dihydroergocristine, dihydroergotamine, diphemanil, domperidone, doxazosin, eburnamonine, ellipticine, emetine, ethotoin, fenbendazole, flumequine, flunarizine, flupentixol, glipizide, harmaline, hydantoin, lidoflazine, lisinopril, mafenide, mefenamic acid, megestrol, mesoridazine, metergoline, methoxy-6-harmalan, meticrane, methixene, mifepristone, nordiazepam, oxethazaine, oxolinic acid, paclitaxel, palmatine, pentoxifylline, pimozide, pinacidil, rescinnamine, SR-95639A, sulfadimethoxine, syrosingopine, tamoxifen, tenoxicam, thioridazine, thiothixene (cis), tomatidine, dipyrone, ethyl loflazepate, clobazam, alimemazine, ebastine, reserpine, paramethasone, hydroxyprogesterone, dydrogesterone, sarpogrelate, demeclocycline, avermectin B1A, solasodine, nanofin (cis), tetrazoline, methylbenzethonium chloride, α-ergocryptine, spiramycin, chloropyramine, bergenin, nafcillin and carboprost, or a derivative thereof capable of binding to target protein Y (described later), or a salt thereof.

Bioactive substances can also be roughly divided, from the viewpoint of the type of activity that can be regulated thereby, into two groups: substances capable of regulating an action associated with bioactive substance X, and substances capable of regulating a function associated with target protein Y.

The target proteins and target genes for the development of bioactive substances can preferably be target proteins and target genes for drug discovery. When a given target protein and a given target gene are intended, they are expressed as target protein Y and target gene Y, respectively, as required. The term protein has the same definition as a translation product, and the term target gene Y has the same definition as a gene that encodes target protein Y; these terms are interchangeably used.

For example, target protein Y can be a target protein for the above-described bioactive substance X. Specifically, target protein Y can be a protein comprising the amino acid sequence shown by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID N0:18, SEQ ID N0:19, SEQ ID NO:20, SEQ ID N0:22, SEQ ID NO:23, or SEQ ID NO:24 (e.g., full-length protein) or a protein homologous thereto or a variant thereof. As mentioned herein, the target proteins of the present invention are not limited to human proteins, but include orthologues of different animal species. Referring to human proteins for reference, information on various aspects and some examples of binding bioactive substances discovered by the present inventors are shown in Tables 1-1 to 1-6 and Tables 2-1 to 2-4, respectively.

TABLE 1-1

| | | | FLJ nucleotide | FLJ amino acid | | | |
|---|---|---|---|---|---|---|---|
| | Sequence | | sequence | sequence | | | Example of bound |
| FLJ number | number | ORF mutation | Accession | Accession | H-InV cDNA ID | H-Inv locus ID | bioactive substances |
| FLJ10420 | 1 | A469G: ACC(Thr)GCC(Ala) | AK001282.1 | BAA91598.1 | HIT000003756 | HIX0000170 | picotamide |
| FLJ10537 | 2 | | AK001399.1 | | HIT000003873 | HIX0018494 | methoxsalen |
| FLJ11211 | 3 | | AK002073.1 | | HIT000004547 | HIX0006815 | terfenadine |
| | | | | | | | cyclosporin A |
| | | | | | | | deferoxamine |
| | | | | | | | mesoridazine |
| | | | | | | | pimozide |
| | | | | | | | eburnamonine |
| FLJ12857 | 4 | | AK022919.1 | | HIT000006193 | HIX0014561 | pancuronium bromide |
| FLJ20972 | 5 | | AK024625.1 | BAB14936.1 | HIT000007899 | HIX0021214 | protriptyline |
| FLJ21841 | 6 | | AK025494.1 | BAB15153.1 | HIT000008768 | HIX0001169 | rifampicin |
| | | | | | | | mafenide |
| | | | | | | | spiramycin |
| FLJ322317 | 7 | | AK025970.1 | | HIT000009244 | HIX0014885 | solanine α |
| | | | | | | | mifepristone |
| FLJ23466 | 8 | A450G: CCA(Pro)CCG(Pro) | AK027119.1 | BAB15663.1 | HIT000010393 | HIX0019290 | cyclosporin A |
| | | | | | | | chlorambucil |
| FLJ25288 | 9 | | AK058017.1 | | HIT000014623 | HIX0002130 | amethopterin |
| FLJ35914 | 10 | | AK093233.1 | | HIT000018100 | HIX0012191 | hydroxocobalamin |
| | | | | | | | hydantoin |

TABLE 1-2

| | | | | | | | |
|---|---|---|---|---|---|---|---|
| FLJ36526 | 11 | C1081T: CTC(Leu)TTC(Phe) | AK093845.1 | | HIT000018711 | HIX0015565 | hydroxocobalamin |
| | | | | | | | acetopromazine |
| | | | | | | | SR-95639A |
| | | | | | | | tetrazoline |
| | | | | | | | diphemanil |
| FLJ37909 | 12 | | AK095228.1 | BAC04505.1 | HIT000020083 | HIX0001013 | cyclosporin A |
| | | | | | | | amikacin |
| | | | | | | | amiodarone |
| | | | | | | | buprenorphine |
| | | | | | | | chlorhexidine |
| | | | | | | | clomiphene |
| | | | | | | | dihydroergocristine |
| | | | | | | | dihydroergotamine |
| | | | | | | | doxazosin |
| | | | | | | | emetine |
| | | | | | | | fenbendazole |
| | | | | | | | flunarizine |
| | | | | | | | flupentixol |
| | | | | | | | lidoflazine |
| | | | | | | | mefloquine |
| | | | | | | | metergoline |
| | | | | | | | oxethazaine |
| | | | | | | | oxolinic acid |
| | | | | | | | perhexiline |

TABLE 1-3

| | | | | | | |
|---|---|---|---|---|---|---|
| FLJ37909 | | | | | | pimozide |
| | | | | | | quinacrine |
| | | | | | | rescinnamine |
| | | | | | | tamoxifen |
| | | | | | | thioproperazine |
| | | | | | | thioridazine |
| | | | | | | thiothixene (cis) |
| | | | | | | alimemazine |
| | | | | | | reserpine |
| | | | | | | hydroxyprogesterone |
| | | | | | | raloxifene |
| FLJ38531 | 13 | | AK095850.1 | HIT000020705 | HIX0011431 | sulfasalazine |
| | | | | | | nalidixic acid |
| | | | | | | flumequine |
| | | | | | | tenoxicam |
| | | | | | | ethotoin |
| FLJ38897 | 14 | G159A: GAG(Glu)GAA(Glu) | AK096216.1 | HIT000021071 | HIX0007844 | astemizole |
| | | | | | | chlorprothixene |
| | | | | | | benztropine |
| | | | | | | loperamide |
| | | | | | | fluphenazine |
| | | | | | | mefloquine |
| | | | | | | perphenazine |
| | | | | | | perhexiline |

TABLE 1-4

| | | | | | | |
|---|---|---|---|---|---|---|
| FLJ38897 | | | | | | raloxifene |
| | | | | | | terfenadine |
| FLJ39553 | 15 | AK096872.1 | BAC04880.1 | HIT000021727 | HIX0007672 | amphotericin B |
| | | | | | | hydroxocobalamin |
| | | | | | | simvastatin |
| | | | | | | celestine blue |
| | | | | | | palmatine |
| | | | | | | sulfadimethoxine |
| | | | | | | sarpogrelate |
| | | | | | | avermectin B1A |
| | | | | | | chloropyramine |
| | | | | | | apigenin |
| | | | | | | cinchonine |
| | | | | | | harmaline |
| | | | | | | methoxy-6-harmalan |
| | | | | | | meticrane |
| | | | | | | paclitaxel |
| | | | | | | solanine α |
| | | | | | | paramethasone |
| | | | | | | demeclocycline |
| | | | | | | bergenin |
| | | | | | | nafcillin |
| | | | | | | carboprost |

TABLE 1-5

| | | | | | | | |
|---|---|---|---|---|---|---|---|
| FLJ40298 | 16 | | AK097617.1 | BAC05122.1 | HIT000022471 | HIX0017202 | benoxinate |
| FLJ40760 | 17 | | AK098079.1 | BAC05229.1 | HIT000022931 | HIX0012473 | pioglitazone |
| | | | | | | | lisinopril |
| FLJ41550 | 18 | | AK123544.1 | | HIT000043417 | HIX0027701 | thioproperazine |
| FLJ41991 | 19 | | AK123985.1 | | HIT000043858 | HIX0007844 | raloxifene |
| | | | | | | | loperamide |
| | | | | | | | mefloquine |
| | | | | | | | perphenazine |
| | | | | | | | quinacrine |
| | | | | | | | GBR12909 |
| | | | | | | | terfenadine |
| | | | | | | | fluphenazine |
| | | | | | | | astemizole |
| | | | | | | | benztropine |
| | | | | | | | chlorprothixene |
| FLJ42665 | 20 | | AK124656.1 | | HIT000044529 | HIX0010392 | benzethonium |
| | | | | | | | nanofin (cis) |
| FLJ41265 | 22 | | AK123259.1 | | HIT000043132 | HIX0006815 | cyproheptadine |
| FLJ20571 | 23 | T896C: TTG(Leu)TCG(Ser) | AK000578.1 | | HIT000003053 | HIX0014885 | domperidone |
| | | | | | | | methylbenzethonium |
| | | | | | | | chloride |

TABLE 1-6

| | | | | | |
|---|---|---|---|---|---|
| FLJ35353 | 24 | AK09672.1 | HIT000017539 | HIX0001013 | albendazole |
| | | | | | benzethonium |
| | | | | | chlorpromazine |
| | | | | | clofazimine |
| | | | | | ellipticine |
| | | | | | glipizide |
| | | | | | mefenamic acid |
| | | | | | megestrol |
| | | | | | methixene |
| | | | | | nordiazepam |
| | | | | | pentoxifylline |
| | | | | | pinacidil |
| | | | | | syrosingopine |
| | | | | | thiothixene (cis) |
| | | | | | tomatidine |
| | | | | | dipyrone |
| | | | | | ethyl loflazepate |
| | | | | | clobazam |
| | | | | | ebastine |
| | | | | | dydrogesterone |
| | | | | | solasodine |
| | | | | | methylbenzethonium |
| | | | | | chloride |
| | | | | | α-ergocryptine |

TABLE 2-1

| | | | Homologous | | |
|---|---|---|---|---|---|
| | | | protein/ | | |
| | | Corresponding | family protein | | |
| FLJ number | Protein name | protein variant | variant | Function/activity | Reference literature |
| FLJ10420 | hypothetical | NP_060560.1 | | | |
| | protein FLJ10420 | | | | |
| FLJ10537 | DIABL0 homolog; | NP_063940.1 | | apoptosis-control action, regulation | Du C et al., Cell, 102, 33 (2000); |
| | SMAC (second | NP_620308.1 | | action of Caspase activity, | Srinivasula SM et al., J Biol Chem., |
| | mitochondria-derived | NP_620307.1 | | IAP (inhibitor of apoptosis | 275, 36152 (2000); Adrain C et al., |
| | activator of | | | protein)-binding activity | EMB0 J., 20, 6627 (2001); Fu J et |
| | Caspase) | | | | al., J Biol Chem., 278, 52660 |
| | | | | | (2003); Kuai J et al., J Biol Chem., |
| | | | | | 278, 14363 (2003) |
| FLJ11211 | Cyclin | NP_066968.2 | AAG35613.1 | transcription control function, cell cycle | Tschan MP et al., J.Biol. Chem., 278, |
| FLJ41265 | D-binding | | AAG35614.1 | control function, cell differentiation | 42750 (2003); Inoue K, Genes & |
| | Myb-like | | NP_005366.2 | control function, regulation of | Development, 14, 1797 (2000); Inoue |
| | transcription | | NP_055335.1 | differentiation or activity of | K., J.Biol. Chem., 273, 29188 |
| | factor 1: | | XP_034274.7 | hematopoietic cell, control function | (1998); Hirai H., Mol.Cell.Biol., |
| | DMTF1: DMP1 | | NP_002457.1 | of differentiation and activity of | 16, 6457 (1996) |
| | | | NP_059523.1 | immune cell, regulating | |
| | | | NP_003209.1 | function of differentiation or | |
| | | | | activation of T-cell and granulocyte | |
| FLJ12857 | Paralemmin; | NP_002570 | NP_443749.3 | formation function of cell membrane | Kutzleb C. et al., J.Cell Biol. 143, |
| | PALM | | NP_060204.1 | morphology, control of lipid raft | 795 (1998); El-Husseini A-D et al., |
| | | | | function, axon and filopodia-guidance | J.Biol.Chem., 276, 44984 (2001); |
| | | | | function, synapse formation function, | Gauthier-Campbell G et al., |
| | | | | scaffolding function for receptor, | Mol.Biol.Cell., 15, 2205 (2004) |
| | | | | control of expression and function | |
| | | | | of dopamine receptor | |
| FLJ20972 | hypothetical | NP_079306.1 | | | |
| | protein | | | | |
| | FLJ20972 | | | | |

TABLE 2-2

| | | | | | |
|---|---|---|---|---|---|
| FLJ21841 | Nestin; NES | NP_006608.1 | | cell differentiation control, induction of | Yaworsky PJ et al., Dev Biol., 205, |
| | | | | nerve cell differentiation, morphogenesis of | 309 (1999); Herrmann H et al., Curr |
| | | | | central nervous system, control of | Opin Cell Biol., 12, 79 (2000) |
| | | | | cytoskeleton organization, control of cell | |
| | | | | membrane morphology | |
| FLJ22317 | HSPC142 protein | NP_054892.1 | | expression of hematopoietic stem cell and | Zhang QH et al., Genome Res., 10, |
| FLJ20571 | | | | hematopoietic precursor cell | 1546 (2000) |
| FLJ23466 | FK506-binding | NP_071393.2 | NP_002005.1 | cell cycle control, radioresistance control, | Robson T et al., Int. J. Radiat. |
| | protein-like; | | NP_004108.1 | DNA-repair control, stress response, control | Biol. 76, 617 (2000); Robson T et |
| | FKBPL | | | of protein transport and localization, | al., Radiat Res., 152, 451 (1999) |
| | | | | promotion of protein folding (immunophilin | |
| | | | | action) | |
| FLJ25288 | Annexin A4; | NP_001144.1 | NP_000691.1 | calcium-dependent phospholipid-binding | Grundmann U et al., Behring Inst |
| | ANXA4: | | NP_001002858.1 | activity, phospholipase-inhibition activity, | Mitt., No. 82, 59 (1988); Kojima K et |
| | Lipocortin IV; | | NP_001002857.1 | ATP-binding activity, anticoagulation | al., J Biol Chem., 271, 7679 |
| | placenta | | NP_004030.1 | activity | (1996); Bandorowicz-Pikula J et |
| | anticoagulant | | NP_005130.1 | | al., Mol Membr Biol., 14, 179 (1997) |
| | protein II | | NP_001145.1 | | |
| | | | NP_001146.1 | | |
| | | | NP_004024.1 | | |
| | | | NP_001147.1 | | |
| | | | NP_004025.1 | | |
| | | | NP_001621.1 | | |
| | | | NP_003559.1 | | |
| | | | NP_009124.2 | | |
| | | | NP_001148.1 | | |
| | | | NP_665875.1 | | |
| | | | NP_665876.1 | | |
| | | | NP_004297.2 | | |
| | | | NP_001003954.1 | | |

TABLE 2-3

| | | | | | |
|---|---|---|---|---|---|
| FLJ35914 | ELL3 | NP_079441.1 | | transcription factor activity, transcription | Miller T et al., J. Biol. Chem., 275, |
| | (elongation | | | regulation function, transcription elongation | 32052 (2000) |
| | factor RNA | | | function | |
| | polymerase | | | | |
| | II-like 3, | | | | |
| | testis | | | | |
| | specific) | | | | |
| FLJ36526 | NSFL1 (p97) | NP_057227.2 | | control of morphology and function of Golgi | Uchiyama K, J Cell Biol., 161, 1067 |
| | cofactor (p47) | NP_061327.2 | | body, cell division regulation, regulation of | (2003) |
| | (NSFL1C), | NP_872289.1 | | membrane fusion and separation | |
| | transcription | | | | |
| | variant 1 | | | | |
| FLJ37909 | Acidic | NP_112182.1 | NP_006296.1 | protein phosphatase inhibition activity, cell | |
| FLJ35353 | leucine-rich | | NP_006392.1 | cycle control function, control function of | |
| | protein-like | | NP_036535.1 | differentiation and morphology of immune | |
| | protein; acidic | | NP_036536.2 | cell or nerve cell, axon control function, | |
| | leucine-rich | | NP_003002.1 | apoptosis control function, nerve cell | |
| | nuclear | | | protection function, | |
| | phosphoprotein | | | transcription/translation control function, | |
| | 32 family | | | RNA transport/localization function, control | |
| | member E; | | | function of synaptic transmission efficiency, | |
| | LANP-like | | | regulation of memory and learning function, | |
| | protein; | | | regulating the function of protein | |
| | LANP-L; ANP32E | | | phosphatase (PPP2, PP2A) complex, | |
| | | | | regulating the function of MAPs | |
| | | | | (microtubule-associated | |
| | | | | proteins), regulating the function of INHAT | |
| | | | | complex, regulating the function of DNA | |
| | | | | repair and DNA nicking | |
| FLJ38531 | hypothetical | XP_373431.3 | | | |
| | protein | | | | |
| | BC004360 | | | | |
| FLJ38897 | Gasdermin | NP_079012.2 | | | |
| | domain-containing | | | | |
| | 1; GSDMDC1; | | | | |
| | DFN5-like | | | | |
| | protein | | | | |
| | FLJ12150 | | | | |
| FLJ39553 | Hypothetical | NP_775820.1 | | | |
| | protein | | | | |
| | FLJ39553 | | | | |

TABLE 2-4

| | | | | |
|---|---|---|---|---|
| FLJ40298 | Hypothetical | NP_775757.1 | | |
| | protein | | | |
| | FLJ40298 | | | |
| FLJ40760 | DNAJA4: DnaJ | NP_061072.2 | | |
| | (Hsp40) | | | |
| | homolog, | | | |
| | subfamily A, | | | |
| | member 4 | | | |
| FLJ41550 | SAM domain and | | | |
| | HD domain 1 | | | |
| | (SAMHD1) | | | |
| FLJ41991 | Gasdermin | NP_079012.2 | | |
| | domain-containing | | | |
| | 1; GSDMDC1; | | | |
| | DFN5-like | | | |
| | protein | | | |
| | FLJ12150 | | | |
| FLJ42665 | EN02; Enolase 2 | NP_001966.1 | energy production function in glycolytic | Chai G et al. J. Mol., Biol., 341, |
| | (in gamma | | pathway Enolase (phosphopyruvate hydratase) | 1015 (2004) |
| | neuron); | | activity, magnesium ion-binding activity | |
| | 2-phospho-D-glycerate | | | |
| | hydrolyase; | | | |
| | Enolase in | | | |
| | neuron; | | | |
| | phosphopyruvate | | | |
| | hydratase | | | |

As used herein, “a homologous protein” means a protein belonging to the same protein family as the above-described protein. Example of homologous proteins are given in Tables 2-1 to 2-4.

As used herein, “a variant” of a protein means an artificial mutant or natural mutant of the protein, and includes splicing variants.

A variant of a protein provided by the present invention can also be, for example, a protein that consists of an amino acid sequence resulting from the substitution, deletion, addition or insertion of one or more amino acids in the amino acid sequence shown by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:22, SEQ ID NO:23 or SEQ ID NO:24, and that interacts with a bioactive substance.

The number of amino acids substituted, deleted, added or inserted can be any one that allows the retention of the function, for example, about 1 to 50, preferably about 1 to 30, more preferably about 1 to 20, further more preferably about 1 to 10, most preferably 1 to 5 or 1 or 2. The site for substitution, deletion, addition or insertion of an amino acid can be any site that allows the retention of the function, for example, a site other than functionally important domains.

Furthermore, a variant of a protein provided by the present invention can be a protein which consists of, for example, an amino acid sequence having a homology of about 50% or more, preferably about 70% or more, more preferably about 80% or more, further more preferably about 90% or more, most preferably about 95% or more (but excluding 100% homology), to the amino acid sequence shown by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:22, SEQ ID NO:23 or SEQ ID NO:24, and which interacts with a bioactive substance. Here, the numerical values of the above-described homology are calculated by, for example, executing the commands for the maximum matching method using the DNASIS sequence analytical software (Hitachi Software Engineering). The parameters for the calculation should be used in default settings (initial settings).

When a target protein of the present invention is used, the protein may be a labeled supply or a non-labeled supply, or a mixture of a labeled supply and a non-labeled supply mixed in a specified ratio. Examples of the labeling substance include fluorescent substances such as FITC and FAM, luminescent substances such as luminol, luciferin and lucigenin, radioisotopes such as ³H, ¹⁴C, ³²P, ³⁵S, and ¹²³I, affinity substances such as biotin and streptavidin, and the like.

The target genes of the present invention may be any ones that encode the target proteins of the present invention. For example, the target genes of the present invention can be those corresponding to proteins comprising the above-described amino acid sequences. For example, proteins comprising the above-described amino acid sequences can be those corresponding to cDNA clones having nucleotide sequences corresponding to the FLJ nucleotide sequence accession numbers shown in Tables 1-1 to 1-6. In the H-Invitational Database (H-InvDB), for example, cDNA clones that share a gene region on the human genome are classified as a cluster; the cDNA clones corresponding to the proteins of the present invention are given respective H-Inv cDNA IDs shown in Tables 1-1 to 1-6, and the gene loci thereof are given respective H-Inv locus IDs. Hence, the target genes of the present invention can be cDNAs of the FLJ nucleotide sequence accession numbers shown in Tables 1-1 to 1-6, a cDNA cluster of H-Inv cDNA IDs in H-InvDB, or genes given H-Inv locus IDs or genes homologous thereto. As used herein, the target genes of the present invention are not limited to human genes, but include orthologues of different animal species.

As used herein, “a homologous gene” means a gene belonging to the same family of genes as the above-described genes. Examples of homologous genes are the genes that encode the homologous proteins shown in Tables 2-1 to 2-4.

As used herein, “a variant” of a gene means an artificial variant or natural variant of the gene, and includes splicing variants transcribed from the gene.

For example, a variant of a gene provided by the present invention can be a cDNA that consists of a nucleotide sequence that hybridizes to a sequence complementary to the nucleotide sequence corresponding to one of the FLJ nucleotide sequence accession numbers shown in Tables 1-1 to 1-6 under stringent conditions, and that corresponds to a protein that interacts with a bioactive substance. Here, “hybridize under stringent conditions” means that a positive hybridization signal remains observable even under conditions of, for example, heating in a solution of 6×SSC, 0.5% SDS and 50% formamide at 42° C., followed by washing in a solution of 0.1×SSC and 0.5% SDS at 68° C.

The target proteins and target genes of the present invention can be used for the development of drugs for diseases or conditions associated with bioactive substance X, or diseases or conditions associated with target gene Y (or target protein Y), or for the development of investigational reagents for the diseases or conditions, and the like. Diseases or conditions associated with bioactive substance X and diseases or conditions associated with target gene Y are described in detail below.

(Diseases or Conditions Associated with Bioactive Substance X) “A disease or condition associated with bioactive substance X” means a disease for which bioactive substance X is used or a disease corresponding to an adverse effect of bioactive substance X, or a condition for which use of bioactive substance X is desired (e.g., a deficiency of bioactive substance X) or an unwanted condition caused by bioactive substance X (e.g., an unwanted condition caused by excess intake of bioactive substance X). A disease or condition associated with bioactive substance X can be ameliorated or exacerbated by bioactive substance X.

“An action associated with bioactive substance X” means an action of the same kind as, or opposite kind to, a kind of action actually exhibited by bioactive substance X (including pharmacological actions and adverse effects). Hence, an action associated with bioactive substance X is an action capable of ameliorating or exacerbating “a disease or condition associated with bioactive substance X”. For example, “an action associated with bioactive substance X” is anti-hyperlipemic action, blood cholesterol decreasing action and the like when bioactive substance X is simvastatin, and is anti-allergic action, allergic action and the like when bioactive substance X is terfenadine.

“A disease or condition associated with bioactive substance X” and “an action associated with bioactive substance X” vary depending on the kind of bioactive substance X. Described below are “diseases or conditions associated with bioactive substance X” with reference to substances that represent bioactive substance X. Because “an action associated with bioactive substance X” is any action capable of ameliorating or exacerbating “a disease or condition associated with bioactive substance X”, the following description of “diseases or conditions associated with bioactive substance X” will surely lead to the clarification of “actions associated with bioactive substance X”.

### Picotamide

A disease associated with picotamide means a disease for which picotamide is used or a disease corresponding to an adverse effect of picotamide. Picotamide is known as an antiplatelet drug. Examples of the disease for which picotamide is used include peripheral vascular occlusion, primary thrombocytosis, enalapril-induced cough, heparin-related thrombocytopenia/thrombosis, albuminuria in diabetic patients, and the like. In contrast, examples of the adverse effect of picotamide include prolonged hemorrhagic time, headache, abdominal discomfort, epigastric pain and other gastrointestinal disorders, sensation of itching and other skin reactions, and the like. An action associated with picotamide can be closely relevant to a target protein (target gene) therefor, for example, a protein comprising the amino acid sequence shown by SEQ ID NO:1 or a protein homologous thereto or a variant thereof.

### Methoxsalen

A disease associated with methoxsalen means a disease for which methoxsalen is used or a disease corresponding to an adverse effect of methoxsalen. Methoxsalen is known as a dermatological drug. Examples of the disease for which methoxsalen is used include leukoderma vulgaris and the like. In contrast, examples of the adverse effect of methoxsalen include gastrointestinal disorders, insomnia, depression, swelling due to overdoses of ultraviolet rays, blisters and the like. An action associated with methoxsalen can be closely relevant to a target protein (target gene) therefor, for example, a protein comprising the amino acid sequence shown by SEQ ID NO:2 or a protein homologous thereto or a variant thereof.

### Terfenadine

A disease associated with terfenadine means a disease for which terfenadine is used or a disease corresponding to an adverse effect of terfenadine. Terfenadine is known as an anti-allergic drug and the like. Examples of the disease for which terfenadine is used include allergic rhinitis, bronchial asthma, eczema, urticaria, dermatitis, skin pruritus and the like. In contrast, examples of the adverse effect of terfenadine include thrombocytopenia, ventricular fibrillation, death, cardiac arrest, hypotension, palpitation, syncope, QT interval prolongation, tachycardia, torsades de pointes (ventricular tachycardia) and the like. Histamine H1 receptor is known as a target for terfenadine. An action associated with terfenadine can be closely relevant to a target protein (target gene) therefor, for example, a protein comprising the amino acid sequence shown by SEQ ID NO:3, SEQ ID NO:14 or SEQ ID NO:19 or a protein homologous thereto or a variant thereof.

### Cyclosporin A

A disease associated with cyclosporin A means a disease for which cyclosporin A is used or a disease corresponding to an adverse effect of cyclosporin A. Cyclosporin A is known as an anti-malignant tumor agent and immunosuppressant. Examples of the disease for which cyclosporin A is used include suppression of graft rejection in kidney, liver, and heart transplantation, suppression of graft rejection and graft-versus-host disease in bone marrow transplantation, Behcet's disease with ocular symptoms, vulgaris psoriasis, pustular psoriasis, erythroderma psoriaticum, psoriasis arthropathica, aplastic anemia, pure red cell aplasia, nephrotic syndrome and the like. Other pharmacological actions of cyclosporin A include nerve cell protection, synaptic transmission efficiency depression or potentiation such as long-term or short-term depression (LTD or STD) and long-term or short-term potentiation (LTP or STP), increase in intracellular Ca release ion channel sensitivity, neurotransmitter release promotion, degranulation suppression, and suppression of release of chemical mediators or cytotoxic factors, and the like. In contrast, examples of the adverse effect of cyclosporin A include shock (injection), renal disorder, hepatic disorder, central nervous system disorder, nervous Behcet's disease symptoms, infectious diseases, acute pancreatitis, thrombotic microvascular disorders, hemolytic anemia, thrombocytopenia, rhabdomyolysis, lymphoma, lymphproliferative disease, malignant tumors (particularly in the skin), increased blood pressure, anemia, leukocytopenia, thrombocytopenia, peptic ulcers, nausea, vomiting, abdominal pain, gastric discomfort, hypertrichosis, tremor, headache, numbness, vertigo, glycosuria, hyperglycemia, hyperkalemia, hyperuricemia and the like. An action associated with cyclosporin A can be closely relevant to a target protein (target gene) therefor, for example, a protein comprising the amino acid sequence shown by SEQ ID NO:3, SEQ ID NO:8 or SEQ ID NO:12 or a protein homologous thereto or a variant thereof.

The interaction between cyclosporin A and a protein comprising the amino acid sequence shown by SEQ ID NO:3 (FLJ11211: cyclin D-binding myb-like transcription factor 1: DMP1) or SEQ ID NO:12 (FLJ37909: acidic leucine-rich nuclear phosphoprotein 32 family member E: ANP32E)) or a protein homologous thereto or a variant thereof can be very important to drug discovery. The interaction between cyclosporin A and a protein comprising the amino acid sequence shown by SEQ ID NO:8 (FLJ23466: FK506 binding protein like: FKBPL) or a protein homologous thereto or a variant thereof is also likely to be important to drug discovery. Hence, these interactions can be particularly interesting subjects in screening methods and the like provided by the present invention. The reason is hereinafter described in detail, but those skilled in the art can modify the present invention (e.g., screening methods of the present invention) based on the findings described below as appropriate.

(1) On SEQ ID NO:3 (FLJ11211): Cyclin D-Binding myb-Like Transcription Factor 1 (DMP1)

The protein shown by SEQ ID NO:3 (FLJ11211), which constitutes an interaction pair with cyclosporin A in the present invention, is one of the splice variants of the gene that encodes cyclin D-binding myb-like transcription factor 1 (DMP1). DMP1 is a protein cloned from mouse T lymphoma cDNA by yeast 2-hybrid screening with cyclin D2 as the bait, serving as a transcription factor having three Myb-like repeat sequences. SEQ ID NO:3 (FLJ11211) corresponds to a partial peptide of human DMP1 on the C-terminal side thereof (303 amino acid residues). Full-length human DMP1α consists of 760 amino acid residues, with a trans-activation domain (TAD) existing on each of the N- and C-termini thereof, and has a cyclin D-binding domain and a Myb-homologous region (MHR) between these TADs. DMP1α consists of 18 exons., with already known two kinds of splice variants resulting from alternative splicing at the 9th intron: DMP1β (272 amino acid residues) and DMP1γ (285 amino acid residues). Because the DMP1β and DMP1γ variants have the stop codon emerging due to frame shift as a result of the alternative splicing, they retain TAD and CBS on the N-terminal side of DMP1α, but do not comprise MHR and TAD on the C-terminal side. On the other hand, SEQ ID NO:3 (FLJ11211) corresponds to the trans-activation domain (TAD) moiety of the full-length variant DMP1α on the C-terminal side thereof, and does not comprise the sequence moieties of the splice variants DMP1β and DMP1γ at all.

The human DMP1 gene is located at the locus 7q21, at which chromosome aberrations (particularly deletion) have been found in acute myelocytic leukemia and the like. DMP1 is highly expressed in the testis, spleen, thymus, peripheral blood leukocytes and the like; the above-described splice variants exhibit expression patterns characteristic of various differentiation stages of hematopoietic cells, respectively. Hence, in cells that are positive for CD34 (a marker for hematopoietic precursor cells, undifferentiated leukemia cells and the like) in the resting phase, and fresh peripheral blood leukocytes, DMP1β (and γ) is expressed at higher levels than DMP1α. In contrast, in differentiated cells of activated T cells, bone marrow cells, macrophages, and granulocytes, DMP1β (and γ) is expressed at low levels with DMP1α being expressed at higher levels. Furthermore, some cases have recently been reported wherein the trans-activation domain or DNA binding domain of transcription factor is removed due to alternative splicing, and the transcription factor functions as a dominant-negative repressor. In the case of this DMP1 as well, it has been suggested that in undifferentiated hematopoietic cells in the resting phase, the DMP1β (and γ) splice variant, which functions as a repressor, may be predominant and suppress the expression of genes necessary for the differentiation and activation of bone marrow cells (CD13/APN and the like), and that in contrast, in activated T cells, bone marrow cells, macrophages, granulocytes and the like, the DMP1α splice variant may be predominant and activate the expression of the genes. Because SEQ ID NO:3 (FLJ11211), to which cyclosporin A binds, has no sequence overlap with the repressor variant DMP1β (and γ), cyclosporin A is incapable of binding to the repressor variant DMP1β (and γ), though it is capable of binding to DMP1α, an activator variant having a transcription activation potential. Hence, the present invention explicitly demonstrates the possibility that cyclosporin A binds to an activator variant having the transcription activation potential of DMP1 to suppress the differentiation and activation of hematopoietic cells.

DMP1, as suggested from the designation, has a cyclin D-binding domain adjacent to a Myb homologous domain that binds to the DNA consensus sequence CCCG(G/T)ATGT, and loses the DNA-binding activity thereof without the involvement of kinases such as CDK by the specific binding of cyclin D per se. Hence, it is known, regarding DMP1, that (1) the splice pattern thereof shifts from a repressor variant to an activator variant with the progress of differentiation, that (2) overexpression of the activator variant suppresses the transition of cell division from G1 phase to S phase, and that (3) the transcription activation potential thereof is suppressed by cell cycle G1-dependent cyclin D. Furthermore, DMP1 binds to the ARF promoter region. Expressed from the INK4a/ARF gene locus by alternative splicing are two cancer suppressor proteins, i.e., INK4a and ARF, which have different first exons. Although these proteins are structurally completely different from each other because of the differences in ORF, both are cancer suppressor gene products closely related to cell proliferation checkpoints. INK4a, a CDK4 kinase inhibitor, suppresses cyclin D-dependent kinase activity to keep the cancer supressor gene product Rb in the active form that acts to terminate cell proliferation (G1 arrest). The other cancer supressor protein, ARF, binds to the p53 inactivation protein HDM2 to stabilize p53. P53 is an important factor for the G1 checkpoint, where the cell fate toward proliferation termination or apoptosis is determined. Hence, the present invention explicitly demonstrates the possibility that cyclosporin A can influence DMP1, an important molecule for the checkpoint mechanism in the cell cycle, to suppress the differentiation and activation of hematopoietic cells. In other words, DMP1 is an essential factor for the immune function wherein precursor cells having ceased cell division in the resting phase begin differentiation, become mature immune cells, and reach the activation stage; the present invention shows that DMP1 and related molecules are essential molecules in understanding all aspects of the pharmacological action of cyclosporin A, and developing effective and safe immunosuppressants, and proposes applications in their screening.

Because DMP1 is a protein of the c-Myb family, the present invention also suggests that cyclosporin A may indirectly influence the functionality of the Myb family by interacting directly with other proteins of the Myb family, or by acting on DMP1. Human c-Myb, like human DMP1, is a transcription factor essential to T cell differentiation and activation. Hence, c-Myb is deemed a transcription factor involved directly in the pathway through which double negative (CD4−, CD8−) precursor cells become double positive (CD4+, CD8+) cells, and then become single positive mature T cells. In particular, regarding CD8-positive cell which is cytotoxic T cells, it has been suggested that an Myb-binding sequence may be present in the silencer region of the CD4 gene, and that the proteins of the c-Myb family, including DMP1, may cooperatively mediate the positive selection of T cells. Hence, the present invention also suggests that cyclosporin A may act on the proteins of the c-Myb family, including DMP1, to influence the immune activity of T cells.

Accordingly, the present invention elucidates action mechanisms of cyclosporin A, particularly mechanisms for its immunosuppressive activity and immune cell differentiation and activation, from the viewpoint of transcriptional regulation, and provides a technique important in designing novel immunosuppressants of high safety and high efficacy, by proposing various screening methods based on novel interactions between cyclosporin A and SEQ ID NO:3 (FLJ11211) and related proteins. Hence, proposed by the present invention is that cyclosporin A is not only capable of binding to the known target protein cyclophilin to inhibit the protein phosphatase activity of calcineurin, and of interfering with the nuclear translocation of the transcription factor NF-AT to suppress T cell activation, but also capable of acting on the Myb family of transcription factors such as DMP1 to influence the activation stage, and immune cell maturation, particularly the mobilization of cytotoxic T cells. Accordingly, the present invention provides a technique particularly useful for the development of therapeutic drugs for various immunity-related diseases such as immunosuppressants, anti-allergic agents, autoimmune disease remedies, and anti-inflammatory agents, and the development of anticancer agents.

### (2) On SEQ ID NO:12 (FLJ37909): Acidic Leucine-Rich Nuclear Phosphoprotein 32 Family Member E (ANP32E)

The protein shown by SEQ ID NO:12 (FLJ37909), which constitutes an interaction pair with cyclosporin A in the present invention, is one of the splice variants of the gene that encodes acidic leucine-rich nuclear phosphoprotein 32 family member E (ANP32E). Acidic leucine-rich nuclear phosphoprotein 32 family member E (ANP32E) is a gene product discovered in a differential display experiment performed to identify genes that control nerve cell differentiation in the mouse cerebellum, which is used as a model for brain morphogenesis and synapse formation. The expression of this gene maximizes in cerebellar cells in the second half of the morphogenetic stage; the expression is suppressed after the synapse formation stage, and this gene was initially designated as Cpd1 (cerebellar postnatal development protein 1). This protein possesses an inhibitor activity to specifically inhibit protein phosphatase 2 (PPP2), exhibits many other functions, and is also called ANP32E as a member of the acidic leucine-rich nuclear phosphoprotein 32 family. The human full-length variant of ANP32E cloned in the human brain cDNA project consists of 268 amino acid residues and, as suggested from the designation, has three leucine-rich repeats (LRRs) in the N-terminal domain thereof and an acidic domain with a very large number of Glu residues and Asp residues on the C-terminal side thereof. As such, ANP32E has also a nuclear-localized NLS (nuclear import signal) in the C-terminal region thereof and, like other members of the ANP32 family, seems to have an NES (nuclear export signal) as well, and is postulated to be capable of shuttling between the inside of the nucleus and cytoplasm as with ANP32A and ANP32B and the like. The protein of the present invention shown by SEQ ID NO:12 (FLJ37909) is a splice variant corresponding to 218 residues on the C-terminal side of ANP32E.

As such, ANP32E is one of the members of ANP32 family, which consists of ANP32A to E, with related proteins belonging to the SET family, which consists of SET A and SET B isoforms (the translocation breakpoint-encoded proteins in acute undifferentiated leukemia). These related proteins of the ANP32 family and SET family are proteins having very similar characteristics, all of which tend to form homo-complexes with each other or hetero-complexes with other proteins. These related proteins all exhibit an inhibitory activity on PPP2 (protein-phosphatase 2), a member of the serine/threonine-phosphatase family. As such, the PPP2 molecule functions in the form of a dimer wherein the catalytic subunit PPP2C is bound to the C-terminal side of the regulatory subunit PPP2R1 (former PR65), or in the form of a trimer wherein one molecule out of the other regulatory subunits PPP2R2 to PPP2R5, which exhibit a tissue-specific expression pattern, is bound to the N-terminal side of the regulatory subunit PPP2R1; these regulatory subunits determine the activity, substrate specificity, and intracellular localization of this enzyme. In particular, the B subunits (PPP2R2A, PPP2R2B, PPP2R2c and the like) are control subunits expressed mainly in the brain. Furthermore, two kinds of inhibitors I₁^{PP2A} and I₂^{PP2A} have been known to specifically inhibit this PPP2, which correspond to the aforementioned ANP32 family (I₁^{PP2A}) and SET family (I₂^{PP2A}) Cyclophilin is known as a target protein for cyclosporin A; this immunophilin inhibits the protein phosphatase activity of calcineurin and interferes with the nuclear transfer of the transcription factor NF-AT to suppress T cell activation. As such, calcineurin is a protein phosphatase of the PPP3 family, whereas the ANP32 family and SET family do not inhibit PPP3.

The protein phosphatase PPP2, a target enzyme for the inhibitor activity of the ANP32 family and SET family, has a large number of action points, some of which are involved in the cell cycle, particularly at the two checkpoints in the G1/S phase and G2/M phase. PPP2 suppresses the progression of the cell cycle from the G2 phase to M phase; this is attributable, in part, to the inactivation of Cdc25 phosphatase, and PPP2 dephosphorylates this Cdc25 to negatively control the phosphatase activity of Cdc25. Hence, the ANP32 family and SET family, which are PPP2 inhibitors, and cyclosporin A, which binds to these families, can influence the progression of the cell cycle. Cyclin G is known to bind to the PPP2C subunit to form a complex retaining the original activity; upon formation of this complex, the progression of the cell cycle is terminated at the G1/S phase. This is viewed as one of the cell cycle termination mechanisms in response to cell differentiation signals. The present invention discloses that the ANP32 family and SET family, which are PPP2 inhibitors, may be involved in the progression of the cell cycle, and that cyclosporin A derivatives that bind thereto can also influence the progression of the cell cycle. Also, cyclin G expression is induced by the cancer suppressive transcription factor p53, concurrently with the expression of HDM2; cyclin G is capable of interacting with HDM2 and further binding to PP2A via PPP2R4 (PP2A regulatory subunit B′). HDM2 is an important instabilization factor for p53 that possesses E3 ubiquitin ligase activity to ubiquitinate p53 and lead it to decomposition through the proteosome pathway, whereas phosphorylated HDM2 is of the inactive type. Therefore, cyclin G is considered to dephosphorylate HDM2 by approximating it to PPP2, and hence to promote p53 degradation. Hence, the present invention discloses that cyclosporin A and derivatives thereof can bind to the ANP32 family or SET family to influence the progression of the cell cycle.

As described above, in particular, cyclosporin A also binds to SEQ ID NO:3 or DMP1α, a full-length variant thereof. The SEQ ID NO:3 full-length variant DMP1α is a transcription factor (transcription activator); overexpression thereof prevents the cell cycle from progressing from the G1 phase to the S phase; one of the regions to which this DMP1α binds is the ARF promoter region, and two cancer suppressor proteins closely related to checkpoints of cell proliferation, i.e., INK4a and ARF, are expressed from the INK4a/ARF gene locus. INK4a is a CDK4 kinase inhibitor, and acts to keep the cancer suppressor gene product Rb in the active form having cell proliferation termination activity by suppressing cyclin D-dependent kinase activity. On the other hand, ARF stabilizes p53 by binding to the p53-inactivating protein HDM2. Hence, the present invention discloses that a cyclosporin A derivative may exhibit binary action on the stability of p53, namely, binding to DMP1α which is associated with SEQ ID NO:3, to act on the expression of INK4a and ARF, thereby to influence the G1 phase of the cell cycle via Rb phosphorylation on one hand and to suppress the expression of the HDM2 inhibitor ARF to shift HDM2 from the ARF-closed state to the free state on the other hand; and by binding to a PPP2 inhibitor, associated with SEQ ID NO:12 to influence the dephosphorylation of HDM2. The effects on p53 stability can be related to an increased risk of onset of squamous cell carcinoma observed in psoriasis patients receiving cyclosporin A. Hence, the present invention shows that a cyclosporin A derivative may interact with a plurality of target protein molecules, and specifically discloses the plurality of target protein molecules and the functions thereof. Hence, the present invention enables compounds capable of acting on the checkpoint mechanism of the cell cycle or, conversely, poorly active on the checkpoint mechanism of the cell cycle, to be discovered through screening, or to be designed in silico, and makes it easier to discover safer and more effective compounds, by utilizing the various screening methods of the present invention, based on an interacting pair of SEQ ID NO:3 (FLJ11211) and cyclosporin A, an interacting pair of SEQ ID NO:8 (FLJ23466) and cyclosporin A, an interacting pair of SEQ ID NO:12 (FLJ37909) and cyclosporin A, and if necessary an interacting pair of known target protein molecule cyclophilin or calcineurin and cyclosporin A, as described above.

In the present invention, “an action associated with bioactive substance X” is not limited to actions in clinical applications, and includes the biological activities, pharmacological actions, adverse actions, and other various actions in non-prescribed clinical applications, possessed by the bioactive substance, as well as the activities and actions possessed by the proteins associated with the sequence identification numbers disclosed herein to interact with bioactive substance X. For example, the actions of cyclosporin A are not limited to immunosuppressive actions already in clinical applications. Regarding its pharmacological actions, cyclosporin A is known to protect nerve cells and act on synaptic transmission efficiency associated with memory and learning, in the central nervous system; some proteins of the aforementioned ANP32 family, which is associated with SEQ ID NO:12, exhibit actions associated with the differentiation stages of nerve cells. For example, ANP32E, whose expression maximizes in the second half of the cerebellar morphogenetic stage, is considered to be relevant to nerve precursor cell proliferation and migration, and ANP32A is considered to be relevant to nerve cell differentiation processes such as neurite elongation in the synapse formation stage. ANP32A is also known as mapmodulin, a synonym derived from the fact that phosphorylated ANP32A binds to the tubulin-binding domains of MAPs (microtubule-associated proteins) and Tau. As such, ANP32A is considered to have its localization shifted from inside of the nucleus to cytoplasm with the genesis of neurites, and to interact with MAP1B to mediate neurite elongation; the activities and functions of the ANP32 family can be correlated with the central actions of cyclosporin A. The ANP32 family and SET family possess PPP2 inhibitor activity; the PPP2 protein phosphatase actions thereof range widely from cell differentiation to MAP kinase activity control, PKC signal transduction, Wnt signal transduction, apoptosis and the like, all of which can also be correlated with the pharmacological actions of cyclosporin A. Furthermore, it is known that PPP2 is bound to the microtubules and intermediate filaments, with particularly high PPP2 activity found in the cells in the S phase. Because tubulin polymerization is inhibited when the microtubule constituent proteins tubulin and MAP2 are phosphorylated, PPP2 is considered to promote tubulin polymerization; the ANP32 family, which is a family of PPP2 inhibitors, is also capable of acting on microtubule genesis regulation to mediate the motility and axon formation of nerve cells. The same applies to Tau, a kind of MAP in nerve cells; when dephosphorylated by PPP2, Tau promotes tubulin polymerization and stabilizes the microtubules. In Alzheimer's disease, it has been suggested that Tau may aggregate due to excess phosphorylation, and that microtubule stability is lost due to tubulin depolymerization, resulting in the axonal degeneration observed in the disease. PPP2 is known to be involved in the phosphorylated state of Tau; the present invention discloses that cyclosporin A and derivatives thereof are capable of binding to PPP2 inhibitors to act on neurodegenerative events such as those in Alzheimer's disease.

The ANP32 family and SET family are also constituent components of complexes possessing histone acetyltransferase inhibitory activity (INHAT) in the nucleus. Histone acetylation by p300/CBP and the like is important to transcriptional regulation and chromatin remodeling; the INHAT complex binds to histone to inhibit this acetylation. It is known that when a member of the ANP32 family and SET family binds to the INHAT complex, the specificity of histone to which the INHAT complex binds is altered; the present invention discloses that cyclosporin A and derivatives thereof can act on the transcriptional regulatory activities of the ANP32 family and SET family.

Furthermore, because the ANP32 family and SET family are capable of interacting with HuR, their association with differentiation has been suggested from the viewpoint of mRNA stabilization. Protein expression levels are known to undergo control not only at the level of transcription to mRNA, but also by stabilization by RNA-binding proteins (mRNA turnovers). In particular, HuR, which binds to AU-rich elements (AREs), which control mRNA instability, is an RNA stabilization factor expressed as a large number of alternative splicing variants in nerve cells, and showing a variant pattern changeable with nerve cell differentiation. As such, HuR is highly expressed in proliferating cells with differentiation of the central nervous system, and is known to be conjugated by members of the ANP32 family and SET family. HuR is capable of shuttling between the nucleus and cytoplasm; this localization may be mediated by the NLS (nuclear import signal) or NES (nuclear export signal) of the ANP32 family and SET family to regulate mRNA trafficking and stability. Hence, the present invention discloses that cyclosporin A and derivatives thereof can act on the mRNA stabilizing function or mRNA localizing function of the ANP32 family and SET family.

PPP2 and inhibitors thereof have also been correlated with apoptosis; in particular, they have been suggested to be involved in the cell death of nerve precursor cells and immature nerve cells in brain differentiation. For example, it is the SET complex that causes single-strand DNA nicking in the activation of the caspase-non-dependent apoptosis pathway by Granzyme A, protease of cytotoxic T cells. The SET complex is normally involved in DNA repair in the form of a conjugate with nuclease, with SET suppressing the DNA nicking activity of repair enzyme, but activation of Granzyme A cleaves SET and causes DNA nicking. Hence, the proteins of the ANP32 family and SET family are likely to be involved in apoptosis in immune cells and nerve cells; this can be correlated with the nerve cell protecting action of cyclosporin A and derivatives thereof.

PPP2 is also known to play a key role in the control of synaptic transmission efficiency. Specifically, it is considered that LTP is induced upon PPP3 activation and PPP1 and PPP2 inhibition, and that PPP2 inhibition is essential to the maintenance of LTP. PPP2 inhibition is dependent on NMDA receptors and is suppressed by inhibition of a calmodulin-dependent pathway. The regulatory subunit B of PPP2 has a CaMK II phosphorylation site, where the subunit B undergoes phosphorylation to lose its PPP2 activity with LTP induction in the hippocampus. In LTD (long-term depression) as well, involvement of PPP1 and PPP2 has been suggested. It has been suggested that the threshold value for pulse frequency that determines whether LTP or LTD is induced may be shifted by inhibition of PPP1 and PPP2, resulting in the suppression of NMDA-dependent induction of LTD. Hence, the present invention discloses that cyclosporin A and derivatives thereof are capable of binding to the ANP32 family and SET family, which are PPP2 inhibitors, to act on synaptic transmission efficiency. Hence, the present invention enables the creation of pharmaceutical compounds associated with memory or dementia by disclosing screening methods based on the central actions of cyclosporin A.

### (3) On SEQ ID NO:8 (FLJ23466): FKBPL: FK506 Binding Protein Like

The protein shown by SEQ ID NO:8 (FLJ23466), which constitutes an interaction pair with cyclosporin A in the present invention, is one of the splice variants of the gene that encodes FKBPL (FK506 binding protein like). As such, FKBPL is a protein of 349 amino acid residues that is homologous to the FK506-binding protein (FKBP) family, a group of immunophilins, and has tetratricopeptide repeats (TPRs) on the C-terminus thereof. SEQ ID NO:8 (FLJ23466), to which cyclosporin A binds, is a TPR-free partial-length variant on the N-terminal side (203 amino acid residues).

It is publicly well known that cyclosporin A binds to another immunophilin, i.e., cyclophilin, to inhibit the protein phosphatase activity of calcineurin and interfere with the nuclear translocation of the transcription factor NF-AT to suppress T cell activation. The aforementioned c-Myb is known to bind to cyclophilin-40 (cyclophilin D), a known target protein for cyclosporin A; c-Myb loses its DNA bindability when cyclophilin-40 binds thereto. This loss of the DNA bindability of c-Myb by cyclophilin-40 is normalized by cyclosporin A. Because immunophilins such as cyclophilin and FKBP have a function as peptidyl-prolyl isomerase, they are capable of interacting with a wide variety of proteins; by altering the conformations of the protein, immunophilins control the activities of some proteins, including nuclear receptors and transcription factors. An example is c-Myb, which is a homologous protein to the aforementioned DMP1 of the present invention; in particular, because cyclophilin-40, which binds to c-Myb, has three tetratricopeptide repeats (TPRs), in addition to the peptidyl-prolyl isomerase domain, it is expected to interact with a DNA-binding homeodomain protein necessary to completion of cell division. FKBPL is similar to cyclophilin-40, which binds to Myb as described above, in that it has TPR; FKBPL, like cyclophilin-40, is expected to interact with a DNA-binding homeodomain protein necessary to completion of cell division, and is considered to be involved in stress responses. Hence, the present invention provides an important technique in designing a compound that regulates stress response actions exemplified by cell cycle control activity and DNA repair activity, particularly radiation resistance control and the like, by proposing various screening methods based on novel interactions between SEQ ID NO:8 (FLJ23466) and cyclosporin A.

As described above, the present invention proposes that the wide variety of actions of cyclosporin A represent a series of complicated events based on its binding to a plurality of target molecules. The present invention gives examples of such the plurality of target proteins, and examples of the functions and actions that are possibly exhibited by the target proteins. Hence, making full use of the facts disclosed by the present invention enables the separation and enhancement of the wide variety of actions of the cyclosporin A molecule. Hence, by utilizing the various screening methods of the present invention based on the interacting pair of SEQ ID NO:3 (FLJ11211) and cyclosporin A, interacting pair of SEQ ID NO:8 (FLJ23466) and cyclosporin A, and interacting pair of SEQ ID NO:12 (FLJ37909) and cyclosporin A disclosed by the present invention, and, if necessary, based on an interacting pair of the known target protein molecule cyclophilin or calcineurin and cyclosporin A, screening and in silico drug design that target a plurality of target protein molecules can be achieved on the basis of “one-compound versus multiple-protein” interactions.

### Pancuronium Bromide

A disease associated with pancuronium bromide means a disease for which pancuronium bromide is used or a disease corresponding to an adverse effect of pancuronium bromide. Pancuronium bromide is known as a muscle relaxant. Examples of the disease for which pancuronium bromide is used include muscle relaxation during surgery at various departments of medicine and the like. In contrast, examples of the adverse effect of pancuronium bromide include shock, anaphylactoid symptoms, prolonged apnea, increased pulse rates, increased blood pressure, hypersalivation, hiccups and the like. An action associated with pancuronium bromide can be closely relevant to a target protein (target gene) therefor, for example, a protein comprising the amino acid sequence shown by SEQ ID NO:4 or a protein homologous thereto or a variant thereof.

Furthermore, the interaction between pancuronium bromide and a protein comprising the amino acid sequence shown by SEQ ID NO:4 (FLJ12857: paralemmin) or a protein homologous thereto or a variant thereof can be very important to drug discovery. Hence, pancuronium bromide can be a particularly interesting subject in the screening methods provided by the present invention and the like. The reason is hereinafter described in detail, but those skilled in the art can modify methods of the present invention (e.g., the screening methods of the present invention) based on the findings described below as appropriate.

### (4) On SEQ ID NO:4 (FLJ12857): Paralemmin

SEQ ID NO:4, which constitutes an interaction pair with the muscle relaxant pancuronium bromide in the present invention, is one of the splice variants of the paralemmin gene, and is classified as a kind of scaffold protein that plays a very important role in the functions and differentiation of central and peripheral nerve cells, particularly in the dynamic functional control of the synapses. Traditionally, receptors and channels on the cell membrane, such as GPCR, have been tending to draw attention as drug discovery targets; the expression and functions of these receptors are controlled by what are called scaffold proteins in the broader sense. As such, however, scaffold proteins do not serve solely as “scaffolds” or “frameworks”. Some scaffold proteins control the functional expression of signal conversion factors such as receptors; paralemmin is considered to belong to this class of proteins. Despite the limited number of receptors, there are a very wide variety of forms and functions of nerve cells in individual tissues, from the central to peripheral nerve cells; numerous synapses occurring in these nerve cells accurately perform their respective roles of unique and complicated signal transduction processing according to the sites and functions thereof. This depends largely on scaffold proteins, which cause cell morphology to be changed per the DNA program, allow a finite number of receptors to be expressed on the synapse-forming lipid raft on the cell membrane in variable combinations on a case-by-case basis, and control a nearly infinite number of signal transduction patterns according to the situation on a real-time basis. Furthermore, paralemmin, a kind of scaffold protein, is expressed in various tissues, and exhibits characteristic splicing patterns in its ORF according to the expression time or expression site; this suggests the involvement of alternative splicing in cell morphology and dynamic functional control on the cell membrane. Hence, if the drug discovery target is to be a signal transduction mechanism in a particular site or tissue, rather than in a particular receptor or channel, it can be an effective approach to choose a scaffold protein as one of the drug discovery targets.

Paralemmin (PALM) is expressed in a wide variety of cells, with particularly high levels of expression observed in the cerebrum, cerebellum, secretory glands, heart and elsewhere. In mice, the expression of PALM in the brain decreases gradually in several tens of days after birth; it can be said that PALM is highly expressed in nerve cells in the embryonic stage. PALM is diverse in sequence length; shorter variants are expressed in the kidneys, and variants by the presence or absence of the 8th exon (corresponding to human residue Nos. 168-211) have been suggested. The longest is a variant having 387 amino acid residues, cloned from the fetal brain; SEQ ID NO:4 (FLJ12857) is a splice variant corresponding to the 270 residues on the C-terminal side of this longest variant. Regarding the presence or absence of the 8th exon, the majority of tissues, including the skeletal muscle, heart and adrenals, tend to have higher ratios of variants retaining the 8th exon, whereas the brain and kidneys tend to have higher ratios of variants not retaining the 8th exon; SEQ ID NO:4 (FLJ12857) corresponds to the type lacking the 8th exon.

The structural features and functionality of paralemmin are described below. Paralemmin has a plurality of conserved phosphorylation sites, and has been proven to undergo phosphorylation by some kinases such as PKA and PKC. The amino acid sequence of paralemmin from the N terminus to the 105th amino acid residue constitutes a coiled-coil region having two Leu zipper candidates and repeats of acidic residue-basic residue-hydrophobic residue-Gln residue, and is likely to mediate protein-protein association. It has also been pointed out that partial sequences of paralemmin are similar to those of the SNARE protein, which mediates ER-Golgi's body transport and membrane fusion to control the transport and release of synaptic vesicles. Furthermore, paralemmin is a highly hydrophilic protein and has a palmitoylation site constituted by two Cys residues adjoining to a cluster of a plurality of basic residues at the C-terminal side thereof. When S-palmitolylated as a result of palmitic acid transfer to the thiol group of these Cys residues in the post-translational modification stage, paralemmin associates itself to the cell membrane. Hence, paralemmin deprived of the palmitoylation site at the C terminus thereof is localized in cytoplasm, whereas paralemmin retaining the palmitoylation site migrates to the cell membrane and accumulates in active sites of the cell membrane, such as microspike clusters, filopodia, and projection apexes. The accumulation of paralemmin retaining the palmitoylation site in the cell membrane also influences cell morphology; fibroblasts transfected with paralemmin flatten and enlarge and, several days later, show morphological changes into astral shape or projection-elongated form.

By the way, palmitoylation is a very important post-translational modification not only in paralemmin, but also in nerve cells. Functional proteins that are present in the synapses, including receptors and transporters, accumulate at high levels on the lipid raft to form a functional complex, achieving effective signal transduction between the output and input sides of the pairing synapses. The lipid raft is a subdomain structure on the cell membrane, which is rich in cholesterol, sphingomyelin and the like, and is also called DIG (detergent-insoluble, glycolipid-enriched complex). When palmitoylated, a protein becomes likely to be incorporated by DIG because of the palmitoyl group moiety thereof. This is a phenomenon characteristic of palmitoylation, and is not observed in the case of myrystoylation such as of neurocalcin 6 (FLJ39196). S-palmitoylation (thioester linkage), unlike N-myrystoylation (acidamide linkage), is reversible; by the transfer and removal of the palmitoyl group, protein localization and functionality are controlled. Proteins having a palmitoylation site include GPCR (rhodopsin, dopamine receptor D1, neuronal acetylcholine receptor, adrenoceptor and the like), cell adhesion molecules (NCAM140 and the like), signal transduction factors (Gα1, H-Ras, Fyn and the like), and scaffold proteins (GAP43, PSD95, KChIP2b, AKAP18 and the like), most of which are localized in DIG. An example demonstrating the importance of palmitoylation in nerve cells is the control of synaptic vesicle transport and release. As described above, it has been suggested that partial sequences of paralemmin may be similar to the SNARE protein (SNAP receptor); including this SNARE protein, most of the proteins that control synaptic vesicle transport and release on the pre-synapse side are palmitoylated proteins, and the palmitoylation thereof is essential to the synaptic vesicle transport from Golgi's body to the synapses and the release of neurotransmitters in chemical synapses.

In some palmitoylated proteins, the Cys residues at their palmitoylation sites also serve as targets for NO (nitrogen oxide). NO is a signal transmitter produced in vascular endothelial cells in response to chemical stimuli (acetylcholine and the like) and physical stimuli (shearing stress); it was discovered with the fact as a momentum that it binds to, and activates, the hem iron of the guanylate cyclase (GC) in vascular smooth muscle, to cause smooth muscle relaxation. However, there are three types of the NO production enzyme NOS: eNOS (vascular endothelial type NOS), nNOS (nerve type NOS), and iNOS (induction type NOS). NO is produced at a wide variety of sites, including nerve cells. A big feature of NO as a signal transmitter resides in that it disperses rapidly and easily passes the cell membrane. Meanwhile, NO is a radical species, so that its life (range of reach) can be controlled in the body; as such, NO is often utilized in the body as a signal transmitter at local sites, and this is also true for the nervous systems, including the synapses. NO not only targets metal proteins, but also Cys residues in certain environments (mostly active groups) are also important targets; a very large number of NO target proteins are known. For example, when the Cys residues at the palmitoylation site are S-nitrosylated (S—N—O) by NO, palmitoyl group transfer is inhibited, so that the ratio of palmitoylated protein decreases; the localization and functionality of the protein are controlled by NO. Like the palmitoylation reaction, the S-nitrosylation reaction with NO is also reversibly controlled. In the synapses, in particular, there thought to be signal transduction (feedback) by NO both on the post-synapse side and on the pre-synapse side.

Because nerve cell exchange information in the synapses and transmit information to other cells via the synapses, output portions (pre-synapse) and input portions (post-synapse) are usually concurrently present on a single nerve cell. Hence, a single nerve cell has a plurality of input portions and output portions; typically, it integrates information (excitatory signal or suppressive signal) from a plurality of synapses (input portions) on dendrites, conducts the resulting electric impulse through axon, and outputs the information from the synapse at the tip of the axon (output portion) to another nerve cell. As described above, both on the output side and on the input side of the synapses, functional proteins, including receptors and transporters, accumulate at high levels on the lipid raft to form a functional complex. It should be noted, however, that the input portions and output portions of synapse have widely different proteins localized therein. Regarding the localization of synapse-related proteins in nerve cells, it has been suggested that the sequence of the palmitoylation site may distinguish polarity between the lipid raft on the output side and the lipid raft on the input side.

Although the detail of this mechanism for determination of polarity remains unknown, a report is available that the sequence of the palmitoylation site is one of the determinants thereof. In GAP43, which mediates G protein signal transduction in the growth cones of elongating axons, a palmitoylation site (MLCCMRRTKQV) is present at the N-terminus thereof; palmitoylation of the two continuous Cys residues promotes the localization to axonal growth cones and the interaction with G protein; this is essential to axonal growth and leading. As in other cases, this palmitoylation is also reversible; GAP43 palmitoylation decreases with the maturation of the axons. This localization of GAP43 to the axons requires the sequence of the palmitoylation site comprising the two continuous Cys residues. On the other hand, PSD95 (membrane-associated guanylyl kinase; MAGUK), which binds to NMDA receptors on the post-synapse side of excitatory synapses, also has a palmitoylation site (MDCLCIVTTKKYR) at the N-terminus thereof, but the localization of this PSD95 to the post-synapse side (dendrites) requires a sequence comprising the two Cys residues with a Leu residue inserted therein; the chimeric PSD95, wherein this sequence is replaced with the sequence of the palmitoylation site of GAP43, is unavoidably localized in the axons. PSD95 also serves as a scaffold protein for NO synthase (NOS) to cause the accumulation of NOS in the synapses. It should also be noted that the Cys residues at the palmitoylation sites of PSD95 and GAP43 serve as target candidates for NO, and that the above-described axon elongating action of GAP43 is suppressed by NO.

Paralemmin has a palmitoylation site (DLDMKKHRCKCCSIM) at the C terminus thereof, wherein the Cys residues to be palmitoylated are continuous as with GAP43, and are localized in the axons as with GAP43. Therefore, like GAP43, paralemmin is postulated to function in axonal growth cones as well; this agrees with the high expression thereof in the fetal and neonatal brains, and suggests that it may play an important role in axonal elongation and synapse formation. Also because paralemmin is expressed in various tissues in adults as well, it seems to be responsible for an important function on the pre-synapse side of peripheral nerves. Additionally, because different tissues exhibit different splice variant patterns, different molecules may interact in respective tissues; paralemmin seems to exhibit characteristic functions in the synapses of the respective tissues, and may be involved in the control of the release of neurotransmitters from synaptic vesicles.

Pancuronium bromide is a peripheral muscle relaxant, and is described as a non-depolarizing muscle relaxant that competitively blocks the acetylcholine receptor existing in the end plate on the muscular side of the nerve-muscle junction. It should be noted, however, that pancuronium bromide has a relatively long half-life for a non-depolarizing muscle relaxant and is often used in long-time surgical operations, such as laparotomic surgery, but it poses a problem with postoperative recovery in some cases and it may be switched to a muscle relaxant of intermediate half-life during surgery. This difference in half-life is explained by the fact that pancuronium bromide is excreted mainly via the kidneys and is hence inferior to the excretion of muscle relaxants of intermediate half-life via the liver-bile duct system. Pancuronium bromide is also reported to produce adverse effects as an antagonist against the muscarinic receptors M2 and M3. Although acetylcholine receptors, which are known target molecules for pancuronium, are located on the post-synapse side, animal experiments suggest that targets are also present on the pre-synapse side, with muscarinic receptors being assumed to be such targets. The present invention discloses that pancuronium bromide may exhibit its pharmacological actions or adverse effects via paralemmin or a protein homologous thereto or a splice variant molecule thereof, in addition to acetylcholine receptors. For example, pancuronium bromide may act on paralemmin to suppress the control of the release of neurotransmitters (acetylcholines) from synaptic vesicles on the pre-synapse side.

A report is available that paralemmin serves as the scaffold protein for the dopamine receptor D3 subtype. Specifically, a study by the yeast two-hybrid method and the GST-pulldown assay method has revealed that paralemmin binds to the third intracellular loop (IC₃) of the D3 receptor. There are two families of dopamine receptors: D1 and D2. The D2 family comprises three subtypes of dopamine receptors: D2 subtype, D3 subtype, and D4 subtype. The dopamine receptors of the D1 family couple with Gs to activate adenylyl cyclase, whereas those of the D2 family couple with G1 to suppress adenylyl cyclase. The D2 family may promote axon elongation and branching and play an important role in the differentiation of the brain dopaminergic pathway, with a remarkable effect exhibited by the D3 subtype; this finding is consistent with the putative functions of GAP43 and paralemmin.

Although the D2 family of dopamine receptors show relatively high homology in the transmembrane domain, their affinity for drugs differs widely. For example, the D3 subtype is 20 times as high in affinity for dopamine as the D2 subtype. Additionally, the individual subtypes of the D2 family have various levels of affinity for antipsychotic drugs. Also, the D2 family forms homodimers or homotetramers, and these multimers increase the affinity for agonists. Furthermore, the D2 family is capable of forming heterodimers between its subtypes or with other receptors, and this is suggested to be a cause of the diversity of signal transduction. When D2 receptors and D1 receptors are concurrently present without dimerization, the promotion and suppression of signal transduction antagonize with each other in some cases and coordinate with each other in other cases. Hence, because dopamine receptors exhibit different functions and different responses to drugs, depending on the combination of a type of receptors and another type of receptors co-expressed on the cell membrane, scaffold proteins associated with the expression of a particular type of dopamine receptors on the cell membrane are likely to control the functions of dopamine receptors, and can be important drug discovery targets.

Due to the presence of the blood-brain barrier, pancuronium bromide usually exhibits no central action. However, if pancuronium bromide acts on the central nervous system, excitation and seizures are observed. This is attributable to increased intracellular Ca ion concentrations as a result of the paradoxical persistent activation of acetylcholine receptor ion channels. All members of the D2 family are known to inhibit inward Ca currents to decrease the intracellular Ca ion concentration. The present invention suggests that the muscle relaxant pancuronium may act on paralemmin, which is associated with dopamine receptors, to exhibit central action. Dopaminergic neurons are associated with the pathogenesis of Parkinson's disease, and in addition, it has been suggested that schizophrenic symptoms may develop in the case of an imbalance in the brain dopamine pathway. Furthermore, as suggested by the fact that D3 receptors are drawing attention as targets for cocaine poisoning remedy and the like, dopamine receptors are associated with various central nervous diseases. Accordingly, the present invention provides a technique important in screening and designing compounds associated with, for example, novel antipsychotic drugs, antidepressants, Parkinson's disease remedies, and drug poisoning remedies, by disclosing novel interactions between the scaffold protein paralemmin for a particular type of dopamine receptors and the compound paralemmin.

(5) On SEQ ID NO:11 (FLJ36526): NSFL1 Cofactor p47 (p97 Cofactor p47)

The protein shown by SEQ ID NO:11 (FLJ36526), which constitutes an interaction pair with diphemanil, tetrazoline, SR-95639A or acetopromazine in the present invention, is one of the splice variants of the gene that encodes the NSFL1 cofactor p47 (p97 cofactor p47). This NSFL1 cofactor p47 forms a complex with VCP (valosin-containing protein p97), which is an ATPase associated with the intracellular vesicle transportation or membrane structure fusion in a variety of cells, mainly nerve cells, to regulate membrane structures such as Golgi membrane. As a result of docking calculations, diphemanil, tetrazoline, SR-95639A and acetopromazine were shown to bind to a site that can influence the interaction of p47 and p97. The expression of nicotinic acetylcholine receptor and the like in the cell membrane and the desensitization by receptor internalization are also dependent on SNAREs (soluble N-ethylmaleimide-sensitive factor attachment protein receptors); the present invention can relate to the actions of diphemanil, tetrazoline, SR-95639A and acetopromazine exemplified below.

### Diphemanil

A disease associated with diphemanil means a disease for which diphemanil is used or a disease corresponding to an adverse effect of diphemanil. Diphemanil is known as an anticholine drug and a sweating suppressant (external application). Examples of the disease for which diphemanil is used include gastric ulcer, hyperchylia, pyloric spasm, hidrosis and the like. An action associated with diphemanil can be closely relevant to a target protein (target gene) therefor, for example, a protein comprising the amino acid sequence shown by SEQ ID NO:11 or a protein homologous thereto or a variant thereof.

### SR-95639A

A disease associated with SR-95639A means a disease for which SR-95639A is used or a disease corresponding to an adverse effect of SR-95639A. SR-95639A is a muscarinic M1 receptor agonist, and is known as a minaprine analogue having brain function improving action. An action associated with SR-95639A can be closely relevant to a target protein (target gene) therefor, for example, a protein comprising the amino acid sequence shown by SEQ ID NO:11 or a protein homologous thereto or a variant thereof.

### Tetrazoline

A disease associated with tetrazoline means a disease for which tetrazoline is used or a disease corresponding to an adverse effect of tetrazoline. Tetrazoline is known as a monoamine oxidase (MAO) inhibitor. An action associated with tetrazoline can be closely relevant to a target protein (target gene) therefor, for example, a protein comprising the amino acid sequence shown by SEQ ID NO:11 or a protein homologous thereto or a variant thereof.

### Acetopromazine

A disease associated with acetopromazine means a disease for which acetopromazine is used or a disease corresponding to an adverse effect of acetopromazine. Acetopromazine is known as an anti-anxiety drug. Examples of the disease for which acetopromazine is used include schizophrenia, senile psychosis, mania, melancholia, sedation and hypnosis in neurosis, and the like. An action associated with acetopromazine can be closely relevant to a target protein (target gene) therefor, for example, a protein comprising the amino acid sequence shown by SEQ ID NO:11 or a protein homologous thereto or a variant thereof.

Likewise, the compounds described below can also be closely relevant to proteins comprising the amino acid sequences shown by sequence identification numbers described herein or proteins homologous thereto or variants thereof, respectively.

### Hydroxocobalamin

A disease associated with hydroxocobalamin means a disease for which hydroxocobalamin is used or a disease corresponding to an adverse effect of hydroxocobalamin. Hydroxocobalamin is known as a vitamin drug. Examples of the disease for which hydroxocobalamin is used include the prophylaxis and treatment of vitamin B12 deficiency, supplementation of vitamin B12 in case of an increased demand and insufficient intake thereof from meals, megaloblastic anemia, diphyllobothriasis, neuropathies in malignant anemia, malabsorption syndrome, nutritional or gestational anemia associated with vitamin B12 deficiency or metabolic disorders, post-gastrectomy anemia, anemia in hepatic disorder, radiation-induced leukocytopenia, neuralgia, peripheral neuritis, peripheral neuroparalysis, muscular pain, arthralgia and the like. In contrast, examples of the adverse effect of hydroxocobalamin include hypersensitivity, eruption and the like. An action associated with hydroxocobalamin can be closely relevant to a target protein (target gene) therefor, for example, a protein comprising the amino acid sequence shown by SEQ ID NO:10, SEQ ID NO:11 or SEQ ID NO:15 or a protein homologous thereto or a variant thereof.

### Amphotericin B

A disease associated with amphotericin B means a disease for which amphotericin B is used or a disease corresponding to an adverse effect of amphotericin B. Amphotericin B is known as a polyene-series antifungal drug. Examples of the disease for which amphotericin B is used include serious infections with Cryptococcus neoformans, Candida albicans, Histoplasma capsulatum, Coccidioides immitis and Aspergillus fumigatus and the like. In contrast, examples of the adverse effect of amphotericin B include eating disorders, nausea, diarrhea, maldigestion, vomiting, general malaise, body weight loss, anaphylaxis, anemia, thrombocytopenia, arrhythmias, hypotension, tachypnea, blurred vision, hypokalemia, nephrotoxicity, epilepsy, thrombophlebitis and the like. An action associated with amphotericin B can be closely relevant to a target protein (target gene) therefor, for example, protein comprising the amino acid sequence shown by SEQ ID NO:15 or a protein homologous thereto or a variant thereof.

### Protriptyline

A disease associated with protriptyline means a disease for which protriptyline is used or a disease corresponding to an adverse effect of protriptyline. Protriptyline is known as a non-sedative cyclic antidepressant. Examples of the disease for which protriptyline is used include depressive symptoms, sleep apnea, narcolepsy and the like. In contrast, examples of the adverse effect of protriptyline include impairment of liver function, body weight gain/loss, sweating, eating disorders, epigastric discomfort, diarrhea, anxiety, agitation, insomnia, panic, motor ataxia, tremor, peripheral neuropathy, anesthesia, tingling, blurred vision, adjustment disorder, ocular hypertension, mydriasis, confusion, delusion, headache, nightmare, constipation, dry mouth, nausea, vomiting, impotence, decrease in sexuality, orthostatic hypotension, tachycardia, palpitation, paresthesia, extrapyramidal symptoms, drowsiness, dizziness, patechial hemorrhage, skin eruption, urticaria, itching, photosensitization, tinnitus, encephalographic changes, sensation of hyposthenia, fatigue, agranulocytosis, leukocytopenia, thrombocytopenia, purpura, myocardial infarction, cerebral stroke, heart block, arrhythmias, adynamic ileus, epilepsy and the like. An action associated with protriptyline can be closely relevant to a target protein (target gene) therefor, for example, a protein comprising the amino acid sequence shown by SEQ ID NO:5 or a protein homologous thereto or a variant thereof.

### Rifampicin

A disease associated with rifampicin means a disease for which rifampicin is used or a disease corresponding to an adverse effect of rifampicin. Rifampicin is known as a drug for pathogenic microorganisms. Examples of the disease for which rifampicin is used include pulmonary tuberculosis, bone-joint tuberculosis, urinary tuberculosis and genital tuberculosis, lymph node tuberculosis, Hansen's disease and the like. In contrast, examples of the adverse effect of rifampicin include serious hepatopathies such as fulminant hepatitis, shock, anaphylactoid symptoms, renal failure, interstitial nephritis, nephrotic syndrome, hemolytic anemia, agranulocytosis, thrombocytopenia, serious forms of colitis such as pseudomembranous colitis, toxic epidermal necrolysis, pemphigus-/pemphigoid-like eruption, lichenoid eruption, erythroderma, interstitial pneumonia, hypersensitivity, gastrointestinal disorders and the like. An action associated with rifampicin can be closely relevant to a target protein (target gene) therefor, for example, a protein comprising the amino acid sequence shown by SEQ ID NO:6 or a protein homologous thereto or a variant thereof.

### Solanine α

A condition associated with solanine α means a condition corresponding to a condition for which use of solanine α is desired or an unwanted condition caused by solanine α. Solanine α is known as a toxic substance that accumulates in the surface layer of greened potatoes. Eating potatoes containing solanine α at high concentrations causes symptomatic poisoning with headache, vomiting, abdominal pain, and sensation of fatigue. In severe cases, cerebral edema can develop and children can suffer clouding of consciousness, coma, and convulsion, and die. An action associated with solanine a can be closely relevant to a target protein (target gene) therefor, for example, a protein comprising the amino acid sequence shown by SEQ ID NO:7 or SEQ ID NO:15 or a protein homologous thereto or a variant thereof.

### Amethopterin

A disease associated with amethopterin means a disease for which amethopterin is used or a disease corresponding to an adverse effect of amethopterin. Amethopterin is known as an anti-malignant tumor drug, anti-inflammatory drug, and rheumatic drug. Examples of the disease for which amethopterin is used include chronic lymphatic leukemia, chronic myelocytic leukemia, chorionic disease, breast cancer, sarcoma, acute leukemia, malignant lymphoma, rheumatic arthritis and the like. In contrast, examples of the adverse effect of amethopterin include shock, anaphylactoid symptoms, myelosuppression, serious hepatic disorder/renal disorder/skin disorder/enteritis, interstitial pneumonia, fibroid lung, pancreatitis, osteoporosis, encephalopathy, Guillain-Barré syndrome, coma, gastrointestinal disorders, alopecia, pigmentation, erythema, headache, gonadal abnormalities and the like. An action associated with amethopterin can be closely relevant to a target protein (target gene) therefor, for example, a protein comprising the amino acid sequence shown by SEQ ID NO:9 or a protein homologous thereto or a variant thereof.

### Benztropine

A disease associated with benztropine means a disease for which benztropine is used or a disease corresponding to an adverse effect of benztropine. Benztropine is known as an anti-histamine anti-cholinergic drug. Examples of the disease for which benztropine is used include Parkinson's disease, drug-induced extrapyramidal symptoms and the like. In contrast, examples of the adverse effect of benztropine include blurred vision, constipation, dry mouth, nausea, ischuria, tachycardia, confusion, psychiatric disease, heatstroke, hyperpyrexia, adynamic ileus and the like. An action associated with benztropine can be closely relevant to a target protein (target gene) therefor, for example, a protein comprising the amino acid sequence shown by SEQ ID NO:14 or SEQ ID NO:19 or a protein homologous thereto or a variant thereof.

### Sulfasalazine

A disease associated with sulfasalazine means a disease for which sulfasalazine is used or a disease corresponding to an adverse effect of sulfasalazine. Sulfasalazine is known as an antipyretic analgesic anti-inflammatory drug or an antirheumatic drug. Examples of the disease for which sulfasalazine is used include ulcerative colitis, regional enteritis, non-specific colitis and the like. In contrast, examples of the adverse effect of sulfasalazine include aplastic anemia, pancytopenia, agranulocytosis, thrombocytopenia, anemia, mucocutaneous ocular syndrome, toxic epidermal necrolysis, erythroderma type drug rash, interstitial pneumonia, drug-induced pneumonia, PIE syndrome, infectious mononucleosis-like symptoms, SLE-like symptoms, acute renal failure, nephrotic syndrome, fibrosing alveolitis, aseptic meningitis (meningoencephalitis), hepatitis, liver dysfunction, jaundice, leukocytopenia, renal calculus, alopecia, gastralgia, nausea and vomiting, eruption, headache, vertigo, reversible oligospermia, fever and the like. An action associated with sulfasalazine can be closely relevant to a target protein (target gene) therefor, for example, a protein comprising the amino acid sequence shown by SEQ ID NO:13 or a protein homologous thereto or a variant thereof.

### Nalidixic Acid

A disease associated with nalidixic acid means a disease for which nalidixic acid is used or a disease corresponding to an adverse effect of nalidixic acid. Nalidixic acid is known as an antibacterial drug and the like. Examples of the disease for which nalidixic acid is used include pyelonephritis, pyelitis, cystitis, urethritis, prostatitis, gonorrhea, bacterial dysentery, enteritis, cystic cholangitis and the like. In contrast, examples of the adverse effect of nalidixic acid include shock, convulsion, hemolytic anemia, visual abnormalities, drowsiness, vertigo, eruption, urticaria, photosensitivity, gastrointestinal/hepatic disorders, leukocytopenia and the like. An action associated with nalidixic acid can be closely relevant to a target protein (target gene) therefor, for example, a protein comprising the amino acid sequence shown by SEQ ID NO:13 or a protein homologous thereto or a variant thereof.

### Astemizole

A disease associated with astemizole means a disease for which astemizole is used or a disease corresponding to an adverse effect of astemizole. Astemizole is known as an anti-allergic drug and the like. Examples of the disease for which astemizole is used include bronchial asthma, urticaria, eczema and dermatitis, skin pruritus, allergic rhinitis and the like. In contrast, examples of the adverse effect of astemizole include death, syncope, cardiac [beat] arrest, QT interval prolongation, torsades de pointes (ventricular tachycardia), premature ventricular twitch, ventricular arrhythmias, lethargy, headache, fatigue, dizziness, sleepiness, torpor, depression, paresthesia, psychomotor disorder convulsion, dry mouth, nausea, abdominal pain, distention, diarrhea, cardiovascular disorders [QT prolongation, ventricular arrhythmias (including torsades de pointes), cardiac arrest (including death) and the like], pancytopenia and the like. As targets for astemizole, histamine H1 receptors are known. An action associated with astemizole can be closely relevant to a target protein (target gene) therefor, for example, a protein comprising the amino acid sequence shown by SEQ ID NO:14 or SEQ ID NO:19 or a protein homologous thereto or a variant thereof.

### Chlorprothixene

A disease associated with chlorprothixene means a disease for which chlorprothixene is used or a disease corresponding to an adverse effect of chlorprothixene. Chlorprothixene is known as a thioxanthine derivative prescribed for schizophrenia. Examples of the disease for which chlorprothixene is used include mental disorders such as schizophrenia, postherpetic neuralgia, and the like. In contrast, examples of the adverse effect of chlorprothixene include agranulocytosis, eosinophilia, leukocytopenia, hemolytic anemia, thrombocytopenia, pancytopenia, postural hypotension, tachycardia, muscular tension abnormalities, acathisia, Parkinsonian symptoms, dry mouth, nasal obstruction, visual abnormalities, constipation, epilepsy, lethargy, insomnia, headache, confusion, polyneuropathy, hyperactivity disorder, tardive dyskinesia, lactorrhea, body weight gain, hyperpyrexia, muscular rigidity, mental state changes, tachyarrhythmia, hypotension, sweating, gastric discomfort, dysuria, cholestasis, obstructive jaundice, xerophthalmia, blurred vision, eruption, contact dermatitis, photosensitivity, systemic erythematosus, drug withdrawal syndrome and the like. An action associated with chlorprothixene can be closely relevant to a target protein (target gene) therefor, for example, a protein comprising the amino acid sequence shown by SEQ ID NO:14 or SEQ ID NO:19 or a protein homologous thereto or a variant thereof.

### Loperamide

A disease associated with loperamide means a disease for which loperamide is used or a disease corresponding to an adverse effect of loperamide. Loperamide is known as a gastrointestinal drug. Examples of the disease for which loperamide is used include diarrhea, acute diarrhea and the like. In contrast, examples of the adverse effect of loperamide include ileus-like symptoms, anaphylactoid symptoms, eruption, impairment of liver function, sensation of abdominal distention, nausea and vomiting, dry mouth, drowsiness, vertigo, sweating and the like. An action associated with loperamide can be closely relevant to a target protein (target gene) therefor, for example, a protein comprising the amino acid sequence shown by SEQ ID NO:14 or SEQ ID NO:19 or a protein homologous thereto or a variant thereof.

### Fluphenazine

A disease associated with fluphenazine means a disease for which fluphenazine is used or a disease corresponding to an adverse effect of fluphenazine. Fluphenazine is known as an antipsychotic drugs, antidepressant, antimanic, and psychostimulant. Examples of the disease for which fluphenazine is used include schizophrenia and the like. In contrast, examples of the adverse effect of fluphenazine include malignant syndrome, sudden death, aplastic anemia, hemolytic anemia, platelet anemia, adynamic ileus, tardive dyskinesia, SIADH, eye disorders, SLE-like symptoms, liver dysfunction, jaundice, hypersensitive symptoms, photosensitivity, leukocytopenia, granulocytopenia, thrombocytopenic purpura, hepatic disorder, fall in BP, tachycardia, extrapyramidal symptoms, miosis, confusion, insomnia and the like. An action associated with fluphenazine can be closely relevant to a target protein (target gene) therefor, for example, a protein comprising the amino acid sequence shown by SEQ ID NO:14 or SEQ ID NO:19 or a protein homologous thereto or a variant thereof.

### Mefloquine

A disease associated with mefloquine means a disease for which mefloquine is used or a disease corresponding to an adverse effect of mefloquine. Mefloquine is known as an antiparasitic drug. Examples of the disease for which mefloquine is used include malaria and the like. In contrast, examples of the adverse effect of mefloquine include mucocutaneous ocular syndrome, toxic epidermal necrolysis, convulsion, confusion, hallucinations, delusion, pneumonia, dyspnea, circulatory insufficiency, heart block, encephalopathy, vertigo, headache, staggers, nausea, sensation of abdominal distention, gastric discomfort, increased eosinophils, increased fibrinogen, urticaria, increased AST, ALT, LDH, TTT, ZTT, and LAP, decreased BUN, increased erythrocyte sedimentation rates and the like. An action associated with mefloquine can be closely relevant to a target protein (target gene) therefor, for example, a protein comprising the amino acid sequence shown by SEQ ID NO:12, SEQ ID NO:14 or SEQ ID NO:19 or a protein homologous thereto or a variant thereof.

### Perphenazine

A disease associated with perphenazine means a disease for which perphenazine is used or a disease corresponding to an adverse effect of perphenazine. Perphenazine is known as a neural drug. Examples of the disease for which perphenazine is used include schizophrenia, preoperative and postoperative nausea and vomiting, Meniere's syndrome and the like. In contrast, examples of the adverse effect of perphenazine include malignant syndrome, sudden death, adynamic ileus, tardive dyskinesia, turbidity of cornea and lens, corneal pigmentation, SLE-like symptoms, fall in BP, tachycardia, leukocyte granulocyte reduction, intestinal measles, hepatic disorder, extrapyramidal symptoms, miosis, skin pigmentation, hypersensitivity, insomnia and the like. An action associated with perphenazine can be closely relevant to a target protein (target gene) therefor, for example, a protein comprising the amino acid sequence shown by SEQ ID NO:14 or SEQ ID NO:19 or a protein homologous thereto or a variant thereof.

### Perhexyline

A disease associated with perhexyline means a disease for which perhexyline is used or a disease corresponding to an adverse effect of perhexyline. Perhexyline is known as an anti-anginal drug and anti-arrhythmic drug. Examples of the disease for which perhexyline is used include intractable angina pectoris, coronary artery revascularization, ventricular arrhythmias and the like in inoperable coronary disease patients. In contrast, examples of the adverse effect of perhexyline include electrocardiographic abnormalities, ventricular repolarization abnormalities, sinus bradycardia, QT interval prolongation, extrasystole, torsades de pointes, unconsciousness, headache, tremor, syncope type vertigo, sensation of hyposthenia, depression, fatigue, dizziness, peripheral neuropathies, paresthesia, body weight loss, polyneuropathies, sensorimotor neuropathies, papilledema, Guillain-Barré syndrome, motor ataxia, Parkinsonian symptoms, hypoglycemia, hyperinsulinemia, nausea, vomiting, eating disorders, upper abdominal pain, body weight loss, hepatic cirrhosis, hepatic encephalopathy, portal hypertension, hepatitis, hepatomegaly, jaundice, keratopathy, bronchial cancer, bronchial convulsion, eruption, myopathy and the like. An action associated with perhexyline can be closely relevant to a target protein (target gene) therefor, for example, a protein comprising the amino acid sequence shown by SEQ ID NO:12, SEQ ID NO:14 or a protein homologous thereto or a variant thereof.

### Raloxifene

A disease associated with raloxifene means a disease for which raloxifene is used or a disease corresponding to an adverse effect of raloxifene. Raloxifene is known as an osteoporosis remedy. Examples of the disease for which raloxifene is used include postmenopausal osteoporosis and the like. In contrast, examples of the adverse effect of raloxifene include venous thromboembolism and the like. An action associated with raloxifene can be closely relevant to a target protein (target gene) therefor, for example, a protein comprising the amino acid sequence shown by SEQ ID NO:12, SEQ ID NO:14 or SEQ ID NO:19 or a protein homologous thereto or a variant thereof.

### Simvastatin

A disease associated with simvastatin means a disease for which simvastatin is used or a disease corresponding to an adverse effect of simvastatin. Simvastatin is known as a hyperlipemia remedy or an anti-dementia drug. Examples of the disease for which simvastatin is used include hyperlipemia, familial hypercholesterolemia and the like. In contrast, examples of the adverse effect of simvastatin include rhabdomyolysis, myopathy, hepatitis, liver dysfunction, jaundice, peripheral neuropathy, thrombocytopenia, hypersensitive symptoms, abdominal pain, nausea, vomiting, increased AST, ALT, LDH, and CK, eruption, itching and the like. An action associated with simvastatin can be closely relevant to a target protein (target gene) therefor, for example, a protein comprising the amino acid sequence shown by SEQ ID NO:15 or a protein homologous thereto or a variant thereof.

### Benoxinate

A disease associated with benoxinate means a disease for which benoxinate is used or a disease corresponding to an adverse effect of benoxinate. Benoxinate is known as a topical anesthetic for opthalmology. Examples of the disease for which benoxinate is used include topical anesthesia and the like in opthalmology. In contrast, examples of the adverse effect of benoxinate include shock and the like. An action associated with benoxinate can be closely relevant to a target protein (target gene) therefor, for example, a protein comprising the amino acid sequence shown by SEQ ID NO:16 or a protein homologous thereto or a variant thereof.

### Pioglitazone

A disease associated with pioglitazone means a disease for which pioglitazone is used or a disease corresponding to an adverse effect of pioglitazone. Pioglitazone is known as a diabetes remedy, hyperlipemia remedy, and gout and hyperuricemia remedy. Examples of the disease for which pioglitazone is used include type 2 diabetes mellitus and the like. In contrast, examples of the adverse effect of pioglitazone include heart failure (onset and exacerbation), liver dysfunction, jaundice, edema, hypoglycemic symptoms (coadministration with other diabetes drugs), recurrence of gastric ulcers, increased LDH, anemia, leukocytopenia, thrombocytopenia, increased blood pressure, increased cardiothoracic ratios, electrocardiographic abnormalities, palpitation, eruption, eczema, itching, nausea, vomiting, gastric discomfort, heartburn, increased AST, ALT, A1-P, and γ-GTP, vertigo, staggers, headache, increased CK, BUN, and K and the like. An action associated with pioglitazone can be closely relevant to a target protein (target gene) therefor, for example, a protein comprising the amino acid sequence shown by SEQ ID NO:17 or a protein homologous thereto or a variant thereof. PPARγ and the like are known as targets for pioglitazone.

### Thioproperazine

A disease associated with thioproperazine means a disease for which thioproperazine is used or a disease corresponding to an adverse effect of thioproperazine. Thioproperazine is known as a phenothiazine-series nerve stabilizer. Examples of the disease for which thioproperazine is used include schizophrenia and the like. In contrast, examples of the adverse effect of thioproperazine include malignant syndrome, extrapyramidal symptoms (Parkinsonian syndrome (digital tremors, muscular rigidity, hypersalivation and the like), dyskinesia (convulsive torticollis, facial and neck twitches, opisthotonos, ocular versive seizure and the like), acathisia, involuntary motions of perioral portion and the like), body weight gain, gynecomastia, lactation, aspermia, menstruation abnormalities, glycosuria, psychoneurotic symptoms (confusion, insomnia, headache, anxiety, excitation, irritability), dry mouth, nasal obstruction, general malaise, fever, edema, ischuria, anuria, pollakiuria, urinary incontinence, skin pigmentation, systemic lupus erythematosus and the like. An action associated with thioproperazine can be closely relevant to a target protein (target gene) therefor, for example, a protein comprising the amino acid sequence shown by SEQ ID NO:12 or SEQ ID NO:18 or a protein homologous thereto or a variant thereof.

### Quinacrine

A disease associated with quinacrine means a disease for which quinacrine is used or a disease corresponding to an adverse effect of quinacrine. Quinacrine is known as an acridine derivative. Examples of the disease for which quinacrine is used include giardiasis, tenial infection, amebiasis, collagen disease, pneumothorax, tumoral exudation, female contraception and the like. In contrast, examples of the adverse effect of quinacrine include aplastic anemia, blood coagulation deficiency, headache, dizziness, nightmare, irritability, nervousness, toxic psychosis, epilepsy, convulsion, nausea, eating disorders, diarrhea, abdominal convulsion, vomiting, hepatitis, corneal edema, retinopathy, interstitial pneumonia, granuloma, skin discoloration, eruption, exfoliative reactions, skin atrophy, alopecia, pigmentary alteration, wart formation, squamous cell carcinoma and the like. An action associated with quinacrine can be closely relevant to a target protein (target gene) therefor, for example, a protein comprising the amino acid sequence shown by SEQ ID NO:12 or SEQ ID NO:19 or a protein homologous thereto or a variant thereof.

A condition associated with GBR12909 means a condition corresponding to a condition for which use of GBR12909 is desired or an unwanted condition caused by GBR12909. GBR12909 is known as a dopamine uptake inhibitory substance. Because GBR12909 is highly selective for dopamine transport proteins, it can be useful in the treatment of diseases such as depression and cocaine poisoning. An action associated with GBR12909 can be closely relevant to a target protein (target gene) therefor, for example, a protein comprising the amino acid sequence shown by SEQ ID NO:19 or a protein homologous thereto or a variant thereof.

### Benzethonium

A disease associated with benzethonium means a disease for which benzethonium is used or a disease corresponding to an adverse effect of benzethonium. Benzethonium is known as a drug for pathogenic microorganisms and drug for dentistry and oral medicine. Examples of the disease for which benzethonium is used include pharyngitis, tonsillitis, stomatitis, acute gingivitis, glossitis, oral wounds and the like. In contrast, examples of the adverse effect of benzethonium include eruption, itching, oral/pharyngeal irritancy, roughness in the mouth and the like. An action associated with benzethonium can be closely relevant to a target protein (target gene) therefor, for example, a protein comprising the amino acid sequence shown by SEQ ID NO:20 or SEQ ID NO:24 or a protein homologous thereto or a variant thereof.

### Albendazole

A disease associated with albendazole means a disease for which albendazole is used or a disease corresponding to an adverse effect of albendazole. Albendazole is known as an anthelmintic and the like. Examples of the disease for which albendazole is used include hydatidosis and the like. In contrast, examples of the adverse effect of albendazole include liver-bile duct system disorders (liver dysfunction, increased AST (GOT), increased ALT (GPT)), pancytopenia and the like. An action associated with albendazole can be closely relevant to a target protein (target gene) therefor, for example, a protein comprising the amino acid sequence shown by SEQ ID NO:24 or a protein homologous thereto or a variant thereof.

### Amikacin

A disease associated with amikacin means a disease for which amikacin is used or a disease corresponding to an adverse effect of amikacin. Amikacin is known as an antibiotic (aminoglycoside-series). Examples of the disease for which amikacin is used include infectious diseases (i.e., sepsis, complicating infection in bronchiectasis, pneumonia, pulmonary suppuration, peritonitis, pyelonephritis, cystitis, urethritis, secondary infections following wounds/burns and surgery) caused by amikacin-sensitive strains of gentamycin-resistant Pseudomonas aeruginosa, myxomycetes, Serratia, Escherichia coli, Klebsiella, Enterobacter, and Citrobacter and the like. In contrast, examples of the adverse effect of amikacin include renal disorders, eruption, deafness, headache and the like. An action associated with amikacin can be closely relevant to a target protein (target gene) therefor, for example, a protein comprising the amino acid sequence shown by SEQ ID NO:12 or a protein homologous thereto or a variant thereof.

### Amiodarone

A disease associated with amiodarone means a disease for which amiodarone is used or a disease corresponding to an adverse effect of amiodarone. Amiodarone is known as an antiarrhythmic drug (group III repolarization retardant). Examples of the disease for which amiodarone is used include recurrent arrhythmia, ventricular fibrillation, ventricular tachycardia, atrial fibrillation that accompanies hypertrophic cardiomyopathy and the like. In contrast, examples of the adverse effect of amiodarone include thyroid dysfunction, corneal pigmentation, lung dysfunction (interstitial pneumonia, fibroid lung, alveolitis), severe aggravation of existing arrhythmia, heart failure, bradycardia, cardiac arrest, complete atrioventricular block, decreased blood pressure, liver dysfunction, adult respiratory distress syndrome and the like. An action associated with amiodarone can be closely relevant to a target protein (target gene) therefor, for example, a protein comprising the amino acid sequence shown by SEQ ID NO:12 or a protein homologous thereto or a variant thereof.

### Apigenin

A disease associated with apigenin means a disease for which apigenin is used or a disease corresponding to an adverse effect of apigenin. Apigenin is known as a plant flavonoid (chamomile and the like) ingredient used in traditional therapy. Examples of the action of apigenin include antibacterial activity, anticancer activity, antiviral activity, anti-inflammatory action, antispasmodic action, sedative action and the like. An action associated with apigenin can be closely relevant to a target protein (target gene) therefor, for example, a protein comprising the amino acid sequence shown by SEQ ID NO:15 or a protein homologous thereto or a variant thereof.

### Buprenorphine

A disease associated with buprenorphine means a disease for which buprenorphine is used or a disease corresponding to an adverse effect of buprenorphine. Buprenorphine is known as a central analgesic and an antipyretic analgesic anti-inflammatory drug. Examples of the disease for which buprenorphine is used include pain relief after surgery and in various cancers and myocardial infarction and the like. In contrast, examples of the adverse effect of buprenorphine include respiratory depression, dyspnea, depression of root of tongue, shock, delirium, delusion and the like. An action associated with buprenorphine can be closely relevant to a target protein (target gene) therefor, for example, a protein comprising the amino acid sequence shown by SEQ ID NO:12 or a protein homologous thereto or a variant thereof or a protein homologous thereto or a variant thereof.

### Celestine Blue

A disease associated with celestine blue means a disease for which celestine blue is used or a disease corresponding to an adverse effect of celestine blue. Celestine blue is known as a pharmaceutical not intended for treatment and a tissue stain. Examples of uses of celestine blue include tissue stains, hematoxylin substitutes and the like. An action associated with celestine blue can be closely relevant to a target protein (target gene) therefor, for example, a protein comprising the amino acid sequence shown by SEQ ID NO:15 or a protein homologous thereto or a variant thereof.

### Chlorambucil

A disease associated with chlorambucil means a disease for which chlorambucil is used or a disease corresponding to an adverse effect of chlorambucil. Chlorambucil is known as an anticancer agent and an antileukemic drug (nitrogen mustard type alkylating agent). Examples of the disease for which chlorambucil is used include chronic lymphocytic leukemia, malignant lymphoma and the like. In contrast, examples of the adverse effect of chlorambucil include bone marrow function suppression, male infertility, nausea, vomiting, diarrhea, tremor, contractions, confusion, motor ataxia, flaccid paralysis and the like. An action associated with chlorambucil can be closely relevant to a target protein (target gene) therefor, for example, a protein comprising the amino acid sequence shown by SEQ ID NO:8 or a protein homologous thereto or a variant thereof.

### Chlorhexidine

A disease associated with chlorhexidine means a disease for which chlorhexidine is used or a disease corresponding to an adverse effect of chlorhexidine. Chlorhexidine is known as a bactericidal disinfectant (drug for the oral cavity). Examples of uses of chlorhexidine include conjunctiva lavage, skin disinfection, hand disinfection for medical personnel, disinfection of medical devices and the like. In contrast, examples of the adverse effect of chlorhexidine include deafness/neuropathy in the case of direct use on auditory nerves and central nerves and the like. An action associated with chlorhexidine can be closely relevant to a target protein (target gene) therefor, for example, a protein comprising the amino acid sequence shown by SEQ ID NO:12 or a protein homologous thereto or a variant thereof.

### Chlorpromazine

A disease associated with chlorpromazine means a disease for which chlorpromazine is used or a disease corresponding to an adverse effect of chlorpromazine. Chlorpromazine is known as an anti-psychotic drug (group B: low-titer group, phenothiazine derivative) and the like. Examples of the disease for which chlorpromazine is used include schizophrenia, mania, anxiety/tension/depression in neurosis, nausea/vomiting, hiccups and the like. Examples of the adverse drug reaction of chlorpromazine include sudden death, aplastic anemia, hemolytic anemia, adynamic ileus, tardive dyskinesia, syndrome of inappropriate secretion of antidiuretic hormone (SIADH), eye disorders, SLE-like symptoms, liver dysfunction, arrhythmias, Parkinsonian syndrome (digital tremors, muscular rigidity, hypersalivation and the like), gynecomastia, lactation, aspermia, menstruation, abnormalities, confusion, insomnia, vertigo, photosensitivity and the like. An action associated with chlorpromazine can be closely relevant to a target protein (target gene) therefor, for example, a protein comprising the amino acid sequence shown by SEQ ID NO:24 or a protein homologous thereto or a variant thereof.

### Cinchonine

A disease associated with cinchonine means a disease for which cinchonine is used or a disease corresponding to an adverse effect of cinchonine. Cinchonine is known as an antiparasitic/antiprotozoal drug, an antimalarial drug, and a quinine-related ingredient of the quinine alkaloid. Examples of the disease for which cinchonine is used include malarial infectious diseases and the like. An action associated with cinchonine can be closely relevant to a target protein (target gene) therefor, for example, a protein comprising the amino acid sequence shown by SEQ ID NO:15 or a protein homologous thereto or a variant thereof.

### Clofazimine

A disease associated with clofazimine means a disease for which clofazimine is used or a disease corresponding to an adverse effect of clofazimine. Clofazimine is known as a Hansen's disease therapeutic drug. Examples of the disease for which clofazimine is used include Hansen's disease (multi-strain type, erythema nodosum leprosum) and the like. In contrast, examples of the adverse effect of clofazimine include skin pigmentation, visual acuity reduction, ileus, splenic infarction, thrombotic embolism and the like. An action associated with clofazimine can be closely relevant to a target protein (target gene) therefor, for example, a protein comprising the amino acid sequence shown by SEQ ID NO:24 or a protein homologous thereto or a variant thereof.

### Clomiphene

A disease associated with clomiphene means a disease for which clomiphene is used or a disease corresponding to an adverse effect of clomiphene. Clomiphene is known as a hormone preparation of other class (ovulation inducer). Examples of the disease for which clomiphene is used include ovulation induction in infertility based on ovulation disturbance, male infertility and the like. In contrast, examples of the adverse effect of clomiphene include ovarian swelling due to overstimulation of the ovary, visual disturbance, nausea, vomiting, headache and the like. An action associated with clomiphene can be closely relevant to a target protein (target gene) therefor, for example, a protein comprising the amino acid sequence shown by SEQ ID NO:12 or a protein homologous thereto or a variant thereof.

### Cyproheptadine

A disease associated with cyproheptadine means a disease for which cyproheptadine is used or a disease corresponding to an adverse effect of cyproheptadine. Cyproheptadine is known as a piperidine-series anti-allergic drug (antihistamine drug). Examples of the disease for which cyproheptadine is used include pruritus (eczema/dermatitis, skin pruritus, drug rash) that accompanies skin diseases, urticaria, vasomotor edema, hay fever, allergic rhinitis, vasomotor rhinitis, sneezing/nasal discharge/cough that accompany upper airway inflammation such as common cold, and the like. In contrast, examples of the adverse effect of cyproheptadine include confusion, hallucination, spasm, agranulocytosis, drowsiness, vertigo, general malaise, leukopenia, thrombocytopenia, increased intra-ocular pressure/gastrointestinal motor suppression/increased sputum viscosity due to anticholine action and the like. An action associated with cyproheptadine can be closely relevant to a target protein (target gene) therefor, for example, a protein comprising the amino acid sequence shown by SEQ ID NO:22 or a protein homologous thereto or a variant thereof.

### Deferoxamine

A disease associated with deferoxamine means a disease for which deferoxamine is used or a disease corresponding to an adverse effect of deferoxamine. Deferoxamine is known as a poisoning therapeutic drug (therapeutic drug for heavy metal poisoning and others) and an iron eliminant. Examples of the disease for which deferoxamine is used include increased iron secretion in urine in primary and secondary hemochromatosis and the like. In contrast, examples of the adverse effect of deferoxamine include visual disturbance, impaired hearing, severe fungal infectious diseases such as Yersinia infection and mucormycosis, hematuria, oliguria, urine pigmentation, thrombocytopenia, diarrhea and the like. An action associated with deferoxamine can be closely relevant to a target protein (target gene) therefor, for example, a protein comprising the amino acid sequence shown by SEQ ID NO:3 or a protein homologous thereto or a variant thereof.

### Dihydroergocristine

A disease associated with dihydroergocristine means a disease for which dihydroergocristine is used or a disease corresponding to an adverse effect of dihydroergocristine. Dihydroergocristine is known as an ingredient of the circulatory disorder ameliorator dihydroergotoxin mesylate. Examples of the disease for which dihydroergocristine is used include incidental symptoms that accompany head trauma sequelae, hypertension (gentle hypotensive action), peripheral circulatory disorders (Burger disease, obstructive arteriosclerosis, arterial embolism/thrombosis, Raynaud's syndrome, chilblain/frostbites, intermittent claudication) and the like. In contrast, examples of the adverse effect of dihydroergocristine include gastrointestinal disorders, eruption/pruritus, headache, vertigo, bradycardia, blood pressure reduction, brain anemia symptoms, facial flush, sensation of hot flash, cardiac palpitation and the like. An action associated with dihydroergocristine can be closely relevant to a target protein (target gene) therefor, for example, a protein comprising the amino acid sequence shown by SEQ ID NO:12 or a protein homologous thereto or a variant thereof.

### Dihydroergotamine

A disease associated with dihydroergotamine means a disease for which dihydroergotamine is used or a disease corresponding to an adverse effect of dihydroergotamine. Dihydroergotamine is known as an antipyretic analgesic anti-inflammatory drug (migraine therapeutic drug). Examples of the disease for which dihydroergotamine is used include migraine (vascular headache), orthostatic hypotension and the like. In contrast, examples of the adverse effect of dihydroergotamine include fibrosis of the pleura/retroperitoneum/heart valve, urticaria, nausea, vomiting, diarrhea, vertigo, drowsiness, blood pressure elevation, palpitation, manual digital psychroesthesia and the like. An action associated with dihydroergotamine can be closely relevant to a target protein (target gene) therefor, for example, a protein comprising the amino acid sequence shown by SEQ ID NO:12 or a protein homologous thereto or a variant thereof.

### Domperidone

A disease associated with domperidone means a disease for which domperidone is used or a disease corresponding to an adverse effect of domperidone. Domperidone is known as a gastrointestinal drug (gastrointestinal function adjusting drug) and a gastrointestinal motor ameliorator. Examples of the disease for which domperidone is used include diseases such as chronic gastritis, gastroptosis, post-gastrectomy syndrome, periodic vomiting, and upper airway infections, and mitigation of gastrointestinal symptoms (nausea, vomiting, anorexia, abdominal distention, abdominal pain, heartburn and the like) during treatment with drugs (anti-malignant tumor agents or levodopa preparations) and the like. In contrast, examples of the adverse effect of domperidone include diarrhea, impulse to defecate, abdominal pain, anaphylactoid symptoms, extrapyramidal symptoms such as tremor and muscle rigidity (Parkinsonian symptoms), liver dysfunction, gynecomastia, elevated prolactin levels, milk secretion, sensation of breast distention, emmeniopathy, cardiac palpitation, sweating, drowsiness, vertigo and the like. An action associated with domperidone can be closely relevant to a target protein (target gene) therefor, for example, a protein comprising the amino acid sequence shown by SEQ ID NO:23 or a protein homologous thereto or a variant thereof.

### Doxazosin

A disease associated with doxazosin means a disease for which doxazosin is used or a disease corresponding to an adverse effect of doxazosin. Doxazosin is known as a hypotensive drug (sympathetic suppressant, α1 blocker). Examples of the disease for which doxazosin is used include hypertension, hypertension due to pheochromocytoma, benign prostatic hyperplasia (BPH) and the like. In contrast, examples of the adverse effect of doxazosin include syncope/loss of consciousness, orthostatic hypotension, arrhythmia, cerebrovascular disorders, angina pectoris, myocardial infarction, agranulocytosis, leukopenia, thrombocytopenia, liver dysfunction and the like. An action associated with doxazosin can be closely relevant to a target protein (target gene) therefor, for example, a protein comprising the amino acid sequence shown by SEQ ID NO:12 or a protein homologous thereto or a variant thereof.

### Eburnamonine

A disease associated with eburnamonine means a disease for which eburnamonine is used or a disease corresponding to an adverse effect of eburnamonine. Eburnamonine is known as an alkaloid contained in Vinca minor extract (common periwinkle), which is used in traditional therapy. Examples of the action of eburnamonine include amelioration of neurological/psychiatric symptoms such as dementia, memory, concentration, tinnitus, vision, and melancholia, and the like. An action associated with eburnamonine can be closely relevant to a target protein (target gene) therefor, for example, a protein comprising the amino acid sequence shown by SEQ ID NO:3 or a protein homologous thereto or a variant thereof.

### Ellipticine

A disease associated with ellipticine means a disease for which ellipticine is used or a disease corresponding to an adverse effect of ellipticine. Ellipticine is a topoisomerase II inhibitor and is known as a plant alkaloid having anticancer action/antiviral action. Examples of the action of ellipticine include anticancer action, anti-viral (anti-HIV) action and the like. An action associated with ellipticine can be closely relevant to a target protein (target gene) therefor, for example, a protein comprising the amino acid sequence shown by SEQ ID NO:24 or a protein homologous thereto or a variant thereof.

### Emetine

A disease associated with emetine means a disease for which emetine is used or a disease corresponding to an adverse effect of emetine. Emetine is known as a plant (ipecac) alkaloid used in antiparasitic/antiprotozoal drugs. Examples of the disease for which emetine is used include anti-amebiasis (anti-Entamoeba histolytica trophozoite) and the like. In contrast, examples of the adverse effect of emetine include diarrhea, nausea, vomiting, arrhythmia, hypotension, congestive heart failure and the like. An action associated with emetine can be closely relevant to a target protein (target gene) therefor, for example, a protein comprising the amino acid sequence shown by SEQ ID NO:12 or a protein homologous thereto or a variant thereof.

### Ethotoin

A disease associated with ethotoin means a disease for which ethotoin is used or a disease corresponding to an adverse effect of ethotoin. Ethotoin is known as an anti-epileptic drug (phenyloin-series, a main therapy used for major seizures). Examples of the disease for which ethotoin is used include epileptic convulsive seizures (major seizures) and the like. In contrast, examples of the adverse effect of ethotoin include muco-cutaneo-ocular syndrome, toxic epidermal necrolysis, SLE-like symptoms, aplastic anemia, interstitial pneumonia, lymphoma, lymph node swelling, rickets, osteomalacia, dental hypoplasia, thyroid dysfunction and the like. An action associated with ethotoin can be closely relevant to a target protein (target gene) therefor, for example, a protein comprising the amino acid sequence shown by SEQ ID NO:13 or a protein homologous thereto or a variant thereof.

### Fenbendazole

A disease associated with fenbendazole means a disease for which fenbendazole is used or a disease corresponding to an adverse effect of fenbendazole. Fenbendazole is known as an antiparasitic/antiprotozoal drug. Examples of uses of fenbendazole include antiparasitic drugs (animal drugs) and the like. An action associated with fenbendazole can be closely relevant to a target protein (target gene) therefor, for example, a protein comprising the amino acid sequence shown by SEQ ID NO:12 or a protein homologous thereto or a variant thereof.

### Flumequine

A disease associated with flumequine means a disease for which flumequine is used or a disease corresponding to an adverse effect of flumequine. Flumequine is known as an antibiotic (quinolone-series). Examples of uses of flumequine include antibiotics, antibacterial drugs and the like. An action associated with flumequine can be closely relevant to a target protein (target gene) therefor, for example, a protein comprising the amino acid sequence shown by SEQ ID NO:13 or a protein homologous thereto or a variant thereof.

### Flunarizine

A disease associated with flunarizine means a disease for which flunarizine is used or a disease corresponding to an adverse effect of flunarizine. Flunarizine is known as a calcium antagonist. Examples of the disease for which flunarizine is used include cerebral circulation improvement, migraine (prophylaxis) and the like. In contrast, examples of the adverse effect of flunarizine include drug-induced Parkinsonian symptoms and the like. An action associated with flunarizine can be closely relevant to a target protein (target gene) therefor, for example, a protein comprising the amino acid sequence shown by SEQ ID NO:12 or a protein homologous thereto or a variant thereof.

### Flupentixol

A disease associated with flupentixol means a disease for which flupentixol is used or a disease corresponding to an adverse effect of flupentixol. Flupentixol is known as an antipsychotic drug. Examples of the action of flupentixol include sedative effects (psychomotor excitation/impulsion suppression), action against unusual experience (amelioration of hallucination/delusion and the like), activation effects (amelioration of decreased mental activity) and the like. In contrast, examples of the adverse effect of flupentixol include drug-induced Parkinsonian symptoms, acute dystonia (ocular supraduction, spasmodic torsion of neck, projection of tongue, dysphagia), akathisia (inability to remain in sitting posture), autonomic nervous symptoms (dry mouth, sweating, constipation, orthostatic hypotension, reflex tachycardia, drowsiness), tardive dyskinesia and the like. An action associated with flupentixol can be closely relevant to a target protein (target gene) therefor, for example, a protein comprising the amino acid sequence shown by SEQ ID NO:12 or a protein homologous thereto or a variant thereof.

### Glipizide

A disease associated with glipizide means a disease for which glipizide is used or a disease corresponding to an adverse effect of glipizide. Glipizide is known as a diabetes mellitus therapeutic drug (sulfonylurea-series). Examples of the disease for which glipizide is used include diabetes mellitus and the like. An action associated with glipizide can be closely relevant to a target protein (target gene) therefor, for example, a protein comprising the amino acid sequence shown by SEQ ID NO:24 or a protein homologous thereto or a variant thereof.

### Harmaline

A disease associated with harmaline means a disease for which harmaline is used or a disease corresponding to an adverse effect of harmaline. Harmaline is known as a narcotic, a plant alkaloid (rue), a hallucinogen, and a monoamine oxidase inhibitor. Examples of the action of harmaline include hallucinogenesis, antidepressive action and the like. An action associated with harmaline can be closely relevant to a target protein (target gene) therefor, for example, a protein comprising the amino acid sequence shown by SEQ ID NO:15 or a protein homologous thereto or a variant thereof.

### Hydantoin

A disease associated with hydantoin means a disease for which hydantoin is used or a disease corresponding to an adverse effect of hydantoin. Hydantoin is known as an antispasmodic drug. Examples of the disease for which hydantoin is used include epilepsy and the like. In contrast, examples of the adverse effect of hydantoin include hydantoin-induced gingival hyperplasia and the like. An action associated with hydantoin can be closely relevant to a target protein (target gene) therefor, for example, a protein comprising the amino acid sequence shown by SEQ ID NO:10 or a protein homologous thereto or a variant thereof.

### Lidoflazine

A disease associated with lidoflazine means a disease for which lidoflazine is used or a disease corresponding to an adverse effect of lidoflazine. Lidoflazine is known as a vasodilator, an anti-anginal drug, and a non-selective calcium channel inhibitor. Examples of the disease for which lidoflazine is used include angina pectoris, arrhythmia and the like. An action associated with lidoflazine can be closely relevant to a target protein (target gene) therefor, for example, a protein comprising the amino acid sequence shown by SEQ ID NO:12 or a protein homologous thereto or a variant thereof.

### Lisinopril

A disease associated with lisinopril means a disease for which lisinopril is used or a disease corresponding to an adverse effect of lisinopril. Lisinopril is known as a hypotensive drug (ACE inhibitor). Examples of the disease for which lisinopril is applied include hypertension or chronic heart failure (mild to moderate) and the like. In contrast, examples of the adverse effect of lisinopril include coughing, pharyngeal discomfort, angioedema, abdominal pain, vomiting, diarrhea, acute renal failure, hyperkalemia, pancreatitis, mucocutaneous ocular syndrome (Stevens-Johnson syndrome), toxic epidermal necrolysis (Lyell syndrome), pemphigus-like symptoms, hemolytic anemia and the like. An action associated with lisinopril can be closely relevant to a target protein (target gene) therefor, for example, a protein comprising the amino acid sequence shown by SEQ ID NO:17 or a protein homologous thereto or a variant thereof.

### Mafenide

A disease associated with mafenide means a disease for which mafenide is used or a disease corresponding to an adverse effect of mafenide. Mafenide is known as a sulfonamide-series antiparasitic/antiprotozoal drug. An action associated with mafenide can be closely relevant to a target protein (target gene) therefor, for example, a protein comprising the amino acid sequence shown by SEQ ID NO:6 or a protein homologous thereto or a variant thereof.

### Mefenamic Acid

A disease associated with mefenamic acid means a disease for which mefenamic acid is used or a disease corresponding to an adverse effect of mefenamic acid. Mefenamic acid is one of non-steroidal anti-inflammatory drug (NSAIDs), and is known as an anthranilic acid-series antipyretic analgesic anti-inflammatory drug. Examples of the action of mefenamic acid include remission of postoperative and post-traumatic inflammation and swelling, inflammation resolution/pain relief/defervescence in osteoarthritis, lumbago, symptomatic neuralgia, headache, sinusitis, menorrhalgia, postpartum pain, toothache, and acute upper airway inflammation, and the like. In contrast, examples of the adverse effect of mefenamic acid include gastrointestinal disorders, shock, hemolytic anemia, bone-marrow hypoplasia, muco-cutaneo-ocular syndrome, acute renal insufficiency, digestive ulcer and the like. An action associated with mefenamic acid can be closely relevant to a target protein (target gene) therefor, for example, a protein comprising the amino acid sequence shown by SEQ ID NO:24 or a protein homologous thereto or a variant thereof.

### Megestrol

A disease associated with megestrol means a disease for which megestrol is used or a disease corresponding to an adverse effect of megestrol. Megestrol is known as a sex hormone preparation (progestin). Examples of the disease for which megestrol is used include postmenopausal advanced or recurrent breast cancer and the like. An action associated with megestrol can be closely relevant to a target protein (target gene) therefor, for example, a protein comprising the amino acid sequence shown by SEQ ID NO:24 or a protein homologous thereto or a variant thereof.

### Mesoridazine

A disease associated with mesoridazine means a disease for which mesoridazine is used or a disease corresponding to an adverse effect of mesoridazine. Mesoridazine is known as an antipsychotic drug. Examples of the disease for which mesoridazine is used include schizophrenia, organic brain syndrome, oligophrenia, alcohol withdrawal and the like. In contrast, examples of the adverse effect of mesoridazine include drowsiness, hypotension, drug-induced Parkinsonian symptoms, akathisia, tardive dyskinesia, dry mouth, nasal obstruction, visual abnormalities and the like. An action associated with mesoridazine can be closely relevant to a target protein (target gene) therefor, for example, a protein comprising the amino acid sequence shown by SEQ ID NO:3 or a protein homologous thereto or a variant thereof.

### Metergoline

A disease associated with metergoline means a disease for which metergoline is used or a disease corresponding to an adverse effect of metergoline. Metergoline is known as a 5-HT₂ antagonist. Examples of the action of metergoline include analgesic action in migraine, hypophysial and hypothalamic hormone action and the like. An action associated with metergoline can be closely relevant to a target protein (target gene) therefor, for example, a protein comprising the amino acid sequence shown by SEQ ID NO:12 or a protein homologous thereto or a variant thereof.

Methoxy-6-harmalan

A disease associated with methoxy-6-harmalan means a disease for which methoxy-6-harmalan is used or a disease corresponding to an adverse effect of methoxy-6-harmalan. Methoxy-6-harmalan is known as a narcotic. Examples of the action of methoxy-6-harmalan include hallucinogenesis action, antidepressive action and the like. An action associated with methoxy-6-harmalan can be closely relevant to a target protein (target gene) therefor, for example, a protein comprising the amino acid sequence shown by SEQ ID NO:15 or a protein homologous thereto or a variant thereof.

### Meticrane

A disease associated with meticrane means a disease for which meticrane is used or a disease corresponding to an adverse effect of meticrane. Meticrane is known as a non-thiazide-series hypotensive agent and a diuretic drug. Examples of the action of meticrane include blood pressure reduction in essential hypertension, diuretic action and the like. In contrast, examples of the adverse effect of meticrane include anorexia, nausea, vomiting, eruption, hyperesthesia optica, thrombocytopenia, interstitial pneumonia, pulmonary edema and the like. An action associated with meticrane can be closely relevant to a target protein (target gene) therefor, for example, a protein comprising the amino acid sequence shown by SEQ ID NO:15 or a protein homologous thereto or a variant thereof.

### Methixene

A disease associated with methixene means a disease for which methixene is used or a disease corresponding to an adverse effect of methixene. Methixene is known as an anti-Parkinsonian drug (anticholine drug). Examples of the disease for which methixene is used include anti-Parkinsonian agents, drug-induced Parkinsonism, idiopathic Parkinsonism, other forms of Parkinsonism (post-encephalitic, arteriosclerotic) and the like. In contrast, examples of the adverse effect of methixene include dry mouth, constipation, visual acuity abnormalities, mental confusion, syndrome malin, vertigo, drowsiness, general malaise and the like. An action associated with methixene can be closely relevant to a target protein (target gene) therefor, for example, a protein comprising the amino acid sequence shown by SEQ ID NO:24 or a protein homologous thereto or a variant thereof.

### Mifepristone

A disease associated with mifepristone means a disease for which mifepristone is used or a disease corresponding to an adverse effect of mifepristone. Mifepristone is a synthetic steroid (antiprogesterone action, antiglucocorticoid action), and is known as an abortion drug. Examples of uses of mifepristone include intrauterine abortion within 49 days of gestation (endometrial detachment due to antiprogesterone action) and the like. In contrast, examples of the adverse effect of mifepristone include nausea, vomiting, diarrhea, abdominal pain, headache, vertigo, general malaise, spasm, hemorrhage, vaginal secretion abnormalities, vaginal discomfort, fever, palpitation, syncope, sepsis and the like. An action associated with mifepristone can be closely relevant to a target protein (target gene) therefor, for example, a protein comprising the amino acid sequence shown by SEQ ID NO:7 or a protein homologous thereto or a variant thereof.

### Nordiazepam

A disease associated with nordiazepam means a disease for which nordiazepam is used or a disease corresponding to an adverse effect of nordiazepam. Nordiazepam is a hypnotic/sedative drug (minor tranquilizer), and is known as an active metabolic intermediate from diazepam to oxazepam. Examples of uses of nordiazepam include mitigation of anxiety/tension/depression in neurosis, anxiety/tension in melancholia, somatic signs and anxiety/tension/depression in psychosomatic disorders (gastrointestinal diseases, circulatory diseases, autonomic imbalance, menopausal disorders, lumbago, cervico-omo-brachial syndrome), and muscle tone during muscle spasm/pain that accompany cerebrospinal diseases, pre-anesthetic medication and the like. In contrast, examples of the adverse effect of nordiazepam include dependency, upper airway obstruction due to depression of root of tongue, respiratory depression, stimulatory excitation, confusion, circulatory shock and the like. An action associated with nordiazepam can be closely relevant to a target protein (target gene) therefor, for example, a protein comprising the amino acid sequence shown by SEQ ID NO:24 or a protein homologous thereto or a variant thereof.

### Oxethazaine

A disease associated with oxethazaine means a disease for which oxethazaine is used or a disease corresponding to an adverse effect of oxethazaine oxethazaine is known as a gastrointestinal drug (stomachic/digestant) and a gastrointestinal mucosal topical anesthetic. Examples of the disease for which oxethazaine is used include pain/acid symptoms/nausea/vomiting/gastric discomfort/urged impulse to defecate that accompany esophagitis/gastritis/gastric/duodenal ulcer/irritable bowel syndrome and the like. In contrast, examples of the adverse effect of oxethazaine include hypersensitivity, constipation, anorexia, dry mouth, headache, vertigo, drowsiness, and sensation of lack of strength. An action associated with oxethazaine can be closely relevant to a target protein (target gene) therefor, for example, a protein comprising the amino acid sequence shown by SEQ ID NO:12 or a protein homologous thereto or a variant thereof.

### Oxolinic Acid

A disease associated with oxolinic acid means a disease for which oxolinic acid is used or a disease corresponding to an adverse effect of oxolinic acid. Oxolinic acid is a quinoline-series antibiotic (animal drug), and is known as an antibacterial agent (enteric Gram-negative bacteria and the like). An action associated with oxolinic acid can be closely relevant to a target protein (target gene) therefor, for example, a protein comprising the amino acid sequence shown by SEQ ID NO:12 or a protein homologous thereto or a variant thereof.

### Paclitaxel

A disease associated with paclitaxel means a disease for which paclitaxel is used or a disease corresponding to an adverse effect of paclitaxel. Paclitaxel is known as an alkaloid-series anticancer agent. Examples of the disease for which paclitaxel is used include ovarian cancer, non-small-cell lung cancer, breast cancer, gastric cancer and the like. In contrast, examples of the adverse effect of paclitaxel include peripheral neuropathy, arthralgia, myalgia, nausea, vomiting, alopecia, fever, bone marrow suppression, peripheral neuropathy, interstitial pneumonia, fibroid lung, acute respiratory distress syndrome, myocardial infarction, congestive heart failure, pulmonary embolism, thrombotic phlebitis, cerebral stroke, pulmonary edema, auditory disorders, gut perforation, gastrointestinal hemorrhage, gastrointestinal ulcer, hemorrhagic colitis, liver dysfunction, pancreatitis, acute renal insufficiency, muco-cutaneo-ocular syndrome, toxic epidermal necrolysis, disseminated intravascular coagulation syndrome (DIC) and the like. An action associated with paclitaxel can be closely relevant to a target protein (target gene) therefor, for example, a protein comprising the amino acid sequence shown by SEQ ID NO:15 or a protein homologous thereto or a variant thereof.

### Palmatine

A disease associated with palmatine means a disease for which palmatine is used or a disease corresponding to an adverse effect of palmatine. Palmatine is a plant (goldthread) alkaloid ingredient (but the primary ingredient is berberine), and is known as a bitter stomachic or an antibiotic. An action associated with palmatine can be closely relevant to a target protein (target gene) therefor, for example, a protein comprising the amino acid sequence shown by SEQ ID NO:15 or a protein homologous thereto or a variant thereof.

### Pentoxifylline

A disease associated with pentoxifylline means a disease for which pentoxifylline is used or a disease corresponding to an adverse effect of pentoxifylline. Pentoxifylline is known as a brain circulation metabolism improver. Examples of the disease for which pentoxifylline is used include microcirculation improvement, headache, vertigo, and numbness due to cerebral thrombosis sequelae and chronic brain circulatory disorders, autism and the like. An action associated with pentoxifylline can be closely relevant to a target protein (target gene) therefor, for example, a protein comprising the amino acid sequence shown by SEQ ID NO:24 or a protein homologous thereto or a variant thereof.

### Pimozide

A disease associated with pimozide means a disease for which pimozide is used or a disease corresponding to an adverse effect of pimozide. Pimozide is known as a butyrophenone-series antipsychotic drug (group C: intermediate/atypical group). Examples of the disease for which pimozide is used include schizophrenia, pediatric autistic disorders, unusual behavior/pathologic symptoms/psychiatric symptoms that accompany mental retardation and the like. In contrast, examples of the adverse effect of pimozide include sleep disorders, tremor, akathisia, ventricular tachycardia, sudden death, syndrome malin, spasmodic seizures, hyponatremia, extrapyramidal symptoms, drug-induced Parkinson syndrome, dyskinesia, hypotension, dysuria, elevation of prolactin level and the like. An action associated with pimozide can be closely relevant to a target protein (target gene) therefor, for example, a protein comprising the amino acid sequence shown by SEQ ID NO:3 or SEQ ID NO:12 or a protein homologous thereto or a variant thereof.

### Pinacidil

A disease associated with pinacidil means a disease for which pinacidil is used or a disease corresponding to an adverse effect of pinacidil. Pinacidil is known as a hypotensive drug. Examples of the disease for which pinacidil is used include hypertension and the like. An action associated with pinacidil can be closely relevant to a target protein (target gene) therefor, for example, a protein comprising the amino acid sequence shown by SEQ ID NO:24 or a protein homologous thereto or a variant thereof.

### Rescinnamine

A disease associated with rescinnamine means a disease for which rescinnamine is used or a disease corresponding to an adverse effect of rescinnamine. Rescinnamine is known as a hypotensive drug (peripheral sympathetic suppressant). Examples of the disease for which rescinnamine is used include essential, renal or other forms of hypertension. In contrast, examples of the adverse effect of rescinnamine include depressive states, gastric ulcer, nightmares, extrapyramidal symptoms, drowsiness, vertigo and the like. An action associated with rescinnamine can be closely relevant to a target protein (target gene) therefor, for example, a protein comprising the amino acid sequence shown by SEQ ID NO:12 or a protein homologous thereto or a variant thereof.

### Sulfadimethoxine

A disease associated with sulfadimethoxine means a disease for which sulfadimethoxine is used or a disease corresponding to an adverse effect of sulfadimethoxine. Sulfadimethoxine is an antibiotic (chemotherapeutic agent), and is known as a persistent sulfur agent. Examples of the disease for which sulfadimethoxine is used include meningitis, pyelonephritis, cystitis, tonsillitis, pharyngitis, laryngitis, chancroid and the like. In contrast, examples of the adverse effect of sulfadimethoxine include anorexia, nausea, vomiting, headache, shock, aplastic anemia, hemolytic anemia, muco-cutaneo-ocular syndrome, toxic epidermal necrolysis and the like. An action associated with sulfadimethoxine can be closely relevant to a target protein (target gene) therefor, for example, a protein comprising the amino acid sequence shown by SEQ ID NO:15 or a protein homologous thereto or a variant thereof.

### Syrosingopine

A disease associated with syrosingopine means a disease for which syrosingopine is used or a disease corresponding to an adverse effect of syrosingopine. Syrosingopine is known as a hypotensive drug (peripheral sympathetic suppressant). Examples of the disease for which syrosingopine is used include essential hypertension, hypotensive action, sedative action and the like. In contrast, examples of the adverse effect of syrosingopine include gastric ulcer, nasal obstruction, drowsiness, vertigo, dry mouth, drug-induced depressive states, suicide and the like. An action associated with syrosingopine can be closely relevant to a target protein (target gene) therefor, for example, a protein comprising the amino acid sequence shown by SEQ ID NO:24 or a protein homologous thereto or a variant thereof.

### Tamoxifen

A disease associated with tamoxifen means a disease for which tamoxifen is used or a disease corresponding to an adverse effect of tamoxifen. Tamoxifen is known as an anticancer agent. Examples of the disease for which tamoxifen is used include breast cancer and the like. In contrast, examples of the adverse effect of tamoxifen include amenorrhea, emmeniopathy, nausea, vomiting, anorexia, leukopenia, anemia, thrombocytopenia, visual acuity abnormalities, visual disturbance, thromboembolism, phlebitis, hepatic disorder, hypercalcemia, hysteromyoma, endometrial polyp, endometrial hyperplasia, endometriosis, interstitial pneumonia, anaphylactoid symptoms, muco-cutaneo-ocular syndrome, bullous pemphigoid, pancreatitis and the like. An action associated with tamoxifen can be closely relevant to a target protein (target gene) therefor, for example, a protein comprising the amino acid sequence shown by SEQ ID NO:12 or a protein homologous thereto or a variant thereof.

### Tenoxicam

A disease associated with tenoxicam means a disease for which tenoxicam is used or a disease corresponding to an adverse effect of tenoxicam. Tenoxicam is one of oxicam-series NSAIDs, and is known as an antipyretic analgesic anti-inflammatory drug. Examples of the disease for which tenoxicam is used include chronic articular rheumatism, osteoarthritis, lumbago, cervico-omo-brachial syndrome, shoulder periarthritis, postoperative and posttraumatic inflammation resolution/pain relief and the like. In contrast, examples of the adverse effect of tenoxicam include gastralgia, gastric discomfort, eruption, edema, stomatitis, muco-cutaneo-ocular syndrome, toxic epidermal necrolysis, gastrointestinal perforation, agranulocytosis, paralytic ileus and the like. An action associated with tenoxicam can be closely relevant to a target protein (target gene) therefor, for example, a protein comprising the amino acid sequence shown by SEQ ID NO:13 or a protein homologous thereto or a variant thereof.

### Thioridazine

A disease associated with thioridazine means a disease for which thioridazine is used or a disease corresponding to an adverse effect of thioridazine. Thioridazine is known as a phenothiazine-series antipsychotic drug (group B: low-titer group). Examples of the disease for which thioridazine is used include schizophrenia, neurosis, suppression of anxiety/tension/depression and excitation/hyperactivity, melancholia, oligophrenia, senile psychosis and the like. In contrast, examples of the adverse effect of thioridazine include drowsiness, dry mouth, general malaise, nasal obstruction, manual digital tremor, vertigo, cardiac palpitation, syndrome malin, arrhythmia, electrocardiogram abnormalities, aplastic anemia, hemolytic anemia, agranulocytosis, tardive dyskinesia, extrapyramidal symptoms, syndrome of inappropriate secretion of antidiuretic hormone, visual disturbance and the like. An action associated with thioridazine can be closely relevant to a target protein (target gene) therefor, for example, a protein comprising the amino acid sequence shown by SEQ ID NO:12 or a protein homologous thereto or a variant thereof.

Thiothixene (cis)

A disease associated with thiothixene (cis) means a disease for which thiothixene (cis) is used or a disease corresponding to an adverse effect of thiothixene (cis). Thiothixene (cis) is known as an antipsychotic drug. Examples of the disease for which thiothixene (cis) is used include schizophrenia and the like. In contrast, examples of the adverse effect of thiothixene (cis) include circulatory collapse, comatose states, drowsiness, vertigo, tardive dyskinesia, hyperreflexia, dry mouth, sweating, liver dysfunction, visual disturbance and the like. An action associated with thiothixene (cis) can be closely relevant to a target protein (target gene) therefor, for example, a protein comprising the amino acid sequence shown by SEQ ID NO:12 or SEQ ID NO:24 or a protein homologous thereto or a variant thereof.

### Tomatidine

A disease associated with tomatidine means a disease for which tomatidine is used or a disease corresponding to an adverse effect of tomatidine. Tomatidine is known as an alkaloid having a steroid skeleton similar to that of cyclopamine. Cyclopamine is a plant alkaloid contained in lilies and the like, and suppresses hedgehog signal transduction, which is important to embryogenesis or growth regulation in adult stem cells. Cyclopamine suppresses hedgehog signal transduction, which is accentuated in various cancer cells, mainly of gastrointestinal cancers, to exhibit anticancer action, whereas tomatidine does not inhibit this hedgehog signal transduction, and is therefore important to the design of a compound that regulates hedgehog signal transduction. An action associated with tomatidine can be closely relevant to a target protein (target gene) therefor, for example, a protein comprising the amino acid sequence shown by SEQ ID NO:24 or a protein homologous thereto or a variant thereof.

### Dipyrone

A disease associated with dipyrone means a disease for which dipyrone is used or a disease corresponding to an adverse effect of dipyrone. Dipyrone is known as a pyrine-series antipyretic analgesic anti-inflammatory drug. Examples of the action of dipyrone include pain relief/defervescence in acute upper airway inflammation and the like, and the like. In contrast, examples of the adverse effect of dipyrone include muco-cutaneo-ocular syndrome, toxic epidermal necrolysis, exfoliative dermatitis, aplastic anemia, agranulocytosis, granulocytopenia, jaundice, acute renal insufficiency and the like. An action associated with dipyrone can be closely relevant to a target protein (target gene) therefor, for example, a protein comprising the amino acid sequence shown by SEQ ID NO:24 or a protein homologous thereto or a variant thereof.

### Ethyl Loflazepate

A disease associated with ethyl loflazepate means a disease for which ethyl loflazepate is used or a disease corresponding to an adverse effect of ethyl loflazepate. Ethyl loflazepate is known as a benzodiazepine-series anti-anxiety drug (ultra-long action type, 90 hours or longer) or a persistent psychosomatic tranquilizer. Examples of the disease for which ethyl loflazepate is used include anxiety/tension/depression/sleep disorders in neurosis or psychosomatic disorders (gastric/duodenal ulcer, chronic gastritis, irritable bowel syndrome, autonomic imbalance) and the like. In contrast, examples of the adverse effect of ethyl loflazepate include excitation/confusion in schizophrenia patients, drowsiness, vertigo, headache, foggy vision, taste perversion, decrease in sexuality, increase in urinary urobilinogen and the like. An action associated with ethyl loflazepate can be closely relevant to a target protein (target gene) therefor, for example, a protein comprising the amino acid sequence shown by SEQ ID NO:24 or a protein homologous thereto or a variant thereof.

### Clobazam

A disease associated with clobazam means a disease for which clobazam is used or a disease corresponding to an adverse effect of clobazam. Clobazam is known as a benzodiazepine-series anti-epileptic drug. Examples of uses of clobazam include combination with other anti-epileptic drugs in partial seizures and generalized seizures and the like. In contrast, examples of the adverse effect of clobazam include drowsiness, vertigo, diplopia, anorexia, drug dependency due to high-dose continuous treatment, respiratory depression, increased sputum, airway hypersecretion, leukopenia, eosinophilia, thrombocytopenia and the like. An action associated with clobazam can be closely relevant to a target protein (target gene) therefor, for example, a protein comprising the amino acid sequence shown by SEQ ID NO:24 or a protein homologous thereto or a variant thereof.

### Alimemazine

A disease associated with alimemazine means a disease for which alimemazine is used or a disease corresponding to an adverse effect of alimemazine. Alimemazine is known as a phenothiazine-series anti-allergic drug (antihistamine drug). Examples of the disease for which alimemazine is used include pruritus that accompany skin diseases (eczema, skin pruritus, pediatric strophulus, toxicoderma, poisoning following pest bites) and urticaria, sneezing/nasal discharge/cough that accompany upper airway inflammations such as cold, allergic rhinitis and the like. In contrast, examples of the adverse effect of alimemazine include eruption, granulocytopenia, drowsiness, vertigo, general malaise, headache, dry mouth and the like. An action associated with alimemazine can be closely relevant to a target protein (target gene) therefor, for example, a protein comprising the amino acid sequence shown by SEQ ID NO:12 or a protein homologous thereto or a variant thereof.

### Ebastine

A disease associated with ebastine means a disease for which ebastine is used or a disease corresponding to an adverse effect of ebastine. Ebastine is a persistently acting basic histamine H₁ antagonist, and is known as an allergy therapeutic drug. Examples of the disease for which ebastine is used include urticaria, eczema/dermatitis, prurigo, skin pruritus, allergic rhinitis and the like. In contrast, examples of the adverse effect of ebastine include drowsiness, vertigo, general malaise, headache, dry mouth, liver dysfunction, anorexia, fever, eruption, itching and the like. An action associated with ebastine can be closely relevant to a target protein (target gene) therefor, for example, a protein comprising the amino acid sequence shown by SEQ ID NO:24 or a protein homologous thereto or a variant thereof.

### Reserpine

A disease associated with reserpine means a disease for which reserpine is used or a disease corresponding to an adverse effect of reserpine. Reserpine is a Rauwolfia alkaloid (R. serpentina), and is known as a hypotensive drug (peripheral sympathetic suppressant). Examples of the disease for which reserpine is used include hypertension (essential or renal and the like), malignant hypertension (in combination with other hypotensive agents), hypertensive emergencies (eclampsia, hypertensive encephalopathy, cerebral hemorrhagic stroke and the like), schizophrenia for which phenothiazine-series drugs cannot be used and the like. In contrast, examples of the adverse effect of reserpine include serious depressive states, suicide, nightmares, drowsiness, decrease in sexuality, nervous irritability, vertigo, extrapyramidal symptoms, bradycardia, edema, gastric ulcer, dry mouth, diarrhea, nausea, vomiting, soft feces, general malaise and the like. An action associated with reserpine can be closely relevant to a target protein (target gene) therefor, for example, a protein comprising the amino acid sequence shown by SEQ ID NO:12 or a protein homologous thereto or a variant thereof.

### Paramethasone

A disease associated with paramethasone means a disease for which paramethasone is used or a disease corresponding to an adverse effect of paramethasone. Paramethasone is known as an adrenocortical hormone preparation. Examples of the disease for which paramethasone is used include chronic adrenocortical dysfunction, acute adrenocortical dysfunction, adrenogenital syndrome, subacute thyroiditis, thyrotoxicosis, chronic articular rheumatism, lupus erythematosus, generalized angitis, multiple myositis, scleroderma, nephrosis, congestive heart failure, bronchial asthma, asthmatic bronchitis, severe infectious diseases (in combination with chemotherapy), hemolytic anemia (immunological), leukemia, granulocytopenia, purpura, aplastic anemia, regional enteritis, improvement of general condition in severe debilitating diseases (including terminal phase of cancer), fulminant hepatitis, cholestatic acute hepatitis, chronic hepatitis, liver cirrhosis, sarcoidosis, diffuse interstitial pneumonia (fibroid lung), severe pulmonary tuberculosis, tuberculous meningitis/pleurisy/peritonitis/pericarditis (in combination with antituberculous agents), encephalomyelitis, peripheral neuritis (including Guillain-Barrë syndrome), muscular ankylosis, multiple sclerosis, chorea minor, facial paralysis, spinal arachnoiditis, malignant lymphoma, eosinophilic granuloma, recurrence and metastasis of breast cancer, idiopathic hypoglycemia, snake venom/insect venom, ankylosing spondylitis, prevention of union after salpingoplasty, ovulation disturbance due to adrenocortical dysfunction, prostatic cancer, eczema/dermatitis group, tympanitis, allergic rhinitis, pollinosis (hay fever), intractable stomatitis and glossitis and the like. In contrast, examples of the adverse effect of paramethasone include moon face, skin symptoms, body weight gain, increased intraocular pressure/glaucoma during continuous treatment, induced infectious diseases, secondary adrenocortical dysfunction, digestive ulcer, diabetes mellitus, mental alteration, thrombosis, myopathy and the like. An action associated with paramethasone can be closely relevant to a target protein (target gene) therefor, for example, a protein comprising the amino acid sequence shown by SEQ ID NO:15 or a protein homologous thereto or a variant thereof.

### Hydroxyprogesterone

A disease associated with hydroxyprogesterone means a disease for which hydroxyprogesterone is used or a disease corresponding to an adverse effect of hydroxyprogesterone. Hydroxyprogesterone is known as a sex hormone preparation (luteinizing hormone preparation, progesterone derivative). Examples of the disease for which hydroxyprogesterone is used include amenorrhea, functional uterine hemorrhage, infertility due to corpus luteum dysfunction, threatened abortion/premature birth, habitual abortion/premature birth and the like. In contrast, examples of the adverse effect of hydroxyprogesterone include eruption, liver dysfunction, edema, body weight gain, headache, drowsiness, general malaise and the like. An action associated with hydroxyprogesterone can be closely relevant to a target protein (target gene) therefor, for example, a protein comprising the amino acid sequence shown by SEQ ID NO:12 or a protein homologous thereto or a variant thereof.

### Dydrogesterone

A disease associated with dydrogesterone means a disease for which dydrogesterone is used or a disease corresponding to an adverse effect of dydrogesterone. Dydrogesterone is known as a sex hormone preparation (luteinizing hormone preparation, progesterone derivative) and a synthetic luteinizing hormone. Examples of the disease for which dydrogesterone is used include threatened abortion/premature birth, habitual abortion/premature birth, amenorrhea, menstrual cycle abnormalities, dysmenorrhea, functional uterine hemorrhage, infertility due to corpus luteum dysfunction, endometriosis and the like. In contrast, examples of the adverse effect of dydrogesterone include nausea, anorexia, vomiting, eruption, liver dysfunction, edema, body weight gain, headache, drowsiness and the like. An action associated with dydrogesterone can be closely relevant to a target protein (target gene) therefor, for example, a protein comprising the amino acid sequence shown by SEQ ID NO:24 or a protein homologous thereto or a variant thereof.

### Sarpogrelate

A disease associated with sarpogrelate means a disease for which sarpogrelate is used or a disease corresponding to an adverse effect of sarpogrelate. Sarpogrelate is known as an antithrombotic agent (platelet aggregation suppressant). Examples of uses of sarpogrelate include amelioration of ischemic symptoms that accompany chronic arterial obstruction, such as ulcers/pain/psychroesthesia and the like. In contrast, examples of the adverse effect of sarpogrelate include nausea, heartburn, abdominal pain, cerebral hemorrhage, gastrointestinal hemorrhage, thrombocytopenia, liver dysfunction, jaundice and the like. An action associated with sarpogrelate can be closely relevant to a target protein (target gene) therefor, for example, a protein comprising the amino acid sequence shown by SEQ ID NO:15 or a protein homologous thereto or a variant thereof.

### Demeclocycline

A disease associated with demeclocycline means a disease for which demeclocycline is used or a disease corresponding to an adverse effect of demeclocycline. Demeclocycline is a tetracycline-series antibiotic, and is known as an inhibitor of the binding of aminoacyl tRNA to an mRNA-ribosome conjugate. Examples of the disease for which demeclocycline is used include infections with Rickettsia, Chlamydia trachomatis and the like, and the like. In contrast, examples of the adverse effect of demeclocycline include eruption, hyperesthesia optica, anorexia, nausea, vomiting, diarrhea, stomatitis, granulocytopenia, eosinophilia, thrombocytopenia and the like. An action associated with demeclocycline can be closely relevant to a target protein (target gene) therefor, for example, a protein comprising the amino acid sequence shown by SEQ ID NO:15 or a protein homologous thereto or a variant thereof.

### Avermectin B1A

A disease associated with avermectin B1A means a disease for which avermectin B1A is used or a disease corresponding to an adverse effect of avermectin B1A. Avermectin B1A is known as an antiparasitic/antiprotozoal drug. Examples of uses of avermectin B1A include insecticides/antiparasitic drugs (animal drugs) and the like. In contrast, examples of the adverse effect of avermectin B1A include nausea, diarrhea, eruption, itching, vertigo and the like. An action associated with avermectin B1A can be closely relevant to a target protein (target gene) therefor, for example, a protein comprising the amino acid sequence shown by SEQ ID NO:15 or a protein homologous thereto or a variant thereof.

### Solasodine

A disease associated with solasodine means a disease for which solasodine is used or a disease corresponding to an adverse effect of solasodine. Solasodine is known as a steroidal alkaloid of a glycoside ingredient contained in the plants of the eggplant family and the like. Examples of the disease for which solasodine is used include anticancer action, anaphylaxis or insulin shock, shock due to burns and the like. An action associated with solasodine can be closely relevant to a target protein (target gene) therefor, for example, a protein comprising the amino acid sequence shown by SEQ ID NO:24 or a protein homologous thereto or a variant thereof.

Nanofin (cis)

A disease associated with nanofin (cis) means a disease for which nanofin (cis) is used or a disease corresponding to an adverse effect of nanofin (cis). Nanofin (cis) is an alkaloid having nicotinic acetylcholine receptor antagonist action, and is known as a ganglionic blocker. An action associated with nanofin (cis) can be closely relevant to a target protein (target gene) therefor, for example, a protein comprising the amino acid sequence shown by SEQ ID NO:20 or a protein homologous thereto or a variant thereof.

### Methylbenzethonium Chloride

A disease associated with methylbenzethonium chloride means a disease for which methylbenzethonium chloride is used or a disease corresponding to an adverse effect of methylbenzethonium chloride. Methylbenzethonium chloride is known as a bactericidal agent and a cationic soap. An action associated with methylbenzethonium chloride can be closely relevant to a target protein (target gene) therefor, for example, a protein comprising the amino acid sequence shown by SEQ ID NO:23 or SEQ ID NO:24 or a protein homologous thereto or a variant thereof.

### α-Ergocryptine

A disease associated with α-ergocryptine means a disease for which α-ergocryptine is used or a disease corresponding to an adverse effect of α-ergocryptine. α-Ergocryptine is an ingredient of ergotoxin, and is known as a brain metabolism/peripheral circulation improver and a vasoconstrictor. Examples of the disease for which α-ergocryptine is used include incidental symptoms that accompany head trauma sequelae, hypertension (elderly patients, patients unable to achieve sufficient hypotensive effect by administration of diuretic hypotensive agents), peripheral circulatory disorders that accompany Burger disease/obstructive arteriosclerosis/arterial embolism/thrombosis/Raynaud's disease and Raynaud's syndrome/acrocyanosis/chilblain/frostbites and intermittent claudication and the like. In contrast, examples of the adverse effect of α-ergocryptine include gastrointestinal disorders, nausea/vomiting, anorexia, eruption/pruritus, headache/dull headache, vertigo, bradycardia, fall in BP, brain anemia-like symptoms, facial flush, sensation of hot flush, cardiac palpitation, chest discomfort and the like. An action associated with α-ergocryptine can be closely relevant to a target protein (target gene) therefor, for example, a protein comprising the amino acid sequence shown by SEQ ID NO:24 or a protein homologous thereto or a variant thereof.

### Spiramycin

A disease associated with spiramycin means a disease for which spiramycin is used or a disease corresponding to an adverse effect of spiramycin. Spiramycin is known as a macrolide-series antibiotic. Examples of the disease for which spiramycin is used include staphylococcus/streptococcus/pneumococcus/Treponema pallidum infections, carbuncles, furuncles, furunculosis, impetigo, felon, cellulitis, infectious atheroma, folliculitis, lymphadenitis, mastitis, myelitis, secondary infections following wounds/burns and surgery, infection with pharyngitis, tonsillitis, bronchitis, and bronchiectasis, pneumonia, pulmonary suppuration, cholecystitis, scarlatina, adnexitis, sties, acute dacryocystitis, tympanitis, syphilis and the like. In contrast, examples of the adverse effect of spiramycin include anorexia, nausea/vomiting, skin eruption/reddening, diarrhea, gastric discomfort and the like. An action associated with spiramycin can be closely relevant to a target protein (target gene) therefor, for example, a protein comprising the amino acid sequence shown by SEQ ID NO:6 or a protein homologous thereto or a variant thereof.

### Chloropyramine

A disease associated with chloropyramine means a disease for which chloropyramine is used or a disease corresponding to an adverse effect of chloropyramine. Chloropyramine is known as a first-generation antihistamine drug. Examples of the disease for which chloropyramine is used include allergic conjunctivitis, allergic rhinitis, bronchial asthma, atopic symptoms, Quincke's edema, various allergic symptoms and the like. In contrast, examples of the adverse effect of chloropyramine include excitation, vertigo, drowsiness, dry mouth, general malaise, constipation, visual disturbance and the like. An action associated with chloropyramine can be closely relevant to a target protein (target gene) therefor, for example, a protein comprising the amino acid sequence shown by SEQ ID NO:15 or a protein homologous thereto or a variant thereof.

### Bergenin

A disease associated with bergenin means a disease for which bergenin is used or a disease corresponding to an adverse effect of bergenin. Bergenin is a plant (strawberry saxifrage and the like) ingredient, and is known as an isocoumarine derivative having anti-ulcerative action. Examples of the disease for which bergenin is used include gastric ulcer and the like. An action associated with bergenin can be closely relevant to a target protein (target gene) therefor, for example, a protein comprising the amino acid sequence shown by SEQ ID NO:15 or a protein homologous thereto or a variant thereof.

### Nafcillin

A disease associated with nafcillin means a disease for which nafcillin is used or a disease corresponding to an adverse effect of nafcillin. Nafcillin is known as a penicillin-series antibiotic. Examples of the disease for which nafcillin is used include penicillin-resistant staphylococcus infections and the like. An action associated with nafcillin can be closely relevant to a target protein (target gene) therefor, for example, a protein comprising the amino acid sequence shown by SEQ ID NO:15 or a protein homologous thereto or a variant thereof.

### Carboprost

A disease associated with carboprost means a disease for which carboprost is used or a disease corresponding to an adverse effect of carboprost. Carboprost is a prostaglandin F₂a analogue, and is known as an abortion drug or an abortifacient. Examples of uses of carboprost include abortion or uterine contraction induction in hydatid mole treatment and the like. In contrast, examples of the adverse effect of carboprost include palpitation, headache, eruption, hysteralgia, hypothermia, spots, pectoralgia, sensation of chest pressure, dyspnea, constipation, diarrhea, vomiting and the like. An action associated with carboprost can be closely relevant to a target protein (target gene) therefor, for example, a protein comprising the amino acid sequence shown by SEQ ID NO:15 or a protein homologous thereto or a variant thereof.

(Diseases or Conditions Associated with Target Gene Y)

“A disease or condition associated with target gene Y” refers to a disease or condition that can be caused as a result of a functional change (e.g., functional changes due to mutations (e.g., polymorphism)), or a change in the expression level, in target gene Y, or in a gene located downstream of target gene Y in the signal transduction system mediated by target gene Y (downstream gene). A functional change in target gene Y or a gene downstream thereof can be caused by, for example, a mutation (e.g., polymorphism) in the gene. Examples of the mutation include a mutation in the coding region, which promotes or suppresses a function of the gene, a mutation in the non-coding region, which promotes or suppresses the expression thereof, and the like. The change in the expression level includes increases or reductions in the expression level. A disease or condition associated with target gene Y can be ameliorated or exacerbated by target protein Y.

“A function associated with target protein Y (target gene Y)” means a function of the same kind as, or opposite kind to, the kind of a function that is actually exhibited by target protein Y. In other words, a function associated with target protein Y is a function capable of ameliorating or exacerbating “a disease or condition associated with target protein Y”. Hence, “a function associated with target protein Y” is a function for promoting or suppressing an immune reaction, and the like, if target protein Y is a factor that promotes an immune reaction and the like. Examples of the function associated with target protein Y include the functions shown in Tables 2-1 to 2-4.

Since target gene Y is considered to mediate a wide variety of physiological functions in the body; as diseases or conditions associated with target protein Y, a very wide variety of diseases or conditions are supposed. Examples of the diseases or condition associated with target protein Y include diseases or conditions associated with the functions shown in Tables 2-1 to 2-4.

Other examples of the disease or condition associated with target protein Y are diseases or conditions postulated from the annotation of target protein Y and target gene Y. Those skilled in the art can postulate such diseases or conditions by identifying homologous proteins or genes by homology search, and subsequently extensively examining the functions of the proteins or genes or the diseases or conditions mediated thereby by a commonly known method. Various methods are available for annotation analysis. Described below are the results of annotation of target genes for bioactive substances in the present application, by various methods using the sequences of human proteins or genes representative of target proteins or genes for bioactive substances as query sequences.

### Amino Acid Analysis 1

### Homology Analysis by BLASTP

The calculation program used was blastall 2.2.6. The target databases used were swiss-prot: 146720 (Mar. 29, 2004), (Refseq)hs: 21170 (May 6, 2004), (Refseq)mouse: 17089 (May 6, 2004), and (Refseq)rat: 4893 (May 6, 2004). The cutoff value was established at 1.00E−05. The following data were processed by filtering:

### For Swiss-prot:

Having a definition beginning with “ALU SUBFAMILY”

Having a definition beginning with “Alu subfamily”

Having a definition beginning with “!!!! ALU SUBFAMILY”

Having a definition beginning with “B-CELL GROWTH FACTOR PRECURSOR”

Having a definition including “NRK2”

Having a definition beginning with “PROLINE-RICH”

Having a definition beginning with “GLYCINE-RICH”

Having a definition beginning with “EXTENSIN PRECURSOR”

Having a definition beginning with “COLLAGEN”

Having a definition beginning with ““OOKD”

Having a definition beginning with “RETROVIRUS-RELATED POL POLYPROTEIN”

Having a definition beginning with “CUTICLE COLLAGEN”

Having a definition beginning with “HYPOTHETICAL”

Having a definition beginning with “Hypothetical”

Having a definition beginning with “SALIVARY PROLINE-RICH PROTEIN”

Having a definition beginning with “IMMEDIATE-EARLY PROTEIN”

Having the accession number “P49646”

### For Ref-seq:

Having a definition beginning with “hypothetical protein FLJ”

Having a definition beginning with “KIAA”

Having a definition beginning with “hypothetical protein DKFZ”

Having a definition beginning with “DKFZ”

Having a definition beginning with “RIKEN cDNA”

Having a definition beginning with “hypothetical protein MGC”

Having a definition beginning with “hypothetical protein”

Having a definition beginning with “hypothetical protein PP”

Having a definition beginning with “neuronal thread protein”

Having a definition beginning with “clone FLB”

Having a definition beginning with “hypothetical protein PRO”

Having a definition as “PRO0483 protein”

Having a definition including “MNC”

Having a definition including “MOST-1”

Having a definition beginning with “similar to”

Having a definition including “TPR gene on Y”

Having a definition beginning with “HSPC”

Having a definition beginning with “CGI-”

ReFSeq sequence composed of self only (information referenced from LL_tmpl)

The annotation information obtained by this analysis is shown in Tables 3-1 to 3-4.

TABLE 3-1

| FLJ No | Accession No and definition | Key words |
|---|---|---|
| FLJ10420 | NP_954527.1 unknown (MGC: 72598 | |
| | protein) [rat] | |
| | NP_060560.1 hypothetical protein | |
| | FLJ10420 [human] | |
| FLJ10537 | Q9NR28 Diablo homolog, mitochondrial | transport peptide; |
| | precursor (second mitochondria-derived | mitochondria; |
| | activator of Caspase) (Smac | apoptosis; |
| | protein) (protein directly binding to | alternative |
| | IAP at low pI) | splicing; 3D- |
| | Q9JIQ3 Diablo homolog, mitochondrial | structure |
| | precursor (second mitochondria-derived | |
| | activator of Caspase) (Smac | |
| | protein) (protein directly binding to | |
| | IAP at low pI) | |
| | Q9NR28 Diablo homolog, mitochondrial | |
| | precursor (second mitochondria-derived | |
| | activator of Caspase) (Smac | |
| | protein) (protein directly binding to | |
| | IAP at low pI) | |
| | NP_620308.1 diablo isoform 2; second | |
| | mitochondria-derived activator of | |
| | Caspase; protein directly binding to | |
| | IAP at low pI; mitochondrial Smac | |
| | protein; 0610041G12Rik [human] | |
| | NP_063940.1 diablo isoform 1 | |
| | precursor; second mitochondria- | |
| | derived activator of Caspase; protein | |
| | directly binding to IAP at low pI; | |
| | mitochondrial Smac protein; | |
| | 0610041G12Rik [human] | |
| | NP_075721.2 protein directly binding | |
| | to IAP at low pI; second mitochondria- | |
| | derived activator of Caspase [mouse] | |
| | NP_620308.1 diablo isoform 2; second | |
| | mitochondria-derived activator of | |
| | Caspase; protein directly binding to | |
| | IAP at low pI; mitochondrial Smac | |
| | protein; 0610041G12Rik [human] | |
| FLJ11211 | NP_066968.1 Cyclin D-binding Myb-like | |
| FLJ41265 | transcription factor 1; Cyclin D- | |
| | binding Myb-like protein [human] | |
| | NP_035936.2 Cyclin D-binding Myb-like | |
| | transcription factor 1; D-interaction | |
| | Myb-like protein; Cyclin D-binding | |
| | Myb-like transcription factor 1; D- | |
| | interaction Myb-like protein [mouse] | |
| | NP_066968.1 Cyclin D-binding Myb-like | |
| | transcription factor 1; Cyclin D- | |
| | binding Myb-like protein [human] | |

TABLE 3-2

| | | |
|---|---|---|
| FLJ12857 | O75781 Paralemmin | cell morphology; |
| | Q9Z0P4 Paralemmin | membrane; |
| | Q9YGL6 Paralemmin | phosphorylation; |
| | O75781 Paralemmin | prenylation; |
| | NP_002570.1 Paralemmin [human] | lipoprotein; |
| | NP_075617.2 Paralemmin [mouse] | palmitate; coiled- |
| | NP_570842.1 Paralemmin [rat] | coil; alternative |
| | | splicing |
| FLJ20972 | NP_079306.1 hypothetical protein | |
| | FLJ20972 [human] | |
| FLJ21841 | P48681 Nestin | intermediate |
| | P21263 Nestin | filament; coiled- |
| | Q8IWN7 Retinitis pigmentosa 1-like 1 | coil; vision; |
| | protein | polymorphism; |
| | NP_006608.1 Nestin [human] | coiled-coil; |
| | NP_057910.2 Nestin; intermediate | repeat; alternative |
| | filament protein [mouse] | splicing |
| | NP_037119.1 Nestin [rat] | |
| FLJ22317 | NP_080912.1 RIKENcDNA5430437P03 | |
| FLJ20571 | [mouse] | |
| FLJ23466 | NP_071393.2 FK506-binding protein-like | |
| | [human] | |
| | NP_063926.1 FK506-binding protein- | |
| | like; Immunophilin-like protein NG7 | |
| | [mouse] | |
| FLJ25288 | P09525 Annexin A4 (Annexin | Annexin; |
| | IV) (Lipocortin IV) (Endonexin | calcium/ |
| | I) (Chromobindin 4) (protein | phospholipid |
| | II) (P32.5) (placenta anticoagulant | binding; repeat; |
| | protein II) (PAP-II) (PP4-X) (35-β | phosphorylation; |
| | Calcimedin) (carbohydrate-binding | acetylation; 3D- |
| | protein P33/P41) (P33/41) | structure |
| | P08132 Annexin A4 (Annexin | |
| | IV) (Lipocortin IV) (Endonexin | |
| | I) (Chromobindin 4) (protein | |
| | II) (P32.5) (placenta anticoagulant | |
| | protein I) (PAP-II) (PP4-X) (35-β | |
| | Calcimedin) | |
| | P13214 Annexin A4 (Annexin | |
| | IV) (Lipocortin IV) (Endonexin | |
| | I) (Chromobindin 4) (protein | |
| | II) (P32.5) (placenta anticoagulant | |
| | protein II) (PAP-II) (PP4-X) (35-β | |
| | Calcimedin) (carbohydrate-binding | |
| | protein P33/P41) (P33/41) | |
| | NP_001144.1 Annexin IV; Annexin IV | |
| | (placenta anticoagulant protein II); | |
| | placenta anticoagulant protein II | |
| | [human] | |
| | NP_077069.2 ZAP36/Annexin IV [rat] | |
| | NP_038499.1 Annexin A4; Annexin IV | |
| | [mouse] | |

TABLE 3-3

| | | |
|---|---|---|
| FLJ35914 | O00472 RNA polymerase II elongation | nuclear protein; |
| | factor ELL2 | transcription |
| | O08856 RNA polymerase II elongation | regulation; |
| | factor ELL (11-19 lysine-rich leukemia | chromosomal |
| | protein) | translocation; |
| | P55199 RNA polymerase II elongation | protooncogene |
| | factor ELL (11-19 lysine-rich leukemia | |
| | protein) | |
| | NP_666085.2 elongation factor RNA | |
| | polymerase II-like 3 [mouse] | |
| | NP_620403.1 elongation factor RNA | |
| | polymerase II 2; ELL-related RNA | |
| | polymerase II, elongation factor; 11-19 | |
| | lysine-rich leukemia gene 2 [mouse] | |
| | NP_079441.1 elongation factor RNA | |
| | polymerase II-like 3 [human] | |
| FLJ36526 | Q9UNZ2 NSFL1 cofactor p47 (p97 | Golgi stack; |
| | cofactor p47) | nuclear protein; |
| | O35987 NSFL1 cofactor p47 (p97 | lipid binding; |
| | cofactor p47) (XY body-related protein | phosphorylation; |
| | XY40) | alternative |
| | Q9CZ44 NSFL1 cofactor p47 (p97 | splicing; |
| | cofactor) | polymorphism; |
| | NP_114187.1 p47 protein [rat] | 3D-structure |
| | NP_938085.1 p47 protein; NSFL1 (p97) | |
| | cofactor (p47) homolog [mouse] | |
| | NP_057227.2 p47 protein isoform a | |
| | [human] | |
| FLJ37909 | P97822 acidic leucine-rich nuclear | leucine-rich |
| FLJ35353 | phosphoprotein 32 family member E | repeat; repeat; |
| | (LANP-like protein) (LANP-L) (cerebellar | nuclear protein; |
| | postnatal development protein 1) | alternative |
| | Q9BTT0 acidic leucine-rich nuclear | splicing; |
| | phosphoprotein 32 family member E | phosphorylation |
| | (LANP-like protein) (LANP-L) | |
| | P39687 acidic leucine-rich nuclear | |
| | phosphoprotein 32 family member A | |
| | (highly thermostable protein | |
| | phosphatase 2A inhibitor I1PP2A) (HLA- | |
| | DR-related protein I) (PHApI) (acidic | |
| | nuclear phosphoprotein | |
| | pp32) (cerebellar leucine-rich acidic | |
| | nuclear protein) | |
| | NP_112182.1 acidic (leucine-rich) | |
| | nuclear phosphoprotein 32 family, | |
| | member E; leucine-rich acidic nuclear | |
| | protein-like [human] | |
| | NP_006296.1 acidic (leucine-rich) | |
| | nuclear phosphoprotein 32 family, | |
| | member A; cerebellar leucine-rich | |
| | acidic nuclear protein; putative human | |
| | HLA-class II-related protein I; | |
| | inhibitor 1 of protein phosphatase-2A; | |
| | Mapmodulin [human] | |
| | NP_075699.2 acidic (leucine-rich) | |
| | nuclear phosphoprotein 32 family, | |
| | member E; cerebellar postnatal | |
| | development protein 1 [mouse] | |
| FLJ38531 | Q9W0Y1 Troponin C-akin-1 protein | hypothetical |
| | Q9W0Y2 hypothetical UPF0131 protein | protein |
| | CG2811 | |
| | NP_663441.1 cDNA sequence BC006662 | |
| | [mouse] | |
| FLJ38897 | P57764 DFN5-like protein FLJ12150 | |
| | NP_081236.1 RIKENcDNA1810036L03 | |
| | [mouse] | |
| FLJ39553 | NP_775597.1 cDNA sequence BC030476 | |
| | [mouse] | |
| | NP_775820.1 hypothetical protein | |
| | FLJ39553 [human] | |

TABLE 3-4

| | | |
|---|---|---|
| FLJ40298 | NP_775757.1 hypothetical protein | |
| | FLJ40298 [human] | |
| FLJ40760 | Q8WW22 DNAJ homolog subfamily | chaperon; |
| | A member 4 | repeat; |
| | Q9JMC3 DNAJ homolog subfamily A | zinc; metal |
| | member 4 (MmDjA4) | binding; |
| | P31689 DNAJ homolog subfamily A | prenylation; |
| | member 1 (heat shock 40 kDa protein 4) | lipoprotein; |
| | (DNAJ protein homolog 2) | multigene family |
| | (HSJ-2) (HSDJ) | |
| | NP_067397.1 heat shock protein, DNAJ- | |
| | like 4 [mouse] | |
| | NP_001530.1 DNAJ (Hsp40) homolog, | |
| | subfamily A, member 1; dj-2; hdj-2; | |
| | heat shock protein, DNAJ-like 2 | |
| | [human] | |
| | NP_061072.2 DNAJ (Hsp40) homolog, | |
| | subfamily A, member 4 [human] | |
| FLJ41550 | NP_570955.1 oxidation resistance 1; | |
| | nucleolus protein C7 [mouse] | |
| | NP_861447.2 nuclear receptor | |
| | coactivator 7; Estrogen receptor- | |
| | related protein 140 kDa [human] | |
| | NP_851999.1 oxidation resistance 1 | |
| | [human] | |
| FLJ41991 | P57764 DFN5-like protein FLJ12150 | |
| | NP_081236.1 RIKENcDNA1810036L03 | |
| | [mouse] | |
| FLJ42665 | P09104 γ Enolase (EC4.2.1.11) (2- | lyase; glycolysis; |
| | phospho-D-glycerate hydrolyase) (neural | magnesium; |
| | Enolase) (NSE) (Enolase 2) | multigene family; |
| | P17183 γ Enolase (EC4.2.1.11) (2- | polymorphism |
| | phospho-D-glycerate hydrolyase) (neural | |
| | Enolase) (NSE) (Enolase 2) | |
| | P07323 γ Enolase (EC4.2.1.11) (2- | |
| | phospho-D-glycerate hydrolyase) (neural | |
| | Enolase) (NSE) (Enolase 2) | |
| | NP_038537.1 Enolase 2, in γ neuron | |
| | [mouse] | |
| | NP_647541.1 Enolase 2, γ; Enolase 2, | |
| | γ, in neuron; Enolase 2 in γ neuron | |
| | [rat] | |
| | NP_001966.1 Enolase2; neuron specific | |
| | Enolase; neuron specific γ Enolase; 2- | |
| | phospho-D-glycerate hydrolyase; neural | |
| | Enolase [human] | |

### Amino Acid Analysis 2

### Motif Analysis by Pfam

The calculation program used was hmmpfam (v2.3.2). The target databases used were Pfam DB entry: 7426 families (Pfam13.0, Pfam_{—}1s). (April 2004). The cutoff value was established at 1E−10. The annotation information obtained by this analysis is shown by Table 4.

TABLE 4

| | PfamID and | |
|---|---|---|
| FLJ No | Pfam name | Pfam description |
| FLJ10420 | | |
| FLJ10537 | | |
| FLJ11211 | | |
| FLJ41265 | | |
| FLJ12857 | PF03285.4 | Paralemmin |
| | Paralemmin | |
| FLJ20972 | | |
| FLJ21841 | | |
| FLJ22317 | | |
| FLJ20571 | | |
| FLJ23466 | | |
| FLJ25288 | PF00191.7 Annexin | Annexin |
| FLJ35914 | PF07303.1 | Occludin and RNA |
| | Occludin_ELL | polymerase II |
| | | elongation factor ELL |
| FLJ36526 | PF00789.9 UBX | UBX domain |
| FLJ37909 | | |
| FLJ35353 | | |
| FLJ38531 | PF03674.4 UPF0131 | uncharacterized |
| | | protein family |
| | | (UPF0131) |
| FLJ38897 | PF04598.2 DFNA5 | DFNA5 protein |
| FLJ39553 | | |
| FLJ40298 | | |
| FLJ40760 | PF00226.13, | DnaJ domain, DnaJ |
| | PF00684.8 DnaJ, | central domain (4 |
| | DnaJ_CXXCXGXG | repeats) |
| FLJ41550 | PF07534.2 TLD | TLD |
| FLJ41991 | | |
| FLJ42665 | PF03952.3, PF00113.9 | Enolase, N-terminus |
| | Enolase_N, | domain |
| | Enolase_C | Enolase, C-turminus |
| | | TIM-barrel domain |

### Amino Acid Analysis 3

### Prediction of Secretory Signal Sequences by Signal IP

The calculation program used was SignalP ver 3.0 (May 18, 2004).

### Amino Acid Analysis 4

### Functional Categorization by GeneOntology

Performed per the procedures described below.

1) Extract results having E-values that meet the following conditions from among the results of homology analysis using BLASTP (RefSeq and SwissProt with filter) that produced three higher BLAST results (six in total).
Condition 1: Use all results having E-values of not more than 1E−50.
Condition 2: Do not use results having E-values of not less than 1E−10.
Condition 3: Use results having E-values exceeding 1E−50, provided that the difference in E-value from Top Hit is within 1E+20.
Condition 4: If the E-value of Top Hit is 0, use results having E-values of not more than 1E−50.
2) Search G0 by the keywords of SwissProt using spkw2go.
3) Search xref.goa by accession numbers of SwissProt to acquire Refseq IDs, further acquire LOCUS IDs by the Refseq IDs using LL_tmpl, and acquire G0 terms by the LOCUS IDs using loc2go.
4) Acquire LOCUS IDs by accession numbers of Refseq using LL_tmpl, and acquire G0 terms by the LOCUS IDs using loc2go.
5) Acquire information on higher categories for each G0 term acquired, with reference to the Molecular Function text file, Biological Process text file, and Cellular Component text file.
6) Remove overlapping information from the G0 term information acquired in 1)-5) above, and make an output.

The annotation information obtained by this analysis is shown in Tables 5-1 and 5-4.

TABLE 5-1

| | GO | |
|---|---|---|
| FLJ No | category | GO No (term) |
| FLJ10420 | MF | |
| | BP | |
| | CC | |
| FLJ10537 | MF | |
| | BP | GO: 0006915¥BP\|apoptosis; |
| | | GO: 0006917¥BP\|apoptosis induction; |
| | | GO: 0008625¥BP\|apoptosis induction via |
| | | death domain receptor; |
| | | GO: 0008635¥BP\|Caspase activation via |
| | | Cytochrome C |
| | CC | GO: 0005739¥CC\|mitochondrion |

TABLE 5-2

| | | |
|---|---|---|
| FLJ11211 | MF | GO: 0003677¥MF\|DNA binding; |
| FLJ41265 | | GO: 0003700¥MF\|transcription factor activity |
| | BP | GO: 0006355¥BP\|transcription regulation |
| | CC | GO: 0005634¥CC\|nucleus |
| FLJ12857 | MF | |
| | BP | GO: 0000902¥BP\|cell morphogenesis; |
| | | GO: 0006928¥BP\|cell motility; |
| | | GO: 0007010¥BP\|cytoskeleton organization and |
| | | biogenesis; GO: 0008360¥BP\|regulation of |
| | | cell shape |
| | CC | GO: 0005886¥CC\|plasma membrane; |
| | | GO: 0005887¥CC\|embedding in plasma membrane; |
| | | GO: 0009898¥CC\|inner membrane side of plasma |
| | | membrane; GO: 0016020¥CC\|membrane; |
| | | GO: 0016023¥CC\|cytoplasmic vesicle |
| FLJ20972 | MF | |
| | BP | |
| | CC | |
| FLJ21841 | MF | GO: 0005198¥MF\|structural molecule activity |
| | BP | GO: 0007417¥BP\|development of central |
| | | nervous system |
| | CC | GO: 0005882¥CC\|intermediate filament |
| FLJ22317 | MF | |
| FLJ20571 | BP | |
| | CC | |
| FLJ23466 | MF | GO: 0005554¥MF\|unknown molecular function |
| | BP | GO: 0009314¥BP\|response to irradiation |
| | CC | GO: 0008372¥CC\|unknown cell constituent |
| FLJ25288 | MF | GO: 0004859¥MF\|phospholipase inhibition |
| | | activity; GO: 0005509¥MF\|calcium ion |
| | | binding; GO: 0005544¥MF\|calcium ion- |
| | | dependent phospholipid binding |
| | BP | GO: 0001822¥BP\|kidney development; |
| | | GO: 0006887¥BP\|exocytosis |
| | CC | GO: 0016324¥CC\|apical plasma membrane |

TABLE 5-3

| | | |
|---|---|---|
| FLJ35914 | MF | GO: 0003746¥MF\|translation elongation factor |
| | | activity; GO: 0005515¥MF\|protein binding; |
| | | GO: 0008159¥MF\|positive transcription |
| | | elongation factor activity; |
| | | GO: 0016944¥MF\|Pol II transcription |
| | | elongation factor activity |
| | BP | GO: 0006354¥BP\|RNA elongation; |
| | | GO: 0006368¥BP\|RNA elongation from Pol II |
| | | promotor; GO: 0007283¥BP\|spermatogenesis; |
| | | GO: 0045817¥BP\|positive regulation of global |
| | | transcription from Pol II promotor |
| | CC | GO: 0005634¥CC\|nucleus; |
| | | GO: 0008023¥CC\|transcription elongation |
| | | factor complex; GO: 0016021¥CC\|embedding in |
| | | membrane |
| FLJ36526 | MF | GO: 0005515¥MF\|protein binding; |
| | | GO: 0008289¥MF\|lipid binding; |
| | | GO: 0008565¥MF\|protein transport activity; |
| | | GO: 0019905¥MF\|Syntaxin binding |
| | BP | GO: 0006886¥BP\|intracellular protein |
| | | transport; GO: 0006944¥BP\|membrane fusion; |
| | | GO: 0007030¥BP\|Golgi organization and |
| | | biogenesis; GO: 0009306¥BP\|protein |
| | | secretion; GO: 0015031¥BP\|protein transport; |
| | | GO: 0015758¥BP\|glucose transport |
| | CC | GO: 0005634¥CC\|nucleus; GO: 0005794¥CC\|Golgi |
| | | apparatus; GO: 0005829¥CC\|cytosol; |
| | | GO: 0005886¥CC\|plasma membrane |
| FLJ37909 | MF | GO: 0019212¥MF\|phosphatase inhibition |
| FLJ35353 | | activity |
| | BP | GO: 0000004¥BP\|unknown biological process; |
| | | GO: 0007242¥BP\|intracellular signaling |
| | | cascade |
| | CC | GO: 0005634¥CC\|nucleus; |
| | | GO: 0005737¥CC\|cytoplasm; |
| | | GO: 0016023¥CC\|cytoplasmic vesicle |
| FLJ38531 | MF | |
| | BP | |
| | CC | |
| FLJ38897 | MF | |
| | BP | |
| | CC | |
| FLJ39553 | MF | |
| | BP | |
| | CC | |
| FLJ40298 | MF | |
| | BP | |
| | CC | |
| FLJ40760 | MF | GO: 0003754¥MF\|chaperon activity; |
| | | GO: 0003773¥MF\|heat shock protein activity |
| | BP | GO: 0006457¥BP\|protein folding |
| | CC | |

TABLE 5-4

| | | |
|---|---|---|
| FLJ41550 | MF | GO: 0004872¥MF\|receptor activity; |
| | | GO: 0005554¥MF\|unknown molecular function |
| | BP | GO: 0006979¥BP\|responsiveness to oxidant |
| | | stress; GO: 0016998¥BP\|cell wall catabolism |
| | CC | GO: 0005730¥CC\|nucleolus; |
| | | GO: 0008372¥CC\|unknown cellular constituent |
| FLJ41991 | MF | |
| | BP | |
| | CC | |
| FLJ42665 | MF | GO: 0000287¥MF\|magnesium ion binding; |
| | | GO: 0004634¥MF\|phosphopyruvate hydrase |
| | | activity; GO: 0016829¥MF\|lyase activity |
| | BP | GO: 0006096¥BP\|glycolysis |
| | CC | GO: 0000015¥CC\|phosphopyruvate hydrase |
| | | complex |

### Nucleic Acid Analysis 1

### Homology Analysis 1 by BLASTX

The calculation program used was blastall 2.2.6. The target database used was nr:1552011(Jul. 16, 2004). The cutoff value was established at 1.00E−05. The following data were processed by filtering:

Having a definition beginning with “ALU SUBFAMILY”

Having a definition including “Alu subfamily”

Having a definition beginning with “!!!! ALU SUBFAMILY”

Beginning with “Drosophila melanogaster genomic scaffold”

Beginning with “Human DNA sequence from”

Including “genomic DNA”

Including “BAC clone”

Including “PAC clone”

Including “cosmid”

Including “complete genome”

Ending with “complete sequence”

Including “genomic sequence”

Including “exon”

A “HIT LENGTH (sequence length of the hit sequence) of not less than 50000 obtained by this analysis

The annotation information obtained by this analysis is shown in Tables 6-1 to 6-5.

TABLE 6-1

| FLJ No | nr accession No and definition |
|---|---|
| FLJ10420 | ref\|NP_060560.1\|hypothetical protein FLJ10420 |
| | [human]¥ dbj\|BAA91598.1\|protein product of |
| | unknown name [human]¥ gb\|AAH17014.1\|hypothetical |
| | protein FLJ10420 [human]¥ gb\|AAH18914.1\| |
| | hypothetical protein FLJ10420 [human] |
| | ref\|NP_079659.1\|RIKEN cDNA 1110005F07 |
| | [mouse]¥ dbj\|BAB22803.1\|protein product of unknown |
| | name [mouse]¥ gb\|AAH37069.1\|RIKEN cDNA 1110005F07 |
| | [mouse]¥ tpg\|DAA01434.1\|TPA: Adaptin-ear-binding |
| | coat-related protein 2; NECAP2 [mouse] |
| | dbj\|BAB14605.1\|protein product of unknown name |
| | [human] |
| | ref\|XP_216564.1\|similar to RIKEN cDNA 1110005F07 |
| | [rat] |
| | dbj\|BAB15758.1\|FLJ00061 protein [human] |
| FLJ10537 | gb\|AAG22077.1\|Smac/DIABLO-S protein [human] |
| | gb\|AAH04417.1\|unknown (protein of IMAGE: 3659131) |
| | [human] |
| | dbj\|BAB14994.1\|protein product of unknown name |
| | [human] |
| | ref\|NP_063940.1\|second mitochondria-derived |
| | activator of Caspase isoform Smac-α, precursor; |
| | IAP-directly binding protein having low pI; |
| | mitochondrial Smac protein |
| | [human]¥ sp\|Q9NR28\|SMAC_human Smac protein, |
| | mitochondrial precursor (second mitochondria- |
| | derived activator of Caspase) (IAP-directly |
| | binding protein having low pI)¥ gb\|AAF87716.1\| |
| | Smac [human]¥ gb\|AAH11909.1\|similar to second |
| | mitochondria-derived activator of Caspase [human] |
| | pdb\|1FEW\|A chain A, crystal structure of |
| | SmacDIABLO |
| FLJ11211 | gb\|AAH07418.2\|unknown (protein of IMAGE: 3010038) |
| FLJ41265 | [human] |
| | ref\|NP_066968.1\|Cyclin D-binding Myb-like |
| | transcription factor 1; Cyclin D-binging Myb-like |
| | protein [human]¥ gb\|AAC33480.1\|Cyclin D-binding |
| | Myb-like protein [human]¥ gb\|AAD43181.1\|Cyclin D- |
| | binding Myb-like protein [human] |
| | gb\|AAH07447.1\|similar to Cyclin D-binding Myb- |
| | like transcription factor 1 [human] |
| | ref\|NP_035936.21 Cyclin D-binding Myb-like |
| | transcription factor 1; D-interacting Myb-like |
| | protein; Cyclin D-binding Myb-like transcription |
| | factor 1 D-interacting Myb-like protein |
| | [mouse]¥ dbj\|BAC26325.1\|protein product of |
| | unknown name [mouse] |
| | gb\|AAC52878.1\|Cyclin D-interacting Myb-like |
| | protein [mouse] |

TABLE 6-2

| | |
|---|---|
| FLJ12857 | emb\|CAB37401.1\|PALM [human] |
| | emb\|CAA76152.1\|Paralemmin [human] |
| | dbj\|BAA13400.1\|KIAA0270 [human] |
| | sp\|O75781\|PALM_human Paralemmin ¥ emb\|CAB37400.1\| |
| | Paralemmin [human]¥ gb\|AAH32449.1\|Paralemmin |
| | [human] |
| | ref\|NP_002570.1\|Paralemmin |
| | [human]¥ emb\|CAA76151.1\|Paralemmin [human] |
| FLJ20972 | ref\|NP_079306.1\|hypothetical protein FLJ20972 |
| | [human]¥ dbj\|BAB14936.1\|protein product of |
| | unknown name [human] |
| | gb\|AAG35515.1\|PRO2550 [human] |
| | dbj\|BAC86300.1\|protein product of unknown name |
| | [human] |
| | dbj\|BAC85568.1\|protein product of unknown name |
| | [human] |
| | dbj\|BAA91131.1\|protein product of unknown name |
| | [human] |
| FLJ21841 | dbj\|BAB15153.1\|protein product of unknown name |
| | [human] |
| | sp\|P48681\|NEST_human Nestin¥ pir\|\|S21424 Nestin- |
| | human¥ emb\|CAA46780.1\|Nestin |
| | [human]¥ gb\|AAB24865.1\|Nestin = intermediate |
| | filament [human, peptide, 1618 aa] |
| | pir\|\|T34518 Nestin - golden hamster |
| | (fragment)¥ gb\|AAC98312.1\|Nestin [golden hamster] |
| | ref\|NP_057910.2\|Nestin; intermediate filament |
| | protein [mouse]\gb\|AAF04456.2\|intermediate |
| | filament protein Nestin [mouse] |
| | gb\|AAN33053.1\|Nestin [rat] |
| FLJ22317 | ref\|NP_054892.1\|HSPC142 protein |
| FLJ20571 | [human]¥ gb\|AAF29106.1\|HSPC142 [human] |
| | dbj\|BAA91268.1\|protein product of unknown name |
| | [human]¥ emb\|CAB66627.1\|hypothetical protein |
| | [human]¥ gb\|AAH00788.1\|HSPC142 protein |
| | [human]¥ gb\|AAH06244.1\|HSPC142 protein [human] |
| | ref\|XP_134254.1\|RIKEN cDNA 5430437P03 [mouse] |
| | dbj\|BAC36785.1\|protein product of unknown name |
| | [mouse] |
| | ref\|NP_080912.1\|RIKEN cDNA 5430437P03 |
| | [mouse]¥ gb\|AAH05692.1\|RIKEN cDNA 5430437P03 |
| | [mouse] |
| FLJ23466 | ref\|NP_071393.2\|FK506-binding protein-like |
| | [human]¥ gb\|AAF04864.1\|NG7 |
| | [human]¥ gb\|AAF67785.1\|DIR1 protein |
| | [human]¥ gb\|AAH04168.1\|FK506-binding protein-like |
| | [human]¥ gb\|AAH11966.1\|FK506-binding protein-like |
| | [human] |
| | dbj\|BAB15663.1\|protein product of unknown name |
| | [human] |
| | ref\|NP_063926.1\|FK506-binding protein-like; |
| | Immunophilin-like protein NG7 |
| | [mouse]¥ ref\|XP_283411.1\|FK506-binding protein- |
| | like [mouse]¥ ref\|XP_289855.1\|similar to FK506- |
| | binding protein-like; Immunophilin-like protein |
| | NG7; hypothetical protein NG7 [mouse]¥ pir\|\|T09068 |
| | Immunophilin-like protein NG7 - |
| | mouse¥ gb\|AAB82013.1\|unknown |
| | [mouse]¥ gb\|AAH46338.1\|FK506-binding protein-like |
| | [mouse] |
| | ref\|XP_218818.2\|similar to expression sequence |
| | AW538196 [rat] |
| | ref\|NP_598713.1\|expression sequence AW538196 |
| | [mouse]¥ gb\|AAH04717.1\|expression sequence |
| | AW538196 [mouse] |

TABLE 6-3

| | |
|---|---|
| FLJ25288 | ref\|NP_001144.1\|Annexin IV; Annexin IV (placenta |
| | anticoagulation protein II); placenta |
| | anticoagulation protein II [human]¥ pir\|\|A42077 |
| | Annexin IV - human¥ gb\|AAA51740.1\|Annexin IV |
| | (placenta anticoagulation protein |
| | II)¥ dbj\|BAA11227.1\|Annexin IV (carbohydrate- |
| | binding protein p33/41) [human]¥ gb\|AAH00182.1\| |
| | Annexin IV [human]¥ gb\|AAH11659.1\|Annexin IV |
| | [human] |
| | gb\|AAC41689.1\|protein PP4-X |
| | sp\|P09525\|ANX4_human Annexin A4 (Annexin IV) |
| | (Lipocortin IV) (Endoxin I) (Chromobindin 4) |
| | (protein II) (P32.5) (carbohydrate protein II) |
| | (PAP-II) (PP4-X) (35β Calcimedin) (carbohydrate- |
| | binding protein P33/P41) (P33/41) |
| | sp\|P08132\|ANX4_swine Annexin A4 (Annexin IV) |
| | (Lipocortin IV) (Endonexin I) (Chromobindin 4) |
| | (protein II) (P32.5) (placenta anticoagulation |
| | protein I) (PAP-II) (PP4-X) (35β Calcimedin) |
| | pir\|\|LUPG4 Annexin IV - swine |
| FLJ35914 | ref\|NP_079441.1\|elongation factor RNA polymerase |
| | II-like 3 [human]¥ dbj\|BAB15432.1\|protein product |
| | of unknown name [human]¥ gb\|AAH06548.1\|elongation |
| | factor RNA polymerase II-like 3 |
| | [human]¥ gb\|AAH19293.1\|elongation factor RNA |
| | polymerase II-like 3 [human] |
| | gb\|AAG13463.1\|RNA polymerase II elongation factor |
| | ELL3 [human] |
| | ref\|NP_666085.2\|elongation factor RNA polymerase |
| | II-like 3 [mouse]¥ gb\|AAH45151.1\|elongation |
| | factor RNA polymerase II-like 3 [mouse] |
| | ref\|XP_230508.2\|similar to hypothetical protein |
| | FLJ22637 [rat]¥ ref\|XP_347327.1\|similar to |
| | hypothetical protein FLJ22637 [rat] |
| | ref\|XP_350984.1\|similar to RIKEN cDNA A930015D22 |
| | [human] |
| FLJ36526 | gb\|AAD44488.1\|p47 [human] |
| | ref\|NP_057227.2\|P47 protein isoform a |
| | [human]¥ emb\|CAB96827.1\|dJ776F14.1 (P47 protein) |
| | [human]¥ gb\|AAH02801.1\|P47 protein, isoform a |
| | [human] |
| | gb\|AAP97139.1\|p47 [human] |
| | ref\|NP_114187.1\|P47 protein |
| | [rat]¥ dbj\|BAA21659.1\|p47 [rat]¥ emb\|CAA71742.1\| |
| | XY40 protein [rat] |
| | dbj\|BAB28604.1\|protein product of unknown name |
| | [mouse] |
| FLJ37909 | ref\|NP_075699.2\|acidic (leucine-rich) nuclear |
| FLJ35353 | phosphoprotein 32 family, member E; postnatal |
| | cerebellar development protein 1 |
| | [mouse]¥ dbj\|BAC33858.1\|protein product of |
| | unknown name [mouse] |
| | sp\|P97822\|A32E_mouse acidic leucine-rich nuclear |
| | phosphoprotein 32 family member E (LANP-like |
| | protein) (LANP-L) (postnatal cerebellar |
| | development protein-1)¥ gb\|AAB49462.2\|postnatal |
| | cerebellar development protein-1 |
| | [mouse]¥ dbj\|BAB03507.1\|LANP-like protein |
| | [mouse]¥ gb\|AAH05690.1\|acidic (leucine-rich) |
| | nuclear phosphoprotein 32 family, member E [mouse] |
| | dbj\|BAC40331.1\|protein product of unknown name |
| | [mouse] |
| | dbj\|BAC36147.1\|protein product of unknown name |
| | [mouse] |
| | ref\|XP_342299.1\|similar to acidic (leucine-rich) |
| | nuclear phosphoprotein 32 family, member E; |
| | leucine-rich acidic nuclear protein-like [rat] |

TABLE 6-4

| | |
|---|---|
| FLJ38531 | gb\|AAH04360.1\|unknown (protein of IMAGE: 2822295) |
| | [human] |
| | ref\|NP_149101.1\|hypothetical protein BC004360 |
| | [human]¥ gb\|AAH01077.2\|hypothetical protein |
| | BC004360 [human] |
| | ref\|NP_663441.1\|cDNA sequence BC006662 |
| | [mouse]¥ ref\|XP_354842.1\|cDNA sequence BC006662 |
| | [mouse]¥ gb\|AAH06662.1\|cDNA sequence BC006662 |
| | [mouse]¥ dbj\|BAC34147.1\|protein product of |
| | unknown name [mouse] |
| | ref\|XP_214256.2\|similar to cDNA sequence BC006662 |
| | [rat] |
| | ref\|XP_311398.1\|ENSANGP00000017416 |
| | [anopheline]¥ gb\|EAA07053.1\|ENSANGP00000017416 |
| | [anopheline str. PEST] |
| FLJ38897 | ref\|NP_079012.1\|hypothetical protein FLJ12150 |
| | [human]¥ sp\|P57764\|DF5L_human DFN5-like protein |
| | FLJ12150¥ dbj\|BAB13986.1\|protein product of |
| | unknown name [human]¥ gb\|AAH08904.1\|hypothetical |
| | protein FLJ12150 [human] |
| | gb\|AAG22861.1\|FKSG10 [human] |
| | ref\|XP_235434.2\|similar to RIKEN cDNA 1810036L03 |
| | [rat] |
| | ref\|NP_081236.1\|RIKEN cDNA 1810036L03 |
| | [mouse]¥ dbj\|BAB25204.1\|protein product of |
| | unknown name [mouse]¥ gb\|AAH29813.1\|RIKEN cDNA |
| | 1810036L03 [mouse] |
| | gb\|AAL55848.1\|unknown [human] |
| FLJ39553 | ref\|NP_775820.1\|hypothetical protein FLJ39553 |
| | [human]¥ dbj\|BAC04880.1\|protein product of |
| | unknown name [human] |
| | ref\|NP_775597.1\|cDNA sequence BC030476 |
| | [mouse]¥ gb\|AAH30476.1\|cDNA sequence BC030476 |
| | [mouse] |
| FLJ40298 | gb\|AAH31669.1\|similar to evidence: NAS-putative- |
| | unable to classify [human] |
| | ref\|XP_059377.6\|hypothetical protein FLJ40298 |
| | [human] |
| | dbj\|BAC05122.1\|protein product of unknown name |
| | [human] |
| | ref\|XP_126074.2\|RIKEN cDNA 4930505A04 [mouse] |
| | gb\|AAH49719.1\|unknown (protein for MGC: 58693) |
| | [mouse] |
| FLJ40760 | dbj\|BAC05229.1\|protein product of unknown name |
| | [human] |
| | ref\|NP_061072.2\|DnaJ (Hsp40) homolog, subfamily |
| | A, member 4 [human] |
| | sp\|Q8WW22\|DJA4_human DnaJ homolog subfamily A |
| | member 4¥ gb\|AAH21720.1\|DnaJ (Hsp40) homolog, |
| | subfamily A, member 4 [human]¥ dbj\|BAC04828.1\| |
| | protein product of unknown name [human] |
| | gb\|AAP22730.1\|pDJA1 chaperon [boar] |
| | ref\|XP_217147.2\|similar to mmDj4 [rat] |
| FLJ41550 | ref\|XP_293104.2\|similar to dJ132F21.2 (containing |
| | a novel protein similar to drosophila L82E) |
| | [human] |
| | emb\|CAC10465.1\|dJ132F21.2 (containing a novel |
| | protein similar to drosophila L82E) [human] |
| | ref\|XP_141508.1\|similar to dJ132F21.2 (containing |
| | a novel protein similar to drosophila L82E) |
| | [human] [mouse] |
| | ref\|XP_357232.1\|similar to dJ132F21.2 (containing |
| | a novel protein similar to drosophila L82E) |
| | [mouse] |
| | gb\|AAG25715.1\|oxidation protection protein |
| | [human] |

TABLE 6-5

| | |
|---|---|
| FLJ41991 | ref\|NP_079012.1\|hypothetical protein FLJ12150 |
| | [human]¥sp\|P57764\|DF5L_HUMAN DFN5-like protein |
| | FLJ12150¥dbj\|BAB13986.1\|protein product of |
| | unknown name [human]¥gb\|AAH08904.1\|hypothetical |
| | protein FLJ12150 [human] |
| | gb\|AAG22861.1\|FKSG10 [human] |
| | ref\|XP_235434.2\|similar to RIKEN cDNA 1810036L03 |
| | [rat] |
| | gb\|AAL55848.1\|unknown [human] |
| | ref\|NP_081236.1\|RIKEN cDNA 1810036L03 |
| | [mouse]¥ dbj\|BAB25204.1\|protein product of |
| | unknown name [mouse]¥ gb\|AAH29813.1\|RIKEN cDNA |
| | 1810036L03 [mouse] |
| FLJ42665 | gb\|AAP88878.1\|Enolase2, (γ, nerve) [synthetic |
| | construct] |
| | ref\|NP_001966.1\|Enolase 2; Enolase-2, γ, nerve; |
| | nerve specific Enolase; nerve specific γ Enolase; γ |
| | Enolase; 2-phospho-D-glycerate hydrolyase; neural |
| | Enolase [human]¥ sp\|P09104\|ENOG_human γ Enolase |
| | (2-phospho-D-glycerate hydrolyase) (neural |
| | Enolase) (NSE) (Enolase 2)¥ pir\|\|NOHUG |
| | phosphopyruvate hydrase (EC 4.2.1.11) γ- |
| | human¥ emb\|CAA36215.1\|human γ Enolase |
| | [human]¥ gb\|AAB59554.1\|Enolase¥ gb\|AAB51320.1\| |
| | nerve specific γ-Enolase [human]¥ gb\|AAH02745.1\| |
| | Enolase 2 [human]¥ gb\|AAP36047.1\|Enolase 2, (γ, |
| | nerve) [human] |
| | emb\|CAA31512.1\|nerve specific Enolase |
| | [human]¥ emb\|CAA32505.1\|γ Enolase [human] |
| | ref\|NP_038537.1\|Enolase 2, γ nerve |
| | [mouse]¥ sp\|P17183\|ENOG_mouse γ Enolase (2- |
| | phospho-D-glycerate hydrolyase) (neural Enolase) |
| | (NSE) (Enolase 2)¥ pir\|\|S10247 phosphopyruvate |
| | hydratase (EC 4.2.1.11) γ-mouse¥ emb\|CAA36606.1\| |
| | protein product of unknown name [house |
| | mouse]¥ gb\|AAC36002.1\|ENO2 |
| | [mouse]¥ dbj\|BAB22533.1\|protein product of |
| | unknown name [mouse]¥ gb\|AAH31739.1\|Enolase 2, γ |
| | nerve [mouse] |
| | ref\|NP_647541.1\|Enolase 2, γ; Enolase 2, γ, nerve |
| | [rat]¥ sp\|P07323\|ENOG_rat γ Enolase (2-phospho-D- |
| | glycerate hydrolyase) (neural Enolase) (NSE) |
| | (Enolase 2)¥ pir\|\|A24742 phosphopyruvate hydratase |
| | (EC 4.2.1.11) γ-rat¥ gb\|AAA41119.1\|nerve specific |
| | Enolase¥ emb\|CAA30556.1\|Enol_cds |
| | [rat]¥ gb\|AAB72088.1\|nerve specific Enolase |
| | [rat]¥ gb\|AAH60310.1\|Enolase 2, γ |
| | [rat]¥ prf\|\|1302225A Enolase γ, nerve specific |

### Nucleic Acid Analysis 2

### Homology Analysis 2 by BLASTX

The calculation program used was blastall 2.2.6. The target databases used were swiss-prot:146720 (Mar. 29, 2004), (Refseq)hs:21170 (May 6, 2004), (Refseq)mouse:17089 (May 6, 2004), and (Refseq)rat:4893 (May 6, 2004). The cutoff value was established at 1.00E−05. The following data were processed by filtering:

### For Swiss-prot:

Having a definition beginning with “ALU SUBFAMILY”

Having a definition beginning with “Alu subfamily”

Having a definition beginning with “!!!! ALU SUBFAMILY”

Having a definition beginning with “B-CELL GROWTH FACTOR PRECURSOR”

Having a definition including “NRK2”

Having a definition beginning with “PROLINE-RICH”

Having a definition beginning with “GLYCINE-RICH”

Having a definition beginning with “EXTENSIN PRECURSOR”

Having a definition beginning with “COLLAGEN”

Having a definition beginning with “100 KD”

Having a definition beginning with “RETROVIRUS-RELATED POL POLYPROTEIN”

Having a definition beginning with “CUTICLE COLLAGEN”

Having a definition beginning with “HYPOTHETICAL”

Having a definition beginning with “Hypothetical”

Having a definition beginning with “SALIVARY PROLINE-RICH PROTEIN”

Having a definition beginning with “IMMEDIATE-EARLY PROTEIN”

Having the accession No “P49646”

### For Ref-seq:

Having a definition beginning with “hypothetical protein FLJ”

Having a definition beginning with “KIAA”

Having a definition beginning with “hypothetical protein DKFZ”

Having a definition beginning with “DKFZ”

Having a definition beginning with “RIKEN cDNA”

Having a definition beginning with “hypothetical protein MGC”

Having a definition beginning with “hypothetical protein”

Having a definition beginning with “hypothetical protein PP”

Having a definition beginning with “neuronal thread protein”

Having a definition beginning with “clone FLB”

Having a definition beginning with “hypothetical protein PRO”

Having a definition as “PRO0483 protein”

Having a definition including “MNC”

Having a definition including “MOST-1”

Having a definition beginning with “similar to”

Having a definition including “TPR gene on Y”

Having a definition beginning with “HSPC”

Having a definition beginning with “CGI-”

ReFSeq sequence composed of self only (information referenced from LL_tmpl)

The annotation information obtained by this analysis is shown in Tables 7-1 to 7-5.

TABLE 7-1

| FLJ No | Accession No and definition | Key words |
|---|---|---|
| FLJ10420 | NP_954527.1 unknown (MGC: 72598 | |
| | protein) [rat] | |
| | NP_060560.1 hypothetical | |
| | protein FLJ10420 [human] | |
| FLJ10537 | Q9NR28 Diablo homolog, | transport |
| | mitochondrial precursor (second | peptide; |
| | mitochondria-derived activator | mitochondria; |
| | of Caspase) (Smac | apoptosis; |
| | protein) (protein directly | alternative |
| | binding to IAP at low pI) | splicing; 3D- |
| | Q9JIQ3 Diablo homolog, | structure; Ubl |
| | mitochondrial precursor (second | conjugation |
| | mitochondria-derived activator | passway; |
| | of Caspase) (Smac | ligase; nuclear |
| | protein) (protein directly | protein; |
| | binding to IAP at low pI) | repeat; |
| | Q96J02 Itchy homolog E3 | phosphorylation |
| | ubiquitin protein ligase | |
| | (EC6.3.2.—) (Itch) (Atrophin 1- | |
| | interacting protein | |
| | 4) (AIP4) (NFE2-related | |
| | polypeptide 1) (NAPP1) | |
| | NP_620308.1 diablo isoform 2; | |
| | second mitochondria-derived | |
| | activator of Caspase; protein | |
| | directly binding to IAP at low | |
| | pI; mitochondrial Smac protein; | |
| | 0610041G12Rik [human] | |
| | NP_063940.1 diablo isoform 1 | |
| | precursor; second mitochondria- | |
| | derived activator of Caspase; | |
| | protein directly binding to IAP | |
| | at low pI; mitochondrial Smac | |
| | protein; 0610041G12Rik [human] | |
| | NP_075721.2 protein directly | |
| | binding to IAP at low pI; | |
| | second mitochondria-derived | |
| | activator of Caspase [mouse] | |

TABLE 7-2

| | | |
|---|---|---|
| FLJ11211 | NP_066968.1 Cyclin D-binding | |
| FLJ41265 | Myb-like transcription factor | |
| | 1; Cyclin D-binding Myb-like | |
| | protein [human] | |
| | NP_035936.2 Cyclin D-binding | |
| | Myb-like transcription factor | |
| | 1; D-interacting Myb-like | |
| | protein; Cyclin D-binding Myb- | |
| | like transcription factor 1D- | |
| | interacting Myb-like protein | |
| | [mouse] | |
| FLJ12857 | O75781 Paralemmin | cell shape; |
| | Q9Z0P4 Paralemmin | membrane; |
| | Q9YGL6 Paralemmin | phosphorylation; |
| | NP_075617.2 Paralemmin [mouse] | prenylation; |
| | NP_570842.1 Paralemmin [rat] | lipoprotein; |
| | NP_002570.1 Paralemmin [human] | palmitate; |
| | | coiled-coil; |
| | | alternative |
| | | splicing |
| FLJ20972 | Q96J02 Itchy homolog E3 | Ubl conjugation |
| | ubiquitin protein ligase | pathway; |
| | (EC6.3.2.—) (Itch) (Atrophin 1- | ligase; nuclear |
| | interacting protein | protein; |
| | 4) (AIP4) (NFE2-related | repeat; |
| | polypeptide 1) (NAPP1) | phosphorylation; |
| | Q92918 mitotic division | alternative |
| | activated protein kinase kinase | splicing; ATP- |
| | kinase kinase | binding; |
| | 1(EC2.7.1.37) (MAPK/ERK kinase | transferase; |
| | kinase kinase 1) (MEK kinase | kinase; |
| | kinase 1) (MEKKK1) (blood | serine/tyrosine |
| | progenitor kinase) | protein kinase |
| | Q99959 Plakophilin 2 | cell adhesion; |
| | P39193 Alu subfamily SP | cytoskeleton; |
| | sequence contamination warning | structure |
| | entry | protein; |
| | NP_858931.1 NFS1 nitrogen | hypothetical |
| | fixation 1 isoform b precursor; | protein |
| | cysteine desulphurase; nitrogen | |
| | fixation bacteria S-like | |
| | protein [human] | |
| | NP_061913.2 elongation protein | |
| | 4 homolog; PAX6 adjucent gene; | |
| | chromosome 11 Open Reading | |
| | Flame 19 [human] | |
| | NP_803191.1 FtsJ homolog 2 | |
| | isoform b; chromosome protein | |
| | FtsJ; rRNA(urigine-2′-O-)- | |
| | methyltransferase [human] | |
| | NP_079306.1 hypothetical | |
| | protein FLJ20972 [human] | |
| FLJ21841 | P48681 Nestin | intermediate |
| | P21263 Nestin | filament; |
| | Q00780 Collagen α1 (VIII) chain | coiled-coil; |
| | precursor | extracellular |
| | NP_057910.2 Nestin; | matrix; |
| | intermediate filament protein | connective |
| | [mouse] | tissue; repeat; |
| | NP_037119.1 Nestin [rat] | hydroxylation; |
| | NP_006608.1 Nestin [human] | glycoprotein; |
| | | cell adhesion; |
| | | collagen; |
| | | signal |
| FLJ22317 | NP_054892.1 HSPC142 protein | |
| FLJ20571 | [human] | |
| FLJ23466 | NP_063926.1 FK506-binding | |
| | protein-like; Immunophilin-like | |
| | protein NG7 [mouse] | |
| | NP_598713.1 expressed sequence | |
| | AW538196 [mouse] | |
| | NP_071393.2 FK506-binding | |
| | protein-like [human] | |

TABLE 7-3

| | | |
|---|---|---|
| FLJ25288 | P09525 Annexin A4 (Annexin | annexin; |
| | IV) (Lipocortin IV) (Endonexin | calcium/phospho |
| | I) (Chromobindin 4) (protein | lipid binding; |
| | II) (P32.5) (placenta | repeat; |
| | anticoagulant protein II) (PAP- | phosphorylation; |
| | II) (PP4-X) (35-β | acetylation; |
| | Calcimedin) (carbohydrate- | 3D-structure |
| | binding protein | |
| | P33/P41) (P33/41) | |
| | P08132 Annexin A4 (Annexin | |
| | IV) (Lipocortin IV) (Endonexin | |
| | I) (Chromobindin 4) (protein | |
| | II) (P32.5) (placenta | |
| | anticoagulant protein I) (PAP- | |
| | II) (PP4-X) (35-β Calcimedin) | |
| | P13214 Annexin A4 (Annexin | |
| | IV) (Lipocortin IV) (Endonexin | |
| | I) (Chromobindin 4) (protein | |
| | II) (P32.5) (placenta | |
| | anticoagulant protein II) (PAP- | |
| | II) (PP4-X) (35-β | |
| | Calcimedin) (carbohydrate- | |
| | binding protein | |
| | P33/P41) (P33/41) | |
| | NP_077069.2 ZAP36/Annexin IV | |
| | [rat] | |
| | NP_038499.1 Annexin A4; Annexin | |
| | IV [mouse] | |
| | NP_001144.1 Annexin IV; Annexin | |
| | IV (placenta anticoagulant | |
| | protein II); placenta | |
| | anticoagulant protein II | |
| | [human] | |
| FLJ35914 | O00472 RNA polymerase II | nuclear |
| | elongation factor ELL2 | protein; |
| | O08856 RNA polymerase II | transcription; |
| | elongation factor ELL (11-19 | chromosomal |
| | lysine-rich leukemia protein) | translocation; |
| | P55199 RNA polymerase II | protooncogene |
| | elongation factor ELL (11-19 | |
| | lysine-rich leukemia protein) | |
| | NP_666085.2 elongation factor | |
| | RNA polymerase II-like 3 | |
| | [mouse] | |
| | NP_036213.1 elongation factor, | |
| | RNA polymerase II, 2; ELL- | |
| | related RNA polymerase II, | |
| | elongation factor [human] | |
| | NP_079441.1 elongation factor | |
| | RNA polymerase II-like 3 | |
| | [human] | |
| FLJ36526 | Q9UNZ2 NSFL1 cofactor p47 (p97 | Golgi stack; |
| | cofactor p47) | nuclear |
| | O35987 NSFL1 cofactor p47 (p97 | protein; lipid |
| | cofactor p47) (XY body-related | binding; |
| | protein XY40) | phosphorylation; |
| | Q9CZ44 NSFL1 cofactor p47 (p97 | alternative |
| | cofactor) | splicing; |
| | NP_114187.1 p47 protein [rat] | polymorphism; |
| | NP_938085.1 p47 protein; NSFL1 | 3D-structure |
| | (p97) cofactor (p47) homolog | |
| | [mouse] | |
| | NP_057227.2 p47 protein isoform | |
| | a [human] | |

TABLE 7-4

| | | |
|---|---|---|
| FLJ37909 | P97822 acidic leucine-rich nuclear | leucine-rich |
| FLJ35353 | phosphoprotein 32 family member E | repeat; |
| | (LANP-like protein) (LANP- | repeat; |
| | L) (postnatal cerebellar development | nuclear |
| | protein 1) | protein; |
| | Q9BTT0 acidic leucine-rich nuclear | alternative |
| | phosphoprotein 32 family member E | splicing; |
| | (LANP-like protein) (LANP-L) | phosphorylation |
| | P51122 acidic leucine-rich nuclear | |
| | phosphoprotein 32 family member A | |
| | (leucine-rich acidic nuclear | |
| | protein) (fragment) | |
| | NP_112182.1 acidic (leucine-rich) | |
| | nuclear phosphoprotein 32 family, | |
| | member E; leucine-rich acidic | |
| | nuclear protein-like [human] | |
| | NP_006296.1 acidic (leucine-rich) | |
| | nuclear phosphoprotein 32 family, | |
| | member A; cerebellar leucine-rich | |
| | acidic nuclear protein; putative | |
| | human HLA class II-related protein | |
| | I; inhibitor of protein phosphatase | |
| | 2A; Mapmodulin [human] | |
| | NP_075699.2 acidic (leucine-rich) | |
| | nuclear phosphoprotein 32 family, | |
| | member E; postnatal cerebellar | |
| | development protein 1 [mouse] | |
| FLJ38531 | Q9W0Y1 Troponin C-akin-1 protein | hypothetical |
| | Q9W0Y2 hypothetical UPF0131 protein | protein |
| | CG2811 | |
| | NP_663441.1 cDNA sequence BC006662 | |
| | [mouse] | |
| FLJ38897 | P57764 DFN5-like protein FLJ12150 | |
| | NP_067322.1 Gasdermin [mouse] | |
| | NP_113603.1 melanoma-derived leucine | |
| | zipper, extranuclear factor [human] | |
| | NP_835465.1 Gasdermin [human] | |
| | NP_081236.1 RIKENcDNA1810036L03 | |
| | [mouse] | |
| FLJ39553 | NP_775597.1 cDNA sequence BC030476 | |
| | [mouse] | |
| | NP_775820.1 hypothetical protein | |
| | FLJ39553 [human] | |
| FLJ40298 | NP_775757.1 hypothetical protein | |
| | FLJ40298 [human] | |
| FLJ40760 | Q8WW22 DnaJ homolog subfamily A | chaperon; |
| | member 4 | repeat; |
| | Q9JMC3 DnaJ homolog subfamily A | zinc; metal |
| | member 4 (MmDjA4) | binding; |
| | P31689 DnaJ homolog subfamily A | prenylation; |
| | member 1 (heat shock 40 kDa protein | lipoprotein; |
| | 4) (DnaJ protein homolog2) (HSJ- | multigene |
| | 2) (HSDJ) | family |
| | NP_067397.1 heat shock protein, | |
| | DNAJ-like 4 [mouse] | |
| | NP_001530.1 DnaJ (Hsp40) homolog, | |
| | subfamily A, member 1; dj-2; hdj-2; | |
| | heat shock protein, DNAJ-like 2 | |
| | [human] | |
| | NP_061072.2 DnaJ (Hsp40) homolog, | |
| | subfamily A, member 4 [human] | |
| FLJ41550 | Q9Y3Z3 SAM domain and HD domain- | interferon |
| | containing protein 1 (dendritic | induction |
| | cell-derived IFNG induced | |
| | protein) (DCIP) (monocyte protein | |
| | 5) (MOP-5) | |
| | NP_570955.1 oxidation resistance 1; | |
| | nucleolus protein C7 [mouse] | |
| | NP_861447.2 nuclear receptor | |
| | coactivator 7; estrogen receptor- | |
| | related protein 140 kDa [human] | |
| | NP_851999.1 oxydation resistance 1 | |
| | [human] | |

TABLE 7-5

| | | |
|---|---|---|
| FLJ41991 | P57764 DFN5-like protein FLJ12150 | |
| | NP_067322.1 Gasdermin [mouse] | |
| | NP_835465.1 Gasdermin [human] | |
| | NP_113603.1 melanoma-derived | |
| | leucine zipper, extranuclear factor | |
| | [human] | |
| | NP_081236.1 RIKENcDNA1810036L03 | |
| | [mouse] | |
| FLJ42665 | P09104 γ Enolase (EC4.2.1.11) (2- | lyase; |
| | phospho-D-glycerate | glycolysis; |
| | hydrolyase) (neural | magnesium; |
| | Enolase) (NSE) (Enolase 2) | multigene |
| | P17183 γ Enolase (EC4.2.1.11) (2- | family; |
| | phospho-D-glycerate | polymorphism |
| | hydrolyase) (neural | |
| | Enolase) (NSE) (Enolase 2) | |
| | P07323 γ Enolase (EC4.2.1.11) (2- | |
| | phospho-D-glycerate | |
| | hydrolyase) (neural | |
| | Enolase) (NSE) (Enolase 2) | |
| | NP_038537.1 Enolase 2, in γ neuron | |
| | [mouse] | |
| | NP_647541.1 Enolase 2, γ; Enolase | |
| | 2, γ, in neuron; Enolase 2 in γ | |
| | neuron [rat] | |
| | NP_001966.1 Enolase 2; nerve | |
| | specific Enolase; nerve specific γ | |
| | Enolase; 2-phospho-D-glycerate | |
| | hydrolyase; neural Enolase [human] | |

Other examples of possible diseases or conditions are the diseases or conditions registered with OMIM. These diseases or conditions can easily be searched by, for example, inputting H-Inv ID numbers or H-Inv cluster ID numbers in H-Inv DB. The chromosomes and gene loci where the target genes for bioactive substances in this application are present, and OMIM information on orphan diseases expected to be associated with these genes, are shown in Tables 8-1 to 8-5.

TABLE 8-1

| | | | OMIM disease information |
|---|---|---|---|
| FLJ No | chromosome | Genome locus | (OMIM Co-localized orphan disease) |
| FLJ10420 | 1 | 16148312-16167654, | OMIM129900: ectrodactyly, ectodermal dysplasia, cleft lip and palate syndrome 1; EEC1 |
| | | plus | OMIM603511: muscular dystrophy, limb-girdle, type 1D; LGMD1D |
| | | | OMIM606595: Charcot-Marie-Tooth disease, axon, type 2F |
| | | | OMIM608027: cerebellar atrophy that accompany progressive microcephalia |
| FLJ10537 | 12 | 121129840-121147923, | OMIM121400: cornea plana 1; CNA1 |
| | | minus | OMIM605583: deafness, autosomal dominant nonsyndromic sensorineural deafness 25; DFNA25 |
| FLJ11211 | 7 | 86429451-86437675, | OMIM608027: cerebellar atrophy that accompany progressive microcephalia |
| FLJ41265 | | plus | OMIM606595: Charcot-Marie-Tooth disease, axon, type 2F |
| | | | OMIM129900: ectrodactyly, ectodermal dysplasia, cleft lip and palate syndrome 1; EEC1 |
| | | | OMIM603511: muscular dystrophy, limb-girdle, type 1D; LGMD1D |
| FLJ12857 | 19 | 678562-699328, plus | none |
| FLJ20972 | 1 | 42361642-42416950, | OMIM606713: Van Der Woude syndrome 2 |
| FLJ21841 | 1 | 153855347-153863991, | OMIM605913: bleeding disease, East Texas type |
| | | minus | OMIM605549: retinal cone-rod dystrophy 8; CORD8 |
| | | | OMIM608091: cerebellooculorenal syndrome 2; CORS2 |
| | | | OMIM601412: deafness, autosomal dominant nonsyndromic sensorineural deafness 7; DFNA7 |

TABLE 8-2

| | | | |
|---|---|---|---|
| FLJ22317 | 19 | 17239252-17251146, | OMIM606482: Charcot-Marie-Tooth disease, dominant-intermediate B |
| FLJ20571 | | plus | OMIM604781: fishskin disease, nonlayered and non-erythrodermic, congenital, autosomal |
| | | | recessive; NNCI |
| | | | OMIM607324: hyperdactylism, postaxial, A3 type |
| FLJ23466 | 6 | 32167952-32169533, | OMIM606766: aspermia, nonobstructive |
| | | minus | OMIM607017: deafness, autosomal dominant nonsyndromic sensorineural deafness 21; DFNA21 |
| | | | OMIM212750: Celiac disease; CD |
| | | | OMIM242650: primary ciliary dyskinesia; PCD |
| | | | OMIM137100: immunoglobulin A deficiency disease susceptibility 1; IGAD1 |
| | | | OMIM601086: lateral ectrodactylia, autosomal dominant |
| | | | OMIM607085: myasthenia gravis associated with hyperplasia of thymus |
| | | | OMIM272370: lysis susceptibility due to congeneric (allogenic) reactive natural killer |
| | | | cell; EC1 |
| | | | OMIM177900: psoriasis susceptibility |
| | | | OMIM271250: spinocerebellar ataxia associated with blindness and deafness |
| FLJ25288 | 2 | 69943800-70028135, | OMIM605711: multiple mitochondria dysfunction syndrome |
| | | plus | OMIM606068: pigmentosa 28; RP28 |
| | | | OMIM604454: Welander distal myopathy; WDM |

TABLE 8-3

| | | | |
|---|---|---|---|
| FLJ35914 | 15 | 41780871-41807387, | OMIM602099: amyotropic lateral sclerosis retinitis 5; ALS5 |
| | | minus | OMIM601228: colon and rectal adenoid tumor and cancer 1; CRAC1 |
| | | | OMIM134600: Fanconi Renotubular syndrome |
| | | | OMIM604329: hypertension, essential, susceptibility, 2 |
| | | | OMIM214900: calcification-lymphedema syndrome |
| | | | OMIM604321: microcephalia, mainly autosomal recessive, 4; MCP |
| | | | OMIM605738: microphthalmia 2; NN02 |
| | | | OMIM605275: Noonan syndrome 2 |
| | | | OMIM604360: spastic paraplegia 11, autosomal recessive; SPG11 |
| FLJ36526 | 20 | 1419184-1443337, | none |
| | | minus | |
| FLJ37909 | 1 | 147407437-147423608, | OMIM605803: dermatitis, atopy, 2; ATOD2 |
| FLJ35353 | | minus | OMIM605549: retinal cone-rod dystrophy 8; CORD8 |
| | | | OMIM608091: cerebellooculorenal syndrome 2; CORS2 |
| | | | OMIM601412: deafness, autosomal dominant non-syndromic perception deafness 7; DFNA7 |
| | | | OMIM174000: medullary cystic renal diseases 1; MCKD1 |
| | | | OMIM603935: bronchocele, papillary, psoriasis susceptible 4; PSORS4 |
| | | | OMIM605642: thyroid adenoma, papillary, accompanied by papillary renal neogenesis |
| FLJ38531 | 13 | 98880520-98939047, | none |
| | | minus | |
| FLJ38897 | 8 | 144745660-144750365, | OMIM600668: cartilage mineralization 1; CCAL1 |
| | | plus | OMIM140300: Hashimoto's thyroiditis |
| | | | OMIM145700: hypertrichosis universalis |
| | | | OMIM601068: benign adult familial myoclonic epilepsy, type 1 |
| | | | OMIM607361: meckel syndrome, type 3; MKS3 |
| | | | OMIM603563: spastic paraplegia 8, autosomal dominant; SPG8 |

TABLE 8-4

| | | | |
|---|---|---|---|
| FLJ39553 | 8 | 99033333-99062421, | OMIM600668: cartilage mineralization 1; CCAL1 |
| | | plus | OMIM148900: Klippel-Feli syndrome |
| FLJ40298 | 2 | 54532605-54563248, | OMIM160980: Carney complex, type 1; CNC1 |
| | | plus | OMIM605244: Carney complex, type II; CNC2 |
| | | | OMIM604254: alexia, specific, 3; DYX3 |
| FLJ40760 | 15 | 76272315-76290355, | OMIM606937: cerebellar ataxia associated with amentia, atrophy of eye, cutaneous |
| | | plus | abnormality |
| | | | OMIM105600: anemia, congenital abnormal hematopoiesis, type III; CDAN3 |
| | | | OMIM606451: deafness, autosomal dominant nonsyndromic perception deafness 30; DFNA30 |
| | | | OMIM607728: porokeratosis, sporadic skin-deep chemical rays, 2 |
| | | | OMIM603204: epilepsy, nocturnal frontal lobe, type 2 |
| | | | OMIM603813: hypercholesterolemia, autosomal recessive; ARH |
| | | | OMIM604329: hypertension, essential, susceptibility, 2 |
| | | | OMIM214900: calcification-lymphedema syndrome |
| | | | OMIM602685: disease associated with amentia, spasticity and pigmented tapetoretinal |
| | | | degeneration |
| FLJ41550 | | | |

TABLE 8-5

| | |
|---|---|
| FLJ41991 | OMIM140300: Hashimoto's thyroiditis |
| | OMIM145700: hypertrichosis universalis |
| | OMIM600668: cartilage mineralization 1; CCAL1 |
| | OMIM601068: epilepsy, myoclonus, benign adult familial, |
| | type 1 |
| | OMIM603563: spastic paraplegia 8, autosomal dominant; |
| | SPG8 |
| | OMIM607361: meckel syndrome, type 3; MKS3 |
| FLJ42665 | OMIM140300: Hashimoto's thyroiditis |
| | OMIM145700: hypertrichosis universalis |
| | OMIM600668: cartilage mineralization 1; CCAL1 |
| | OMIM601068: epilepsy, myoclonus, benign adult familial, |
| | type 1 |
| | OMIM603563: spastic paraplegia 8, autosomal dominant; |
| | SPG8 |
| | OMIM607361: meckel syndrome, type 3; MKS3 |

Other possible diseases or conditions are diseases or conditions accompanied by abnormalities at expression sites of target gene Y, or in tissues from which the library for target gene Y is derived. The expression sites and tissues can easily be searched by, for example, inputting H-Inv cDNA ID numbers or H-Inv locus ID numbers in H-Inv DB, whereby those skilled in the art are able to postulate the diseases or conditions.

For example, some of target gene Y are expressed at the sites shown below.

FLJ10420-derived protein can be expressed in brain stem, cerebellum, corpus callosum, glia, aorta, bone marrow, lymph nodes, blood, spleen, veins, ovary, placenta, prostate, testis, muscles, colon, liver, lungs, kidneys, adrenals, mammary glands, pancreas and the like.

FLJ10537-derived protein can be expressed in brain stem, cerebrum, eyes, bone marrow, spleen, thymus, placenta, prostate, testis, small intestine, adrenals and the like.

FLJ11211- and FLJ41265-derived protein can be expressed in brain stem, cerebellum, corpus callosum, glia, spinal cord, lymph nodes, blood, spleen, thymus, bone, skin, uterus, placenta, prostate, testis, muscles, colon, stomach, lungs, adrenals, mammary glands, pancreas, pituitary, thyroid and the like.

FLJ12857-derived protein can be expressed in brain stem, cerebrum, cerebellum, corpus callosum, glia, eyes, spinal cord, spleen, adipose tissue, skin, placenta, prostate, testis, liver, kidneys, pancreas, pituitary and the like.

FLJ20972-derived protein can be expressed in brain stem, cerebellum, lymph nodes, thymus, uterus, muscles, colon, lungs, kidneys and the like.

FLJ21841-derived protein can be expressed in cerebellum, corpus pineale, spinal cord, aorta, spleen, skin, uterus, ovary, prostate, heart, muscles, colon, stomach, urinary bladder, kidneys, adrenals and the like.

FLJ22317- and FLJ20571-derived protein can be expressed in brain stem, cerebrum, cerebellum, eyes, corpus pineale, aorta, bone marrow, blood, spleen, veins, bone, skin, uterus, ovary, placenta, prostate, testis, heart, muscles, colon, small intestine, liver, lungs, kidneys, adrenals, mammary glands, pancreas and the like.

FLJ23466-derived protein can be expressed in corpus callosum, glia, eyes, spinal cord, skin, placenta, prostate, testis, muscles, colon, stomach, liver, kidneys, adrenals, mammary glands, salivary gland and the like.

FLJ25288-derived protein can be expressed in corpus callosum, glia, placenta, prostate, muscles, colon, small intestine, stomach, liver, lungs, urinary bladder, kidneys, mammary glands, pancreas and the like.

FLJ35914-derived protein can be expressed in cerebellum, lymph nodes, skin, testis, colon, small intestine, liver, lungs, kidneys, adrenals, mammary glands, thyroid and the like.

FLJ36526-derived protein can be expressed in cerebellum, corpus callosum, glia, eyes, corpus pineale, bone marrow, blood, adipose tissue, bone, skin, uterus, placenta, prostate, heart, muscles, colon, small intestine, liver, lungs, kidneys, adrenals, mammary glands, pituitary, salivary gland and the like.

FLJ37909- and FLJ35353-derived protein can be expressed in cerebrum, cerebellum, spinal cord, aorta, bone marrow, blood, ovary, placenta, prostate, testis, heart, muscles, colon, liver, lungs, kidneys, adrenals, mammary glands, pancreas and the like.

FLJ38531-derived protein can be expressed in aorta, ovary, testis, colon, liver, mammary glands and the like.

FLJ38897-derived protein can be expressed in eyes, aorta, bone marrow, lymph nodes, spleen, thymus, adipose tissue, placenta, muscles, colon, small intestine, lungs, kidneys, mammary glands, pancreas and the like.

FLJ39553-derived protein can be expressed in eyes, placenta, mammary glands and the like.

FLJ40298-derived protein can be expressed in kidneys and the like.

FLJ40760-derived protein can be expressed in brain stem, cerebellum, corpus callosum, glia, spinal cord, bone marrow, spleen, skin, placenta, prostate, testis, heart, muscles, colon, stomach, liver, lungs, kidneys, adrenals, mammary glands and the like.

Still other examples of possible diseases or conditions are diseases or conditions mediated by genes that are homologous to target gene Y or a gene downstream thereof. Those skilled in the art are able to postulate such diseases or conditions by identifying homologous genes by homology search, and then extensively investigating the diseases or conditions involved by the homologous genes by a commonly known method.

The target proteins and target genes of the present invention are useful for, for example, the development of drugs for specified diseases or conditions, or the development of investigational reagents for the diseases or conditions.

### 2. Screening Methods and Products Obtained by the Methods

The present invention provides screening methods for bioactive substances, each of which comprises determining whether or not a test substance is capable of regulating the expression or function of a target protein for the bioactive substance or a gene that encodes the protein (hereinafter sometimes referred to as “target protein Y” or “target gene Y” as required), and a product thereof. The screening methods of the present invention can be roughly divided into two types, from the viewpoint of the kind of bioactive substance screened: screening methods for substances capable of regulating an action associated with bioactive substance X, and screening methods for substances capable of regulating a function associated with target protein Y. The screening methods of the present invention can also be performed in vitro, in vivo or in silico. The individual screening methods are hereinafter described in detail.

2.1. Screening Methods for Substances Capable of Regulating an Action Associated with Bioactive Substance X (Screening Method I)

The present invention provides screening methods for substances capable of regulating an action associated with bioactive substance X, each of which comprises determining whether or not a test substance is capable of regulating the expression or function of target protein Y.

The screening methods of this type are generically referred to as “screening method I” as required.

Screening method I can be roughly divided into two types: a screening method for a substance capable of regulating an action associated with bioactive substance X, which comprises determining whether or not a test substance is capable of regulating the expression or function of target protein Y, and selecting a test substance capable of regulating the expression or function of target protein Y (screening method Ia), and a screening method for a substance capable of regulating an action associated with bioactive substance X (particularly an action associated with a known target molecule), which comprises determining whether or not a test substance is capable of regulating the expression or function of target protein Y, and selecting a test substance that is incapable of regulating the expression or function of target protein Y (screening method Ib). Screening method Ia can be useful for the development of regulators of diseases or conditions associated with bioactive substance X and the like. Screening method Ib can be useful for the development of drugs capable of regulating an action associated with a known target molecule, and showing decreased adverse effects of bioactive substance X and the like.

2.1.1. Screening Method for Substances Capable of Regulating an Action Associated with Bioactive Substance X, which Comprises Selecting a Test Substance Capable of Regulating the Expression or Function of Target Protein Y (Screening Method Ia)

The present invention provides a screening method for substances capable of regulating an action associated with bioactive substance X, which comprises determining whether or not a test substance is capable of regulating the expression or function of target protein Y, and selecting a test substance capable of regulating the expression or function of target protein Y.

The test substance subjected to this screening method may be any known compound or new compound; examples include nucleic acids, carbohydrates, lipids, proteins, peptides, organic small compounds, compound libraries prepared using combinatorial chemistry technique, random peptide libraries prepared by solid phase synthesis or the phage display method, or natural components derived from microorganisms, animals, plants, marine organisms and the like, and the like. The test substance may be a labeled supply or a non-labeled supply, or a mixture of a labeled supply and a non-labeled supply mixed in a specified ratio. The labeling substance is the same as described above.

In one embodiment, screening method Ia comprises the following steps (a), (b) and (c):

(a) a step for bringing the test substance into contact with target protein Y;
(b) a step for measuring the functional level of the protein in the presence of the test substance, and comparing this functional level with the functional level of the protein in the absence of the test substance;
(c) a step for selecting a test substance that alters the functional level of the protein on the basis of the result of the comparison in step (b) above.

The methodology comprising the above-described steps (a) to (c) is referred to as “methodology I” as required.

In step (a) of methodology I, a test substance is so brought into contact with target protein Y. Contact of the test substance with the protein can be performed by contact of isolated target protein Y and the test substance in solution, or contact of cells or tissue capable of expressing target protein Y and the test substance.

Target protein Y can be prepared by a method known per se. For example, target protein Y can be isolated and purified from the above-described expression tissue. However, to prepare target protein Y quickly, easily, and in large amounts, and to prepare human target protein Y, it is preferable to prepare a recombinant protein by gene recombination technology. The recombinant protein may be prepared using a cell system or a cell-free system.

The cells capable of expressing target protein Y can be any cells that express target protein Y; examples include cells derived from the tissue in which target protein Y is expressed, cells transformed with target protein Y expression vector and the like. Those skilled in the art are able to easily identify or prepare these cells; useful cells include primary culture cells, cell lines derivatively prepared from the primary culture cells, commercially available cell lines, cell lines available from cell banks, and the like. As the tissue capable of expressing target protein Y, the above-described expression tissues can be used.

In step (b) of methodology I, the functional level of the protein in the presence of the test substance is measured. A measurement of the functional level can be performed according to the kind of protein by a method known per se. For example, provided that target protein Y is a transcription factor, a substance that regulates a function associated with target protein Y can be screened by performing a reporter assay using target protein Y and a transcription regulatory region to which it binds.

Provided that target protein Y is an enzyme, the functional level can also be measured on the basis of a change in the catalytic activity of the enzyme. The catalytic activity of the enzyme can be measured by a method known per se using a substrate, coenzyme and the like chosen as appropriate according to the kind of enzyme.

Furthermore, provided that target protein Y is a membrane protein (e.g., receptors, transporters), the functional level can be measured on the basis of a change in a function of the membrane protein. For example, provided that target protein Y is a receptor, a screening method of the present invention can be performed on the basis of an intracellular event mediated by the receptor (e.g., inositol phospholipid production, intracellular pH change, intracellular behavior of ions such as calcium ion and chlorine ion). Provided that target protein Y is a transporter, a screening methods of the present invention can be performed on the basis of a change in the intracellular concentration of a substrate for the transporter.

The functional level may also be measured on the basis of the functional level of target protein Y to each isoform (e.g., splicing variant) or the isoform-isoform functional level ratio, rather than on the basis of the total functional level of target protein Y.

Next, the functional level of target protein Y in the presence of the test substance is compared with the functional level of target protein Y in the absence of the test substance. This comparison of functional level is preferably performed on the basis of the presence or absence of a significant difference. Although the functional level of target protein Y in the absence of the test substance may be measured prior to, or simultaneously with, the measurement of the functional level of target protein Y in the presence of the test substance, it is preferable, from the viewpoint of experimental accuracy and reproducibility, that the functional level be measured simultaneously.

In step (c) of methodology I, a test substance that alters the functional level of the protein is selected. The test substance that alters the functional level of the protein is capable of promoting or suppressing a function of target protein Y. The test substance thus selected can be useful for the regulation of a disease or condition associated with bioactive substance X.

Methodology I may be performed not only in the presence of target protein Y but also with a coupling factor thereof. For example, when target protein Y inhibitory factor is used in combination as the coupling factor of target protein Y, a substance that interferes with the interaction between target protein Y and the coupling factor is considered to be capable of promoting a function of target protein Y. When target protein Y activation factor is used in combination as the coupling factor for target protein Y, a substance that interferes with the interaction between target protein Y and the coupling factor is considered to be capable of suppressing a function of target protein Y. Hence, it is also beneficial to perform methodology I in the presence of a coupling factor of target protein Y.

In another embodiment, screening method Ia comprises the following steps (a), (b) and (c):

(a) a step for bringing the test substance and cells enabling a measurement of the expression of target protein Y or a gene that encodes the protein into contact with each other;
(b) a step for measuring the expression level in the cells in contact with the test substance, and comparing this expression level with the expression level in control cells not in contact with the test substance;
(c) a step for selecting a test substance that regulates the expression level on the basis of the result of the comparison in step (b) above.

The methodology comprising the above-described steps (a) to (c) is referred to as “methodology II” as required.

In step (a) of methodology II, a test substance is brought into contact with cells enabling a measurement of the expression of target protein Y. Contact of the test substance with the cells enabling a measurement of the expression of target protein Y can be performed in culture medium.

“Cells enabling a measurement of the expression of target protein Y or a gene that encodes the protein (referred to as “target gene Y” as required)” refers to cells enabling a direct or indirect evaluation of the expression level of a product of target gene Y, for example, a transcription product or translation product (i.e., protein). The cells enabling a direct evaluation of the expression level of a product of target gene Y can be cells capable of naturally expressing target gene Y, whereas the cells enabling an indirect evaluation of the expression level of a product of target gene Y can be cells enabling a reporter assay on target gene Y transcription regulatory region.

The cells capable of naturally expressing target gene Y can be any cells that potentially express target gene Y; examples include cells showing constitutive expression of target gene Y, cells that express target gene Y under inductive conditions (e.g., drug treatment) and the like. Those skilled in the art are able to easily identify these cells; useful cells include primary culture cells, cell lines derivatively prepared from the primary culture cells, commercially available cell lines, cell lines available from cell banks, and the like.

The cells enabling a reporter assay on target gene Y transcription regulatory region are cells incorporating target gene Y transcription regulatory region and a reporter gene functionally linked to the region. The target gene Y transcription regulatory region and reporter gene are inserted in an expression vector.

The target gene Y transcription regulatory region may be any region enabling the control of the expression of target gene Y; examples include a region from the transcription initiation point to about 2 kbp upstream thereof, and a region consisting of a base sequence wherein one or more bases are deleted, substituted or added in the base sequence of the region, and that is capable of controlling the transcription of target gene Y, and the like.

The reporter gene may be any gene that encodes a detectable protein or enzyme; examples include the GFP (green fluorescent protein) gene, GUS (β-glucuronidase) gene, LUS (luciferase) gene, CAT (chloramphenicol acetyltransferase) gene and the like.

The cells transfected with target gene Y transcription regulatory region and a reporter gene functionally linked to the region are not subject to limitation, as long as they enable an evaluation of target gene Y transcription regulatory function, that is, as long as they enable a quantitative analysis of the expression level of the reporter gene. However, the cells transfected are preferably cells capable of naturally expressing target gene Y because they are considered to express a physiological transcription regulatory factor for target gene Y, and to be more appropriate for the evaluation of the regulation of the expression of target gene Y.

The culture medium in which a test substance and cells enabling a measurement of the expression of target gene Y are brought into contact with each other is chosen as appropriate according to the kind of cells used and the like; examples include minimal essential medium (MEM) containing about 5 to 20% fetal bovine serum, Dulbecco's modified minimal essential medium (DMEM), RPMI1640 medium, 199 medium and the like. Culture conditions are also determined as appropriate according to the kind of cells used and the like; for example, the pH of the medium is about 6 to about 8, culture temperature is normally about 30 to about 40° C., and culture time is about 12 to about 72 hours.

In step (b) of methodology II, first, the expression level of target gene Y in the cells in contact with the test substance is measured. This measurement of expression level can be performed by a method known per se in view of the kind of cells used and the like.

For example, when cells capable of naturally expressing target gene Y are used as the cells enabling a measurement of the expression of target gene Y, the expression level can be measured by a method known per se with a product of target gene Y, for example, a transcription product or translation product, as the subject. For example, the expression level of a transcription product can be measured by preparing total RNA from the cells, and performing RT-PCR, Northern blotting and the like. The expression level of a translation product can be measured by preparing an extract from the cells, and performing an immunological technique. Useful immunological techniques include radioimmunoassay (RIA) method, ELISA method (Methods in Enzymol. 70: 419-439 (1980)), fluorescent antibody technique and the like.

When cells enabling a reporter assay on target gene Y transcription regulatory region are used as the cells enabling a measurement of the expression of target gene Y, the expression level can be measured on the basis of the signal intensity of the reporter.

The expression level may also be measured on the basis of the expression level of target gene Y to each isoform (e.g., splicing variant) or the isoform-isoform expression ratio, rather than on the basis of the total expression level of target gene Y.

Next, the expression level of target gene Y in the cells in contact with the test substance is compared with the expression level of target gene Y in control cells not in contact with the test substance. This comparison of expression level is preferably performed on the basis of the presence or absence of a significant difference. Although the expression level of target gene Y in the control cells not in contact with the test substance may be measured prior to, or simultaneously with, the measurement of the expression level of target gene Y in the cells in contact with the test substance, it is preferable, from the viewpoint of experimental accuracy and reproducibility, that the expression level be measured simultaneously.

In step (c) of methodology II, test substance that regulates the expression level of target gene Y is selected. The regulation of the expression level of target gene Y can be the promotion or suppression of the expression level. The test substance thus selected can be useful for the regulation of an action associated with bioactive substance X.

Methodology II can further comprise (d) (i) a step for confirming that the selected test substance is capable of regulating, for example, promoting or suppressing, an action associated with bioactive substance X (confirmation step), or (ii) a step for identifying the kind of action exhibited by the selected test substance (identification step). The confirmation step or identification step can be performed by, for example, administering the selected test substance to a normal animal, or to an animal with “a disease or condition associated with bioactive substance X” or model animal. According to this identification step, the kind of “action associated with bioactive substance X” exhibited by the selected test substance can be determined, and whether or not the selected test substance can be used as either a drug or an investigational reagent, or both, and the kind of drug or investigational reagent to which the test substance is applicable can be confirmed.

In another embodiment, screening method Ia comprises the following steps (a), (b) and (c):

(a) a step for bringing the test substance into contact with target protein Y;
(b) a step for measuring the ability of the test substance to bind to the protein;
(c) a step for selecting a test substance capable of binding to the protein on the basis of the results of step (b) above.

The methodology comprising the above-described steps (a) to (c) is referred to as “methodology III” as required.

In step (a) of methodology III, a test substance is brought into contact with target protein Y. Contact of the test substance with the protein can be performed by mixing the test substance and the protein in solution.

Target protein Y can be prepared by a method known per se. For example, target protein Y can be isolated and purified from the above-described target gene Y expression tissue. However, to prepare target protein Y quickly, easily, and in large amounts, and to prepare human target protein Y, it is preferable to prepare a recombinant protein by gene recombination technology. The recombinant protein may be prepared using a cell system or a cell-free system.

In step (b) of methodology III, the ability of the test substance to bind to the protein is measured. “a binding ability” measured may be any one that enables an evaluation of the binding of the protein and the test substance; examples include binding amount, binding strength (including parameters such as affinity constant, binding rate constant, and dissociation rate constant), and binding mode (including dose-dependent binding).

A measurement of the binding ability can be performed by, for example, the SEC/MS method (size exclusion chromatography/mass analysis) (see Moy, F. J. et al., Anal. Chem., 2001, 73, 571-581). The SEC/MS method comprises (1) a step for adding a mixed multiplexed compound sample to the purified protein, and then separating the free compound and the protein by SEC, and (2) an analytical step for identifying the bound compound contained in the protein fraction by MS. The SEC/MS method is advantageous in that the binding ability can be analyzed while both the protein and the test substance are in non-modified and non-immobilized state. In the SEC/MS method, not only the ability of the test substance to bind to the protein, but also the dose dependency of the test substance in the binding to the protein and the like can be measured simultaneously.

A measurement of the binding ability can also be performed using a means for measurement based on surface plasmon resonance, for example, Biacore. Using Biacore, the binding and dissociation of a test substance to a protein immobilized on a chip are measured, and the measured values are compared with those obtained when a solution not containing the test substance is loaded on the chip. Subsequently, a test substance capable of binding to the protein is selected on the basis of the result for the binding and dissociation rate or binding amount. Biacore also enables simultaneous measurements of binding strength (e.g., Kd value) and the like, in addition to the ability of a test substance to bind to a protein.

Other methods for measuring the binding ability include, for example, SPR-based methods or optical methods such as the quartz crystal microbalance (QCM) method, the dual polarization interferometer (DPI) method, and the coupled waveguide plasmon resonance method, immunoprecipitation, isothermal titration and differential scanning calorimetry, capillary electrophoresis, energy transfer, fluorescent analytical methods such as fluorescent correlation analysis, and structural analytical methods such as X-ray crystallography and nuclear magnetic resonance (NMR).

In measuring the binding ability, a target protein Y-binding substance can also be used as a control.

“A target protein Y-binding substance” is a compound capable of interacting directly with target protein Y or a mutated protein thereof, and can be, for example, a protein, a nucleic acid, a carbohydrate, a lipid, or a small organic compound. Preferably, the target protein Y-binding substance can be selected from among picotamide, methoxsalen, terfenadine, cyclosporin A, pancuronium bromide, hydroxocobalamin, amphotericin B, protriptyline, rifampicin, solanine α, amethopterin, benztropine, sulfasalazine, nalidixic acid, astemizole, chlorprothixene, loperamide, fluphenazine, mefloquine, perphenazine, perhexyline, raloxifene, simvastatin, benoxinate, pioglitazone, thioproperazine, quinacrine, GBR12909, benzethonium, albendazole, acetopromazine amikacin, amiodarone, apigenin, buprenorphine, celestine blue, chlorambucil, chlorhexidine, chlorpromazine, cinchonine, clofazimine, clomiphene, cyproheptadine, deferoxamine, dihydroergocristine, dihydroergotamine, diphemanil, domperidone, doxazosin, eburnamonine, ellipticine, emetine, ethotoin, fenbendazole, flumequine, flunarizine, flupentixol, glipizide, harmaline, hydantoin, lidoflazine, lisinopril, mafenide, mefenamic acid, megestrol, mesoridazine, metergoline, methoxy-6-harmalan, meticrane, methixene, mifepristone, nordiazepam, oxethazaine, oxolinic acid, paclitaxel, palmatine, pentoxifylline, pimozide, pinacidil, rescinnamine, SR-95639A, sulfadimethoxine, syrosingopine, tamoxifen, tenoxicam, thioridazine, thiothixene (cis), tomatidine, dipyrone, ethyl loflazepate, clobazam, alimemazine, ebastine, reserpine, paramethasone, hydroxyprogesterone, dydrogesterone, sarpogrelate, demeclocycline, avermectin B1A, solasodine, nanofin (cis), tetrazoline, methylbenzethonium chloride, α-ergocryptine, spiramycin, chloropyramine, bergenin, nafcillin and carboprost, and derivatives thereof capable of binding to target protein Y (determined according to the kind of bioactive substance X) (described later), and salts thereof.

Although the salts may be any salts, pharmaceutically acceptable salts are preferable; examples include salts with inorganic bases (e.g., alkali metals such as sodium and potassium; alkaline earth metals such as calcium and magnesium; aluminum, ammonium), salts with organic bases (e.g., trimethylamine, triethylamine, pyridine, picoline, ethanolamine, diethanolamine, triethanolamine, dicyclohexylamine, N,N-dibenzylethylenediamine), salts with inorganic acids (e.g., hydrochloric acid, hydrobromic acid, nitric acid, sulfuric acid, phosphoric acid), salt with organic acids (e.g., formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid), salts with basic amino acids (e.g., arginine, lysine, ornithine) or salts with acidic amino acids (e.g., aspartic acid, glutamic acid) and the like.

Furthermore, the binding ability may also be measured on the basis of the binding ability of target protein Y to each isoform (e.g., splicing variant) or the isoform-isoform binding ability ratio, rather than on the basis of the total binding ability of target protein Y.

The binding ability can also be measured in silico. For example, a measurement of the binding ability can be performed on the basis of SBDD (structure-based drug design: SBDD) or CADD (computer-aided drug design). Examples of such screening include virtual screening, de novo design, pharmacophore analysis, QSAR (quantitative structure activity relationship) and the like. If information on the steric structure of the protein or the target site of the protein is required during such screening, the information on the steric structure is used, provided that the steric structure is known by a structural analytical technique such as NMR, X-ray crystallographic analysis, or synchrotron radiation analysis. If the steric structure is unknown, information obtained by a structural prediction method such as the homology method or the threading method is used. In virtual screening, a program known per se can be used; examples of the program include DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AutoDock (Morris, G. M. et al., J. Comput. Chem., 1998, 19, 1639), ICM (Abagyan, R. A. et al., J. Comput. Chem., 1994, 15, 488) and the like.

In step (c) of methodology III, a test substance capable of binding to target protein Y is selected. The test substance capable of binding to the protein is capable of promoting or suppressing a function of target protein Y. The test substance thus selected can be useful for the regulation of a disease or condition associated with bioactive substance X.

Methodology III can further comprise (d) (i) a step for confirming that the selected test substance is capable of regulating, for example, promoting or suppressing, an action associated with bioactive substance X (confirmation step), or (ii) a step for identifying the kind of action exhibited by the selected test substance (identification step). The confirmation step or identification step can be performed by, for example, administering the selected test substance to a normal animal, or to an animal with “a disease or condition associated with bioactive substance X” or model animal. According to this identification step, the kind of “action associated with bioactive substance X” possessed by the selected test substance can be determined, and whether or not the selected test substance can be used as either a drug or an investigational reagent, or both, and the kind of drug or investigational reagent to which the test substance is applicable can be confirmed.

In still another mode of embodiment, screening method Ia comprises the following steps (a), (b) and (c):

(a) a step for bringing the test substance and a target protein Y-binding substance into contact with target protein Y;
(b) a step for measuring the ability of the target protein Y-binding substance to bind to the protein in the presence of the test substance, and comparing this binding ability with the ability of the target protein Y-binding substance to bind to the protein in the absence of the test substance;
(c) a step for selecting a test substance that alters the ability of the target protein Y-binding substance to bind to the protein on the basis of the result of the comparison in step (b) above.

The methodology comprising the above-described steps (a) to (c) is referred to as “methodology IV” as required.

In step (a) of methodology IV, both a test substance and a target protein Y-binding substance are brought into contact with target protein Y. Contact of the test substance and the target protein Y-binding substance with the protein can be performed by mixing the test substance, the target protein Y-binding substance, and the protein in solution. The order of bringing the test substance and the target protein Y-binding substance into contact with the protein is not subject to limitation; one of them may be brought into contact with the protein at a time lag or at the same time.

Target protein Y can be prepared by a method known per se. For example, preparation of the protein can be performed by a method described in methodology III above.

The target protein Y-binding substance may be a labeled supply or a non-labeled supply, or a mixture of a labeled supply and a non-labeled supply mixed in a specified ratio. The labeling substance is the same as described above.

In step (b) of methodology IV, first, the ability of the target protein Y-binding substance to bind to the protein is measured in the presence of the test substance. “A binding ability” measured may be any one that enables an evaluation of the binding of the protein and the test substance; examples include binding amount, binding strength (including parameters such as affinity constant, binding rate constant, and dissociation rate constant), and binding mode (including dose-dependent binding).

A measurement of the binding ability can be performed using, for example, a labeled target protein Y-binding substance. The target protein Y-binding substance bound to the protein and the unbound target protein Y-binding substance may be separated before measuring the binding ability. More specifically, a measurement of the binding ability can be performed in the same manner as methodology III.

The binding ability may also be measured on the basis of the binding ability of target protein Y to each isoform (e.g., splicing variant) or the isoform-isoform binding ability ratio, rather than on the basis of the total amount of target protein Y bound.

Next, the binding ability of the target protein Y-binding substance to the protein in the presence of the test substance is compared with the binding ability of the target protein Y-binding substance to the protein in the absence of the test substance. This comparison of the binding ability is preferably performed on the basis of a significant difference. Although the binding ability of the target protein Y-binding substance to the protein in the absence of the test substance may be measured prior to, or simultaneously with, the measurement of the binding ability of the target protein Y-binding substance to the protein in the presence of the test substance, it is preferable, from the viewpoint of experimental accuracy and reproducibility, that the binding ability be measured simultaneously.

In step (c) of methodology IV, a test substance that alters the ability of the target protein Y-binding substance to bind to the protein is selected. The change in the binding ability can be, for example, a reduction or increase of binding ability, with preference given to a reduction of binding ability. The test substance thus selected can be useful for the regulation of an action associated with bioactive substance X.

Methodology IV can further comprise (d) (i) a step for confirming that the selected test substance is capable of regulating, for example, promoting or suppressing, an action associated with bioactive substance X (confirmation step), or (ii) a step for identifying the kind of action exhibited by the selected test substance (identification step). The confirmation step or identification step can be performed by, for example, administering the selected test substance to a normal animal or an animal with “a disease or condition associated with bioactive substance X” or model animal. According to this identification step, the kind of “action associated with bioactive substance X” exhibited by the selected test substance can be determined, and whether or not the selected test substance can be used as either a drug or an investigational reagent, or both, and the kind of drug or investigational reagent to which the test substance is applicable can be confirmed.

Screening method Ia can also be performed using an animal. Examples of the animal include mammals such as mice, rats, hamsters, guinea pigs, rabbits, dogs, and monkeys, and birds such as chickens. When a screening method of the present invention is performed using an animal, for example, a test substance that regulates the expression level of target gene Y can be selected.

Screening method Ia can also be performed by various methodologies suitable to the kind of target gene Y. For example, provided that target gene Y is a gene for an intracellularly localized factor, screening method I can be performed on the basis of a change in the intracellular localization of target protein Y. The amount of target protein Y localized in a specified organelle can be measured by a method known per se. For example, target gene Y, previously fused with a gene that encodes a fluorescent protein, such as the GFP gene, is introduced to an appropriate cell and cultured in culture medium in the presence of a test substance. Next, a fluorescence signal in the specified organelle is examined using a confocal microscope, and this signal is compared with the fluorescence signal in the absence of the test substance in the same organelle. The amount of target protein Y localized in the specified organelle can also be measured by immunostaining using an antibody against target protein Y.

Furthermore, provided that target gene Y is a gene for a soluble (secretory) factor, screening method Ia can be performed on the basis of a change in the blood concentration of the factor in the animal. Administration of the test substance to the animal, blood drawing from the animal, and the measurement of the blood concentration of the factor can be performed by a method known per se.

Screening method Ia enables screening of a substance capable of regulating an action associated with bioactive substance X. Hence, screening method Ia is useful for the development of a prophylactic or therapeutic agent for a disease or condition associated with bioactive substance X, an investigational reagent for the disease or the condition, and the like.

2.1.2. Screening Method for Substances Capable of Regulating an Action Associated with Bioactive Substance X, which Comprises Selecting a Test Substance Incapable of Regulating the Expression or Function of Target Protein Y (Screening Method Ib)

The present invention provides a screening method for test substances capable of regulating an action associated with bioactive substance X (particularly an action associated with a known target molecule), which comprises determining whether or not a test substance is capable of regulating the expression or function of target protein Y, and selecting a test substance incapable of regulating the expression or function of target protein Y.

Screening method Ib can be performed in the same manner as methodologies I to IV except that a test substance that does not cause a change or does not have the binding ability or regulatory capacity in step (c) of the above-described methodologies I to IV is selected.

In screening method Ib, the test substance used can be one capable of regulating the expression or function of a known target molecule (see, e.g., Tables 9-1 to 9-9). Hence, screening method Ib can be used in combination with a screening method for substances capable of regulating an action associated with a known target molecule (e.g., substances having the pharmacological effects shown in Tables 9-1 to 9-9), which comprises determining whether or not the test substance is capable of regulating the expression or function of the known target molecule. The screening method for substances capable of regulating an action associated with a known target molecule can be performed in the same manner as the above-described screening method Ia.

TABLE 9-1

| | | Example of action to | | Example of known |
|---|---|---|---|---|
| | | known target candidate | | target candidate |
| Compound name | Drug efficacy | molecule | Example of known target candidate molecule | protein sequence |
| picotamide | antiplatelet drug, | Thromboxane synthetase | Thromboxane A synthase | NP_001052.1 |
| | thromboxane synthesis | inhibitor; Thromboxane | Thromboxane A2 receptor | NP_112246.1 |
| | suppressant, inhibitor | receptor antagonist | | NP_001051.1 |
| | of synthesis and action | | | NP_963998.1 |
| | of TxA2 | | | |
| methoxsalen | dermatological drug | DNA adduct | DNA | |
| | (therapeutic drug for | | | |
| | vitiligo vulgaris) | | | |
| cyclosporin A | immunosuppressant, | Cyclophilin binder, | PPP3CA: protein phosphatase 3 (formerly | NP_000935.1 |
| | anti-malignant tumor | Calcineurin Inhibitor | 2B), catalytic subunit, alpha isoform | NP_066955.1 |
| | agent | | (calcineurin A alpha) | NP_005596.2 |
| | | | PPP3CB: protein phosphatase 3 (formerly | NP_000936.1 |
| | | | 2B), catalytic subunit, beta isoform | NP_671709.1 |
| | | | (calcineurin A beta) | NP_066953.1 |
| | | | PPP3CC: protein phosphatase 3 (formerly | NP_982254.1 |
| | | | 2B), catalytic subunit, gamma isoform | NP_982255.1 |
| | | | (calcineurin A gamma) | NP_000933.1 |
| | | | PPP3R1: protein phosphatase 3 (formerly | NP_000934.1 |
| | | | 2B), regulatory subunit B, 19 kDa, alpha | NP_005029.1 |
| | | | isoform (calcineurin B, type I) | NP_006103.1 |
| | | | PPP3R2: protein phosphatase 3 (formerly | NP_982281.1 |
| | | | 2B), regulatory subunit B, 19 kDa, beta | NP_982282.1 |

TABLE 9-2

| | | |
|---|---|---|
| cyclosporin A | isoform (calcineurin B, type II) | NP_005720.1 |
| | PPIA: peptidylprolyl isomerase A | NP_004783.2 |
| | (cyclophilin A) isoform 1 | NP_006338.1 |
| | PPIA: peptidylprolyl isomerase A | NP_057143.1 |
| | (cyclophilin A) isoform 2 | NP_055152.1 |
| | PPIB: peptidylprolyl isomerase B | NP_680480.1 |
| | (cyclophilin B) | NP_680481.1 |
| | PPIC: peptidylprolyl isomerase C | NP_115861.1 |
| | (cyclophilin C) | NP_570981.1 |
| | PPID: peptidylprolyl isomerase D | NP_572028.1 |
| | (cyclophilin D) | NP_624311.1 |
| | PPIE: peptidylprolyl isomerase E | NP_689542.2 |
| | (cyclophilin E) isoform 1 | NP_982291.1 |
| | PPIE: peptidylprolyl isomerase E | NP_982292.1 |
| | (cyclophilin E) isoform 2 | NP_775943.1 |
| | PPIE: peptidylprolyl isomerase E | |
| | (cyclophilin E) isoform 3 | |
| | PPIF: peptidylprolyl isomerase F | |
| | (cyclophilin F) | |
| | PPIG: peptidyl-prolyl isomerase G | |
| | (cyclophilin G) | |

TABLE 9-3

| | |
|---|---|
| cyclosporin A | PPIH: peptidyl prolyl isomerase H |
| | (cyclophilin H) |
| | PPIL1: peptidylprolyl isomerase |
| | (cyclophilin)-like 1 |
| | PPIL2: peptidylprolyl isomerase |
| | (cyclophilin)-like 2 Isoform a |
| | PPIL2: peptidylprolyl isomerase |
| | (cyclophilin)-like 2 Isoform b |
| | PPIL3: peptidylprolyl isomerase |
| | (cyclophilin)-like 3 Isoform a |
| | PPIL3: peptidylprolyl isomerase |
| | (cyclophilin)-like 3 Isoform b |
| | PPIL4: peptidylprolyl isomerase |
| | (cyclophilin)-like 4 |
| | PPIL5: peptidylprolyl isomerase |
| | (cyclophilin)-like 5 Isoform 1 |
| | PPIL5: peptidylprolyl isomerase |
| | (cyclophilin)-like 5 Isoform 2 |
| | PPIL5: peptidylprolyl isomerase |
| | (cyclophilin)-like 5 Isoform 3 |
| | PPIL6: peptidylprolyl isomerase |
| | (cyclophilin)-like 6 |

TABLE 9-4

| | | | | |
|---|---|---|---|---|
| terfenadine | bronchodilating agent | Histamine H1 receptor | HRH1: histamine receptor H1 | NP_000852.1 |
| | and therapeutic drug | antagonist; Blocker of | KCNH: potassium voltage-gated channel, | NP_000229.1 |
| | for asthma | HERG | subfamily H (eag-related) | NP_742053.1 |
| | | | | NP_742054.1 |
| | | | | NP_150375.2 |
| | | | | NP_775185.1 |
| pancuronium | muscle relaxant | Open channel and | CHRNA1: cholinergic receptor, nicotinic, | NP_000070.1 |
| bromide | (injection drug for | competitive blocker of | alpha polypeptide 1 (muscle) | NP_000738.2 |
| | anesthesia and operative | nicotinic acetyl | CHRNB1: cholinergic receptor, nicotinic, | |
| | treatment) | choline receptors | beta polypeptide 1 (muscle) | |
| albendazole | antiparasitic/antiprotozoal | Tubulin inhibitor | tubulin | |
| | drug (agent | | | |
| | destructive to hydatid) | | | |
| amphotericin B | antifungal drug | Ergosterol binder | | |
| | (polyenmacrolide-series | | | |
| | antibiotic) | | | |
| protriptyline | antidepressant, | Neurotransmitter | | |
| | mood-stabilizing drug, | norepinephrine | | |
| | psychoanaleptic, | inhibitor | | |
| | nonsedative cyclic | | | |
| | antidepressant | | | |
| rifampicin | antibiotic | Plastid RNA polymerase | | |
| | (antiphthisic) | beta-subunit binder | | |

TABLE 9-5

| | | | | |
|---|---|---|---|---|
| solanine α | steroid-series alkaloid | Acetylcholine•esterase | ACHE: acetylcholinesterase | NP_000656.1 |
| | glycoside, protoplasmic | inhibitor; | BCHE: butyrylcholinesterase | NP_056646.1 |
| | poison (poisonous | Butyrylcholinesterase | | NP_000046.1 |
| | substance accumulating | (BuChE) inhibitor | | |
| | on a surface of greened | | | |
| | potato) | | | |
| amethopterin | anti-malignant tumor | Dihydrofolate reductase | DHFR: dihydrofolate reductase | NP_000782.1 |
| | drug, antirheumatic drug, | inhibitor | | |
| | antipyretic analgesic | | | |
| | anti-inflammatory drug | | | |
| hydroxocobalamin | vitamin B12 preparation | | | |
| sulfasalazine | antipyretic analgesic | Mitochondrial | ACAT1: acetyl-Coenzyme A acetyltransferase | NP_000010.1 |
| | anti-inflammatory drug, | Acetyl-CoA | 1 (acetoacetyl Coenzyme A thiolase) | NP_000689.1 |
| | antirheumatic drug | acetyltransferase | ALOX5: arachidonate 5-lipoxygenase | NP_055146.1 |
| | | inhibitor; Arachidonate | SLC7: solute carrier family 7, (cationic | |
| | | 5-lipoxygenase | amino acid transporter) | |
| | | inhibitor; | | |
| | | Cystine/glutamate | | |
| | | transporter binder | | |
| nalidixic acid | chemotherapeutic agent, | DNA topoisomerase II | TOP2A: topoisomerase (DNA) II alpha | NP_001058.2 |
| | quinolone drug | (DNA Gyrase) inhibitor | TOP2B: topoisomerase (DNA) II beta | NP_001059.2 |
| | (pyridonecarboxylic | | | |
| | acid) | | | |
| chlorprothixene | antipsychotic drug | Dopamine receptor | DR: dopamine receptor | |
| | (group A: high-titer | antagonist | | |
| | group, phenothiazine | | | |
| | derivative) | | | |

TABLE 9-6

| | | | | |
|---|---|---|---|---|
| loperamide | antidiarrheal and | Mu-type opioid | OPRM1: opioid receptor, mu 1 | NP_000905.2 |
| | intestinal remedy | receptor Agonist; | OPRD1: opioid receptor, delta 1 | NP_001008503.1 |
| | | Delta-type opioid | CACN: calcium channel | NP_001008504.1 |
| | | receptor binder; Non- | | NP_001008505.1 |
| | | selective Ca2+ channel | | NP_000902.2 |
| | | Blocker | | |
| benztropine | antiparkinsonian drug | Muscarinic | CHRM1-5: cholinergic receptor 1-5, | NP_000729.2 |
| | (anticholinergic drug) | acetylcholine receptor | muscarinic | NP_000730.1 |
| | | antagonist | | NP_001006627.1 |
| | | | | NP_001006628.1 |
| | | | | NP_001006630.1 |
| | | | | NP_001006631.1 |
| | | | | NP_001006632.1 |
| | | | | NP_001006633.1 |
| | | | | NP_001006634.1 |
| | | | | NP_000731.1 |
| | | | | NP_000732.2 |
| | | | | NP_036257.1 |
| fluphenazine | antipsychotic drug | Dopamin D2 receptor | DRD2: dopamine receptor D2 | NP_000786.1 |
| | (group A: high-titer | antagonist | | NP_057658.2 |
| | group, phenothiazine | | | |
| | derivative), | | | |
| | antischizophrenic drug, | | | |
| | antidepressant, | | | |
| | antimanic drug, | | | |
| | psychoanaleptic | | | |

TABLE 9-7

| | | | | |
|---|---|---|---|---|
| mefloquine | antiparasitic/antiprotozoal | Phospholipid binder | | |
| | drug, therapeutic drug | | | |
| | for malaria | | | |
| perhexiline | antianginal drug, | Carnitine | CPT1A: carnitine palmitoyltransferase 1A | NP_001867.1 |
| | antiarrhythmic drug, | palmitoyltransferase-1 | (liver) | NP_004368.1 |
| | coronary vasodilator | (CPT-1) inhibitor; Blocker | TCPT1B: carnitine palmitoyltransferase 1B | NP_689451.1 |
| | | of HERG | (muscle) | NP_689452.1 |
| | | | TCPT1C: carnitine palmitoyltransferase 1C | NP_689453.1 |
| | | | KCNH: potassium voltage-gated channel, | NP_689572.1 |
| | | | subfamily H (eag-related) | NP_000229.1 |
| | | | | NP_742053.1 |
| | | | | NP_742054.1 |
| | | | | NP_150375.2 |
| | | | | NP_775185.1 |
| raloxifene | remedy for osteoporosis and | Estrogen receptor beta | ESR2: estrogen receptor 2 (ER beta) | NP_001428.1 |
| | bone metabolism, bone | Antagonist | | |
| | metabolism | | | |
| | modulator_tamoxifen | | | |
| | derivative, estrogen | | | |
| | receptor modulator | | | |

TABLE 9-8

| | | | | |
|---|---|---|---|---|
| astemizole | therapeutic drug for | Histamine H1 receptor | HRH1: histamine receptor H1 | NP_000852.1 |
| | allergya, | Antagonist; Blocker of HERG | KCNH: potassium voltage-gated channel, | NP_000229.1 |
| | antihistamine drug | | subfamily H (eag-related) | NP_742053.1 |
| | (basic histamine H1 | | | NP_742054.1 |
| | antagonist) | | | NP_150375.2 |
| | | | | NP_775185.1 |
| simvastatin | therapeutic drug for | 3-hydroxy-3-methylglutaryl-coenzyme | HMGCR: 3-hydroxy-3-methylglutaryl-Coenzyme A | NP_000850.1 |
| | hyperlipidemia | A (HMG-CoA) reductase | reductase | |
| | (HMG-CoA reductase | inhibitor | | |
| | inhibitor) | | | |
| benoxinate | ophthalmologic drug | | | |
| | (topical | | | |
| | anesthetics), | | | |
| | ophthalmologic | | | |
| | surface anesthetic | | | |
| pioglitazone | therapeutic drug for | Peroxisome proliferator | PPARG: peroxisome proliferative activated | NP_005028.3 |
| | diabetes (oral | activated receptor gamma | receptor, gamma | NP_056953.2 |
| | antihyperglycaemic | agonist | | NP_619725.1 |
| | drug, insulin | | | NP_619726.1 |
| | sensitizer) | | | |
| thioproperazine | antipsychotic drug, | Dopamine receptor antagonist | DR: dopamine receptor | |
| | phenothiazine | | | |
| | derivative, | | | |
| | antiemetic | | | |

TABLE 9-9

| | | | | |
|---|---|---|---|---|
| GBR12909 | cellular membrane | Synaptosomal dopamine uptake | SLC6: solute carrier family 6 (neurotransmitter | NP_001035.1 |
| | dopamine transporter | inhibitor; Dopamine carrier | transporter, dopamine) | |
| | inhibitor, dopamine | complex binder | | |
| | reuptake inhibitor | | | |
| perphenazine | antipsychotic drug | Dopamin D2 receptor antagonist | DRD2: dopamine receptor D2 | NP_000786.1 |
| | (group A: high-titer | | | NP_057658.2 |
| | group, phenothiazine derivative) | | | |
| quinacrine | antiparasitic/antiprotozoal | DNA Intercalator; | ALOX5: arachidonate 5-lipoxygenase | NP_000689.1 |
| | drug, | Arachidonate 5-lipoxygenase | CHRNA2: cholinergic receptor, nicotinic, alpha | NP_000733.1 |
| | therapeutic drug for | Inhibitor; Noncompetitive | polypeptide 2 (neuronal) | NP_000739.1 |
| | malaria | inhibitor of the nicotinic | CHRNB2: cholinergic receptor, nicotinic, beta | |
| | | acetylcholine receptor | polypeptide 2 (neuronal) | |
| | | (nAChR); Phospholipase A2 | PLA2: phospholipase A2 | |
| | | (PLA2) inhibitor | | |
| benzethonium | bactericidal | Neuronal nicotinic | CHRNA2: cholinergic receptor, nicotinic, alpha | NP_000733.1 |
| | disinfectant, agent | acetylcholine receptor | polypeptide 2 (neuronal) | NP_000739.1 |
| | for dentistry and | inhibitor | CHRNB2: cholinergic receptor, nicotinic, beta | |
| | oral cavity | | polypeptide 2 (neuronal) | |

Screening method Ib enables the development of drugs capable of regulating an action associated with a known target molecule, and showing decreased adverse effects of bioactive substance X. Hence, screening method Ib is useful for the improvement of existing drugs capable of regulating an action associated with a known target molecule and the like.

2.2. Screening Method for Substances Capable of Regulating a Function Associated with Target Protein Y (Screening Method II)

The present invention provides a screening method for substances capable of regulating a function associated with target protein Y, which comprises comparing the ability of a test substance to bind to target protein Y or the action associated with the test compound, with the ability of bioactive substance X to bind to target protein Y or the action associated with the bioactive substance.

This screening method is referred to as “screening method II” as required.

In one embodiment, screening method II comprises the following steps (a), (b) and (c):

(a) a step for bringing the test substance into contact with target protein Y;
(b) a step for measuring the functional level of the protein in the presence of the test substance, and comparing this functional level with the functional level of the protein in the presence of bioactive substance X;
(c) a step for selecting a test substance that alters the functional level of the protein on the basis of the result of the comparison in step (b) above.

The methodology comprising the above-described steps (a) to (c) is the same as methodology I except that the reference control for step (b) is not “the functional level of target protein Y in the absence of the test substance” but “the functional level of target protein Y in the presence of bioactive substance X”.

In another embodiment, screening method II comprises the following steps (a), (b) and (c):

(a) a step for bringing the test substance and cells enabling a measurement of the expression of target protein Y or a gene that encodes the protein into contact with each other;
(b) a step for measuring the expression level in the cells in contact with the test substance, and comparing this expression level with the expression level in control cells in contact with bioactive substance X;
(c) a step for selecting a test substance that regulates the expression level on the basis of the result of the comparison in step (b) above.

The methodology comprising the above-described steps (a) to (c) is the same as methodology II except that the reference control for step (b) is not “the expression level in control cells not in contact with the test substance” but “the expression level in control cells in contact with bioactive substance X”.

In still another mode of embodiment, screening method II comprises the following steps (a), (b) and (c):

(a) a step for bringing the test substance into contact with target protein Y;
(b) a step for measuring the ability of the test substance to bind to the protein, and comparing this binding ability with the ability of bioactive substance X to bind to the protein;
(c) a step for selecting a test substance capable of binding to the protein on the basis of the result of step (b) above.

The methodology comprising the above-described steps (a) to (c) is the same as methodology III except that the reference control for step (b) is “the ability of bioactive substance X to bind to target protein Y”.

Screening method II enables, for example, screening of substances capable of regulating a function associated with target protein Y, probes for target protein Y, and the like. Hence, screening method II is useful for the screening of prophylactic or therapeutic agents for diseases or conditions associated with target gene Y, screening of investigational reagents for the diseases or conditions, and the like.

### 2.3. Products Obtained by Screening Methods

The present invention provides products obtained by the above-described screening methods, for example, screening methods I and II.

A product provided by a screening method of the present invention can be a substance obtained by a screening method of the present invention, or a bioactivity regulator comprising a substance obtained by the screening method (described later).

A product provided by a screening method of the present invention is useful for, for example, the prevention or treatment of a disease or condition associated with bioactive substance X, or a disease or condition associated with target gene Y, or as an investigational reagent for the disease or the condition, and the like.

### 3. Regulators

The present invention provides bioactivity regulators each comprising a substance that regulates the expression or function of a target gene for a bioactive substance. The regulators of the present invention can be roughly divided into two types from the viewpoint of the bioactivity regulated: regulators of actions associated with bioactive substance X, and regulators of functions associated with target protein Y. The individual regulators are hereinafter described in detail.

3.1. Regulators of Actions Associated with Bioactive Substance X (Regulator I)

The present invention provides a type of regulators of actions associated with bioactive substance X, each of which comprises a substance that regulates the expression or function of target gene Y.

The regulators of this type are generically referred to as “regulator I” as required.

The substance that regulates the expression or function of target gene Y can be, for example, a substance that suppresses the expression of target gene Y. The expression refers to a state in which target gene Y translation product is produced and is localized at the action site thereof in a functional condition. Hence, the substance that suppresses the expression may be one that acts in any stage of gene transcription, post-transcriptional regulation, translation, post-translational modification, localization and protein folding and the like.

Specifically, the substance that suppresses the expression of target gene Y is exemplified by transcription suppressor, RNA polymerase inhibitor, RNA-degrading enzyme, protein synthesis inhibitor, nuclear translocation inhibitor, protein-degrading enzyme, protein denaturant and the like; to minimize the adverse effects on other genes and proteins expressed in the cells, it is important that the substance that suppresses the expression of target gene Y be capable of specifically acting on the target molecule.

An example of the substance that suppresses the expression of target gene Y is an antisense nucleic acid to a transcription product of target gene Y, specifically mRNA or initial transcription product. “An antisense nucleic acid” refers to a nucleic acid that consists of a base sequence capable of hybridizing to the target mRNA (initial transcription product) under physiological conditions for cells that express target mRNA (initial transcription product), and capable of inhibiting the translation of the polypeptide encoded by the target mRNA (initial transcription product) in a hybridized state. The kind of antisense nucleic acid may be DNA or RNA, or a DNA/RNA chimera. Because a natural type antisense nucleic acid easily undergoes degradation of the phosphoric acid diester bond thereof by a nucleolytic enzyme present in the cells, an antisense nucleic acid of the present invention can also be synthesized using a modified nucleotide of the thiophosphate type (P═O in phosphate linkage replaced with P═S), 2′-O-methyl type and the like which are stable to degrading enzymes. Other important factors for the designing of antisense nucleic acid include increases in water-solubility and cell membrane permeability and the like; these can also be cleared by choosing appropriate dosage forms such as those using liposome or microspheres.

The length of antisense nucleic acid is not subject to limitation, as long as the antisense nucleic acid is capable of specifically hybridizing to the transcription product of target gene Y; the antisense nucleic acid may be of a sequence complementary to a sequence of about 15 bases for the shortest, or the entire sequence of the mRNA (initial transcription product) for the longest. Considering the ease of synthesis, antigenicity and other issues, for example, oligonucleotides consisting of about 15 bases or more, preferably about 15 to about 30 bases, can be mentioned.

The target sequence for the antisense nucleic acid may be any sequence that inhibits the translation of target gene Y or a functional fragment thereof by being hybridized to the antisense nucleic acid, and may be the entire sequence or a partial sequence of mRNA, or the intron moiety of the initial transcription product; when an oligonucleotide is used as the antisense nucleic acid, it is desirable that the target sequence be located between the 5′ terminus of the mRNA of target gene Y and the C terminus of the coding region thereof.

Furthermore, the antisense nucleic acid may be not only capable of hybridizing to a transcription product of target gene Y to inhibit its translation, but also binding to target gene Y in the form of double-stranded DNA to form a triple-strand (triplex) and inhibit the transcription to mRNA.

Another example of the substance that suppresses the expression of target gene Y is a ribozyme capable of specifically cleaving a transcription product of target gene Y, specifically mRNA or initial transcription product in the coding region (including the intron portion in the case of initial transcription product). “A ribozyme” refers to an RNA possessing enzyme activity to cleave nucleic acids. Because it has recently been shown that an oligo-DNA having the base sequence of the enzyme activity site also possesses nucleic acid cleavage activity, this term is herein used to mean a concept including DNA, as long as sequence specific nucleic acid cleavage activity is possessed. The most versatile ribozyme is self-splicing RNA, found in infectious RNAs such as those of viroid and virusoid; this self-splicing RNA is known to occur in some types, including hammerhead type and hairpin type. When ribozyme is used in the form of an expression vector comprising a DNA that encodes the same, a hybrid ribozyme wherein a sequence modified from tRNA is further linked to promote localization to cytoplasm may be used [Nucleic Acids Res., 29(13): 2780-2788 (2001)].

A still another example of the substance that suppresses the expression of target gene Y is a decoy nucleic acid. A decoy nucleic acid refers to a nucleic acid molecule that mimics a region to which a transcription regulatory factor binds; the decoy nucleic acid, which is the substance that suppresses the expression of target gene Y, can be a nucleic acid molecule that mimics a region to which a transcription activation factor for target gene Y binds.

Examples of the decoy nucleic acid include oligonucleotides modified to make them unlikely to undergo degradation in a body, such as oligonucleotides having a thiophosphoric diester bond wherein an oxygen atom in the phosphoric diester bond moiety is replaced with a sulfur atom (S-oligo), and oligonucleotides wherein the phosphoric diester bond is replaced with an uncharged methyl phosphate group, and the like. Although the decoy nucleic acid may completely match with the region to which a transcription activation factor binds, the degree of matching may be such that the transcription activation factor for target gene Y can bind the decoy nucleic acid. The length of the decoy nucleic acid is not subject to limitation, as long as the transcription activation factor binds thereto. The decoy nucleic acid may comprise a repeat of the same region.

Still another example of the substance that suppresses the expression of target gene Y is a double-stranded oligo-RNA, i.e. siRNA, which is complementary to a partial sequence (including the intron portion in the case of an initial transcription product) in the coding region of a transcription product of target gene Y, specifically, the mRNA or initial transcription product. It has been known that so-called RNA interference (RNAi), which is a phenomenon that if short double stranded RNA is introduced into cells, mRNA complementary to the RNA is degraded, occurs in nematodes, insects, plants and the like; recently, it has been found that this phenomenon also occurs in animal cells [Nature, 411(6836): 494-498 (2001)], which is drawing attention as an alternative technique to ribozymes. The siRNA used may be internally synthesized as described below, and a commercially available one may be used.

An antisense oligonucleotide and ribozyme can be prepared by determining the target sequence for a transcription product of target gene Y, specifically the mRNA or initial transcription product on the basis of the cDNA sequence or genomic DNA sequence of target gene Y, and by synthesizing a sequence complementary thereto using a commercially available automated DNA/RNA synthesizer (Applied Biosystems Company, Beckman Instruments Company and the like). A decoy nucleic acid and siRNA can be prepared by synthesizing a sense strand and an antisense strand in an automated DNA/RNA synthesizer, respectively, denaturing the chains in an appropriate annealing buffer solution at about 90 to about 95° C. for about 1 minute, and then annealing the chains at about 30 to about 70° C. for about 1 to about 8 hours. A longer double-stranded polynucleotide can be prepared by synthesizing a complementary oligonucleotide chain in alternative overlaps, annealing them, and then ligating them with ligase.

Another example of the substance that suppresses the expression of target gene Y is an antibody against target protein Y. The antibody may be a polyclonal antibody or a monoclonal antibody, and can be prepared by a well-known immunological technique. The antibody may also be a fragment of an antibody (e.g., Fab, F(ab′)₂), or a recombinant antibody (e.g., single-chain antibody). Furthermore, the nucleic acid that encodes the antibody (one functionally linked to a nucleic acid having promoter activity) is also preferable as the substance that suppresses the expression of target gene Y.

The polyclonal antibody can be acquired by, for example, subcutaneously or intraperitoneally administering target protein Y or a fragment thereof (as required, may be prepared as a complex crosslinked to a carrier protein such as bovine serum albumin or KLH (keyhole limpet hemocyanin)) as the antigen, along with a commercially available adjuvant (e.g., Freund's complete or incomplete adjuvant) to an animal about 2 to 4 times at intervals of 2 to 3 weeks (the antibody titer of partially drawn serum has been determined by a known antigen-antibody reaction and its elevation has been confirmed in advance), collecting whole blood about 3 to about 10 days after final immunization, and purifying the antiserum. As the animal to receive the antigen, mammals such as rats, mice, rabbits, goat, guinea pigs, and hamsters can be mentioned.

The monoclonal antibody can be prepared by, for example, a cell fusion method (e.g., Takeshi Watanabe, Saibou Yugouhou No Genri To Monokuronaru Koutai No Sakusei, edited by Akira Taniuchi and Toshitada Takahashi, “Monokuronaru Koutai To Gan—Kiso To Rinsho—”, pages 2-14, Science Forum Shuppan, 1985). For example, the factor is administered subcutaneously or intraperitoneally along with a commercially available adjuvant to a mouse 2 to 4 times, and about 3 days after final administration, the spleen or lymph nodes are collected, and leukocytes are collected. These leukocytes and myeloma cells (e.g., NS-1, P3X63Ag8 and the like) are cell-fused to obtain a hybridoma that produces a monoclonal antibody against the factor. This cell fusion may be performed by the PEG method [J. Immunol. Methods, 81(2): 223-228 (1985)], or by the voltage pulse method [Hybridoma, 7(6): 627-633 (1988)]. A hybridoma that produces the desired monoclonal antibody can be selected by detecting an antibody that binds specifically to the antigen from the culture supernatant using a widely known EIA or RIA method and the like. Cultivation of the hybridoma that produces the monoclonal antibody can be performed in vitro, or in vivo such as in mouse or rat ascitic fluid, preferably in mouse ascitic fluid, and the antibody can be acquired from the culture supernatant of the hybridoma and the ascitic fluid of the animal, respectively.

However, in view of therapeutic efficacy and safety in humans, the antibody of the present invention may be a chimeric antibody or a humanized or human type antibody. The chimeric antibody can be prepared with reference to, for example, “Jikken Igaku (extra issue), Vol. 6, No. 10, 1988”, Japanese Patent Kokoku Publication No. HEI-3-73280 and the like. The humanized antibody can be prepared with reference to, for example, Japanese Patent Kohyo Publication No. HEI-4-506458, Japanese Patent Kokai Publication No. SHO-62-296890 and the like. The human antibody can be prepared with reference to, for example, “Nature Genetics, Vol. 15, p. 146-156, 1997”, “Nature Genetics, Vol. 7, p. 13-21, 1994”, Japanese Patent Kohyo Publication No. HEI-4-504365, International Patent Application Publication No. WO94/25585, “Nikkei Science, June issue, pp. 40 to 50, 1995”, “Nature, Vol. 368, pp. 856-859, 1994”, Japanese Patent Kohyo Publication No. HEI-6-500233 and the like.

The substance that regulates the expression or function of target gene Y can also be a substance that suppresses a function of target gene Y.

Although the substance that suppresses a function of target gene Y is not subject to limitation, as long as it is capable of interfering with an action of target gene Y, it is important that the substance be capable of specifically acting on the target molecule to minimize the adverse effect on other genes and proteins. Examples of the substance that specifically suppresses a function of target gene Y include a dominant negative mutant of target protein Y and a nucleic acid that encodes the mutant (one functionally linked to a nucleic acid having promoter activity).

A dominant negative mutant of target protein Y refers to a mutant having the activity thereof reduced as a result of mutagenesis to target protein Y. The dominant negative mutant can indirectly inhibit the activity of natural target protein Y by competing therewith. The dominant negative mutant can be prepared by introducing a mutation to a nucleic acid that encodes target gene Y. Examples of the mutation include amino acid mutations in a functional domain that result in a decrease in the function responsible for the domain (e.g., deletion, substitution, and addition of one or more amino acids). The mutation can be introduced by a method known per se using PCR or a commonly known kit.

Provided that the substance that suppresses the expression of target gene Y is a nucleic acid molecule, the regulator of the present invention can have an expression vector that encodes the nucleic acid molecule as the active ingredient thereof. In the expression vector, an oligonucleotide or polynucleotide that encodes the above-described nucleic acid molecule must be functionally linked to a promoter capable of exhibiting promoter activity in the cells of the recipient mammal. Any promoter capable of functioning in the recipient mammal can be used; examples include viral promoters such as the SV40-derived early promoter, cytomegalovirus LTR, Rous sarcoma virus LTR, MoMuLV-derived LTR, and adenovirus-derived early promoter, and mammalian structural protein gene promoters such as the β-actin gene promoter, PGK gene promoter, and transferrin gene promoter, and the like.

The expression vector preferably comprises a transcription termination signal, that is, a terminator region, downstream of the oligo(poly)nucleotide that encodes the nucleic acid molecule. The expression vector may further comprise a selection marker gene for selecting transformant cells (genes that confer resistance to drugs such as tetracycline, ampicillin, kanamycin, hygromycin, and phosphinothricin, gene that compensate for auxotrophic mutation, and the like).

Although the basic backbone vector used as the expression vector is not subject to limitation, vectors suitable for administration to mammals such as humans include viral vectors such as retrovirus, adenovirus, adeno-associated virus, herpesvirus, vaccinia virus, poxvirus, poliovirus, Sindbis virus, and Sendai virus. Adenovirus has advantageous features, including the very high efficiency of gene introduction and possibility of introduction to non-dividing cells. Because incorporation of the introduced gene to host chromosome is very rare, however, gene expression is transient, usually lasting for about 4 weeks. In view of the sustainability of therapeutic effect, it is also preferable to use adeno-associated virus, which offers relatively high gene transduction efficiency, which can be introduced to non-dividing cells, and which can be incorporated in chromosomes via a inverted terminal repeat sequence (ITR).

The substance that regulates the expression or function of target protein Y can also be picotamide, methoxsalen, terfenadine, cyclosporin A, pancuronium bromide, hydroxocobalamin, amphotericin B, protriptyline, rifampicin, solanine α, amethopterin, benztropine, sulfasalazine, nalidixic acid, astemizole, chlorprothixene, loperamide, fluphenazine, mefloquine, perphenazine, perhexyline, raloxifene, simvastatin, benoxinate, pioglitazone, thioproperazine, quinacrine, GBR12909, benzethonium, albendazole, acetopromazine, amikacin, amiodarone, apigenin, buprenorphine, celestine blue, chlorambucil, chlorhexidine, chlorpromazine, cinchonine, clofazimine, clomiphene, cyproheptadine, deferoxamine, dihydroergocristine, dihydroergotamine, diphemanil, domperidone, doxazosin, eburnamonine, ellipticine, emetine, ethotoin, fenbendazole, flumequine, flunarizine, flupentixol, glipizide, harmaline, hydantoin, lidoflazine, lisinopril, mafenide, mefenamic acid, megestrol, mesoridazine, metergoline, methoxy-6-harmalan, meticrane, methixene, mifepristone, nordiazepam, oxethazaine, oxolinic acid, paclitaxel, palmatine, pentoxifylline, pimozide, pinacidil, rescinnamine, SR-95639A, sulfadimethoxine, syrosingopine, tamoxifen, tenoxicam, thioridazine, thiothixene (cis), tomatidine, dipyrone, ethyl loflazepate, clobazam, alimemazine, ebastine, reserpine, paramethasone, hydroxyprogesterone, dydrogesterone, sarpogrelate, demeclocycline, avermectin B1A, solasodine, nanofin (cis), tetrazoline, methylbenzethonium chloride, α-ergocryptine, spiramycin, chloropyramine, bergenin, nafcillin and carboprost or a derivative thereof capable of binding to target protein Y (described later), or a salt thereof.

Regulator I, in addition to a substance that regulates the expression or function of target gene Y, can comprise any carrier, for example, a pharmaceutically acceptable carrier.

Examples of the pharmaceutically acceptable carrier include, but are not limited to, excipients such as sucrose, starch, mannitol, sorbitol, lactose, glucose, cellulose, talc, calcium phosphate, and calcium carbonate; binders such as cellulose, methylcellulose, hydroxypropylcellulose, polypropylpyrrolidone, gelatin, gum arabic, polyethylene glycol, sucrose, and starch; disintegrants such as starch, carboxymethylcellulose, hydroxypropylstarch, sodium-glycol-starch, sodium hydrogen carbonate, calcium phosphate, and calcium citrate; lubricants such as magnesium stearate, Aerosil, talc, and sodium lauryl sulfate; flavoring agents such as citric acid, menthol, glycyrrhizin ammonium salt, glycine, and orange powder; preservatives such as sodium benzoate, sodium hydrogen sulfite, methyl paraben, and propyl paraben; stabilizers such as citric acid, sodium citrate, and acetic acid; suspending agents such as methylcellulose, polyvinylpyrrolidone, and aluminum stearate; dispersing agents such as surfactants; diluents such as water, physiological saline, and orange juice; base waxes such as cacao fat, polyethylene glycol, and kerosene, and the like.

Preparations suitable for oral administration include liquids comprising an effective amount of substance dissolved in a diluent such as water, physiological saline, or orange juice, capsules, sachets or tablets comprising an effective amount of substance in the form of solid or granules, suspensions comprising an effective amount of substance suspended in an appropriate dispersant, emulsions comprising a solution of an effective amount of substance dispersed in an appropriate dispersant and the like.

Preparations suitable for parenteral administration (e.g., subcutaneous injection, intramuscular injection, topical injection, intraperitoneal injection, and the like) include aqueous and non-aqueous isotonic sterile injection liquids, which may comprise an antioxidant, a buffer solution, a bacteriostatic agent, an isotonizing agent and the like. Other examples are aqueous and non-aqueous sterile suspensions, which may comprise a suspending agent, a solubilizer, a thickening agent, a stabilizer, an antiseptic and the like. The preparation can be included in a container in a unit dose or multiple doses like an ampoule or vial. It is also possible to lyophilize the active ingredient and a pharmaceutically acceptable carrier and preserve them in a state that only requires dissolving or suspending in a suitable sterile vehicle immediately before use.

The dose of regulator I varies depending on the activity and kind of the active ingredient, severity of the disease, the animal species to be the administration subject, drug acceptability, body weight and age of the administration subject, and the like, it is generally about 0.001 to about 500 mg/kg a day for an adult based on the amount of the active ingredient.

Regulator I enables the regulation, for example, suppression or promotion, of an action associated with bioactive substance X. Hence, regulator I is useful for the prophylaxis and treatment of a disease or condition associated with bioactive substance X, and as an investigational reagent for the disease or the condition, and the like.

3.2. Regulator of a Function Associated with Target Protein Y (Regulator II)

The present invention provides a regulator of a function associated with target protein Y, which comprises bioactive substance X.

This regulator is referred to as “regulator II” as required.

Bioactive substance X can be picotamide, methoxsalen, terfenadine, cyclosporin A, pancuronium bromide, hydroxocobalamin, amphotericin B, protriptyline, rifampicin, solanine α, amethopterin, benztropine, sulfasalazine, nalidixic acid, astemizole, chlorprothixene, loperamide, fluphenazine, mefloquine, perphenazine, perhexyline, raloxifene, sinvastatin, benoxinate, pioglitazone, thioproperazine, quinacrine, GBR12909, benzethonium, albendazole, acetopromazine amikacin, amiodarone, apigenin, buprenorphine, celestine blue, chlorambucil, chlorhexidine, chlorpromazine, cinchonine, clofazimine, clomiphene, cyproheptadine, deferoxamine, dihydroergocristine, dihydroergotamine, diphemanil, domperidone, doxazosin, eburnamonine, ellipticine, emetine, ethotoin, fenbendazole, flumequine, flunarizine, flupentixol, glipizide, harmaline, hydantoin, lidoflazine, lisinopril, mafenide, mefenamic acid, megestrol, mesoridazine, metergoline, methoxy-6-harmalan, meticrane, methixene, mifepristone, nordiazepam, oxethazaine, oxolinic acid, paclitaxel, palmatine, pentoxifylline, pimozide, pinacidil, rescinnamine, SR-95639A, sulfadimethoxine, syrosingopine, tamoxifen, tenoxicam, thioridazine, thiothixene (cis), tomatidine, dipyrone, ethyl loflazepate, clobazam, alimemazine, ebastine, reserpine, paramethasone, hydroxyprogesterone, dydrogesterone, sarpogrelate, demeclocycline, avermectin B1A, solasodine, nanofin (cis), tetrazoline, methylbenzethonium chloride, α-ergocryptine, spiramycin, chloropyramine, bergenin, nafcillin and carboprost or a derivative thereof capable of binding to target protein Y (described later), or a salt thereof.

Regulator II can comprise, in addition to bioactive substance X, any carrier, for example, a pharmaceutically acceptable carrier. The dose of regulator II is the same as that of regulator I.

Regulator II enables the regulation, for example, suppression or promotion, of a function associated with target protein Y. Hence, regulator II is useful for the prophylaxis and treatment of a disease or condition associated with target gene Y, and as an investigational reagent for the disease, and the like.

### 4. Derivative Production Method and Product Obtained by the Method

### 4.1. Derivative Production Method

The present invention provides a method of producing a bioactive substance derivative, which comprises derivatizing a bioactive substance so as to be able to regulate the expression or function of the target gene.

Derivatization means that a compound obtained by replacing a particular atom or group in a lead compound with another atom or group, or a compound obtained by subjecting a lead compound to an addition reaction, is virtually or actually synthesized. For example, the lead compound can be bioactive substance X.

The derivatization of bioactive substance X can be performed so that the regulatory capability for the expression or function of target gene Y is retained, and as required, in view of other properties of the derivative obtained, such as water-solubility/lipid-solubility, stability, dynamics, bioavailability, toxicity and the like. The derivatization of bioactive substance X can be performed so that, for example, the regulatory capability for the expression or function of target gene Y can be increased. The derivatization of bioactive substance X can also be performed so that a function associated with target protein Y can be regulated.

The derivatization of bioactive substance X such that the regulatory capability for the expression or function of target gene Y is retained can be performed on the basis of, for example, SBDD (structure-based drug design) and CADD (computer-aided drug design). Examples of the design include virtual screening, de novo design, pharmacophore analysis, QSAR (quantitative structure activity relationship) and the like. If information on the steric structure of the protein itself or the target site of the protein is required during such designing, information on the steric structure is used provided that the steric structure is known by a structural analytical technique such as NMR, X-ray crystallographic analysis, or synchrotron radiation analysis. If the steric structure is unknown, information obtained by a structural predictive method such as the homology method or the threading method is used. In virtual screening, a program known per se is used; examples of the program include DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AtutoDock (Morris, G. M. et al., J. Comput. Chem., 1998, 19, 1639), ICM (Abagyan, R. A. et al., J. Comput. Chem., 1994, 15, 488) and the like.

The derivatization of bioactive substance X such that the regulatory capacity for the expression or function of target gene Y is retained can also be performed on the basis of, for example, biological verification (in vitro or in vivo method). In this case, for example, the above-described methodologies I to IV can be used. Furthermore, one of the above-described methods such as SBDD and CADD, and biological verification may be used in combination.

The particular atom in bioactive substance X (a lead compound), which is substituted for producing the derivative, may be any atom present in the lead compound, exemplified by a hydrogen atom, a halogen atom (e.g., fluorine atom, chlorine atom, bromine atom, iodine atom), an oxygen atom, a sulfur atom, a nitrogen atom, a carbon atom and the like.

The particular group in bioactive substance X, which is substituted for producing the derivative, may be any group present in bioactive substance X, and can, for example, be a group having a molecular weight of 1 to 500, preferably 1 to 300, more preferably 1 to 200, most preferably 1 to 100. Examples of the particular group include an optionally substituted C₁ to C₈ hydrocarbon group, an optionally substituted C₁ to C₈ acyl group, an optionally substituted aromatic or non-aromatic C₃ to C₁₄ hydrocarbon cyclic group, or an optionally substituted aromatic or non-aromatic C₃ to C₁₄ heterocyclic group, an amino group, an amino group mono- or di-substituted by an alkyl group having 1 to 4 carbon atoms or an acyl group having 2 to 8 carbon atoms, an amidino group, a carbamoyl group, a carbamoyl group mono- or di-substituted by an alkyl group having 1 to 4 carbon atoms, a sulfamoyl group, a sulfamoyl group mono- or di-substituted by an alkyl group having 1 to 4 carbon atoms, a carboxyl group, an alkoxycarbonyl group having 2 to 8 carbon atoms, a hydroxy group, an alkoxy group having 1 to 6 carbon atoms optionally substituted by 1 to 3 halogen atoms, an alkenyloxy group having 2 to 5 carbon atoms optionally substituted by 1 to 3 halogen atoms, a cycloalkyloxy group having 3 to 7 carbon atoms, an aralkyloxy group having 7 to 9 carbon atoms, an aryloxy group having 6 to 14 carbon atoms, a thiol group, an alkylthio group having 1 to 6 carbon atoms optionally substituted by 1 to 3 halogen atoms, an aralkylthio group having 7 to 9 carbon atoms, an arylthio group having 6 to 14 carbon atoms, a sulfo group, a cyano group, an azido group, a nitro group, a nitroso group and the like.

The optionally substituted C₁ to C₈ hydrocarbon group can, for example, be an optionally substituted C₁ to C₈ alkyl group, an optionally substituted C₂ to CB alkenyl group, or an optionally substituted C₂ to CB alkynyl group.

The C₁ to C₈ alkyl group in the optionally substituted C₁ to C₈ alkyl group may be linear or branched, preferably having 1 to 6 carbon atoms; examples include methyl, ethyl, propyl, isopropyl, butyl, isobutyl, sec-butyl, tert-butyl and the like.

The C₂ to CB alkenyl group in the optionally substituted C₂ to CB alkenyl group may be linear or branched, preferably having 2 to 6 carbon atoms; examples include ethenyl, 1-propenyl, 2-propenyl, 2-methyl-1-propenyl, 1-butenyl, 2-butenyl, 3-butenyl and the like.

The C₂ to C₈ alkynyl group in the optionally substituted C₂ to C₈ alkynyl group may be linear or branched, preferably having 2 to 6 carbon atoms; examples include ethynyl, 1-propynyl, 2-propynyl, 1-buthynyl, 2-buthynyl, 3-buthynyl and the like.

The C₁ to C₈ acyl group in the optionally substituted C₁ to C₈ acyl group may be linear or branched, preferably having 2 to 6 carbon atoms; examples include formyl, acetyl, propinoyl, butanoyl, 2-methylpropinoyl and the like.

The aromatic C₃ to C₁₄ hydrocarbon cyclic group in the optionally substituted aromatic C₃ to C₁₄ hydrocarbon cyclic group may be monocyclic, bicyclic or tricyclic, preferably having 3 to 12 carbon atoms; examples include phenyl and naphthyl.

The non-aromatic C₃ to C₁₄ hydrocarbon cyclic group in the optionally substituted non-aromatic C₃ to C₁₄ hydrocarbon cyclic group may be saturated or unsaturated monocyclic, bicyclic or tricyclic, preferably having 3 to 12 carbon atoms; examples include cycloalkyl groups (e.g., cyclopropyl, cyclobutyl, cyclopentyl, cyclohexyl, cycloheptyl, cyclooctyl), cycloalkenyl groups (e.g., 2-cyclopenten-1-yl, 3-cyclopenten-1-yl, 2-cyclohexen-1-yl, 3-cyclohexen-1-yl), cycloalkadienyl groups (e.g., 2,4-cyclopentadien-1-yl, 2,4-cyclohexadien-1-yl, 2,5-cyclohexadien-1-yl) and the like.

The aromatic C₃ to C₁₄ heterocyclic group in the optionally substituted aromatic C₃ to C₁₄ heterocyclic group is a monocyclic, bicyclic or tricyclic aromatic heterocyclic group containing 1 to 5 hetero atoms selected from among oxygen atoms, sulfur atoms and nitrogen atoms, in addition to carbon atoms, as the ring-forming atoms, preferably having 3 to 12 carbon atoms. Examples of the monocyclic aromatic C₃ to C₁₄ heterocyclic group include furyl, thienyl, pyrrolyl, oxazolyl, isooxazolyl, thiazolyl, isothiazolyl, imidazolyl, pyrazolyl, oxadiazolyl, furazanyl, thiadiazolyl, triazolyl, tetrazolyl, pyridyl, pyrimidinyl, pyridazinyl, pyrazinyl, triazinyl and the like. Examples of the bicyclic or tricyclic aromatic heterocyclic group include benzofuranyl, isobenzofuranyl, benzo[b]thienyl, indolyl, isoindolyl, 1H-indazolyl, benzimidazolyl, benzooxazolyl, benzothiazolyl, 1H-benzotriazolyl, quinolyl, isoquinolyl, cinnolyl, quinazolyl, quinoxalinyl, phthaladinyl, naphthylizinyl, purinyl, pteridinyl, carbazolyl, α-carbonylyl, α-carbonylyl, γ-carbonylyl, acrydinyl, phenoxazinyl, phenothiazinyl, phenadinyl, phenoxathiinyl, thianthrenyl, indolidinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl and the like.

The non-aromatic C₃ to C₁₄ heterocyclic group in the optionally substituted non-aromatic C₃ to C₁₄ heterocyclic group is a monocyclic, bicyclic or tricyclic saturated or unsaturated heterocyclic group containing 1 to 5 hetero atoms selected from among oxygen atoms, sulfur atoms and nitrogen atoms, in addition to carbon atoms, as the ring-forming atoms, preferably having 3 to 12 carbon atoms; examples include oxiranyl, azetidinyl, oxetanyl, thietanyl, pyrrolidinyl, tetrahydrofuryl, tetrahydropyranyl, morpholinyl, thiomorpholinyl, piperazinyl, pyrrolidinyl, piperidino, morpholino, thiomorpholino and the like.

The kind of the substituent in any group optionally substituted can be the same as the particular group in bioactive substance X (described above), which is substituted for producing the derivative.

The number of particular atoms or groups in bioactive substance X, which is substituted for producing the derivative is any one, as long as the derivative produced is capable of regulating the expression or function of the gene Y, for example, as long as it is capable of binding to target protein Y, and can be, for example, 1 to 10, preferably 1 to 5, more preferably 1 to 3, further more preferably 1 to 2, most preferably 1.

The kind of a particular atom or group used for substitution (i.e., an atom or group introduced to the substitution site) can be the same as the particular atom or group in bioactive substance X, which is substituted for producing the derivative.

The atom or group added to bioactive substance X for producing the derivative (i.e., an atom or group used in the addition reaction) is an atom permitting an addition reaction, for example, an atom such as the hydrogen atom or the halogen atom, or a group capable of acting as a nucleophile or electrophile, out of the particular atoms or groups in bioactive substance X (described above), which is substituted for producing the derivative.

The number of atoms or groups added to bioactive substance X for producing the derivative is any one, as long as the derivative produced is capable of regulating the expression or function of the gene Y, for example, as long as it is capable of binding to target protein Y, and can be, for example, less than 6, preferably less than 4, more preferably less than 2.

The production method of the present invention is useful for, for example, the development of prophylactic or therapeutic agents for diseases or conditions associated with bioactive substance X or diseases or conditions associated with target gene Y, or investigational reagents for the diseases or the conditions, and the like.

### 4.2. Products Obtained by the Derivative Production Method

The present invention provides a product obtained by the above-described method of producing a derivative.

The product provided by the above-described production method can be bioactive substance X derivative obtained by the production method of the present invention, and a bioactivity regulator comprising the derivative (described above).

A product provided by the above-described production method is useful for, for example, the prophylaxis or treatment of a disease or condition associated with bioactive substance X, or a disease or condition associated with target gene Y, or as investigational reagents for the disease or the condition, and the like.

### 5. Complex and a Method of Producing the Same

The present invention provides a complex comprising a bioactive substance and a target protein therefor.

The bioactive substance can be, for example, the above-described bioactive substance X. Specifically, bioactive substance X can be picotamide, methoxsalen, terfenadine, cyclosporin A, pancuronium bromide, hydroxocobalamin, amphotericin B, protriptyline, rifampicin, solanine α, amethopterin, benztropine, sulfasalazine, nalidixic acid, astemizole, chlorprothixene, loperamide, fluphenazine, mefloquine, perphenazine, perhexyline, raloxifene, simvastatin, benoxinate, pioglitazone, thioproperazine, quinacrine, GBR12909, benzethonium, albendazole, acetopromazine, amikacin, amiodarone, apigenin, buprenorphine, celestine blue, chlorambucil, chlorhexidine, chlorpromazine, cinchonine, clofazimine, clomiphene, cyproheptadine, deferoxamine, dihydroergocristine, dihydroergotamine, diphemanil, domperidone, doxazosin, eburnamonine, ellipticine, emetine, ethotoin, fenbendazole, flumequine, flunarizine, flupentixol, glipizide, harmaline, hydantoin, lidoflazine, lisinopril, mafenide, mefenamic acid, megestrol, mesoridazine, metergoline, methoxy-6-harmalan, meticrane, methixene, mifepristone, nordiazepam, oxethazaine, oxolinic acid, paclitaxel, palmatine, pentoxifylline, pimozide, pinacidil, rescinnamine, SR-95639A, sulfadimethoxine, syrosingopine, tamoxifen, tenoxicam, thioridazine, thiothixene (cis), tomatidine, dipyrone, ethyl loflazepate, clobazam, alimemazine, ebastine, reserpine, paramethasone, hydroxyprogesterone, dydrogesterone, sarpogrelate, demeclocycline, avermectin B1A, solasodine, nanofin (cis), tetrazoline, methylbenzethonium chloride, α-ergocryptine, spiramycin, chloropyramine, bergenin, nafcillin and carboprost or a derivative thereof capable of binding to target protein Y. The kind of bioactive substance X can be selected as appropriate according to the kind of target protein Y.

The target protein for the bioactive substance can be, for example, the above-described target protein Y. Specifically, target protein Y can be a protein comprising the amino acid sequence shown by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID Nb:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:22, SEQ ID NO:23 or SEQ ID NO:24 or a protein homologous thereto or a variant thereof. The kind of target protein Y used to form the complex can be selected as appropriate according to the kind of bioactive substance X.

In one embodiment, the complex of the present invention can be a complex according to a combination of picotamide, methoxsalen, terfenadine, cyclosporin A, pancuronium bromide, hydroxocobalamin, amphotericin B, protriptyline, rifampicin, solanine α, amethopterin, benztropine, sulfasalazine, nalidixic acid, astemizole, chlorprothixene, loperamide, fluphenazine, mefloquine, perphenazine, perhexyline, raloxifene, simvastatin, benoxinate, pioglitazone, thioproperazine, quinacrine, GBR12909, benzethonium, albendazole, acetopromazine, amikacin, amiodarone, apigenin, buprenorphine, celestine blue, chlorambucil, chlorhexidine, chlorpromazine, cinchonine, clofazimine, clomiphene, cyproheptadine, deferoxamine, dihydroergocristine, dihydroergotamine, diphemanil, domperidone, doxazosin, eburnamonine, ellipticine, emetine, ethotoin, fenbendazole, flumequine, flunarizine, flupentixol, glipizide, harmaline, hydantoin, lidoflazine, lisinopril, mafenide, mefenamic acid, megestrol, mesoridazine, metergoline, methoxy-6-harmalan, meticrane, methixene, mifepristone, nordiazepam, oxethazaine, oxolinic acid, paclitaxel, palmatine, pentoxifylline, pimozide, pinacidil, rescinnamine, SR-95639A, sulfadimethoxine, syrosingopine, tamoxifen, tenoxicam, thioridazine, thiothixene (cis), tomatidine, dipyrone, ethyl loflazepate, clobazam, alimemazine, ebastine, reserpine, paramethasone, hydroxyprogesterone, dydrogesterone, sarpogrelate, demeclocycline, avermectin B1A, solasodine, nanofin (cis), tetrazoline, methylbenzethonium chloride, α-ergocryptine, spiramycin, chloropyramine, bergenin, nafcillin and carboprost or a derivative thereof capable of binding to a target protein and a target protein therefor.

In another embodiment, the complex of the present invention can be a complex according to a combination of a protein comprising the amino acid sequence shown by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:22, SEQ ID NO:23 or SEQ ID NO:24 or a protein homologous thereto or a variant thereof and a bioactive substance capable of binding to the protein.

The complex of the present invention can be preferably a complex according to any combination of (a1) to (a110) above or (b1) to (b23) above, and more preferably a complex according to any combination of (c1) to (c110) below:

(c1) a combination of picotamide and a protein comprising the amino acid sequence shown by SEQ ID NO:1;
(c2) a combination of methoxsalen and a protein comprising the amino acid sequence shown by SEQ ID NO:2;
(c3) a combination of terfenadine and a protein comprising the amino acid sequence shown by SEQ ID NO:3, SEQ ID NO:14 or SEQ ID NO:19;
(c4) a combination of cyclosporin A and a protein comprising the amino acid sequence shown by SEQ ID NO:3, SEQ ID NO:8 or SEQ ID NO:12;
(c5) a combination of pancuronium bromide and a protein comprising the amino acid sequence shown by SEQ ID NO:4;
(c6) a combination of hydroxocobalamin and a protein comprising the amino acid sequence shown by SEQ ID NO:10, SEQ ID NO:11 or SEQ ID NO:15;
(c7) a combination of amphotericin B and a protein comprising the amino acid sequence shown by SEQ ID NO:15;
(c8) a combination of protriptyline and a protein comprising the amino acid sequence shown by SEQ ID NO:5;
(c9) a combination of rifampicin and a protein comprising the amino acid sequence shown by SEQ ID N0:6;
(c10) a combination of solanine α and a protein comprising the amino acid sequence shown by SEQ ID NO:7 or SEQ ID NO:15;
(c11) a combination of amethopterin and a protein comprising the amino acid sequence shown by SEQ ID NO:9;
(c12) a combination of benztropine and a protein comprising the amino acid sequence shown by SEQ ID NO:14 or SEQ ID NO:19;
(c13) a combination of sulfasalazine and a protein comprising the amino acid sequence shown by SEQ ID NO:13;
(c14) a combination of nalidixic acid and a protein comprising the amino acid sequence shown by SEQ ID NO:13;
(c15) a combination of astemizole and a protein comprising the amino acid sequence shown by SEQ ID NO:14 or SEQ ID NO:19;
(c16) a combination of chlorprothixene and a protein comprising the amino acid sequence shown by SEQ ID NO:14 or SEQ ID NO:19;
(c17) a combination of loperamide and a protein comprising the amino acid sequence shown by SEQ ID NO:14 or SEQ ID NO:19;
(c18) a combination of fluphenazine and a protein comprising the amino acid sequence shown by SEQ ID NO:14 or SEQ ID NO:19;
(c19) a combination of mefloquine and a protein comprising the amino acid sequence shown by SEQ ID NO:12, SEQ ID NO:14 or SEQ ID NO:19;
(c20) a combination of perphenazine and a protein comprising the amino acid sequence shown by SEQ ID NO:14 or SEQ ID NO:19;
(c21) a combination of perhexyline and a protein comprising the amino acid sequence shown by SEQ ID NO:12 or SEQ ID NO:14;
(c22) a combination of raloxifene and a protein comprising the amino acid sequence shown by SEQ ID NO:12, SEQ ID NO:14 or SEQ ID NO:19;
(c23) a combination of simvastatin and a protein comprising the amino acid sequence shown by SEQ ID NO:15;
(c24) a combination of benoxinate and a protein comprising the amino acid sequence shown by SEQ ID NO:16;
(c25) a combination of pioglitazone and a protein comprising the amino acid sequence shown by SEQ ID NO:17;
(c26) a combination of thioproperazine and a protein comprising the amino acid sequence shown by SEQ ID NO:12 or SEQ ID NO:18;
(c27) a combination of quinacrine and a protein comprising the amino acid sequence shown by SEQ ID NO:12 SEQ ID NO:19;
(c28) a combination of GBR12909 and a protein comprising the amino acid sequence shown by SEQ ID NO:19;
(c29) a combination of benzethonium and a protein comprising the amino acid sequence shown by SEQ ID NO:20 or SEQ ID NO:24;
(c30) a combination of albendazole and a protein comprising the amino acid sequence shown by SEQ ID NO:24;
(c31) a combination of acetopromazine and a protein comprising the amino acid sequence shown by SEQ ID NO:11;
(c32) a combination of amikacin and a protein comprising the amino acid sequence shown by SEQ ID NO:12;
(c33) a combination of amiodarone and a protein comprising the amino acid sequence shown by SEQ ID NO:12;
(c34) a combination of apigenin and a protein comprising the amino acid sequence shown by SEQ ID NO:15;
(c35) a combination of buprenorphine and a protein comprising the amino acid sequence shown by SEQ ID NO:12;
(c36) a combination of celestine blue and a protein comprising the amino acid sequence shown by SEQ ID NO:15;
(c37) a combination of chlorambucil and a protein comprising the amino acid sequence shown by SEQ ID NO:8;
(c38) a combination of chlorhexidine and a protein comprising the amino acid sequence shown by SEQ ID NO:12;
(c39) a combination of chlorpromazine and a protein comprising the amino acid sequence shown by SEQ ID NO:24;
(c40) a combination of cinchonine and a protein comprising the amino acid sequence shown by SEQ ID NO:15;
(c41) a combination of clofazimine and a protein comprising the amino acid sequence shown by SEQ ID NO:24;
(c42) a combination of clomiphene and a protein comprising the amino acid sequence shown by SEQ ID NO:12;
(c43) a combination of cyproheptadine and a protein comprising the amino acid sequence shown by SEQ ID NO:22;
(c44) a combination of deferoxamine and a protein comprising the amino acid sequence shown by SEQ ID NO:3;
(c45) a combination of dihydroergocristine and a protein comprising the amino acid sequence shown by SEQ ID NO:12;
(c46) a combination of dihydroergotamine and a protein comprising the amino acid sequence shown by SEQ ID NO:12;
(c47) a combination of diphemanil and a protein comprising the amino acid sequence shown by SEQ ID NO:11;
(c48) a combination of domperidone and a protein comprising the amino acid sequence shown by SEQ ID NO:23;
(c49) a combination of doxazosin and a protein comprising the amino acid sequence shown by SEQ ID NO:12;
(c50) a combination of eburnamonine and a protein comprising the amino acid sequence shown by SEQ ID NO:3;
(c51) a combination of ellipticine and a protein comprising the amino acid sequence shown by SEQ ID NO:24;
(c52) a combination of emetine and a protein comprising the amino acid sequence shown by SEQ ID NO:12;
(c53) a combination of ethotoin and a protein comprising the amino acid sequence shown by SEQ ID NO:13;
(c54) a combination of fenbendazole and a protein comprising the amino acid sequence shown by SEQ ID NO:12;
(c55) a combination of flumequine and a protein comprising the amino acid sequence shown by SEQ ID NO:13;
(c56) a combination of flunarizine and a protein comprising the amino acid sequence shown by SEQ ID NO:12;
(c57) a combination of flupentixol and a protein comprising the amino acid sequence shown by SEQ ID NO:12;
(c58) a combination of glipizide and a protein comprising the amino acid sequence shown by SEQ ID NO:24;
(c59) a combination of harmaline and a protein comprising the amino acid sequence shown by SEQ ID NO:15;
(c60) a combination of hydantoin and a protein comprising the amino acid sequence shown by SEQ ID NO:10;
(c61) a combination of lidoflazine and a protein comprising the amino acid sequence shown by SEQ ID NO:12;
(c62) a combination of lisinopril and a protein comprising the amino acid sequence shown by SEQ ID NO: 17;
(c63) a combination of mafenide and a protein comprising the amino acid sequence shown by SEQ ID NO:6;
(c64) a combination of mefenamic acid and a protein comprising the amino acid sequence shown by SEQ ID NO:24;
(c65) a combination of megestrol and a protein comprising the amino acid sequence shown by SEQ ID NO:24;
(c66) a combination of mesoridazine and a protein comprising the amino acid sequence shown by SEQ ID NO:3;
(c67) a combination of metergoline and a protein comprising the amino acid sequence shown by SEQ ID NO:12;
(c68) a combination of methoxy-6-harmalan and a protein comprising the amino acid sequence shown by SEQ ID NO:15;
(c69) a combination of meticrane and a protein comprising the amino acid sequence shown by SEQ ID NO:15;
(c70) a combination of methixene and a protein comprising the amino acid sequence shown by SEQ ID NO:24;
(c71) a combination of mifepristone and a protein comprising the amino acid sequence shown by SEQ ID NO:7;
(c72) a combination of nordiazepam and a protein comprising the amino acid sequence shown by SEQ ID NO: 24;
(c73) a combination of oxethazaine and a protein comprising the amino acid sequence shown by SEQ ID NO:12;
(c74) a combination of oxolinic acid and a protein comprising the amino acid sequence shown by SEQ ID NO:12;
(c75) a combination of paclitaxel and a protein comprising the amino acid sequence shown by SEQ ID NO:15;
(c76) a combination of palmatine and a protein comprising the amino acid sequence shown by SEQ ID NO:15;
(c77) a combination of pentoxifylline and a protein comprising the amino acid sequence shown by SEQ ID NO:24;
(c78) a combination of pimozide and a protein comprising the amino acid sequence shown by SEQ ID NO:3 or SEQ ID NO:12;
(c79) a combination of pinacidil and a protein comprising the amino acid sequence shown by SEQ ID NO:24;
(c80) a combination of rescinnamine and a protein comprising the amino acid sequence shown by SEQ ID NO:12;
(c81) a combination of SR-95639A and a protein comprising the amino acid sequence shown by SEQ ID NO:11;
(c82) a combination of sulfadimethoxine and a protein comprising the amino acid sequence shown by SEQ ID NO:15;
(c83) a combination of syrosingopine and a protein comprising the amino acid sequence shown by SEQ ID NO:24;
(c84) a combination of tamoxifen and a protein comprising the amino acid sequence shown by SEQ ID NO:12;
(c85) a combination of tenoxicam and a protein comprising the amino acid sequence shown by SEQ ID NO:13;
(c86) a combination of thioridazine and a protein comprising the amino acid sequence shown by SEQ ID NO:12;
(c87) a combination of thiothixene (cis) and a protein comprising the amino acid sequence shown by SEQ ID NO:12 or SEQ ID NO:24;
(c88) a combination of tomatidine and a protein comprising the amino acid sequence shown by SEQ ID NO:24;
(c89) a combination of dipyrone and a protein comprising the amino acid sequence shown by SEQ ID NO:24;
(c90) a combination of ethyl loflazepate and a protein comprising the amino acid sequence shown by SEQ ID NO:24;
(c91) a combination of clobazam and a protein comprising the amino acid sequence shown by SEQ ID NO:24;
(c92) a combination of alimemazine and a protein comprising the amino acid sequence shown by SEQ ID NO:12;
(c93) a combination of ebastine and a protein comprising the amino acid sequence shown by SEQ ID NO:24;
(c94) a combination of reserpine and a protein comprising the amino acid sequence shown by SEQ ID NO:12;
(c95) a combination of paramethasone and a protein comprising the amino acid sequence shown by SEQ ID NO:15;
(c96) a combination of hydroxyprogesterone and a protein comprising the amino acid sequence shown by SEQ ID NO:12;
(c97) a combination of dydrogesterone and a protein comprising the amino acid sequence shown by SEQ ID 30. NO:24;
(c98) a combination of sarpogrelate and a protein comprising the amino acid sequence shown by SEQ ID NO:15;
(c99) a combination of demeclocycline and a protein comprising the amino acid sequence shown by SEQ ID NO: 15;
(c100) a combination of avermectin B1A and a protein comprising the amino acid sequence shown by SEQ ID NO:15;
(c101) a combination of solasodine and a protein comprising the amino acid sequence shown by SEQ ID NO:24;
(c102) a combination of nanofin (cis) and a protein comprising the amino acid sequence shown by SEQ ID NO:20;
(c103) a combination of tetrazoline and a protein comprising the amino acid sequence shown by SEQ ID NO:11;
(c104) a combination of methylbenzethonium chloride and a protein comprising the amino acid sequence shown by SEQ ID NO:23 or SEQ ID NO:24;
(c105) a combination of α-ergocryptine and a protein comprising the amino acid sequence shown by SEQ ID NO:24;
(c106) a combination of spiramycin and a protein comprising the amino acid sequence shown by SEQ ID NO:6;
(c107) a combination of chloropyramine and a protein comprising the amino acid sequence shown by SEQ ID NO:15;
(c108) a combination of bergenin and a protein comprising the amino acid sequence shown by SEQ ID NO:15;
(c109) a combination of nafcillin and a protein comprising the amino acid sequence shown by SEQ ID NO:15;
(c110) a combination of carboprost and a protein comprising the amino acid sequence shown by SEQ ID NO:15.

The present invention also provides a method of producing a complex comprising a bioactive substance and a target protein therefor, which comprises bringing the bioactive substance and the target protein therefor into contact with each other. This contact can be performed by, for example, mixing the bioactive substance and the target protein in solution.

The complex of the present invention and the method of producing the complex can be useful in, for example, performing the screening methods of the present invention or the derivative production method of the present invention, or in cases where the complex is structurally analyzed to extensively investigate the mode of interaction between a bioactive substance and a target protein thereof, and the like.

### 6. Kit

The present invention provides a kit comprising a bioactive substance or a salt thereof.

In one embodiment, the kit of the present invention comprises the following (i) and (ii):

(i) a bioactive substance or a salt thereof;
(ii) a target protein for a bioactive substance, a nucleic acid that encodes the protein, an expression vector comprising the nucleic acid, cells enabling a measurement of the expression of a target gene for the bioactive substance, or an expression vector comprising the transcription regulatory region of a target gene for the bioactive substance and a reporter gene functionally linked to the region.

Provided that the kit of the present invention comprises a target protein for a bioactive substance, the protein is not in the form of a complex with the bioactive substance.

The bioactive substance, the target protein and target gene therefor, and the combination of bioactive substance and target protein therefor are the same as those described above (see, e.g., “5. Complex, and a method of producing the same”). The expression vector, the cells enabling a measurement of the expression of a target gene for a bioactive substance, the transcription regulatory region of the target gene for the bioactive substance, and the reporter gene functionally linked to the region, are the same as those described above (see, e.g., “2. Screening method, and product obtained by the method”).

The above-described kit of the present invention can be useful in, for example, performing the screening methods of the present invention, the derivative production method of the present invention, and the complex production method of the present invention and the like.

### 7. Determination Methods and Determination Kits for the Onset or Risk of Onset of Disease or Condition

The present invention provides determination methods and determination kits for the onset or risk of onset of a specified disease or condition. The determination methods and determination kits of the present invention can be roughly divided into determination methods and determination kits based on measurement of the expression level, and determination methods and determination kits based on identification of the polymorphism. Furthermore, they can be classified into determination methods and determination kits for the onset or risk of onset of a disease or condition associated with bioactive substance X, and determination methods and determination kits for the onset or risk of onset of a disease or condition associated with target gene Y, from the viewpoint of the disease or condition for which a determination of the onset or risk of onset is desired. The individual determination methods and determination kits are hereinafter described in detail. As required, “the expression of target protein Y or the gene that encodes the protein” is sometimes referred to as “expression of target protein Y” or “expression of target gene Y”, and “function of target protein Y or a gene that encodes the protein” is sometimes referred to as “function of target protein Y” or “function of target gene Y” as required.

### 7.1. Determination Methods and Determination Kits for the Onset or Risk of Onset of Disease or Condition on the Basis of Measurement of the Expression Level of Target Gene Y

7.1.1. Determination Method for the Onset or Risk of Onset of Disease or Condition Associated with Bioactive Substance X on the Basis of Measurement of the Expression Level of Target Gene Y (Determination Method I)

The present invention provides a determination method for the onset or risk of onset of a disease or condition associated with bioactive substance X, which comprises measuring the expression level of target gene Y.

This determination method is referred to as “determination method I” as required.

In one embodiment, determination method I comprises the following steps (a) and (b):

(a) a step for measuring the expression level of target gene Y in a biological sample collected from an animal;
(b) a step for evaluating the onset or likelihood of onset of a disease or condition associated with bioactive substance X on the basis of the expression level of target gene Y.

The methodology comprising the above-described steps (a) to (b) is referred to as “methodology V” as required.

In step (a) of methodology V, the expression level of target gene Y in a biological sample collected from an animal is measured. Although the animal is not particularly limited, a mammal or a bird is preferable, with greater preference given to a mammal. Examples of the mammal include laboratory animals such as mice, rats, hamsters, guinea pigs, and rabbits, domestic animals such as swine, bovine, goat, horses, and sheep, companion animals such as dogs and cats, and primates such as monkeys, orangutans, chimpanzees, and humans. Examples of the bird include chicken, partridges, turkeys, and ostriches.

The biological sample may be any sample containing a tissue expressing target gene Y, or any sample containing secreted target protein Y. The sample containing a tissue expressing target gene Y differs according to the kind of target gene Y. The tissue expressing target gene Y can be examined using, for example, H-Inv DB. The sample containing secreted target protein Y differs according to the kind of target gene Y, and can, for example, be blood, plasma, serum, saliva, cerebrospinal fluid, tear, or urine.

In this step, a biological sample collected from an animal in advance is used; of course, this methodology V can further comprise a step for collecting a biological sample from an animal. Collection of a biological sample from an animal can be performed by a method known per se.

The expression level of target gene Y can be measured by a method known per se with a product, for example, a transcription product or translation product, of target gene Y, as the subject. For example, the expression level of a transcription product can be measured by preparing total RNA from the cells, and performing RT-PCR, Northern blotting and the like. The expression level of a translation product can also be measured by preparing an extract from the cells, and performing an immunological technique. Useful immunological techniques include radioimmunoassay (RIA) method, ELISA method (Methods in Enzymol. 70: 419-439 (1980)), fluorescent antibody technique, and the like.

In step (b) of methodology V, a determination is made whether or not the animal is suffering from a disease or condition associated with bioactive substance X on the basis of the expression level of target gene Y. Specifically, first, the measured expression level of target gene Y is compared with the expression level of target gene Y in an animal that has not contracted the disease or condition associated with bioactive substance X (e.g., a normal animal). This comparison of expression level is preferably performed on the basis of the presence or absence of a significant difference. The expression level of target gene Y in an animal that has not contracted the disease or condition associated with bioactive substance X can be determined by a method known per se.

Next, on the basis of the result of the comparison of the expression level of target gene Y, a judgement is made whether or not the animal is possibly suffering from a disease or condition associated with bioactive substance X, or is likely or unlikely to suffer from the same in the future. The combination of a disease or condition associated with bioactive substance X and target gene Y is the same as described above. It is known that in animals that have contracted a particular disease, a change in the expression of the gene associated with the disease is often observed. It is also known that prior to the onset of a particular disease, a change in the expression of the particular gene is often observed. Hence, by analyzing the expression level of target gene Y, it is possible to determine the onset or likelihood of onset of the disease or condition associated with bioactive substance X.

Determination method I enables a determination of the presence or absence of a disease or condition associated with bioactive substance X, or the likelihood of contracting the disease or condition. Hence, determination method I is useful for, for example, the easy and early detection of the disease or condition and the like.

7.1.2. Determination Kit for the Onset or Risk of Onset of Disease or Condition Associated with Bioactive Substance X on the Basis of Measurement of Expression Level of Target Gene Y (Determination Kit I)

The present invention provides a determination kit that enables the easy conduct of determination method I.

This determination kit is referred to as “determination kit I” as required.

In one embodiment, determination kit I comprises the following (i) and (ii):

(i) a means capable of measuring the expression level of target gene Y;
(ii) a medium recording the relationship between a disease or condition associated with bioactive substance X and the expression level of target gene Y.

The kit may further comprise a means capable of collecting a biological sample from an animal, or a transcription product of target gene Y or target protein Y and the like.

The means capable of measuring the expression level of target gene Y is not subject to limitation, as long as it allows a quantitation of the expression level of target gene Y; for example, such means are roughly divided into means capable of quantifying target protein Y, and means capable of quantifying a transcription product of target gene Y. The means may be labeled with a labeling substance. Provided that the means is not labeled with a labeling substance, the determination kit of the present invention may further comprise the labeling substance. The labeling substance is the same as described above.

Specifically, the means capable of quantifying target protein Y include an antibody against target protein Y (described above), bioactive substance X and the like. The antibody against target protein Y and bioactive substance X may be provided in a form immobilized on a chip such as a plate.

Examples of the means capable of quantifying a transcription product of target gene Y include a nucleic acid probe for a transcription product of target gene Y, a primer pair capable of amplifying a transcription product of target gene Y and the like. The nucleic acid probe and primer pair may be provided along with a reagent for transcription product extraction.

The nucleic acid probe for the transcription product of target gene Y is not subject to limitation, as long as it enables a measurement of the amount of the transcription product of target gene Y. Although the probe may be any of DNA and RNA, preference is given to DNA in view of stability and the like. The probe may be single-stranded or double-stranded. Although the probe size is not subject to limitation, as long as it enables detection of the transcription product of target gene Y, the size is preferably about 15 to 1000 bp, more preferably about 50 to 500 bp. The probe may be provided in a form immobilized on a chip like a microarray.

A primer pair enabling the amplification of target gene Y is selected so that a nucleotide fragment of detectable size is amplified. The nucleotide fragment of detectable size can have a length of, for example, about 100 bp or more, preferably about 200 bp or more, more preferably about 500 bp or more. Although the primer size is not subject to limitation, as long as target gene Y can be amplified, it can be preferably about 15 to 100 bp, more preferably about 18 to 50 bp, further more preferably about 20 to 30 bp. Provided that the means capable of quantifying a transcription product of target gene Y is a primer pair capable of amplifying target gene Y, the determination kit can further comprise a reverse transcriptase.

The medium recording the relationship between a disease or condition associated with bioactive substance X and the expression level of target gene Y can be one recording the difference in the expression level of target gene Y between an animal suffering from a disease or condition associated with bioactive substance X and a non-suffering animal. The medium can be a document or a computer-readable recording medium, for example, a flexible disc, CD, DVD, hard disk and the like. The expression level of target gene Y in an animal suffering from a disease or condition associated with bioactive substance X can be increased or decreased compared to an animal not suffering from the disease or the condition.

Any means can be used to collect a biological sample from an animal, as long as it allows the obtainment of the biological sample from the animal; examples include blood drawing instruments such as injectors, biopsy instruments such as biopsy needles and biopsy forceps, surgical instruments such as surgical knives and scissors, and the like.

The transcription product of target gene Y or target protein Y can be used as, for example, a control.

Determination kit I enables a determination of the presence or absence of a disease or condition associated with bioactive substance X, or the likelihood of contracting the disease or condition. Hence, determination kit I is useful for, for example, the easy and early detection of the disease or condition and the like.

### 7.2. Determination Methods and Determination Kits for the Risk of Onset of Disease or Condition on the Basis of Identification of Polymorphism of Target Gene Y

7.2.1. Determination Method for the Risk of Onset of Disease or Condition Associated with Bioactive Substance X on the Basis of Identification of Polymorphism of Target Gene Y (Determination Method II)

The present invention provides a determination method for the risk of onset of a disease or condition associated with bioactive substance X, which comprises identifying the polymorphism of target gene Y.

This determination method is referred to as “determination method II” as required.

In one embodiment, determination method II comprises the following steps (a) and (b):

(a) a step for identifying the polymorphism of target gene Y in a biological sample collected from an animal;
(b) a step for evaluating the likelihood of the onset of a disease or condition associated with bioactive substance X on the basis of the type of polymorphism.

The methodology comprising the above-described steps (a) to (b) is referred to as “methodology VI” as required.

In step (a) of methodology VI, the type of polymorphism of target gene Y in a biological sample collected from an animal is identified. The animal is the same as described above.

Although the biological sample used may be one described with respect to methodology V above, this methodology VI enables the use of any tissue containing genomic DNA such as hair, nails, skin or mucosa as the biological sample. In view of the ease of procurement, burden on the human body and the like, the biological sample is preferably a sample of hair, nails, skin, mucosa, blood, plasma, serum, saliva and the like.

In this step, a biological sample previously collected from an animal is used, but of course this methodology VI can further comprise a step for collecting a biological sample from an animal. Collection of a biological sample from an animal can be performed by a method known per se.

A polymorphism of target gene Y means a mutation found at a frequency in the nucleotide sequence of the genomic DNA comprising target gene Y in a certain population, and can be one or more DNA substitutions, deletions, or additions (e.g., SNP, haplotype) in the genomic DNA comprising target gene Y, and a repeat, inversion, translocation and the like of the genomic DNA. Various types of polymorphism of target gene Y are registered with known databases, for example, H-Inv DB and the like. The type of polymorphism of target gene Y used in this determination method is a mutation in a nucleotide sequence whose frequency differs between animals suffering from a disease or condition associated with bioactive substance X and non-suffering animals out of all types of polymorphism in target gene Y, and can be, for example, one that alters the expression of target gene Y or alters a function associated with target protein Y (e.g., the ability of target protein Y to bind to bioactive substance X). Such types of polymorphism can be determined by a method known per se such as linkage analysis.

A determination of the type of polymorphism can be performed by a method known per se. For example, the RFLP (restriction fragment length polymorphism) method, the PCR-SSCP (single-stranded DNA conformation polymorphism) analysis method, the ASO (allele specific oligonucleotide) hybridization method, the direct sequencing method, the ARMS (amplification refracting mutation system) method, the denaturing gradient gel electrophoresis method, the RNase A cleavage method, the DOL (dye-labeled oligonucleotide ligation) method, the TaqMan PCR method, the invader method, the MALDI-TOF/MS method (matrix assisted laser desorption-time of flight/mass spectrometry) method), the TDI (template-directed dye-terminator incorporation) method and the like can be used.

In step (b) of methodology VI, a determination of the likelihood of contracting a disease or condition associated with bioactive substance X in an animal is made on the basis of the type of polymorphism. The combination of a disease or condition associated with bioactive substance X and target gene Y is the same as described above. It is known that animals susceptible to a particular disease often have a particular type of polymorphism in the gene associated with the disease. Hence, it is possible to determine the likelihood of the onset of a disease or condition associated with bioactive substance X by polymorphism analysis.

Determination method II enables a determination of the likelihood of contracting a disease or condition associated with bioactive substance X. Hence, determination method II is useful for the provision of an incentive for improving one's lifestyle for the purpose of preventing the disease or condition and the like.

7.2.2. Determination Kit for the Risk of Onset of Disease or Condition Associated with Bioactive Substance X on the Basis of Identification of Polymorphism of Target Gene Y (Determination Kit II)

The present invention also provides a determination kit that enables the easy conduct of determination method II.

This determination kit is referred to as “determination kit II” as required.

In one embodiment, determination kit II comprises the following (i) and (ii):

(i) a means capable of identifying the polymorphism of target gene Y;
(ii) a medium recording the relationship between a disease or condition associated with bioactive substance X and the polymorphism of target gene Y.

The kit may further comprise a means capable of collecting of a biological sample from an animal, or a nucleic acid that encodes target gene Y having a particular type of polymorphism, a nucleic acid that encodes target gene Y not having a particular type of polymorphism and the like.

The means capable of identifying the polymorphism of target gene Y is not subject to limitation, as long as it is capable of determining the polymorphism of target gene Y. The means may be labeled with a labeling substance. Provided that the means is not labeled with a labeling substance, this kit may further comprise the labeling substance. The labeling substance is the same as described above.

Specifically, the means capable of identifying the polymorphism of target gene Y can be a nucleic acid probe enabling a specific identification of target gene Y having a particular type of polymorphism, or a primer pair capable of specifically amplifying target gene Y having a particular type of polymorphism. The nucleic acid probe and primer pair can be ones for a genomic DNA comprising target gene Y or for a transcription product of target gene Y. The nucleic acid probe and primer pair may be provided along with a transcription product or a reagent for genomic DNA extraction.

The nucleic acid probe enabling a specific identification of target gene Y having a particular type of polymorphism is not subject to limitation, as long as target gene Y having a particular type of polymorphism can be selected. Although the probe may be any of DNA and RNA, preference is given to DNA in view of stability and the like. The probe may be any of single-stranded and double-stranded. The probe size is preferably as short as possible to enable selecting of target gene Y having a particular type of polymorphism, and can be, for example, a size of about 15 to 30 bp. The probe may be provided in a form immobilized on a chip like a microarray. The probe enables, for example, ASO (allele specific oligonucleotide) hybridization method.

The primer pair capable of specifically amplifying target gene Y having a particular type of polymorphism is selected so that a nucleotide fragment of measurable size is amplified. Such a primer pair is designed so that, for example, a polymorphism site is present at the 3′ terminus of either primer. The nucleotide fragment of measurable size can, for example, have a length of about 100 bp or more, preferably about 200 bp or more, more preferably about 500 bp or more. The primer size is not subject to limitation, as long as target gene Y can be amplified, and can be preferably about 15 to 100 bp, more preferably about 18 to 50 bp, further more preferably about 20 to 30 bp. Provided that the means capable of identifying the polymorphism of target gene Y is a primer pair for a transcription product of target gene Y, the determination kit can further comprise a reverse transcription enzyme.

As another means capable of identifying the polymorphism of target gene Y, a restriction enzyme that recognizes a site of a particular type of polymorphism can be mentioned. This means enables polymorphism analysis by RFLP.

The medium recording the relationship between a disease or condition associated with bioactive substance X and the polymorphism of target gene Y can be one recording the difference in the nucleotide sequence of the genomic DNA comprising target gene Y between an animal suffering from the disease or condition associated with bioactive substance X and a non-suffering animal. For example, the medium can be a document or a computer-readable recording medium such as a flexible disk, CD, DVD, and hard disk.

The means capable of collecting a biological sample from an animal is the same as described above.

A nucleic acid that encodes target gene Y having a particular type of polymorphism, and a nucleic acid that encodes target gene Y not having a particular type of polymorphism can, for example, be used as controls.

Determination kit II enables a determination of the likelihood of contracting a disease or condition associated with bioactive substance X. Hence, determination kit II is useful for the provision of an incentive for improving one's lifestyle for the purpose of preventing the disease or condition and the like.

7.2.3. Method of Determining the Risk of Onset of Disease or Condition Associated with Target Gene Y on the Basis of Identification of Polymorphism of Target Gene Y (Determination Method III)

The present invention provides a determination method for the risk of onset of a disease or condition associated with target gene Y, which comprises identifying the polymorphism of target gene Y.

This determination method is referred to as “determination method III” as required.

In one embodiment, determination method III comprises the following steps (a) and (b):

(a) a step for determining the type of the polymorphism of target protein Y in a biological sample collected from an animal;
(b) a step for evaluating the likelihood of the onset of a disease or condition associated with target gene Y on the basis of the type of polymorphism.

In determination method III, the type of polymorphism used to determine the risk of onset alters the ability of target protein Y to bind to bioactive substance X. The type of polymorphism can be determined by a method known per se such as binding assay.

The methodology comprising steps (a) and (b) above in determination method III is the same as methodology VI except for the type of polymorphism of target gene Y to be identified.

Determination method III enables a determination of the likelihood of contracting a disease or condition associated with target gene Y. Hence, determination method III is useful for the provision of an incentive for improving one's lifestyle for the purpose of preventing the disease or condition and the like.

7.2.4. Determination Kit for the Risk of Onset of Disease or Condition Associated with Target Gene Y on the Basis of Identification of Polymorphism of Target Gene Y (Determination Kit III)

The present invention also provides a determination kit that enables the easy conduct of determination method III.

This determination kit is referred to as “determination kit III” as required.

In one embodiment, determination kit III comprises the following (i) and (ii):

(i) a means capable of identifying the polymorphism of target gene Y;
(ii) a medium recording the relationship between a disease or condition associated with target gene Y and the polymorphism of target gene Y.

The kit may further comprise a means capable of collecting of a biological sample from an animal, or a nucleic acid that encodes target gene Y having a particular type of polymorphism, a nucleic acid that encodes target gene Y not having a particular type of polymorphism and the like.

In determination kit III, the type of polymorphism used to determine the risk of onset is one that alters the ability of target protein Y to bind to bioactive substance X. The type of polymorphism can be determined by a method known per se such as binding assay.

The constituents of determination kit III are the same as those of determination kit II except for the type of polymorphism of target gene Y to be identified.

Determination kit III enables a determination of the likelihood of contracting a disease or condition associated with target gene Y. Hence, determination kit III is useful for the provision of an incentive for improving one's lifestyle for the purpose of preventing the disease or condition and the like.

### 8. Determination Methods and Determination Kits for Susceptibility to Bioactive Substances

The present invention provides determination methods and determination kits for susceptibility to a bioactive substance. The determination methods and determination kits of the present invention can be roughly divided into determination methods and determination kits based on measurement of expression level, and determination methods and determination kits based on identification of polymorphism. Furthermore, they are classified into determination methods and determination kits for a disease or condition associated with bioactive substance X, and determination methods and determination kits for a disease or condition associated with target gene Y, from the viewpoint of a disease or condition for which a determination of susceptibility is desired. The individual determination methods and determination kits are hereinafter described in detail.

### 8.1. Determination Methods and Determination Kits for Susceptibility to Bioactive Substances on the Basis of Measurement of the Expression Level of Target Gene Y

8.1.1. Determination Method for Susceptibility to Bioactive Substance X in Disease or Condition Associated with Bioactive Substance X on the Basis of Measurement of the Expression Level of Target Gene Y (Determination Method IV)

The present invention provides a determination method for susceptibility to bioactive substance X in a disease or condition associated with bioactive substance X, which comprises measuring the expression level of target gene Y.

This determination method is referred to as “determination method IV” as required.

In one embodiment, determination method IV comprises the following steps (a) and (b):

(a) a step for measuring the expression level of target gene Y in a biological sample collected from an animal;
(b) a step for predicting the effect of bioactive substance X on the basis of the expression level of target gene Y.

The methodology comprising the above-described steps (a) to (b) is referred to as “methodology VII” as required.

Step (a) of methodology VII is the same as step (a) of methodology V.

In step (b) of methodology VII, the possible effect of bioactive substance X on animals is evaluated on the basis of the expression level of target gene Y. Specifically, first, the measured expression level of target gene Y is checked against data on the correlation of the expression level of target gene Y and susceptibility to bioactive substance X. The correlation between the expression level of target gene Y and susceptibility to bioactive substance X can be determined by a method known per se.

Next, from the result of the comparison, susceptibility to bioactive substance X is estimated. The combination of bioactive substance X and target gene Y are the same as described above. It is considered that in animals expressing a target gene for a bioactive substance at high levels, their susceptibility to the bioactive substance is high (or low), and that in animals expressing the same at low levels, their susceptibility is low (or high). Hence, it is possible to determine the susceptibility of an animal to bioactive substance X by analyzing the expression level of target gene Y. For example, provided that bioactive substance X is a drug, the likelihood or unlikelihood of obtainment of desired effect of the drug, or the probability of onset of adverse effect of a drug, can be determined.

Determination method IV enables a determination of susceptibility to bioactive substance X. Hence, determination method IV is useful for, for example, the evaluation of an action of bioactive substance X on a particular animal, and the like.

8.1.2. Determination Kit for Susceptibility to Bioactive Substance X in Disease or Condition Associated with Bioactive Substance X on the Basis of Measurement of the Expression Level of Target Gene Y (Determination Kit IV)

The present invention provides a determination kit that enables the easy conduct of determination method IV.

This determination kit is referred to as “determination kit IV” as required.

In one embodiment, determination kit IV comprises the following (i) and (ii):

(i) a means capable of measuring the expression level of target gene Y;
(ii) a medium recording the relationship between the effect of bioactive substance X and the expression level of target gene Y.

The kit may further comprise a means capable of collecting of a biological sample from an animal, or a transcription product of target gene Y or target protein Y and the like.

The constituents of determination kit IV are the same as those of determination kit I except medium (ii).

The medium recording the relationship between the effect of bioactive substance X and the expression level of target gene Y can be one incorporating data on the correlation of the expression level of target gene Y and susceptibility to bioactive substance X. The expression level of target gene Y in an animal highly susceptible to bioactive substance X can increase (or decrease) compared to a less susceptible animal.

Determination kit IV enables the easy determination of susceptibility to bioactive substance X. Hence, determination method IV is useful for, for example, the evaluation of an action of bioactive substance X on a particular animal and the like.

### 8.2. Determination Methods and Determination Kits for Susceptibility to Bioactive Substance X on the Basis of Identification of Polymorphism of Target Gene Y

8.2.1. Determination Method for Susceptibility to Bioactive Substance X in Disease or Condition Associated with Bioactive Substance X on the Basis of Identification of Polymorphism of Target Gene Y (Determination Method V)

The present invention provides a determination method for susceptibility to bioactive substance X in a disease or condition associated with bioactive substance X, which comprises identifying the polymorphism of target gene Y.

This determination method is referred to as “determination method V” as required.

In one embodiment, determination method V comprises the following steps (a) and (b):

(a) a step for identifying the polymorphism of target gene Y in a biological sample collected from an animal;
(b) a step for predicting the effect of bioactive substance X in a disease or condition associated with target gene Y on the basis of the presence or absence of a particular type of polymorphism.

The methodology comprising the above-described steps (a) to (b) is referred to as “methodology VIII” as required.

Step (a) of methodology VIII is the same as step (a) of methodology VII.

In step (b) of methodology VIII, the effect of bioactive substance X in a disease or condition associated with bioactive substance X is evaluated on the basis of the type of polymorphism of target gene Y. Specifically, first, the identified type of polymorphism of target gene Y is checked against data on the correlation of the type of polymorphism of target gene Y and susceptibility to bioactive substance X in a disease or condition associated with bioactive substance X. This correlation can be determined by a method known per se.

Next, from the result of the comparison, susceptibility to bioactive substance X in a disease or condition associated with bioactive substance X is estimated. The combination of bioactive substance X and target gene Y are the same as described above. It is known that in animals that are highly susceptible to a bioactive substance, a particular type of polymorphism is often observed in a target gene for the bioactive substance. Hence, it is possible to determine the susceptibility of an animal to bioactive substance X by analyzing polymorphism. For example, provided that bioactive substance X is a drug, the likelihood or unlikelihood of obtainment of desired effect of the drug, or the probability of onset of adverse reaction of a drug, can be determined.

Determination method V enables the easy determination of susceptibility to bioactive substance X in a disease or condition associated with bioactive substance X. Hence, determination method V is useful for, for example, the evaluation of an action of bioactive substance X in a disease or condition associated with bioactive substance X and the like.

8.2.2. Determination Kit for Susceptibility to Bioactive Substance X in Disease or Condition Associated with Bioactive Substance X on the Basis of Identification of Polymorphism of Target Gene Y (Determination Kit V)

The present invention also provides a determination kit that enables the easy conduct of determination method V.

This determination kit is referred to as “determination kit V” as required.

In one embodiment, determination kit V comprises the following (i) and (ii):

(i) a means capable of identifying the polymorphism of target gene Y;
(ii) a medium recording the relationship between the effect of bioactive substance X and the polymorphism of gene Y.

The kit may further comprise a means capable of collecting a biological sample from an animal, or a nucleic acid that encodes target gene Y having a particular type of polymorphism, a nucleic acid that encodes target gene Y not having a particular type of polymorphism and the like.

The constituents of determination kit V are the same as those of determination kit II except medium (ii).

The medium recording the relationship between the effect of active substance X and the polymorphism of gene Y can be one incorporating data on the correlation of susceptibility to bioactive substance X in a disease or condition associated with bioactive substance X and the type of polymorphism of target gene Y. The type of polymorphism of target gene Y in animals that are highly susceptible to bioactive substance X in a disease or condition associated with bioactive substance X can be one that encodes a protein that is more (or less) bindable to bioactive substance X than in animals that are less susceptible.

Determination kit V enables a determination of susceptibility to bioactive substance X in a disease or condition associated with bioactive substance X. Hence, determination kit V is useful for, for example, the evaluation of an action of bioactive substance X in a disease or condition associated with bioactive substance X and the like.

8.2.3. Determination Method for Susceptibility to Bioactive Substance X in Disease or Condition Associated with Target Gene Y on the Basis of Identification of Polymorphism of Target Gene Y (Determination Method VI)

The present invention provides a determination method for susceptibility to bioactive substance X in a disease or condition associated with target gene Y, which comprises identifying the polymorphism of target gene Y.

This determination method is referred to as “determination method VI” as required.

In one embodiment, determination method VI comprises the following steps (a) and (b):

(a) a step for determining the type of polymorphism of target protein Y in a biological sample collected from an animal;
(b) a step for predicting the effect of bioactive substance X in a disease or condition associated with target gene Y on the basis of the presence or absence of a particular type of polymorphism.

In this determination method, the type of polymorphism used to determine the susceptibility is one that alters the ability of target protein Y to bind to bioactive substance X. The type of polymorphism can be determined by a method known per se such as binding assay. Animals having a target gene comprising the type of polymorphism that potentiates or reduces the binding ability to the bioactive substance are thought to be highly (or poorly) susceptible to the bioactive substance; animals having a target gene comprising a type of polymorphism that reduces the binding ability are considered to be less (or more) susceptible. Hence, the susceptibility of an animal to bioactive substance X can be determined by analyzing the type of polymorphism.

The methodology comprising steps (a) and (b) above in determination method VI is the same as methodology VIII except for the type of polymorphism of target gene Y to be identified.

Determination method VI enables the easy determination of susceptibility to bioactive substance X in a disease or condition associated with bioactive substance X. Hence, determination method VI is useful for, for example, the evaluation of an action of bioactive substance X in a disease or condition associated with bioactive substance X and the like.

8.2.4. Determination Kit for Susceptibility to Bioactive Substance X in Disease or Condition Associated with Target Gene Y on the Basis of Identification of Polymorphism of Target Gene Y (Determination Kit VI)

The present invention also provides a determination kit that enables the easy conduct of determination method VI.

This determination kit is referred to as “determination kit VI” as required.

In one embodiment, determination kit VI comprises the following (i) and (ii):

(i) a means capable of identifying the polymorphism of target gene Y;
(ii) a medium recording the relationship between a disease or condition associated with target gene Y and the polymorphism of target gene Y.

The kit may further comprise a means capable of collecting a biological sample from an animal, or a nucleic acid that encodes target gene Y having a particular type of polymorphism, a nucleic acid that encodes target gene Y not having a particular type of polymorphism and the like.

In determination kit VI, the type of polymorphism used to determine the risk of onset is one that alters the ability of target protein Y to bind to bioactive substance X. The type of polymorphism can be determined by a method known per se such as binding assay.

The constituents of determination kit VI are the same as those of determination kit V except for the type of polymorphism of target gene Y to be identified.

Determination kit VI enables a determination of susceptibility to bioactive substance X in a disease or condition associated with bioactive substance X. Hence, determination kit VI is useful for, for example, the evaluation of an action of bioactive substance X in a disease or condition associated with bioactive substance X and the like.

The disclosures in all publications mentioned herein, including patents and patent application specifications, are incorporated by reference herein to the extent that all of them have been given expressly.

The present invention is hereinafter described in more detail by means of the following examples, which, however, are not to be construed as limiting the technical scope of the present invention.

### EXAMPLES

### Reference Example 1

### Method of Expressing Proteins from Human Full-Length cDNA Clone

Expression of Escherichia coli

BP-reaction was performed on human full-length cDNA clone and the cloning vector Gateway pDONR201 by the PCR cloning method using the Invitrogen Gateway system to yield an entry clone. LR-reaction was performed on this entry clone with the destination vector pDEST17 (Gateway System) and LR Clonase at 25° C. for 60 minutes to yield an expression plasmid. The protein expressed in Escherichia coli had a His-tag fused to the N-terminus thereof. Escherichia coli competent cell BL21star(DE3)pLysS were transformed with this expression plasmid, a clone incorporating the expression vector was selected, and a frozen stock was prepared. The transformant was inoculated into LB medium and precultured, after which it was transferred into SB medium and cultured to induce the expression of IPTG, and the cells were stored frozen.

### Reference Example 2

### Method of Purifying the Expressed Protein of Human Full-Length cDNA Clone

A human full-length cDNA clone was expressed as a protein with an N-terminal His tag. This clone was purified using BioRobot 8000 (Qiagen) or AKTA Crystal (Amersham). In the purification with BioRobot 8000, the expression-induced frozen stock cells in Reference Example 1 was thawed and lysed with lysozyme, after which the cells were affinity-purified using Ni-NTA Superflow 96 BioRobot Kit (Qiagen). In the purification with AKTA Crystal, affinity purification using a HisTrap HP column was followed by gel filtration purification using the Gel Filtration Column HiLoad 16/60 or a 10/30 Superdex 75 prep grade column. The purified fraction was used for interaction analysis after being subjected to SDS-PAGE to verify the estimated molecular weight and purity.

Regarding the protein for Biacore assay, harvested Escherichia coli was suspended in lysis buffer [50 mM NaH₂PO₄ pH 8.0, 0.3 M NaCl, 10 mM Imidazole, Benzoase, rLysozyme, complete EDTA free (Roche Diagnostics, cat no. 1873580)], and disrupted by sonication (2-second treatment+2 seconds, 5 minutes, on ice). Ni-NTA-agarose was added to the cell disruption liquid, the His-tag protein was bound, and the Ni-NTA-agarose was washed several times with NPI-30 buffer [50 mM NaH₂PO₄ pH 8.0, 0.3M NaCl, 30 mM Imidazole]. The purified recombinant protein was eluted from the Ni-NTA-agarose using an NPI-500 buffer containing a high concentration of imidazole [50 mM NaH₂PO₄ pH 8.0, 0.3 M NaCl, 500 mM Imidazole], after which dialysis against PBS was performed, and the imidazole was removed. After the concentration of the protein obtained was measured, its purity was measured by SDS-PAGE, and it was stored at 4° C. Expression in silkworm pupa and purification

A portion of the protein was expressed and purified by means of the contract protein production service “Superworm” of Katakura Industries Co., Ltd., which is based on a silkworm pupa expression system. A gene with a His tag added to the C-terminus thereof was inserted to a recombinant baculovirus and inoculated to a silkworm pupa. A milled product of the expressed silkworm pupa was sonicated, the centrifugal supernatant was filtered, and Ni-NTA resin or affinity purification was performed in the same manner as the Escherichia coli expression product.

### Reference Example 3

### Method of analyzing human protein-Drug Interactions Using Size Exclusion Chromatography

To analyze the interactions between commonly used drugs and proteins expressed from human full-length cDNA clones while keeping both the proteins and the compounds in non-modified, non-immobilized state, size exclusion chromatography (SEC) and mass analysis were used in combination. The specific procedures are shown below.

### Step 1

A solution of a single drug or a multiplexed compound solution comprising a mixture of a plurality of drugs (e.g., 8 kinds, 16 kinds, 24 kinds) was added to the protein purified in Reference Example 2.

### Step 2

The compound-protein mixture prepared in step 1 was subjected to chromatography using an SEC column, the compound and the protein were separated by SEC, and the compound that interacted with the bound compound or protein contained in the protein fraction was analyzed using a mass analyzer.

The purified protein standard was concentrated by ultrafiltration and subjected to buffer solution exchange, and finally concentrated to obtain a concentration of 25 μM or higher in an aqueous solution of 10 mM ADA (N-(2-acetamido)iminodiacetic acid) buffer (pH 6.5)-300 mM NaCl-100 μM mineral ion cocktail (Ca(OAc)₂, Zn(OAc)₂.2H₂O, Cu(OAc)₂—H₂O, Co(OAc)₂.4H₂O, Mn(OAc)₂.4H₂O, Mg(OAc)₂.4H₂O, FeCl₃.6H₂O). Protein concentrations were measured using BCA Protein Assay (PIERCE), in consideration of the purity calculated by SDS-PAGE.

A solution of a single pharmaceutical compound at a concentration of 1.25 mM in DMSO (dimethyl sulfoxide) or a multiplexed compound solution of a plurality (8 or 16 kinds) of compounds in DMSO was prepared, and these solutions were used for interaction analysis. In reproducibility confirmation experiments or dose dependency determination experiments, a solution of various concentrations of a single compound in DMSO (dimethyl sulfoxide) was used.

Mass analysis was performed using LCQ DECA XP (Thermoelectron) or Q-TOF micro (Micromass), equipped with an ESI probe. The LC pump used was Agilent 1100 (Yokogawa Analytical Systems), and the autosampler used was HTC-PAL (CTC Analytics) equipped with a cooling stacker. The SEC column used was a micro-LC column or a 384-well spin column.

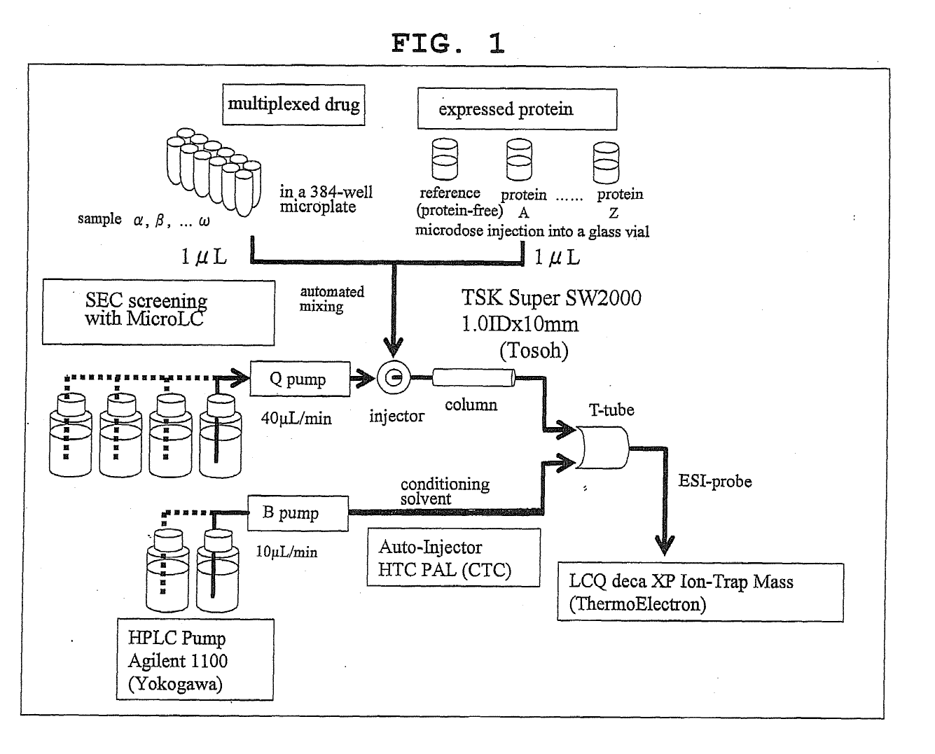

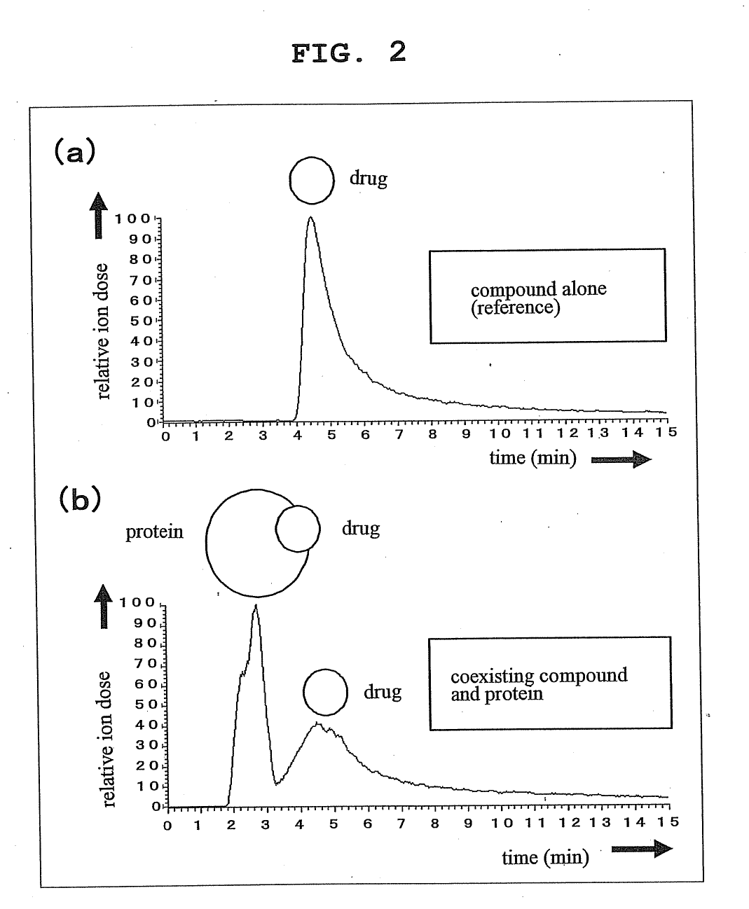

### 1. Micro-LC Method (FIGS. 1 and 2)

The SEC column used for the micro-LC method was a TSKgel super SW2000 (TOSOH) cartridge column (1.0 mm ID×10 mm) or a TSKgel super SW2000 (TOSOH) column (1.0 mm ID×30 mm), with 10 mM ammonium acetate aqueous solution serving as the mobile phase. A conditioning solvent (0.5% formic acid/methanol solution in the positive ion mode, 0.5% ammonia/methanol solution in the negative ion mode) was mixed downstream of the SEC column via a Tee connector; the column was connected to the ESI of the mass analyzer. The flow rate was set at 40 μL/min for the SEC column side, and 10 μL/min for the conditioning solvent side.

A protein standard prepared to have a concentration of 25 μM or higher and a multiplied liquid comprising 25 μM of each pharmaceutical compound (5% DMSO aqueous solution) were set onto an auto-injector; 1 μL of the compound solution and 1 μL of the protein solution were continuously and sequentially injected into the SEC column; interactions were determined by the in-the-column mixing method (Japanese Patent Application No. 2003-35400). Specifically, if the protein and the compound interact with each other, the acceleration of the elution of the compound molecules that are bound to the protein when the protein is overtaking the compound in the SEC column occurs, resulting in a change in the mass chromatogram of the compound depending on the presence or absence of the protein. If this difference in the mass chromatogram of the compound depending on the presence or absence of the protein was observed, particularly an increase in the mass spectral intensity of the compound in the protein elution fraction, the protein and the compound were judged to have interacted with each other.

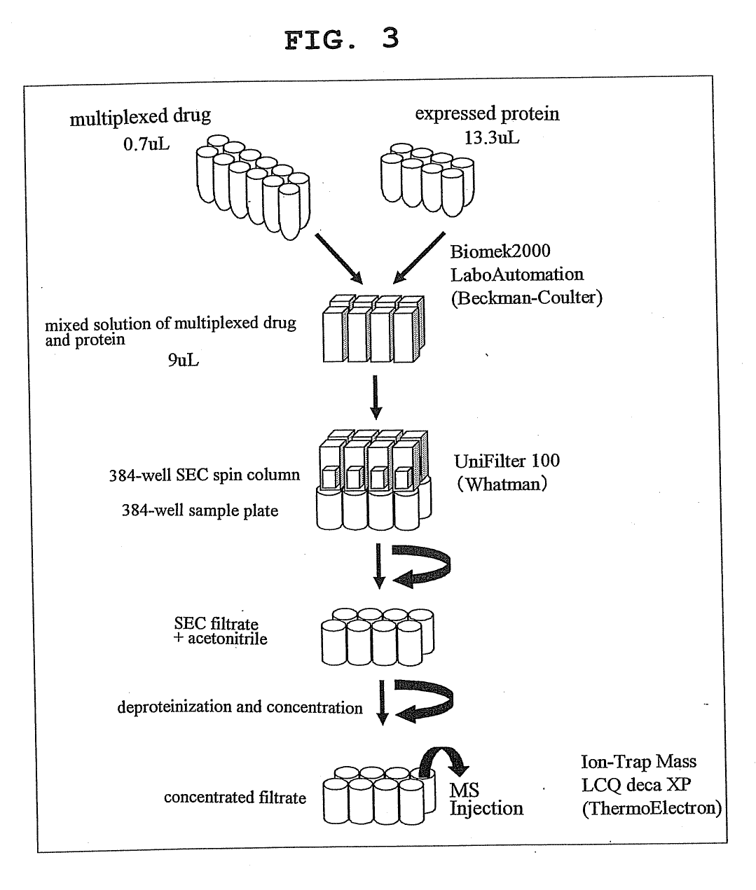

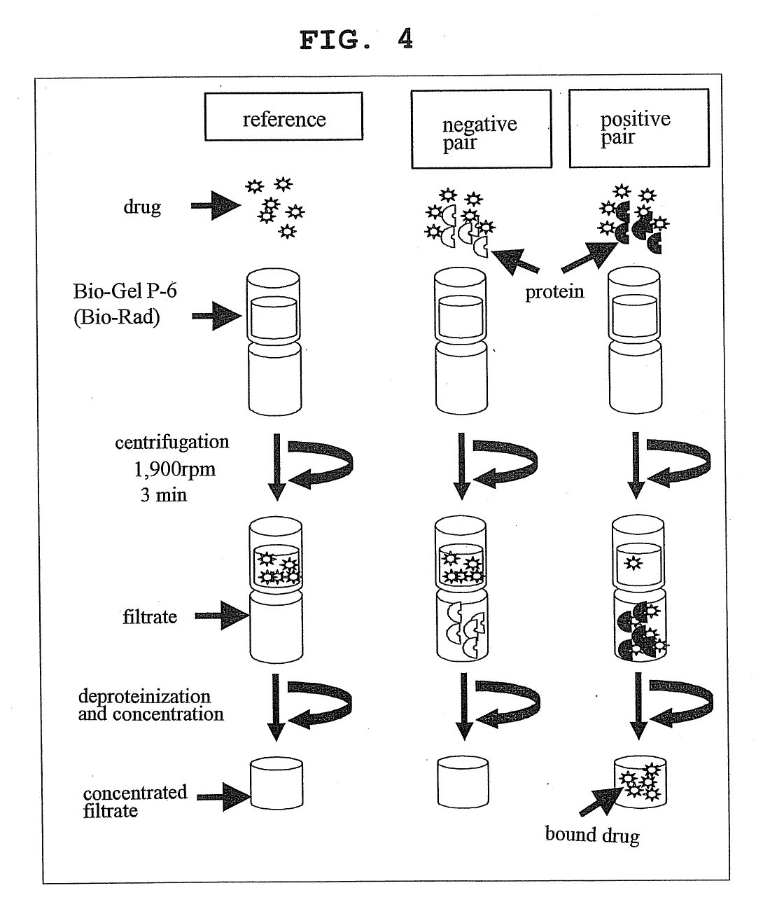

### 2. Spin Column Method (FIGS. 3 and 4)

In the 384-well spin column method, Unifilter 100 (Whatman), packed with 10 μL (dry volume) of Bio-Gel P6 (BIO-RAD) and swollen with milliQ water, was used as the SEC column. 13.3 μL of a protein-free reference standard or a 25 μM protein standard and 0.7 μL of a multiplexed solution comprising 25 μM of each pharmaceutical compound (5% DMSO aqueous solution) were mixed; 9 μL of this mixture was aliquoted into the SEC spin columns. The SEC spin column was mounted on an acetonitrile-aliquoted 384-well U-bottom plate and centrifuged; the SEC spin column filtrate, which is a protein fraction, was recovered in 50% acetonitrile. The protein precipitate produced by the acetonitrile was removed via centrifugation and filtration for deproteinization; the resulting filtrate was concentrated by centrifugal concentration and re-dissolved in 10 μL of 50% methanol to obtain a mass analysis sample. The mobile phase supplied to the mass analyzer was 0.1% formic acid/50% methanol solution in the positive ion mode, and 0.1% ammonia/50% methanol solution in the negative ion mode; these mobile phases were used at a flow rate of 40 μL/min. 2-μL of mass analysis samples were injected using an autosampler at 2-minute intervals; the mass spectral intensity of the compound was measured to retrieve the spectral intensity of the pharmaceutical compound contained in the SEC spin column filtrate (protein fraction eluted from SEC). The protein and the compound were judged to have interacted with each other if the spectral intensity of the compound in a mass analysis sample obtained from an SEC sample supplemented with a protein sample was greater than the spectral intensity of the compound in a mass analysis sample of reference SEC standard not supplemented with the protein. In the experiments for examining dose dependency, the protein and the compound were judged to have interacted with each other dose-dependently if the spectral intensity of the pharmaceutical compound contained in the SEC spin column filtrate (protein fraction eluted from SEC) increased as the compound concentration or/and protein concentration of the SEC sample was increased.

### 3. Measurement of Dissociation Constant Using

### Immobilization of Protein:

The protein was diluted with PBS to about 20 μg/mL to 40 μg/mL, and immobilized to a CM5-Sencer chip having NTA immobilized thereon by the affinity-amine-coupling method or a commercially available NTA sensor chip.

For the affinity-amine-coupling method, 0.5 M NiCl₂ was injected for 1 minute, and an EDC:NHS mixed liquid (manufactured by BIACORE) was then injected for 10 minutes to activate the sensor chip; subsequently, the protein solution was injected for 10 to 15 minutes to achieve immobilization. After the immobilization, 1M ethanolamine was injected for 7 minutes to achieve deactivation. The amount of protein immobilized varied depending on the protein, and was about 6,800 RU on average, 1,452 RU at the lowest, and 16,655 RU at the highest.

### Dilution of Compound:

As the buffer solution for assay, a Tris buffered Saline (10 mM Tris/HCl pH 7.4, 150 mM NaCl) (TBS) supplemented with 2% DMSO was mainly used; however, for the reasons of the solubility of the compound and the like, PBS or HEPES buffered Saline (10 mM HEPES/HCl pH 7.4, 150 mM NaCl) (HBS) was also used. If a trace amount of metal ion was required because of the nature of the protein-compound assayed, the buffer solution was used with the addition of 10 μM or 100 μM calcium acetate and magnesium acetate, and 1 μM zinc acetate. Because some of the compounds have low solubility, Surfactant P-20 (manufactured by BIACORE), a kind of surfactant, was added at 0.005%.

Each compound was diluted basically at six dilution rates: 100 μM, 33.3 μM, 11.1 μM, 3.7 μM, 1.23 μM, and 0.41 μM; for 33.3 μM, two measurements were taken and the reproducibility of the assay was verified.

Particularly, if a KD value of not more than 1×10⁻⁵M is obtained, the compound was diluted at 10 dilution rates: 100 μM, 50 μM, 25 μM, 10 μM, 5 μM, 2.5 μM, 1 μM, 0.5 μM, 0.25 μM, and 0.1 μM; for 100 μM, 50 μM, 25 μM, 10 μM, 5 μM, 2.5 μM, 1 μM, and 0.5 μM, two measurements were taken and the reproducibility of the assay was verified.

If the non-specific adsorption of the compound to the sensor surface was suspected from the results of the ordinary investigation, another investigation was performed with the addition of 1×10⁻⁴ M to 1×10⁻³ M ethanolamine to the buffer solution for assay.

Measurements were performed using BIACORE3000, and each compound was injected with the KINJECT command. The flow rate was 50 μL/min, injection was continued for 3 minutes, and dissociation was then measured for 3 minutes.

After injection of the compound, the sensor surface was washed by sequentially injecting 10 mM HCl (6 seconds), 1 mM NaOH (6 seconds), and 40 mM Octyl-glucose (10 seconds). This washing operation was repeated as required.

### Correction of Measured Values and Method of Calculating KD Value:

Before each measurement, DMSO was injected to the buffer solution for assay at different concentrations (1.25%, 1.75%, 2.0%, 2.25%, 2.5%, 2.75% and the like) several times, and using the values obtained, a correction for the bulk effect of DMSO (DMSO correction) was performed. The buffer solution used to dilute the compound alone was injected, and this was used for correction of the noise and the like of the apparatus (0 correction). Assay results with the DMSO correction and 0 correction were analyzed using BIAevaluation version 4.1. If binding in a stationary state is observed for each dilution as a result of a measurement, the steady state affinity was calculated and the KD value was determined. If dissociation could be observed for several minutes after the binding, or if no stationary state was reached during injection of the compound, analysis was performed using the Kinetics analysis (Simultaneous ka/kd, 1:1 binding model), and the KD value was calculated.

### Example 1

### Analysis of interaction between FLJ10420-Derived Protein and Picotamide

The FLJ10420-derived protein was expressed and purified according to the methods of Reference Examples 1 and 2, and the interaction between the protein expressed and purified from FLJ10420 and picotamide was analyzed according to the method of Reference Example 3. The results are shown in Table 10. The spectrum intensity of the pharmaceutical compound contained in the SEC spin column filtrate (protein fraction eluted from SEC) increased depending on the doses of both the small compound and the FLJ10420-derived protein, respectively, from which we determined that there was a concentration-dependent interaction between picotamide and the FLJ10420-derived protein.

Hence, the FLJ10420-derived protein was found to be a target protein for picotamide. Therefore, a new drug can be screened by making screening candidate substances interact with the FLJ10420-derived protein. Specifically, a new drug can be screened by constructing a system which detects the interaction between the FLJ10420-derived protein and a candidate substance according to, for example, the method of Reference Example 3.

TABLE 10
Mass Range for retrieval m/z = 377.2-377.5

| | |
|---|---|
| FJL10420- | compound (μM) |

| | | | | |
|---|---|---|---|---|
| picotamide | 0 | 12.5 | 25 | 62.5 |

| | | | | | |
|---|---|---|---|---|---|
| protein | 0 | 183154 | 412892 | 441470 | 841101 |
| (μM) | 5 | 229422 | 323916 | 490309 | 1376939 |
| | 10 | 416735 | 294069 | 647010 | 1450464 |
| | 15 | 190404 | 237571 | 496587 | 1862334 |

### Example 2

### Analysis of Interaction Between FLJ10537-Derived Protein and Methoxsalen

The FLJ10537-derived protein was expressed and purified according to the methods of Reference Examples 1 and 2, and the interaction between the protein expressed and purified from FLJ10537 and methoxsalen was analyzed according to the method of Reference Example 3 (data not shown). As a result, the mass chromatogram of methoxsalen in TSKgel super SW2000 micro-LC column (1.0 mm ID×30 mm) revealed earlier elution thereof in the presence of the FLJ10537-derived protein than in its absence. This shows that methoxsalen eluted earlier from the SEC micro-LC column as a result of an interaction with the FLJ10537-derived protein, from which we determined that there was an interaction between methoxsalen and the FLJ10537-derived protein.

Hence, the FLJ10537-derived protein was found to be a target protein for methoxsalen. Therefore, a new drug can be screened by making screening candidate substances interact with the FLJ10537-derived protein. Specifically, a new drug can be screened by constructing a system which detects the interaction between the FLJ10537-derived protein and a candidate substance according to, for example, the method of Reference Example 3.

### Example 3

### Example of Analysis of Interaction Between FLJ11211-Derived Protein and Terfenadine

The FLJ11211-derived protein was expressed and purified according to the methods of Reference Examples 1 and 2, and the interaction between the aforementioned protein and terfenadine was analyzed according to the method of Reference Example 3 (data not shown) As a result, it was shown that the mass spectrum peak intensity of terfenadine in the spin column filtrate was higher when terfenadine was applied to the spin column in a mixture with the FLJ11211-derived protein than in a mixture with the buffer solution alone. Hence, when FLJ11211-derived protein co-exists, terfenadine was shown to be eluted into the spin column filtrate in the form of a conjugate with the co-existing FLJ11211-derived protein, from which we determined that there was an interaction between terfenadine and the FLJ11211-derived protein.

Hence, the FLJ11211-derived protein was found to be a target protein for terfenadine. Therefore, a new drug can be screened by making the FLJ11211-derived protein interact with screening candidate substances. Specifically, a new drug can be screened by constructing a system which detects the interaction between the FLJ11211-derived protein and a candidate substance according to, for example, the method of Reference Example 3.

### Example 4

### Example of Analysis of Interaction Between FLJ11211-Derived Protein and Cyclosporin A

The FLJ11211-derived protein was expressed and purified according to the methods of Reference Examples 1 and 2, and the interaction between the aforementioned protein and cyclosporin A was analyzed according to the method of Reference Example 3 (data not shown). As a result, it was shown that the mass spectrum peak intensity of cyclosporin A in the spin column filtrate was higher when cyclosporin A was applied to the spin column in a mixture with the FLJ11211-derived protein than in a mixture with the buffer solution alone. Hence, when FLJ11211-derived protein co-exists, cyclosporin A was shown to be eluted into the spin column filtrate in the form of a conjugate with the co-existing FLJ11211-derived protein, from which we determined that there was an interaction between cyclosporin A and the FLJ11211-derived protein.

Hence, the FLJ11211-derived protein was found to be a target protein for cyclosporin A. Therefore, a new drug can be screened by making the FLJ11211-derived protein interact with screening candidate substances. Specifically, a new drug can be screened by constructing a system which detects the interaction between the FLJ11211-derived protein and a candidate substance according to, for example, the method of Reference Example 3.

### Example 5

### Analysis of Interaction Between FLJ12857-Derived Protein and Pancuronium Bromide

The FLJ12857-derived protein was expressed and purified according to the methods of Reference Examples 1 and 2, and the interaction between the protein expressed and purified from FLJ12857 and pancuronium bromide was analyzed according to the method of Reference Example 3. The results are shown in Table 11. The spectrum intensity of the pharmaceutical compound contained in the SEC spin column filtrate (protein fraction eluted from SEC) increased, depending on the doses of both the small compound and the FLJ12857-derived protein, respectively, from which we determined that there was a concentration-dependent interaction between pancuronium bromide and the FLJ12857-derived protein.

Hence, the FLJ12857-derived protein was found to be a target protein for pancuronium bromide. Therefore, a new drug can be screened by making the FLJ12857-derived protein interact with screening candidate substances. Specifically, a new drug can be screened by constructing a system which detects the interaction between the FLJ12857-derived protein and a candidate substance according to, for example, the method of Reference Example 3.

TABLE 11
Mass Range for retrieval m/z = 286.5-286.8(/200-400)

| | |
|---|---|
| FLJ12857- | |
| pancuronium | compound (μM) |

| bromide | | 0 | 12.5 | 25 | 62.5 |
|---|---|---|---|---|---|
| protein | 0 | 9682 | 50699 | 170740 | 1417386 |
| (μM) | 5 | 10687 | 65210 | 491313 | 1918601 |
| | 10 | 10562 | 163033 | 747644 | 2231237 |
| | 25 | 6965 | 193021 | 725722 | 2511342 |

### Example 6

### Analysis of Interaction Between FLJ20972-Derived Protein and Protriptyline

The FLJ20972-derived protein was expressed and purified according to the methods of Reference Examples 1 and 2, and the interaction between the protein expressed and purified from FLJ20972 and protriptyline was analyzed according to the method of Reference Example 3. The results are shown in Table 12. The spectrum intensity of the pharmaceutical compound contained in the SEC spin column filtrate (protein fraction eluted from SEC) increased depending on the doses of both the small compound and the FLJ20972-derived protein, respectively, from which we determined that there was a concentration-dependent interaction between protriptyline and the FLJ20972-derived protein.

Hence, the FLJ20972-derived protein was found to be a target protein for protriptyline. Therefore, a new drug can be screened by making the FLJ20972-derived protein interact with screening candidate substances. Specifically, a new drug can be screened by constructing a system which detects the interaction between the FLJ20972-derived protein and a candidate substance according to, for example, the method of Reference Example 3.

TABLE 12
Mass Range for retrieval m/z = 472.25-472.55

| | |
|---|---|
| FLJ20972- | compound (μM) |

| | | | | |
|---|---|---|---|---|
| protriptyline | 0 | 12.5 | 25 | 62.5 |

| | | | | | |
|---|---|---|---|---|---|
| protein | 0 | 21492 | 39179 | 53214 | 239324 |
| (μM) | 5 | 20116 | 27260 | 76771 | 514113 |
| | 10 | 34208 | 39545 | 105694 | 390017 |
| | 25 | 30876 | 44522 | 132099 | 754873 |

### Example 7

### Analysis of Interaction Between FLJ21841-Derived Protein and Rifampicin

The FLJ21841-derived protein was expressed and purified according to the methods of Reference Examples 1 and 2, and the interaction between the protein expressed and purified from FLJ21841 and rifampicin was analyzed according to the method of Reference Example 3 (data not shown). As a result, the mass chromatogram of rifampicin in TSKgel super SW2000 micro-LC column (1.0 mm ID×30 mm) revealed earlier elution thereof in the presence of the FLJ21841-derived protein than in its absence. This shows that rifampicin eluted earlier from the SEC micro-LC column as a result of an interaction with the FLJ21841-derived protein, from which we determined that there was an interaction between rifampicin and the FLJ21841-derived protein.

Hence, the FLJ21841-derived protein was found to be a target protein for rifampicin. Therefore, a new drug can be screened by making the FLJ21841-derived protein interact with screening candidate substances. Specifically, a new drug can be screened by constructing a system which detects the interaction between the FLJ21841-derived protein and a candidate substance according to, for example, the method of Reference Example 3.

### Example 8

### Analysis of Interaction Between FLJ22317-Derived Protein and Solanine A

The FLJ22317-derived protein was expressed and purified according to the methods of Reference Examples 1 and 2, and the interaction between the protein expressed and purified from FLJ22317 and solanine α was analyzed according to the method of Reference Example 3 (data not shown). As a result, the mass chromatogram of solanine α in TSKgel super SW2000 micro-LC column (1.0 mm ID×30 mm) revealed earlier elution thereof in the presence of the FLJ22317-derived protein than in its absence. This shows that solanine α eluted earlier from the SEC micro-LC column as a result of an interaction with the FLJ22317-derived protein, from which we determined that there was an interaction between solanine α and the FLJ22317-derived protein.

Hence, the FLJ22317-derived protein was found to be a target protein for solanine α. Therefore, a new drug can be screened by making the FLJ22317-derived protein interact with screening candidate substances. Specifically, a new drug can be screened by constructing a system which detects the interaction between the FLJ22317-derived protein and a candidate substance according to, for example, the method of Reference Example 3.

### Example 9

### Analysis of Interaction Between FLJ23466-Derived Protein and Cyclosporin A

The FLJ23466-derived protein was expressed and purified according to the methods of Reference Examples 1 and 2, and the interaction between the protein expressed and purified from FLJ23466 and cyclosporin A was analyzed according to the method of Reference Example 3. The results are shown in Table 13. the spectrum intensity of the pharmaceutical compound contained in the SEC spin column filtrate (protein fraction eluted from SEC) increased depending on the doses of both the small compound and the FLJ23466-derived protein, respectively, from which we determined that there was a concentration-dependent interaction between cyclosporin A and the FLJ23466-derived protein.

Hence, the FLJ23466-derived protein was found to be a target protein for cyclosporin A. Therefore, a new drug can be screened by making the FLJ23466-derived protein interact with screening candidate substances. Specifically, a new drug can be screened by constructing a system which detects the interaction between the FLJ23466-derived protein and a candidate substance according to, for example, the method of Reference Example 3.

TABLE 13
Mass Range for Msbat-retrieval m/z = 1124.7-1125(m/z)

| FLJ23466- | compound (μM) | | | |
|---|---|---|---|---|
| cyclosporin A | 0 | 12.5 | 25 | 62.5 |

| | | | | | |
|---|---|---|---|---|---|
| protein | 0 | 74979 | 85660 | 60168 | 112532 |
| (μM) | 5 | 135102 | 94538 | 755861 | 262294 |
| | 10 | 115141 | 179998 | 217427 | 384273 |
| | 25 | 237640 | 266228 | 513738 | 629580 |

### Example 10

### Analysis of Interaction Between FLJ25288-Derived Protein and Amethopterin (R, S)

The FLJ25288-derived protein was expressed and purified according to the methods of Reference Examples 1 and 2, and the interaction between the protein expressed and purified from FLJ25288 and amethopterin (R, S) was analyzed according to the method of Reference Example 3 (data not shown). As a result, the mass chromatogram of amethopterin (R, S) in TSKgel super SW2000 micro-LC column (1.0 mm ID×30 mm) revealed earlier elution thereof in the presence of the FLJ25288-derived protein than in its absence. This shows that amethopterin (R, S) eluted earlier from the SEC micro-LC column as a result of an interaction with the FLJ25288-derived protein, from which we determined that there was an interaction between amethopterin (R, S) and the FLJ25288-derived protein.

Hence, the FLJ25288-derived protein was found to be a target protein for amethopterin (R, S). Therefore, a new drug can be screened by making the FLJ25288-derived protein interact with screening candidate substances. Specifically, a new drug can be screened by constructing a system which detects the interaction between the FLJ25288-derived protein and a candidate substance according to, for example, the method of Reference Example 3.

### Example 11

### Analysis of Interaction Between FLJ35914-Derived Protein and Hydroxocobalamin

The FLJ35914-derived protein was expressed and purified according to the methods of Reference Examples 1 and 2, and the interaction between the protein expressed and purified from FLJ35914 and hydroxocobalamin was analyzed according to the method of Reference Example 3 (data not shown). As a result, the mass chromatogram of hydroxocobalamin in TSKgel super SW2000 micro-LC column (1.0 mm ID×30 mm) revealed earlier elution thereof in the presence of the FLJ35914-derived protein than in its absence. This shows that hydroxocobalamin eluted earlier from the SEC micro-LC column as a result of an interaction with the FLJ35914-derived protein, from which we determined that there was an interaction between hydroxocobalamin and the FLJ35914-derived protein.

Hence, the FLJ35914-derived protein was found to be a target protein for hydroxocobalamin. Therefore, a new drug can be screened by making the FLJ35914-derived protein interact with screening candidate substances. Specifically, a new drug can be screened by constructing a system which detects the interaction between the FLJ35914-derived protein and a candidate substance according to, for example, the method of Reference Example 3.

### Example 12

### Analysis of Interaction Between FLJ36526-Derived Protein and Hydroxocobalamin

The FLJ36526-derived protein was expressed and purified according to the methods of Reference Examples 1 and 2, and the interaction between the protein expressed and purified from FLJ36526 and hydroxocobalamin was analyzed according to the method of Reference Example 3 (data not shown). As a result, the mass chromatogram of hydroxocobalamin in TSKgel super SW2000 micro-LC column (1.0 mm ID×30 mm) revealed earlier elution thereof in the presence of the FLJ36526-derived protein than in its absence. This shows that hydroxocobalamin eluted earlier from the SEC micro-LC column as a result of an interaction with the FLJ36526-derived protein, from which we determined that there was an interaction between hydroxocobalamin and the FLJ36526-derived protein.

Hence, the FLJ36526-derived protein was found to be a target protein for hydroxocobalamin. Therefore, a new drug can be screened by making the FLJ36526-derived protein interact with screening candidate substances. Specifically, a new drug can be screened by constructing a system which detects the interaction between the FLJ36526-derived protein and a candidate substance according to, for example, the method of Reference Example 3.

### Example 13

### Example of Analysis of Interaction Between FLJ37909-Derived Protein and Cyclosporin A

The FLJ37909-derived protein was expressed and purified according to the methods of Reference Examples 1 and 2, and the interaction between the aforementioned protein and cyclosporin A was analyzed according to the method of Reference Example 3 (data not shown). As a result, it was shown that the mass spectrum peak intensity of cyclosporin A in the spin column filtrate was higher when cyclosporin A was applied to the spin column in a mixture with the FLJ37909-derived protein than in a mixture with the buffer solution alone. Hence, when FLJ37909-derived protein co-exists, cyclosporin A was shown to be eluted into the spin column filtrate in the form of a conjugate with the co-existing FLJ37909-derived protein, from which we determined that there was an interaction between cyclosporin A and the FLJ37909-derived protein.

Hence, the FLJ37909-derived protein was found to be a target protein for cyclosporin A. Therefore, a new drug can be screened by making the FLJ37909-derived protein interact with screening candidate substances. Specifically, a new drug can be screened by constructing a system which detects the interaction between the FLJ37909-derived protein and a candidate substance according to, for example, the method of Reference Example 3.

### Example 14

### Analysis of Interaction Between FLJ38531-Derived Protein and Sulfasalazine

The FLJ38531-derived protein was expressed and purified according to the methods of Reference Examples 1 and 2, and the interaction between the protein expressed and purified from FLJ38531 and sulfasalazine was analyzed according to the method of Reference Example 3. The results are shown in Table 14. The spectrum intensity of the pharmaceutical compound contained in the SEC spin column filtrate (protein fraction eluted from SEC) increased depending on the doses of both the small compound and the FLJ38531-derived protein, respectively, from which we determined that there was a concentration-dependent interaction between sulfasalazine and the FLJ38531-derived protein.

Hence, the FLJ38531-derived protein was found to be a target protein for sulfasalazine. Therefore, a new drug can be screened by making the FLJ38531-derived protein interact with screening candidate substances. Specifically, a new drug can be screened by constructing a system which detects the interaction between the FLJ38531-derived protein and a candidate substance according to, for example, the method of Reference Example 3.

TABLE 14
Mass Range for retrieval m/z = 399.1-399.4

| FLJ38531- | | compound (μM) | | | |
|---|---|---|---|---|---|
| sulfasalazine | | 0 | 12.5 | 25 | 62.5 |
| protein | 0 | 53492 | 59203 | 53384 | 179387 |
| (μM) | 5 | 42774 | 261679 | 401351 | 688076 |
| | 10 | 46343 | 686531 | 946518 | 1418687 |
| | 25 | 50491 | 2048630 | 2600573 | 3155516 |

### Example 15

### Analysis of Interaction Between FLJ38531-Derived Protein and Nalidixic Acid

The FLJ38531-derived protein was expressed and purified according to the methods of Reference Examples 1 and 2, and the interaction between the protein expressed and purified from FLJ38531 and nalidixic acid was analyzed according to the method of Reference Example 3 (data not shown). The spectrum intensity of the pharmaceutical compound contained in the SEC spin column filtrate (protein fraction eluted from SEC) increased depending on the doses of both of each small compound and the FLJ38531-derived protein, respectively, from which we determined that there was a concentration-dependent interaction between nalidixic acid and the FLJ38531-derived protein.

Hence, the FLJ38531-derived protein was found to be a target protein for nalidixic acid. Therefore, a new drug can be screened by making the FLJ38531-derived protein interact with screening candidate substances. Specifically, a new drug can be screened by constructing a system which detects the interaction between the FLJ38531-derived protein and a candidate substance according to, for example, the method of Reference Example 3.

### Example 16

### Example of Analysis of Interaction Between FLJ38897-Derived Protein and Astemizole

The FLJ38897-derived protein was expressed and purified according to the methods of Reference Examples 1 and 2, and the interaction between the aforementioned protein and astemizole was analyzed according to the method of Reference Example 3 (data not shown). As a result, it was shown that the mass spectrum peak intensity of Astemizole in the spin column filtrate was higher when Astemizole was applied to the spin column in a mixture with the FLJ38897-derived protein than in a mixture with the buffer solution alone. Hence, when FLJ38897-derived protein co-exists, astemizole was shown to be eluted into the spin column filtrate in the form of a conjugate with the co-existing FLJ38897-derived protein, from which we determined that there was an interaction between astemizole and the FLJ38897-derived protein.

Hence, the FLJ38897-derived protein was found to be a target protein for astemizole. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 17

### Example of Analysis of Interaction Between FLJ38897-Derived Protein and Chlorprothixene

The FLJ38897-derived protein was expressed and purified according to the methods of Reference Examples 1 and 2, and the interaction between the aforementioned protein and chlorprothixene was analyzed according to the method of Reference Example 3 (data not shown). As a result, it was shown that the mass spectrum peak intensity of chlorprothixene in the spin column filtrate was higher when chlorprothixene was applied to the spin column in a mixture with the FLJ38897-derived protein than in a mixture with the buffer solution alone. Hence, when FLJ38897-derived protein co-exists, chlorprothixene was shown to be eluted into the spin column filtrate in the form of a conjugate with the co-existing FLJ38897-derived protein, from which we determined that there was an interaction between chlorprothixene and the FLJ38897-derived protein.

Hence, the FLJ38897-derived protein was found to be a target protein for chlorprothixene. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 18

### Example of Analysis of Interaction Between FLJ38897-Derived Protein and Benztropine

The FLJ38897-derived protein was expressed and purified according to the methods of Reference Examples 1 and 2, and the interaction between the aforementioned protein and benztropine was analyzed according to the method of Reference Example 3 (data not shown). As a result, it was shown that the mass spectrum peak intensity of benztropine in the spin column filtrate was higher when benztropine was applied to the spin column in a mixture with the FLJ38897-derived protein than in a mixture with the buffer solution alone. Hence, when FLJ38897-derived protein co-exists, benztropine was shown to be eluted into the spin column filtrate in the form of a conjugate with the co-existing FLJ38897-derived protein, from which we determined that there was an interaction between benztropine and the FLJ38897-derived protein.

Hence, the FLJ38897-derived protein was found to be a target protein for benztropine. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 19

### Example of Analysis of Interaction Between FLJ38897-Derived Protein and Loperamide

The FLJ38897-derived protein was expressed and purified according to the methods of Reference Examples 1 and 2, and the interaction between the aforementioned protein and loperamide was analyzed according to the method of Reference Example 3 (data not shown). As a result, it was shown that the mass spectrum peak intensity of loperamide in the spin column filtrate was higher when loperamide was applied to the spin column in a mixture with the FLJ38897-derived protein than in a mixture with the buffer solution alone. Hence, when FLJ38897-derived protein co-exists, loperamide was shown to be eluted into the spin column filtrate in the form of a conjugate with the co-existing FLJ38897-derived protein, from which we determined that there was an interaction between loperamide and the FLJ38897-derived protein.

Hence, the FLJ38897-derived protein was found to be a target protein for loperamide. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 20

### Example of Analysis of Interaction Between FLJ38897-Derived Protein and Fluphenazine

The FLJ38897-derived protein was expressed and purified according to the methods of Reference Examples 1 and 2, and the interaction between the aforementioned protein and fluphenazine was analyzed according to the method of Reference Example 3 (data not shown). As a result, it was shown that the mass spectrum peak intensity of fluphenazine in the spin column filtrate was higher when fluphenazine was applied to the spin column in a mixture with the FLJ38897-derived protein than in a mixture with the buffer solution alone. Hence, when FLJ38897-derived protein co-exists, fluphenazine was shown to be eluted into the spin column filtrate in the form of a conjugate with the co-existing FLJ38897-derived protein, from which we determined that there was an interaction between fluphenazine and the FLJ38897-derived protein.

Hence, the FLJ38897-derived protein was found to be a target protein for fluphenazine. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 21

### Example of Analysis of Interaction Between FLJ38897-Derived Protein and Mefloquine

The FLJ38897-derived protein was expressed and purified according to the methods of Reference Examples 1 and 2, and the interaction between the aforementioned protein and mefloquine was analyzed according to the method of Reference Example 3 (data not shown). As a result, it was shown that the mass spectrum peak intensity of mefloquine in the spin column filtrate was higher when mefloquine was applied to the spin column in a mixture with the FLJ38897-derived protein than in a mixture with the buffer solution alone. Hence, when FLJ38897-derived protein co-exists, mefloquine was shown to be eluted into the spin column filtrate in the form of a conjugate with the co-existing FLJ38897-derived protein, from which we determined that there was an interaction between mefloquine and the FLJ38897-derived protein.

Hence, the FLJ38897-derived protein was found to be a target protein for mefloquine. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 22

### Example of Analysis of Interaction Between FLJ38897-Derived Protein and Perphenazine

The FLJ38897-derived protein was expressed and purified according to the methods of Reference Examples 1 and 2, and the interaction between the aforementioned protein and perphenazine was analyzed according to the method of Reference Example 3 (data not shown). As a result, it was shown that the mass spectrum peak intensity of perphenazine in the spin column filtrate was higher when perphenazine was applied to the spin column in a mixture with the FLJ38897-derived protein than in a mixture with the buffer solution alone. Hence, when FLJ38897-derived protein co-exists, perphenazine was shown to be eluted into the spin column filtrate in the form of a conjugate with the co-existing FLJ38897-derived protein, from which we determined that there was an interaction between perphenazine and the FLJ38897-derived protein.

Hence, the FLJ38897-derived protein was found to be a target protein for perphenazine. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 23

### Example of Analysis of Interaction Between FLJ38897-Derived Protein and Perhexyline

The FLJ38897-derived protein was expressed and purified according to the methods of Reference Examples 1 and 2, and the interaction between the aforementioned protein and perhexyline was analyzed according to the method of Reference Example 3 (data not shown). As a result, it was shown that the mass spectrum peak intensity of perhexyline in the spin column filtrate was higher when perhexyline was applied to the spin column in a mixture with the FLJ38897-derived protein than in a mixture with the buffer solution alone. Hence, when FLJ38897-derived protein co-exists, perhexyline was shown to be eluted into the spin column filtrate in the form of a conjugate with the co-existing FLJ38897-derived protein, from which we determined that there was an interaction between perhexyline and the FLJ38897-derived protein.

Hence, the FLJ38897-derived protein was found to be a target protein for perhexyline. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 24

### Example of Analysis of Interaction Between FLJ38897-Derived Protein and Raloxifene

The FLJ38897-derived protein was expressed and purified according to the methods of Reference Examples 1 and 2, and the interaction between the aforementioned protein and raloxifene was analyzed according to the method of Reference Example 3 (data not shown). As a result, it was shown that the mass spectrum peak intensity of raloxifene in the spin column filtrate was higher when raloxifene was applied to the spin column in a mixture with the FLJ38897-derived protein than in a mixture with the buffer solution alone. Hence, when FLJ38897-derived protein co-exists, raloxifene was shown to be eluted into the spin column filtrate in the form of a conjugate with the co-existing FLJ38897-derived protein, from which we determined that there was an interaction between raloxifene and the FLJ38897-derived protein.

Hence, the FLJ38897-derived protein was found to be a target protein for raloxifene. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 25

### Example of Analysis of Interaction Between FLJ38897-Derived Protein and Terfenadine

The FLJ38897-derived protein was expressed and purified according to the methods of Reference Examples 1 and 2, and the interaction between the aforementioned protein and terfenadine was analyzed according to the method of Reference Example 3 (data not shown). As a result, it was shown that the mass spectrum peak intensity of terfenadine in the spin column filtrate was higher when terfenadine was applied to the spin column in a mixture with the FLJ38897-derived protein than in a mixture with the buffer solution alone. Hence, when FLJ38897-derived protein co-exists, terfenadine was shown to be eluted into the spin column filtrate in the form of a conjugate with the co-existing FLJ38897-derived protein, and an interaction was observed between terfenadine and the FLJ38897-derived protein.

Hence, the FLJ38897-derived protein was found to be a target protein for terfenadine. Therefore, a new drug can be screened by making the FLJ38897-derived protein interact with screening candidate substances. Specifically, a new drug can be screened by constructing a system which detects the interaction between the FLJ38897-derived protein and a candidate substance according to, for example, the method of Reference Example 3.

### Example 26

### Analysis of Interaction Between FLJ39553-Derived Protein and Amphotericin B

The FLJ39553-derived protein was expressed and purified according to the methods of Reference Examples 1 and 2, and the interaction between the protein expressed and purified from FLJ39553 and amphotericin B was analyzed according to the method of Reference Example 3 (data not shown). As a result, the mass chromatogram of amphotericin B in TSKgel super SW2000 micro-LC column (1.0 mm ID×30 mm) revealed earlier elution thereof in the presence of the FLJ39553-derived protein than in its absence. This shows that amphotericin B eluted earlier from the SEC micro-LC column as a result of an interaction with the FLJ39553-derived protein, from which we determined that there was an interaction between amphotericin B and the FLJ39553-derived protein.

Hence, the FLJ39553-derived protein was found to be a target protein for amphotericin B. Therefore, a new drug can be screened by making the FLJ39553-derived protein interact with screening candidate substances. Specifically, a new drug can be screened by constructing a system which detects the interaction between the FLJ39553-derived protein and a candidate substance according to, for example, the method of Reference Example 3.

### Example 27

### Analysis of Interaction Between FLJ39553-Derived Protein and Hydroxocobalamin

The FLJ39553-derived protein was expressed and purified according to the methods of Reference Examples 1 and 2, and the interaction between the protein expressed and purified from FLJ39553 and hydroxocobalamin was analyzed according to the method of Reference Example 3 (data not shown). As a result, the mass chromatogram of hydroxocobalamin in TSKgel super SW2000 micro-LC column (1.0 mm ID×30 mm) revealed earlier elution thereof in the presence of the FLJ39553-derived protein than in its absence. This shows that hydroxocobalamin eluted earlier from the SEC micro-LC column as a result of an interaction with the FLJ39553-derived protein, from which we determined that there was an interaction between hydroxocobalamin and the FLJ39553-derived protein.

Hence, the FLJ39553-derived protein was found to be a target protein for hydroxocobalamin. Therefore, a new drug can be screened by making the FLJ39553-derived protein interact with screening candidate substances. Specifically, a new drug can be screened by constructing a system which detects the interaction between the FLJ39553-derived protein and a candidate substance according to, for example, the method of Reference Example 3.

### Example 28

### Analysis of Interaction Between FLJ39553-Derived Protein and Simvastatin

The FLJ39553-derived protein was expressed and purified according to the methods of Reference Examples 1 and 2, and the interaction between the protein expressed and purified from FLJ39553 and simvastatin was analyzed according to the method of Reference Example 3 (data not shown). As a result, the mass chromatogram of simvastatin in TSKgel super SW2000 micro-LC column (1.0 mm ID×30 mm) revealed earlier elution thereof in the presence of the FLJ39553-derived protein than in its absence. This shows that simvastatin eluted earlier from the SEC micro-LC column as a result of an interaction with the FLJ39553-derived protein, from which we determined that there was an interaction between simvastatin and the FLJ39553-derived protein.

Hence, the FLJ39553-derived protein was found to be a target protein for simvastatin. Therefore, a new drug can be screened by making the FLJ39553-derived protein interact with screening candidate substances. Specifically, a new drug can be screened by constructing a system which detects the interaction between the FLJ39553-derived protein and a candidate substance according to, for example, the method of Reference Example 3.

### Example 29

### Analysis of Interaction Between FLJ40298-Derived Protein and Benoxinate

The FLJ40298-derived protein was expressed and purified according to the methods of Reference Examples 1 and 2, and the interaction between the protein expressed and purified from FLJ40298 and benoxinate was analyzed according to the method of Reference Example 3. The results are shown in Table 15. The spectrum intensity of the pharmaceutical compound contained in the SEC spin column filtrate (protein fraction eluted from SEC) increased depending on the doses of both the small compound and the FLJ40298-derived protein, respectively, from which we determined that there was a concentration-dependent interaction between benoxinate and the FLJ40298-derived protein.

Hence, the FLJ40298-derived protein was found to be a target protein for benoxinate. Therefore, a new drug can be screened by making the FLJ40298-derived protein interact with screening candidate substances. Specifically, a new drug can be screened by constructing a system which detects the interaction between the FLJ40298-derived protein and a candidate substance according to, for example, the method of Reference Example 3.

TABLE 15
Mass Range for Msbat-retrieval = 379.20-50(m/z)

| | |
|---|---|
| FLJ40298 - | compound (μM) |

| | | | | |
|---|---|---|---|---|
| benoxinate | 0 | 12.5 | 25 | 62.5 |

| | | | | | |
|---|---|---|---|---|---|
| protein | 0 | 58845 | 202235 | 557220 | 815144 |
| (μM) | 5 | 65465 | 285949 | 1519215 | 225597 |
| | 10 | 53181 | 263958 | 552560 | 1509221 |
| | 25 | 80978 | 299357 | 583103 | 2027848 |

### Example 30

### Analysis of Interaction Between FLJ40760-Derived Protein and Pioglitazone

The FLJ40760-derived protein was expressed and purified according to the methods of Reference Examples 1 and 2, and the interaction between the protein expressed and purified from FLJ40760 and pioglitazone was analyzed according to the method of Reference Example 3. The results are shown in Table 16. The spectrum intensity of the pharmaceutical compound contained in the SEC spin column filtrate (protein fraction eluted from SEC) increased, depending on the doses of both the small compound and the FLJ40760-derived protein, respectively, from which we determined that there was a concentration-dependent interaction between pioglitazone and the FLJ40760-derived protein.

Hence, the FLJ40760-derived protein was found to be a target protein for pioglitazone. Therefore, a new drug can be screened by making the FLJ40760-derived protein interact with screening candidate substances. Specifically, a new drug can be screened by constructing a system which detects the interaction between the FLJ40760-derived protein and a candidate substance according to, for example, the method of Reference Example 3.

TABLE 16
Mass Range for retrieval m/z = 357.3-357.7(/300-400)

| | |
|---|---|
| FLJ40760 - | compound (μM) |

| | | | | |
|---|---|---|---|---|
| pioglitazone | 0 | 12.5 | 25 | 62.5 |

| | | | | | |
|---|---|---|---|---|---|
| protein | 0 | 24397 | 32753 | 34360 | 32269 |
| (μM) | 5 | 29475 | 31189 | 35442 | 40960 |
| | 10 | 32257 | 30333 | 40021 | 50827 |
| | 25 | 22634 | 28828 | 40902 | 61590 |

### Example 31

### Analysis of Interaction Between FLJ41550-Derived Protein and Thioproperasine

The FLJ41550-derived protein was expressed and purified according to the methods of Reference Examples 1 and 2, and the interaction between the protein expressed and purified from FLJ41550 and thioproperasine was analyzed according to the method of Reference Example 3. The results are shown in Table 17. The spectrum intensity of the pharmaceutical compound contained in the SEC spin column filtrate (protein fraction eluted from SEC) increased depending on the doses of both the small compound and the FLJ41550-derived protein, respectively, from which we determined that there was a concentration-dependent interaction between thioproperasine and the FLJ41550-derived protein.

Hence, the FLJ41550-derived protein was found to be a target protein for thioproperasine. Therefore, a new drug can be screened by making the FLJ41550-derived protein interact with screening candidate substances. Specifically, a new drug can be screened by constructing a system which detects the interaction between the FLJ41550-derived protein and a candidate substance according to, for example, the method of Reference Example 3.

TABLE 17
Mass Range for retrieval m/z = 447.1-447.4

| | |
|---|---|
| FLJ41550 - | compound (μM) |

| | | | | |
|---|---|---|---|---|
| thioproperazine | 0 | 12.5 | 25 | 62.5 |

| | | | | | |
|---|---|---|---|---|---|
| protein | 0 | 261344 | 252410 | 262646 | 1132948 |
| (μM) | 5 | 280789 | 377673 | 778927 | 1457362 |
| | 10 | 397491 | 478699 | 1198533 | 2445959 |
| | 25 | 524011 | 941513 | 983016 | 2997892 |

### Example 32

### Analysis of Interaction Between FLJ41991-Derived Protein and Raloxifene

The FLJ41991-derived protein was expressed and purified according to the methods of Reference Examples 1 and 2, and the interaction between the protein expressed and purified from FLJ41991 and raloxifene was analyzed according to the method of Reference Example 3. The results are shown in Table 18. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between raloxifene and the FLJ41991-derived protein.

Hence, the FLJ41991-derived protein was found to be a target protein for raloxifene. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 18
Mass Range for Msbat-retrieval = 474.30-60(m/z)

| | |
|---|---|
| FLJ41991 - | compound (μM) |

| | | | | |
|---|---|---|---|---|
| raloxifene | 0 | 12.5 | 25 | 62.5 |

| | | | | | |
|---|---|---|---|---|---|
| protein | 0 | 29622 | 19251 | 37961 | 58164 |
| (μM) | 5 | 23435 | 32265 | 52442 | 153267 |
| | 10 | 23456 | 67187 | 119306 | 1091123 |
| | 25 | 17348 | 331748 | 1312562 | 3195671 |

### Example 33

### Analysis of Interaction Between FLJ41991-Derived Protein and Loperamide

The FLJ41991-derived protein was expressed and purified according to the methods of Reference Examples 1 and 2, and the interaction between the protein expressed and purified from FLJ41991 and loperamide was analyzed according to the method of Reference Example 3. The results are shown in Table 19. The spectrum intensity of the pharmaceutical compound contained in the SEC spin column filtrate (protein fraction eluted from SEC) increased depending on the doses of both of each small compound and the FLJ41991-derived protein, respectively, from which we determined that there was a concentration-dependent interaction between loperamide and the FLJ41991-derived protein.

Hence, the FLJ41991-derived protein was found to be a target protein for loperamide. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 19
Mass Range for Msbat-retrieval = 477.15-45(m/z)

| | |
|---|---|
| FLJ41991 - | compound (μM) |

| | | | | |
|---|---|---|---|---|
| loperamide | 0 | 12.5 | 25 | 62.5 |

| | | | | | |
|---|---|---|---|---|---|
| protein | 0 | 57131 | 75412 | 263744 | 1579322 |
| (μM) | 5 | 23462 | 200285 | 863800 | 3520629 |
| | 10 | 15318 | 950587 | 1856141 | 6073080 |
| | 25 | 31705 | 1688197 | 4462655 | 9496680 |

### Example 34

### Analysis of Interaction Between FLJ41991-Derived Protein and Mefloquine

The FLJ41991-derived protein was expressed and purified according to the methods of Reference Examples 1 and 2, and the interaction between the protein expressed and purified from FLJ41991 and mefloquine was analyzed according to the method of Reference Example 3. The results are shown in Table 20. The spectrum intensity of the pharmaceutical compound contained in the SEC spin column filtrate (protein fraction eluted from SEC) increased depending on the doses of both of each small compound and the FLJ41991-derived protein, respectively, from which we determined that there was a concentration-dependent interaction between mefloquine and the FLJ41991-derived protein.

Hence, the FLJ41991-derived protein was found to be a target protein for mefloquine. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 20
Mass Range for Msbat-retrieval = 379.20-50(m/z)

| | |
|---|---|
| FLJ41991 - | compound (μM) |

| | | | | |
|---|---|---|---|---|
| mefloquine | 0 | 12.5 | 25 | 62.5 |

| | | | | | |
|---|---|---|---|---|---|
| protein | 0 | 16816 | 16334 | 27931 | 97153 |
| (μM) | 5 | 21128 | 46239 | 100287 | 528117 |
| | 10 | 14216 | 101775 | 273106 | 2017157 |
| | 25 | 15988 | 453818 | 1788847 | 4385022 |

### Example 35

### Analysis of Interaction Between FLJ41991-Derived Protein and Perphenazine

The FLJ41991-derived protein was expressed and purified according to the methods of Reference Examples 1 and 2, and the interaction between the protein expressed and purified from FLJ41991 and perphenazine was analyzed according to the method of Reference Example 3. The results are shown in Table 21. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between perphenazine and the FLJ41991-derived protein.

Hence, the FLJ41991-derived protein was found to be a target protein for perphenazine. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 21

| Mass Range for Msbat-retrieval = 404.25-55(m/z) | |
|---|---|
| FLJ41991 - | compound (μM) |

| | | | | |
|---|---|---|---|---|
| perphenazine | 0 | 12.5 | 25 | 62.5 |

| | | | | | |
|---|---|---|---|---|---|
| protein | 0 | 26895 | 23424 | 38186 | 35061 |
| (μM) | 5 | 32057 | 43607 | 71806 | 208220 |
| | 10 | 37133 | 83672 | 229107 | 695158 |
| | 25 | 22593 | 314285 | 799962 | 2872912 |

### Example 36

### Analysis of Interaction Between FLJ41991-Derived Protein and Quinacrine

The FLJ41991-derived protein was expressed and purified according to the methods of Reference Examples 1 and 2, and the interaction between the protein expressed and purified from FLJ41991 and quinacrine was analyzed according to the method of Reference Example 3. The results are shown in Table 22. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between quinacrine and the FLJ41991-derived protein.

Hence, the FLJ41991-derived protein was found to be a target protein for quinacrine. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 22
Mass Range for Msbat-retrieval = 400.25-55(m/z)

| | |
|---|---|
| FLJ41991 - | compound (μM) |

| | | | | |
|---|---|---|---|---|
| quinacrine | 0 | 12.5 | 25 | 62.5 |

| | | | | | |
|---|---|---|---|---|---|
| protein | 0 | 28457 | 36421 | 40978 | 123977 |
| (μM) | 5 | 13213 | 77607 | 125609 | 598920 |
| | 10 | 20744 | 225680 | 481486 | 1080431 |
| | 25 | 20007 | 439131 | 457713 | 1854355 |

### Example 37

### Analysis of Interaction Between FLJ41991-Derived Protein and GBR12909

The FLJ41991-derived protein was expressed and purified according to the methods of Reference Examples 1 and 2, and the interaction between the protein expressed and purified from FLJ41991 and GBR12909 was analyzed according to the method of Reference Example 3. The results are shown in Table 23. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between GBR12909 and the FLJ41991-derived protein.

Hence, the FLJ41991-derived protein was found to be a target protein for GBR12909. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 23
Mass Range for Msbat-retrieval m/z = 451.15-45(m/z)

| | |
|---|---|
| FLJ41991 - | compound (μM) |

| | | | | |
|---|---|---|---|---|
| GBR12909 | 0 | 12.5 | 25 | 62.5 |

| | | | | | |
|---|---|---|---|---|---|
| protein | 0 | 35456 | 8662 | 27810 | 8881 |
| (μM) | 5 | 16658 | 18718 | 26447 | 173210 |
| | 10 | 23474 | 52282 | 332712 | 1937631 |
| | 25 | 10590 | 971864 | 951832 | 5676666 |

### Example 38

### Analysis of Interaction Between FLJ41991-Derived Protein and Terfenadine

The FLJ41991-derived protein was expressed and purified according to the methods of Reference Examples 1 and 2, and the interaction between the protein expressed and purified from FLJ41991 and terfenadine was analyzed according to the method of Reference Example 3. The results are shown in Table 24. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction was observed between terfenadine and the FLJ41991-derived protein.

Hence, the FLJ41991-derived protein was found to be a target protein for terfenadine. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 24
Mass Range for Msbat-retrieval m/z = 472.35-65(m/z)

| FLJ41991 - | | compound (μM) | | | |
|---|---|---|---|---|---|
| terfenadine | | 0 | 12.5 | 25 | 62.5 |
| protein | 0 | 14362 | 26814 | 87636 | 231173 |
| (μM) | 5 | 26000 | 200843 | 311313 | 1759965 |
| | 10 | 21302 | 376865 | 949465 | 5619658 |
| | 25 | 20066 | 1734729 | 4190413 | 9678283 |

### Example 39

### Analysis of Interaction Between FLJ41991-Derived Protein and Fluphenazine

The FLJ41991-derived protein was expressed and purified according to the methods of Reference Examples 1 and 2, and the interaction between the protein expressed and purified from FLJ41991 and fluphenazine was analyzed according to the method of Reference Example 3. The results are shown in Table 25. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction was observed between fluphenazine and the FLJ41991-derived protein.

Hence, the FLJ41991-derived protein was found to be a target protein for fluphenazine. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 25
Mass Range for Msbat-retrieval = 438.25-55(m/z)

| FLJ41991 - | | compound (μM) | | | |
|---|---|---|---|---|---|
| fluphenazine | | 0 | 12.5 | 25 | 62.5 |
| protein | 0 | 47297 | 87861 | 193639 | 200313 |
| (μM) | 5 | 42295 | 162795 | 276524 | 1055670 |
| | 10 | 54789 | 434899 | 843648 | 3173659 |
| | 25 | 35344 | 1101486 | 1778376 | 6405132 |

### Example 40

### Analysis of Interaction Between FLJ41991-Derived Protein and Astemizole

The FLJ41991-derived protein was expressed and purified according to the methods of Reference Examples 1 and 2, and the interaction between the protein expressed and purified from FLJ41991 and astemizole was analyzed according to the method of Reference Example 3. The results are shown in Table 26. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between astemizole and the FLJ41991-derived protein.

Hence, the FLJ41991-derived protein was found to be a target protein for astemizole. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 26
Mass Range for Msbat-retrieval = 459.25-55(m/z)

| FLJ41991 - | | compound (μM) | | | |
|---|---|---|---|---|---|
| astemizole | | 0 | 12.5 | 25 | 62.5 |
| protein | 0 | 20106 | 24899 | 19274 | 27212 |
| (μM) | 5 | 24065 | 60014 | 89714 | 361364 |
| | 10 | 25922 | 160855 | 732296 | 2309029 |
| | 25 | 29185 | 908771 | 2056843 | 5171023 |

### Example 41

### Analysis of Interaction Between FLJ41991-Derived Protein and Benztropine

The FLJ41991-derived protein was expressed and purified according to the methods of Reference Examples 1 and 2, and the interaction between the protein expressed and purified from FLJ41991 and benztropine was analyzed according to the method of Reference Example 3. The results are shown in Table 27. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between benztropine and the FLJ41991-derived protein.

Hence, the FLJ41991-derived protein was found to be a target protein for benztropine. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 27
Mass Range for Msbat-retrieval = 308.30-60(m/z)

| | |
|---|---|
| FLJ41991 - | compound (μM) |

| | | | | |
|---|---|---|---|---|
| benztropine | 0 | 12.5 | 25 | 62.5 |

| | | | | | |
|---|---|---|---|---|---|
| protein | 0 | 15849 | 143943 | 152947 | 911727 |
| (μM) | 5 | 13742 | 220928 | 491863 | 1861766 |
| | 10 | 11453 | 285013 | 560686 | 2757526 |
| | 25 | 6829 | 489071 | 1352077 | 3061987 |

### Example 42

### Analysis of Interaction Between FLJ41991-Derived Protein and Chlorprothixene

The FLJ41991-derived protein was expressed and purified according to the methods of Reference Examples 1 and 2, and the interaction between the protein expressed and purified from FLJ41991 and chlorprothixene was analyzed according to the method of Reference Example 3. The results are shown in Table 28. The spectrum intensity of the pharmaceutical compound contained in the SEC spin column filtrate (protein fraction eluted from SEC) increased depending on the doses of both the small compound and the FLJ41991-derived protein, respectively, from which we determined that there was a concentration-dependent interaction between chlorprothixene and the FLJ41991-derived protein.

Hence, the FLJ41991-derived protein was found to be a target protein for chlorprothixene. Therefore, a new drug can be screened by making the FLJ41991-derived protein interact with screening candidate substances. Specifically, a new drug can be screened by constructing a system which detects the interaction between the FLJ41991-derived protein and a candidate substance according to, for example, the method of Reference Example 3.

TABLE 28
Mass Range for Msbat-retrieval = 316.15-45(m/z)

| | |
|---|---|
| FLJ41991 - | compound (μM) |

| | | | | |
|---|---|---|---|---|
| chlorprothixene | 0 | 12.5 | 25 | 62.5 |

| | | | | | |
|---|---|---|---|---|---|
| protein | 0 | 128663 | 178622 | 161141 | 156021 |
| (μM) | 5 | 167275 | 175333 | 149738 | 210998 |
| | 10 | 156942 | 226245 | 277798 | 626223 |
| | 25 | 144626 | 393132 | 490640 | 2564975 |

### Example 43

### Analysis of Interaction Between FLJ42665-Derived Protein and Benzethonium

The FLJ42665-derived protein was expressed and purified according to the methods of Reference Examples 1 and 2, and the interaction between the protein expressed and purified from FLJ42665 and benzethonium was analyzed according to the method of Reference Example 3. The results are shown in Table 29. The spectrum intensity of the pharmaceutical compound contained in the SEC spin column filtrate (protein fraction eluted from SEC) increased depending on the doses of both the small compound and the FLJ42665-derived protein, respectively, from which we determined that there was a dose-dependent interaction between benzethonium and the FLJ42665-derived protein.

Hence, the FLJ42665-derived protein was found to be a target protein for benzethonium. Therefore, a new drug can be screened by making the FLJ42665-derived protein interact with screening candidate substances. Specifically, a new drug can be screened by constructing a system which detects the interaction between the FLJ42665-derived protein and a candidate substance according to, for example, the method of Reference Example 3.

TABLE 29
Mass Range for Msbat-retrieval = 412.40-70(m/z)

| | |
|---|---|
| FLJ42665 - | compound (μM) |

| | | | | |
|---|---|---|---|---|
| benzethonium | 0 | 12.5 | 25 | 62.5 |

| | | | | | |
|---|---|---|---|---|---|
| protein | 0 | 25833 | 19475 | 27192 | 14420 |
| (μM) | 5 | 23969 | 26605 | 21193 | 28967 |
| | 10 | 27317 | 47666 | 31214 | 32809 |
| | 25 | 20041 | 130631 | 124057 | 108557 |

### Example 44

Proteins derived from FLJXXXXX (FLJXXXXX corresponds to an FLJ number in Table 30-1 to Table 30-4 below) were expressed and purified according to the methods of Reference Example 1 and Reference Example 2, and interactions of the proteins expressed and purified from FLJXXXXX and various compounds were analyzed according to the method of Reference Example 3. The results are shown in Table 30-1 to Table 30-4. Depending on the doses of both each compound and FLJXXXXX expression protein, an increase in the spectral intensity of the pharmaceutical compound contained in the SEC spin column filtrate (protein elution fraction from SEC) was observed; it was judged to exhibit a concentration-dependent interaction.

TABLE 30-1

| SEQ ID NO | FLJ No | name of compound |
|---|---|---|
| 1 | FLJ10420 | picotamide |
| 2 | FLJ10537 | methoxsalen |
| 3 | FLJ11211 | cyclosporin A |
| 3 | FLJ11211 | terfenadine |
| 3 | FLJ11211 | deferoxamine |
| 3 | FLJ11211 | mesoridazine |
| 3 | FLJ11211 | pimozide |
| 3 | FLJ11211 | eburnamonine |
| 4 | FLJ12857 | pancuronium bromide |
| 5 | FLJ20972 | protriptyline. |
| 6 | FLJ21841 | rifampicin |
| 6 | FLJ21841 | mafenide |
| 6 | FLJ21841 | spiramycin |
| 7 | FLJ22317 | solanine α |
| 7 | FLJ22317 | mifepristone |
| 8 | FLJ23466 | cyclosporin A |
| 8 | FLJ23466 | chlorambucil |

TABLE 30-2

| | | |
|---|---|---|
| 9 | FLJ25288 | amethopterin |
| 10 | FLJ35914 | hydroxocobalamin |
| 10 | FLJ35914 | hydantoin |
| 11 | FLJ36526 | hydroxocobalamin |
| 11 | FLJ36526 | acetopromazine |
| 11 | FLJ36526 | SR-95639A |
| 11 | FLJ36526 | tetrazoline |
| 11 | FLJ36526 | diphemanil |
| 12 | FLJ37909 | cyclosporin A |
| 12 | FLJ37909 | amikacin |
| 12 | FLJ37909 | amiodarone |
| 12 | FLJ37909 | buprenorphine |
| 12 | FLJ37909 | chlorhexidine |
| 12 | FLJ37909 | clomiphene |
| 12 | FLJ37909 | dihydroergocristine |
| 12 | FLJ37909 | dihydroergotamine |
| 12 | FLJ37909 | doxazosin |
| 12 | FLJ37909 | emetine |
| 12 | FLJ37909 | fenbendazole |
| 12 | FLJ37909 | flunarizine |
| 12 | FLJ37909 | flupentixol |
| 12 | FLJ37909 | lidoflazine |
| 12 | FLJ37909 | mefloquine |
| 12 | FLJ37909 | metergoline |
| 12 | FLJ37909 | oxethazaine |
| 12 | FLJ37909 | oxolinic acid |
| 12 | FLJ37909 | perhexiline |
| 12 | FLJ37909 | pimozide |
| 12 | FLJ37909 | quinacrine |
| 12 | FLJ37909 | rescinnamine |
| 12 | FLJ37909 | tamoxifen |
| 12 | FLJ37909 | thioproperazine |
| 12 | FLJ37909 | thioridazine |
| 12 | FLJ37909 | thiothixene (cis) |
| 12 | FLJ37909 | alimemazine |
| 12 | FLJ37909 | reserpine |
| 12 | FLJ37909 | hydroxyprogesterone |
| 12 | FLJ37909 | raloxifene |
| 13 | FLJ38531 | sulfasalazine |
| 13 | FLJ38531 | nalidixic acid |

TABLE 30-3

| | | |
|---|---|---|
| 13 | FLJ38531 | flumequine |
| 13 | FLJ38531 | tenoxicam |
| 13 | FLJ38531 | ethotoin |
| 14 | FLJ38897 | chlorprothixene |
| 14 | FLJ38897 | loperamide |
| 14 | FLJ38897 | benztropine |
| 14 | FLJ38897 | fluphenazine |
| 14 | FLJ38897 | mefloquine |
| 14 | FLJ38897 | perphenazine |
| 14 | FLJ38897 | perhexiline |
| 14 | FLJ38897 | raloxifene |
| 14 | FLJ38897 | terfenadine |
| 14 | FLJ38897 | astemizole |
| 15 | FLJ39553 | amphotericin B |
| 15 | FLJ39553 | hydroxocobalamin |
| 15 | FLJ39553 | simvastatin |
| 15 | FLJ39553 | celestine blue |
| 15 | FLJ39553 | palmatine |
| 15 | FLJ39553 | sulfadimethoxine |
| 15 | FLJ39553 | sarpogrelate |
| 15 | FLJ39553 | Avermectin B1A |
| 15 | FLJ39553 | chloropyramine |
| 15 | FLJ39553 | apigenin |
| 15 | FLJ39553 | cinchonine |
| 15 | FLJ39553 | harmaline |
| 15 | FLJ39553 | methoxy-6-harmalan |
| 15 | FLJ39553 | meticrane |
| 15 | FLJ39553 | paclitaxel |
| 15 | FLJ39553 | solanine α |
| 15 | FLJ39553 | paramethasone |
| 15 | FLJ39553 | demeclocycline |
| 15 | FLJ39553 | bergenin |
| 15 | FLJ39553 | nafcillin |
| 15 | FLJ39553 | carboprost |
| 16 | FLJ40298 | benoxinate |
| 17 | FLJ40760 | pioglitazone |
| 17 | FLJ40760 | lisinopril |
| 18 | FLJ41550 | thioproperazine |
| 19 | FLJ41991 | GBR12909 |
| 19 | FLJ41991 | loperamide |

TABLE 30-4

| | | |
|---|---|---|
| 19 | FLJ41991 | mefloquine |
| 19 | FLJ41991 | perphenazine |
| 19 | FLJ41991 | quinacrine |
| 19 | FLJ41991 | raloxifene |
| 19 | FLJ41991 | terfenadine |
| 19 | FLJ41991 | fluphenazine |
| 19 | FLJ41991 | astemizole |
| 19 | FLJ41991 | benztropine |
| 19 | FLJ41991 | chlorprothixene |
| 20 | FLJ42665 | benzethonium |
| 20 | FLJ42665 | Nanofin (cis) |
| 22 | FLJ41265 | cyproheptadine |
| 23 | FLJ20571 | domperidone |
| 23 | FLJ20571 | methylbenzethonium |
| | | chloride |
| 24 | FLJ35353 | albendazole |
| 24 | FLJ35353 | benzethonium |
| 24 | FLJ35353 | chlorpromazine |
| 24 | FLJ35353 | clofazimine |
| 24 | FLJ35353 | ellipticine |
| 24 | FLJ35353 | glipizide |
| 24 | FLJ35353 | mefenamic acid |
| 24 | FLJ35353 | megestrol |
| 24 | FLJ35353 | methixene |
| 24 | FLJ35353 | nordiazepam |
| 24 | FLJ35353 | pentoxifylline |
| 24 | FLJ35353 | pinacidil |
| 24 | FLJ35353 | syrosingopine |
| 24 | FLJ35353 | Thiothixene (cis) |
| 24 | FLJ35353 | tomatidine |
| 24 | FLJ35353 | dipyrone |
| 24 | FLJ35353 | ethyl loflazepate |
| 24 | FLJ35353 | clobazam |
| 24 | FLJ35353 | ebastine |
| 24 | FLJ35353 | dydrogesterone |
| 24 | FLJ35353 | solasodine |
| 24 | FLJ35353 | methylbenzethonium |
| | | chloride |
| 24 | FLJ35353 | α-ergocryptine |

Thus, each FLJXXXXX-derived protein was proven to be a target protein for these various compounds. From this, it is evident that by reacting an FLJXXXXX-derived protein and a screening candidate substance, screening for a novel pharmaceutical can be performed. Hence, by constructing a system for detecting the interactions of FLJXXXXX-derived proteins and candidate substances by, for example, the method of Example 1, screening for a novel pharmaceutical can be performed.

### Example 45

According to the method of Reference Example 3-3, the binding intensities (Kd values) for the specific interactions of various compounds and proteins confirmed in this Example were calculated. The results are shown in Table 31.

TABLE 31

| SEQ ID | | | Kd value |
|---|---|---|---|
| NO | FLJ No. | compound | (Biacore) |
| 2 | FLJ10537 | methoxsalen | 6.0E−03 |
| 8 | FLJ23466 | cyclosporin A | 8.5E−03 |
| 9 | FLJ25288 | amethopterin | 1.4E−03 |
| 10 | FLJ35914 | hydroxocobalamin | 5.4E−03 |
| 11 | FLJ36526 | hydroxocobalamin | 6.0E−04 |
| 12 | FLJ37909 | cyclosporin A | 1.5E−03 |
| 13 | FLJ38531 | sulfasalazine | 8.9E−06 |
| | | nalidixic acid | 1.2E−03 |
| 14 | FLJ38897 | loperamide | 2.3E−04 |
| | | fluphenazine | 2.3E−04 |
| | | chlorprothixene | 3.1E−04 |
| | | mefloquine | 7.6E−04 |
| | | raloxifene | 7.8E−04 |
| | | benztropine | 1.6E−03 |
| | | terfenadine | 1.7E−03 |
| | | perphenazine | 1.9E−03 |
| | | astemizole | 1.0E−02 |
| | | perhexiline | 1.1E−02 |
| 15 | FLJ39553 | hydroxocobalamin | 9.9E−03 |
| 16 | FLJ40298 | benoxinate | 4.5E−03 |
| 17 | FLJ40760 | pioglitazone | 9.9E−03 |
| 19 | FLJ41991 | fluphenazine | 1.4E−04 |
| | | raloxifene | 9.5E−04 |
| | | quinacrine | 2.1E−03 |
| | | mefloquine | 3.3E−03 |
| | | terfenadine | 6.2E−03 |
| | | astemizole | 1.0E−02 |
| | | benztropine | 1.4E−02 |
| | | perphenazine | 1.4E−02 |
| 23 | FLJ20571 | domperidone | 3.5E−04 |

### INDUSTRIAL APPLICABILITY

The target proteins and target genes of the present invention are useful for the development of bioactive substances, for example, drug discovery and the like. The screening methods of the present invention and the derivative production methods of the present invention are useful for the development of prophylactic or therapeutic agents for various diseases or conditions, and investigational reagents for the diseases or the conditions and the like. The regulators and derivatives of the present invention are useful for the prevention and treatment of various diseases or conditions, and as investigational reagents for the diseases or the conditions and the like. The complexes and kits of the present invention are useful for the screening methods of the present invention, the derivative production methods of the present invention and the like. The determination methods and determination kits of the present invention are useful for the evaluation of the onset or likelihood of onset of various diseases or conditions, evaluation of susceptibility to bioactive substances, and the like in animals.

The target proteins and target genes of the present invention enable the development of bioactive substances, for example, drug discovery and the like. The screening methods of the present invention and the derivative production method of the present invention enable the development of prophylactic or therapeutic agents for various diseases or conditions, and investigational reagents for the diseases or the conditions, and the like. The regulators and derivatives of the present invention enable the prophylaxis and treatment of various diseases or conditions, and the development of investigational reagents for the diseases or the conditions, and the like. The complexes and kits of the present invention enable the implementation of the screening methods of the present invention, the derivative production methods of the present invention and the like. The determination methods and determination kits of the present invention enable the evaluation of the onset or likelihood of onset of various diseases or conditions in animals, and the evaluation of the susceptibility of animals to bioactive substances and the like.

This application is based on a patent application No. 2005-089609 filed in Japan (filing date: Mar. 25, 2005), the contents of which are incorporated in full herein by this reference.

1. A method for screening a substance capable of regulating an action associated with bioactive substance X, which comprises determining whether or not a test substance is capable of regulating the expression or function of target protein Y or a gene that encodes the protein, wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination, of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

2. The method according to claim 1, which comprises the following steps (a) to (c):
(a) a step for bringing the test substance into contact with target protein Y;
(b) a step for measuring the functional level of the protein in the presence of the test substance, and comparing said functional level with the functional level of the protein in the absence of the test substance;
(c) a step for selecting a test substance that alters the functional level of the protein on the basis of the result of the comparison in (b) above.

3. The method according to claim 1, which comprises the following steps (a) to (c):
(a) a step for bringing the test substance and cells allowing a measurement of the expression of target protein Y or a gene that encodes the protein into contact with each other;
(b) a step for measuring the expression level of the gene in the cells in contact with the test substance, and comparing said expression level with the expression level of the gene in control cells not in contact with the test substance;
(c) a step for selecting a test substance that regulates the expression level of the gene on the basis of the result of the comparison in (b) above.

4. The method according to claim 1, which comprises the following steps (a) to (c):
(a) a step for bringing the test substance into contact with target protein Y;
(b) a step for measuring the ability of the test substance to bind to the protein;
(c) a step for selecting a test substance capable of binding to the protein on the basis of the result from (b) above.

5. The method according to claim 1, which comprises the following steps (a) to (c):
(a) a step for bringing the test substance and a target protein Y-binding substance into contact with target protein Y;
(b) a step for measuring the ability of the target protein Y-binding substance to bind to the protein in the presence of the test substance, and comparing said ability with the ability of the target protein Y-binding substance to bind to the protein in the absence of the test substance;
(c) a step for selecting a test substance that alters the ability of the target protein Y-binding substance to bind to the protein on the basis of the result of the comparison in (b) above.

6. A method for screening a substance capable of regulating a function associated with target protein Y, which comprises comparing the ability of a test substance to bind to target protein Y or the action associated with the test compound, with the ability of bioactive substance X to bind to target protein Y or the action associated with the bioactive substance, wherein the combination of target protein Y and bioactive substance X is any of the following (b11) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b 11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

7. A substance obtained by the method according to claim 1.

8. An agent of regulating a bioactivity, which comprises a substance obtained by the method according to claim 1.

9. An agent of regulating an action associated with bioactive substance X, which comprises a substance that regulates the expression or function of target protein Y or a gene that encodes the protein, wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avernectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

10. The agent according to claim 9, wherein the substance that regulates the expression or function of target protein Y or a gene that encodes the protein is a substance that suppresses the expression or function of the gene.

11. The agent according to claim 10, wherein the substance that suppresses the expression or function of target protein Y or a gene that encodes the protein is an antisense nucleic acid, ribozyme, decoy nucleic acid, siRNA, antibody or a dominant negative mutant, or an expression vector thereof.

12. The agent according to claim 9, which comprises target protein Y, or an expression vector comprising a nucleic acid that encodes the protein.

13. An agent of regulating a function associated with target protein Y, which comprises bioactive substance X, wherein the combination of target protein Y and bioactive substance X is any of the following (b1) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b112) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

14. A method of producing a derivative of bioactive substance X, which comprises the derivatizing bioactive substance X so as to be able to regulate the expression or function of target protein Y or a gene that encodes the protein, wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

15. A method of producing a derivative of a substance capable of regulating a function associated with target protein Y, which comprises derivatizing bioactive substance X so as to be able to regulate the ability of bioactive substance X to bind to target protein Y, wherein the combination of target protein Y and bioactive substance X is any of the following (b1) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

16. A bioactive substance derivative obtained by the method according to claim 14.

17. An agent of regulating a bioactivity, which comprises a bioactive substance derivative obtained by the method according to claim 14 or IS.

18. A complex comprising bioactive substance X and target protein Y thereof, wherein the combination of bioactive substance X and target protein Y is any of the following (a1) to (a110) or (b1) to (b23):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of ebumamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

19. A method of producing the complex according to claim 18, which comprises bringing the bioactive substance and the target protein therefor into contact with each other.

20. A kit comprising the following (i) and (ii):
(i) bioactive substance X or a salt thereof;
(ii) target protein Y, a nucleic acid that encodes the protein, an expression vector comprising the nucleic acid, cells that enable a measurement of the expression of target protein Y or a gene that encodes the protein, or an expression vector comprising the transcription regulatory region of a gene that encodes target protein Y and a reporter gene functionally linked thereto;
wherein the combination of bioactive substance X and target protein Y is any of the following (a1) to (a110) or (b1) to (b23):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof,
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b115) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

21. A method for determining the onset or risk of onset of a disease or condition associated with an action of bioactive substance X, which comprises the following steps (a) and (b):
(a) a step for measuring the expression level and/or polymorphism of target protein Y or a gene that encodes the protein in a biological sample collected from an animal;
(b) a step for evaluating the onset or likelihood of onset of the disease or condition on the basis of the measured expression level and/or polymorphism;
wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

22. A method for determining the onset or risk of onset of a disease or condition associated with a function of target protein Y, which comprises the following steps (a) and (b):
(a) a step for identifying the polymorphism of the gene that encodes target protein Y in a biological sample collected from an animal;
(b) a step for evaluating the onset or likelihood of onset of the disease or condition on the basis of the presence or absence of a particular type of polymorphism;
wherein the particular type of polymorphism alters the ability of target protein Y to bind with bioactive substance X,
wherein the combination of target protein Y and bioactive substance X is any of the following (b1) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b118) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

23. A kit for determining the onset or risk of onset of a disease or condition associated with an action of bioactive substance X, which comprises the following (i) and (ii):
(i) a means capable of measuring the expression level and/or polymorphism of target protein Y or a gene that encodes the protein;
(ii) a medium recording the relationship between the disease or condition and the expression level and/or polymorphism of the gene;
wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

24. A kit for determining the onset or risk of onset of a disease or condition associated with a function of target protein Y, which comprises the following steps (i) and (ii):
(i) a means capable of identifying the polymorphism of a gene that encodes target protein Y;
(ii) a medium recording the relationship between the disease or condition and the polymorphism of the gene;
wherein the particular type of polymorphism alters the ability of target protein Y to bind with bioactive substance X,
wherein the combination of target protein Y and bioactive substance X is wherein the combination of target protein Y and bioactive substance X is any of the following (b1) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, ebumamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

25. A method for determining susceptibility to bioactive substance X in a disease or condition associated with an action of bioactive substance X, which comprises the following steps (a) and (b):
(a) a step for measuring the expression level and/or polymorphism of target protein Y or a gene that encodes the protein in a biological sample collected from an animal;
(b) a step for predicting the effect of the bioactive substance on the basis of the measured expression level and/or polymorphism;
wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof;
(a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof;
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

26. A method for determining susceptibility to bioactive substance X in a disease or condition associated with a function of target protein Y, which comprises the following steps (a) and (b):
(a) a step for determining the type of the polymorphism of the gene that encodes target protein Y in a biological sample collected from an animal;
(b) a step for predicting the effect of bioactive substance X in the disease or condition on the basis of the presence or absence of a particular type of polymorphism;
wherein the particular type of polymorphism alters the ability of target protein Y to bind with bioactive substance X,
wherein the combination of target protein Y and bioactive substance X is any of the following (b1) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, lopetamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

27. A kit for determining susceptibility to bioactive substance X in a disease or condition associated with an action of bioactive substance X, which comprises the following (i) and (ii):
(i) a means capable of measuring the expression level and/or polymorphism of a gene that encodes target protein Y;
(ii) a medium recording the relationship between the effect of bioactive substance X and the expression level and/or polymorphism of the gene;
wherein the combination of bioactive substance X and target protein Y is any of the following (a1) to (a110):
(a1) a combination of picotamide and a protein comprising the amino acid sequence shown by SEQ ID NO:1 or a protein homologous thereto or a variant thereof;
(a2) a combination of methoxsalen and a protein comprising the amino acid sequence shown by SEQ ID NO:2 or a protein homologous thereto or a variant thereof;
(a3) a combination of terfenadine and a protein comprising the amino acid sequence shown by SEQ ID NO:3, SEQ ID NO:14 or SEQ ID NO:19 or a protein homologous thereto or a variant thereof;
(a4) a combination of cyclosporin A and a protein comprising the amino acid sequence shown by SEQ ID NO:3, SEQ ID NO:8 or SEQ ID NO:12 or a protein homologous thereto or a variant thereof;
(a5) a combination of pancuronium bromide and a protein comprising the amino acid sequence shown by SEQ ID NO:4 or a protein homologous thereto or a variant thereof;
(a6) a combination of hydroxocobalamin and a protein comprising the amino acid sequence shown by SEQ ID NO:10, SEQ ID NO:11 or SEQ ID NO:15 or a protein homologous thereto or a variant thereof;
(a7) a combination of amphotericin B and a protein comprising the amino acid sequence shown by SEQ ID NO:15 or a protein homologous thereto or a variant thereof;
(a8) a combination of protriptyline and a protein comprising the amino acid sequence shown by SEQ ID NO:5 or a protein homologous thereto or a variant thereof;
(a9) a combination of rifampicin and a protein comprising the amino acid sequence shown by SEQ ID NO:6 or a protein homologous thereto or a variant thereof;
(a10) a combination of solanine α and a protein comprising the amino acid sequence shown by SEQ ID NO:7 or SEQ ID NO:15 or a protein homologous thereto or a variant thereof;
(a11) a combination of amethopterin and a protein comprising the amino acid sequence shown by SEQ ID NO:9 or a protein homologous thereto or a variant thereof;
(a12) a combination of benztropine and a protein comprising the amino acid sequence shown by SEQ ID NO:14 or SEQ ID NO:19 or a protein homologous thereto or a variant thereof;
(a13) a combination of sulfasalazine and a protein comprising the amino acid sequence shown by SEQ ID NO:13 or a protein homologous thereto or a variant thereof;
(a14) a combination of nalidixic acid and a protein comprising the amino acid sequence shown by SEQ ID NO:13 or a protein homologous thereto or a variant thereof;
(a15) a combination of astemizole and a protein comprising the amino acid sequence shown by SEQ ID NO:14 or SEQ ID NO:19 or a protein homologous thereto or a variant thereof;
(a16) a combination of chlorprothixene and a protein comprising the amino acid sequence shown by SEQ ID NO:14 or SEQ ID NO:19 or a protein homologous thereto or a variant thereof;
(a17) a combination of loperamide and a protein comprising the amino acid sequence shown by SEQ ID NO:14 or SEQ ID NO:19 or a protein homologous thereto or a variant thereof;
(a18) a combination of fluphenazine and a protein comprising the amino acid sequence shown by SEQ ID NO:14 or SEQ ID NO:19 or a protein homologous thereto or a variant thereof;
(a19) a combination of mefloquine and a protein comprising the amino acid sequence shown by SEQ ID NO:12, SEQ ID NO:14 or SEQ ID NO:19 or a protein homologous thereto or a variant thereof;
(a20) a combination of perphenazine and a protein comprising the amino acid sequence shown by SEQ ID NO:14 or SEQ ID NO:19 or a protein homologous thereto or a variant thereof;
(a21) a combination of perhexyline and a protein comprising the amino acid sequence shown by SEQ ID NO:12 or SEQ ID NO:14 or a protein homologous thereto or a variant thereof;
(a22) a combination of raloxifene and a protein comprising the amino acid sequence shown by SEQ ID NO:12, SEQ ID NO:14 or SEQ ID NO:19 or a protein homologous thereto or a variant thereof;
(a23) a combination of simvastatin and a protein comprising the amino acid sequence shown by SEQ ID NO:15 or a protein homologous thereto or a variant thereof;
(a24) a combination of benoxinate and a protein comprising the amino acid sequence shown by SEQ ID NO:16 or a protein homologous thereto or a variant thereof;
(a25) a combination of pioglitazone and a protein comprising the amino acid sequence shown by SEQ ID NO:17 or a protein homologous thereto or a variant thereof, (a26) a combination of thioproperazine and a protein comprising the amino acid sequence shown by SEQ ID NO:12 or SEQ ID NO:18 or a protein homologous thereto or a variant thereof;
(a27) a combination of quinacrine and a protein comprising the amino acid sequence shown by SEQ ID NO:12 or SEQ ID NO:19 or a protein homologous thereto or a variant thereof;
(a28) a combination of GBR12909 and a protein comprising the amino acid sequence shown by SEQ ID NO:19 or a protein homologous thereto or a variant thereof;
(a29) a combination of benzethonium and a protein comprising the amino acid sequence shown by SEQ ID NO:20 or SEQ ID NO:24 or a protein homologous thereto or a variant thereof;
(a30) a combination of albendazole and a protein comprising the amino acid sequence shown by SEQ ID NO:24 or a protein homologous thereto or a variant thereof;
(a31) a combination of acetopromazine and a protein comprising the amino acid sequence shown by SEQ ID NO:11 or a protein homologous thereto or a variant thereof;
(a32) a combination of amikacin and a protein comprising the amino acid sequence shown by SEQ ID NO:12 or a protein homologous thereto or a variant thereof;
(a33) a combination of amiodarone and a protein comprising the amino acid sequence shown by SEQ ID NO:12 or a protein homologous thereto or a variant thereof;
(a34) a combination of apigenin and a protein comprising the amino acid sequence shown by SEQ ID NO:15 or a protein homologous thereto or a variant thereof;
(a35) a combination of buprenorphine and a protein comprising the amino acid sequence shown by SEQ ID NO:12 or a protein homologous thereto or a variant thereof;
(a36) a combination of celestine blue and a protein comprising the amino acid sequence shown by SEQ ID NO:15 or a protein homologous thereto or a variant thereof;
(a37) a combination of chlorambucil and a protein comprising the amino acid sequence shown by SEQ ID NO:8 or a protein homologous thereto or a variant thereof;
(a38) a combination of chlorhexidine and a protein comprising the amino acid sequence shown by SEQ ID NO:12 or a protein homologous thereto or a variant thereof;
(a39) a combination of chlorpromazine and a protein comprising the amino acid sequence shown by SEQ ID NO:24 or a protein homologous thereto or a variant thereof;
(a40) a combination of cinchonine and a protein comprising the amino acid sequence shown by SEQ ID NO:15 or a protein homologous thereto or a variant thereof;
(a41) a combination of clofazimine and a protein comprising the amino acid sequence shown by SEQ ID NO:24 or a protein homologous thereto or a variant thereof;
(a42) a combination of clomiphene and a protein comprising the amino acid sequence shown by SEQ ID NO:12 or a protein homologous thereto or a variant thereof;
(a43) a combination of cyproheptadine and a protein comprising the amino acid sequence shown by SEQ ID NO:22 or a protein homologous thereto or a variant thereof;
(a44) a combination of deferoxamine and a protein comprising the amino acid sequence shown by SEQ ID NO:3 or a protein homologous thereto or a variant thereof;
(a45) a combination of dihydroergocristine and a protein comprising the amino acid sequence shown by SEQ ID NO:12 or a protein homologous thereto or a variant thereof;
(a46) a combination of dihydroergotamine and a protein comprising the amino acid sequence shown by SEQ ID NO:12 or a protein homologous thereto or a variant thereof;
(a47) a combination of diphemanil and a protein comprising the amino acid sequence shown by SEQ ID NO:11 or a protein homologous thereto or a variant thereof;
(a48) a combination of domperidone and a protein comprising the amino acid sequence shown by SEQ ID NO:23 or a protein homologous thereto or a variant thereof;
(a49) a combination of doxazosin and a protein comprising the amino acid sequence shown by SEQ ID NO:12 or a protein homologous thereto or a variant thereof;
(a50) a combination of eburnamonine and a protein comprising the amino acid sequence shown by SEQ ID NO:3 or a protein homologous thereto or a variant thereof;
(a51) a combination of ellipticine and a protein comprising the amino acid sequence shown by SEQ ID NO:24 or a protein homologous thereto or a variant thereof;
(a52) a combination of emetine and a protein comprising the amino acid sequence shown by SEQ ID NO:12 or a protein homologous thereto or a variant thereof;
(a53) a combination of ethotoin and a protein comprising the amino acid sequence shown by SEQ ID NO:13 or a protein homologous thereto or a variant thereof;
(a54) a combination of fenbendazole and a protein comprising the amino acid sequence shown by SEQ ID NO:12 or a protein homologous thereto or a variant thereof;
(a55) a combination of flumequine and a protein comprising the amino acid sequence shown by SEQ ID NO:13 or a protein homologous thereto or a variant thereof;
(a56) a combination of flunarizine and a protein comprising the amino acid sequence shown by SEQ ID NO:12 or a protein homologous thereto or a variant thereof;
(a57) a combination of flupentixol and a protein comprising the amino acid sequence shown by SEQ ID NO:12 or a protein homologous thereto or a variant thereof;
(a58) a combination of glipizide and a protein comprising the amino acid sequence shown by SEQ ID NO:24 or a protein homologous thereto or a variant thereof;
(a59) a combination of harmaline and a protein comprising the amino acid sequence shown by SEQ ID NO:15 or a protein homologous thereto or a variant thereof;
(a60) a combination of hydantoin and a protein comprising the amino acid sequence shown by SEQ ID NO:10 or a protein homologous thereto or a variant thereof;
(a61) a combination of lidoflazine and a protein comprising the amino acid sequence shown by SEQ ID NO:12 or a protein homologous thereto or a variant thereof;
(a62) a combination of lisinopril and a protein comprising the amino acid sequence shown by SEQ ID NO:17 or a protein homologous thereto or a variant thereof;
(a63) a combination of mafenide and a protein comprising the amino acid sequence shown by SEQ ID NO:6 or a protein homologous thereto or a variant thereof;
(a64) a combination of mefenamic acid and a protein comprising the amino acid sequence shown by SEQ ID NO:24 or a protein homologous thereto or a variant thereof;
(a65) a combination of megestrol and a protein comprising the amino acid sequence shown by SEQ ID NO:24 or a protein homologous thereto or a variant thereof;
(a66) a combination of mesoridazine and a protein comprising the amino acid sequence shown by SEQ ID NO:3 or a protein homologous thereto or a variant thereof;
(a67) a combination of metergoline and a protein comprising the amino acid sequence shown by SEQ ID NO:12 or a protein homologous thereto or a variant thereof;
(a68) a combination of methoxy-6-harmalan and a protein comprising the amino acid sequence shown by SEQ ID NO:15 or a protein homologous thereto or a variant thereof;
(a69) a combination of meticrane and a protein comprising the amino acid sequence shown by SEQ ID NO:15 or a protein homologous thereto or a variant thereof;
(a70) a combination of methixene and a protein comprising the amino acid sequence shown by SEQ ID NO:24 or a protein homologous thereto or a variant thereof;
(a71) a combination of mifepristone and a protein comprising the amino acid sequence shown by SEQ ID NO:7 or a protein homologous thereto or a variant thereof;
(a72) a combination of nordiazepam and a protein comprising the amino acid sequence shown by SEQ ID NO:24 or a protein homologous thereto or a variant thereof;
(a73) a combination of oxethazaine and a protein comprising the amino acid sequence shown by SEQ ID NO:12 or a protein homologous thereto or a variant thereof;
(a74) a combination of oxolinic acid and a protein comprising the amino acid sequence shown by SEQ ID NO:12 or a protein homologous thereto or a variant thereof;
(a75) a combination of paclitaxel and a protein comprising the amino acid sequence shown by SEQ ID NO:15 or a protein homologous thereto or a variant thereof;
(a76) a combination of palmatine and a protein comprising the amino acid sequence shown by SEQ ID NO:15 or a protein homologous thereto or a variant thereof;
(a77) a combination of pentoxifylline and a protein comprising the amino acid sequence shown by SEQ ID NO:24 or a protein homologous thereto or a variant thereof;
(a78) a combination of pimozide and a protein comprising the amino acid sequence shown by SEQ ID NO:3 or SEQ ID NO:12 or a protein homologous thereto or a variant thereof;
(a79) a combination of pinacidil and a protein comprising the amino acid sequence shown by SEQ ID NO:24 or a protein homologous thereto or a variant thereof;
(a80) a combination of rescinnamine and a protein comprising the amino acid sequence shown by SEQ ID NO:12 or a protein homologous thereto or a variant thereof;
(a81) a combination of SR-95639A and a protein comprising the amino acid sequence shown by SEQ ID NO:11 or a protein homologous thereto or a variant thereof;
(a82) a combination of sulfadimethoxine and a protein comprising the amino acid sequence shown by SEQ ID NO:15 or a protein homologous thereto or a variant thereof;
(a83) a combination of syrosingopine and a protein comprising the amino acid sequence shown by SEQ ID NO:24 or a protein homologous thereto or a variant thereof;
(a84) a combination of tamoxifen and a protein comprising the amino acid sequence shown by SEQ ID NO:12 or a protein homologous thereto or a variant thereof;
(a85) a combination of tenoxicam and a protein comprising the amino acid sequence shown by SEQ ID NO:13 or a protein homologous thereto or a variant thereof;
(a86) a combination of thioridazine and a protein comprising the amino acid sequence shown by SEQ ID NO:12 or a protein homologous thereto or a variant thereof;
(a87) a combination of thiothixene (cis) and a protein comprising the amino acid sequence shown by SEQ ID NO:12 or SEQ ID NO:24 or a protein homologous thereto or a variant thereof;
(a88) a combination of tomatidine and a protein comprising the amino acid sequence shown by SEQ ID NO:24 or a protein homologous thereto or a variant thereof,
(a89) a combination of dipyrone and a protein comprising the amino acid sequence shown by SEQ ID NO:24 or a protein homologous thereto or a variant thereof;
(a90) a combination of ethyl loflazepate and a protein comprising the amino acid sequence shown by SEQ ID NO:24 or a protein homologous thereto or a variant thereof;
(a91) a combination of clobazam and a protein comprising the amino acid sequence shown by SEQ ID NO:24 or a protein homologous thereto or a variant thereof;
(a92) a combination of alimemazine and a protein comprising the amino acid sequence shown by SEQ ID NO:12 or a protein homologous thereto or a variant thereof;
(a93) a combination of ebastine and a protein comprising the amino acid sequence shown by SEQ ID NO:24 or a protein homologous thereto or a variant thereof;
(a94) a combination of reserpine and a protein comprising the amino acid sequence shown by SEQ ID NO:12 or a protein homologous thereto or a variant thereof;
(a95) a combination of paramethasone and a protein comprising the amino acid sequence shown by SEQ ID NO:15 or a protein homologous thereto or a variant thereof;
(a96) a combination of hydroxyprogesterone and a protein comprising the amino acid sequence shown by SEQ ID NO:12 or a protein homologous thereto or a variant thereof;
(a97) a combination of dydrogesterone and a protein comprising the amino acid sequence shown by SEQ ID NO:24 or a protein homologous thereto or a variant thereof;
(a98) a combination of sarpogrelate and a protein comprising the amino acid sequence shown by SEQ ID NO:15 or a protein homologous thereto or a variant thereof;
(a99) a combination of demeclocycline and a protein comprising the amino acid sequence shown by SEQ ID NO:15 or a protein homologous thereto or a variant thereof;
(a100) a combination of avermectin B1A and a protein comprising the amino acid sequence shown by SEQ ID NO:15 or a protein homologous thereto or a variant thereof;
(a101) a combination of solasodine and a protein comprising the amino acid sequence shown by SEQ ID NO:24 or a protein homologous thereto or a variant thereof;
(a102) a combination of nanofin (cis) and a protein comprising the amino acid sequence shown by SEQ ID NO:20 or a protein homologous thereto or a variant thereof;
(a103) a combination of tetrazoline and a protein comprising the amino acid sequence shown by SEQ ID NO:11 or a protein homologous thereto or a variant thereof;
(a104) a combination of methylbenzethonium chloride and a protein comprising the amino acid sequence shown by SEQ ID NO:23 or SEQ ID NO:24 or a protein homologous thereto or a variant thereof;
(a105) a combination of α-ergocryptine and a protein comprising the amino acid sequence shown by SEQ ID NO:24 or a protein homologous thereto or a variant thereof;
(a106) a combination of spiramycin and a protein comprising the amino acid sequence shown by SEQ ID NO:6 or a protein homologous thereto or a variant thereof;
(a107) a combination of chloropyramine and a protein comprising the amino acid sequence shown by SEQ ID NO:15 or a protein homologous thereto or a variant thereof;
(a108) a combination of bergenin and a protein comprising the amino acid sequence shown by SEQ ID NO:15 or a protein homologous thereto or a variant thereof;
(a109) a combination of nafcillin and a protein comprising the amino acid sequence shown by SEQ ID NO:15 or a protein homologous thereto or a variant thereof;
(a110) a combination of carboprost and a protein comprising the amino acid sequence shown by SEQ ID NO:15 or a protein homologous thereto or a variant thereof.

28. A kit for determining susceptibility to bioactive substance X in a disease or condition associated with a function of target protein Y, which comprises the following (i) and (ii):
(i) a means capable of identifying the polymorphism of a gene that encodes target protein Y;
(ii) a medium recording the relationship between the effect of bioactive substance X and a particular type of the polymorphism of the gene;
wherein the particular type of polymorphism alters the ability of target protein Y to bind with bioactive substance X,
wherein the combination of target protein Y and bioactive substance X is wherein the combination of target protein Y and bioactive substance X is any of the following (b1) to (b23):
(b1) a combination of a protein comprising the amino acid sequence shown by SEQ ID NO:1 or a protein homologous thereto or a variant thereof and picotamide or a derivative thereof capable of binding to the protein;
(b2) a combination of a protein comprising the amino acid sequence shown by SEQ ID NO:2 or a protein homologous thereto or a variant thereof and methoxsalen or a derivative thereof capable of binding to the protein;
(b3) a combination of a protein comprising the amino acid sequence shown by SEQ ID NO:3 or a protein homologous thereto or a variant thereof and terfenadine, cyclosporin A, deferoxamine, eburnamonine, mesoridazine, pimozide or a derivative thereof capable of binding to the protein;
(b4) a combination of a protein comprising the amino acid sequence shown by SEQ ID NO:4 or a protein homologous thereto or a variant thereof and pancuronium bromide or a derivative thereof capable of binding to the protein;
(b5) a combination of a protein comprising the amino acid sequence shown by SEQ ID NO:5 or a protein homologous thereto or a variant thereof and protriptyline or a derivative thereof capable of binding to the protein;
(b6) a combination of a protein comprising the amino acid sequence shown by SEQ ID NO:6 or a protein homologous thereto or a variant thereof and rifampicin, mafenide, spiramycin or a derivative thereof capable of binding to the protein;
(b7) a combination of a protein comprising the amino acid sequence shown by SEQ ID NO:7 or a protein homologous thereto or a variant thereof and solanine α, mifepristone or a derivative thereof capable of binding to the protein;
(b8) a combination of a protein comprising the amino acid sequence shown by SEQ ID NO:8 or a protein homologous thereto or a variant thereof and cyclosporin A, chlorambucil or a derivative thereof capable of binding to the protein;
(b9) a combination of a protein comprising the amino acid sequence shown by SEQ ID NO:9 or a protein homologous thereto or a variant thereof and amethopterin or a derivative thereof capable of binding to the protein;
(b10) a combination of a protein comprising the amino acid sequence shown by SEQ ID NO:10 or a protein homologous thereto or a variant thereof and hydroxocobalamin, hydantoin or a derivative thereof capable of binding to the protein;
(b11) a combination of a protein comprising the amino acid sequence shown by SEQ ID NO:11 or a protein homologous thereto or a variant thereof and hydroxocobalamin, acetopromazine, diphemanil, SR-95639A, tetrazoline or a derivative thereof capable of binding to the protein;
(b12) a combination of a protein comprising the amino acid sequence shown by SEQ ID NO:12 or a protein homologous thereto or a variant thereof and cyclosporin A, mefloquine, perhexyline, raloxifene, thioproperazine, quinacrine, amikacin, amiodarone, buprenorphine, chlorhexidine, clomiphene, dihydroergocristine, dihydroergotamine, doxazosin, emetine, fenbendazole, flunarizine, flupentixol, lidoflazine, metergoline, oxethazaine, oxolinic acid, pimozide, rescinnamine, tamoxifen, thioridazine, thiothixene (cis), alimemazine, reserpine, hydroxyprogesterone or a derivative thereof capable of binding to the protein;
(b13) a combination of a protein comprising the amino acid sequence shown by SEQ ID NO:13 or a protein homologous thereto or a variant thereof and sulfasalazine, nalidixic acid, ethotoin, flumequine, tenoxicam or a derivative thereof capable of binding to the protein;
(b14) a combination of a protein comprising the amino acid sequence shown by SEQ ID NO:14 or a protein homologous thereto or a variant thereof and astemizole, chlorprothixene, benztropine, loperamide, fluphenazine, mefloquine, perphenazine, perhexyline, raloxifene, terfenadine or a derivative thereof capable of binding to the protein;
(b15) a combination of a protein comprising the amino acid sequence shown by SEQ ID NO:15 or a protein homologous thereto or a variant thereof and amphotericin B, hydroxocobalamin, simvastatin, solanine α, apigenin, celestine blue, cinchonine, harmaline, methoxy-6-harmalan, meticrane, paclitaxel, palmatine, sulfadimethoxine, paramethasone, sarpogrelate, demeclocycline, avermectin B1A, chloropyramine, bergenin, nafcillin, carboprost or a derivative thereof capable of binding to the protein;
(b16) a combination of a protein comprising the amino acid sequence shown by SEQ ID NO:16 or a protein homologous thereto or a variant thereof and benoxinate or a derivative thereof capable of binding to the protein;
(b17) a combination of a protein comprising the amino acid sequence shown by SEQ ID NO:17 or a protein homologous thereto or a variant thereof and pioglitazone, lisinopril or a derivative thereof capable of binding to the protein;
(b18) a combination of a protein comprising the amino acid sequence shown by SEQ ID NO:18 or a protein homologous thereto or a variant thereof and thioproperazine or a derivative thereof capable of binding to the protein;
(b19) a combination of a protein comprising the amino acid sequence shown by SEQ ID NO:19 or a protein homologous thereto or a variant thereof and raloxifene, loperamide, mefloquine, perphenazine, quinacrine, GBR12909, terfenadine, fluphenazine, astemizole, benztropine, chlorprothixene or a derivative thereof capable of binding to the protein;
(b20) a combination of a protein comprising the amino acid sequence shown by SEQ ID NO:20 or a protein homologous thereto or a variant thereof and benzethonium, nanofin (cis) or a derivative thereof capable of binding to the protein;
(b21) a combination of a protein comprising the amino acid sequence shown by SEQ ID NO:22 or a protein homologous thereto or a variant thereof and cyproheptadine or a derivative thereof capable of binding to the protein;
(b22) a combination of a protein comprising the amino acid sequence shown by SEQ ID NO:23 or a protein homologous thereto or a variant thereof and domperidone, methylbenzethonium chloride or a derivative thereof capable of binding to the protein;
(b23) a combination of a protein comprising the amino acid sequence shown by SEQ ID NO:24 or a protein homologous thereto or a variant thereof and benzethonium, albendazole, chlorpromazine, clofazimine, ellipticine, glipizide, mefenamic acid, megestrol, methixene, nordiazepam, pentoxifylline, pinacidil, syrosingopine, thiothixene (cis), tomatidine, dipyrone, ethyl loflazepate, clobazam, ebastine, dydrogesterone, solasodine, methylbenzethonium chloride, α-ergocryptine or a derivative thereof capable of binding to the protein.

29. A substance obtained by the method according to claim 6.

30. An agent of regulating a bioactivity, which comprises a substance obtained by the method according to claim 6.

31. A bioactive substance derivative obtained by the method according to claim 15.

32. An agent of regulating a bioactivity, which comprises a bioactive substance derivative obtained by the method according to claim 15.


SEQUENCE LISTING (24 sequences)

SEQ ID NO: 1 - protein, 263 aa, Homo sapiens
MEESGYESVLCVKPDVHVYRIPPRATNRGYRAAEWQLDQPSWSGRLRITAKGQMAYIKLEDRTSGELFAQAPVDQFPGTAVESVTDSSRYFVIRIEDGNGRRAFIGIGFGDRGDAFDFNVALQDHFKWVKQQCEFAKQAQNPDQGPKLDLGFKEGQAIKLNIANMKKKEGAAGNPRVRPASTGGLSLLPPPPGGKTSTLIPPPGEQLAVGGSLVQPAVAPSSGGAPVPWPQPNPATADIWGDFTKSTGSTSSQTQPGTGWVQF

SEQ ID NO: 2 - protein, 186 aa, Homo sapiens
MKSDFYFQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 3 - protein, 303 aa, Homo sapiens [sequence omitted]

SEQ ID NO: 4 - protein, 270 aa, Homo sapiens [sequence omitted]

SEQ ID NO: 5 - protein, 141 aa, Homo sapiens
MEQVAKKLGVAHEEIQRLTDELQVKEKEQCKLDSALKKAQLEIDKLKENLVKLKENDAADLQKAKEQNQRLDEEILALRNRVRSLDSEKKVLGEMVERLKGEVCESQENKQLGNHSPGKTVGGEQREQIEASMLEPELSVT

SEQ ID NO: 6 - protein, 430 aa, Homo sapiens
MAGVENEDEAELNLREQGGFTGKEEVVEQGELNATEEVWIPGEGHPESPEPKEQRGLVEGASVKGGAEGLQDPEGQSQQVGAPGLQAPQGLPEAIEPLVEDDVAPGGDQASPEVMLGSEPAMGESAAGAEPGLGQGVGGLGDPGHLTREEVMEPPLEEESLEAKRVQGLEGPRKDLEEAGGLGTEFSELPGKSRDPWEPPREGREESEAEAPRGAEEAFPAETLGHTGSDAPSPWPLGSEEAEEDVPPVLVSPSPTYTPILEDAPGPQPQAEGSQEASWGVQGRAEALGKVESEQEELGSGEIPEGLQEEGEESREESEEDQEEGREPGAGRWGPGSSVGSLQALSSSQRGEFLESDSVSVSVPWDDSLRGAVAGAPKTALETESQDSAEPSGSEEESDPVSLERENKVPGPLEIPSGMEDAARGRHHWC

SEQ ID NO: 7 - protein, 329 aa, Homo sapiens
MEVAEPSSPTEEEEEEEEHSAEPRPRTRSNPEGAEDRAVGAQASVGSRSEGEGEAASADDGSLNTSGAGPKSWQVPPPAPEVQIRTPRVNCPEKVIICLDLSEEMSLPKLESFNGSKTNALNVSQKMIEMFVRTKHKIDKSHEFALVVVNDDTAWLSGLTSDPRELCSCLYDLETASCSTFNLEGLFSLIQQKTELPVTENVQTIPPPYVVRTILVYSRPPCQPQFSLTEPMKKMFQCPYFFFDVVYIHNGTEEKEEEMSWKDMFAFMGSLDTKGTSYKYEVALAGPALELHNCMAKLLAHPLQRPCQSHASYSLLEEEDEAIEVEATV

SEQ ID NO: 8 - protein, 203 aa, Homo sapiens [sequence omitted]

SEQ ID NO: 9 - protein, 321 aa, Homo sapiens [sequence omitted]

SEQ ID NO: 10 - protein, 281 aa, Homo sapiens
MDSIPAPSSVQGHNLTEDARHPESWQNTGGYSEGDAVSQPQMALEEVSVSDPLASNQGQSLPGSSREHMAQWEVRSQTHVPNREPVQALPSSASRKRLDKKRSVPVATVELEEKRFRTLPLVPSPLQGLTNQDLQEGEDWEQEDEDMDPRLEHSSSVQEDSESPSPEDIPDYLLQYRAIHSAEQQHAYEQDFETDYAEYRILHARVGTASQRFIELGAEIKRVRRGTPEYKVLEDKIIQEYKKFRKYPSYREEKRRCEYLHQKLSHIKGLILEFEEKNRGS

SEQ ID NO: 11 - protein, 370 aa, Homo sapiens
MAAERQEALREFVAVTGAEEDRARFFLESAGWDLQIALASFYEDGGDEDIVTISQATPSSVSRGTAPSDNRVTSFRDLIHDQDEDEEEEEGQRFYAGGSERSGQQIVGPPRKKSPNELVDDLFKGAKEHGAVAVERVTKSPGETSKPRPFAGGGYRLGAAPEEESAYVAGEKRQHSSQDVHVVLKLWKSGFSLDNGELRSYQDPSNAQFLESIRRGEVPAELRRLAHGGQVNLDMEDHRDEDFVKPKGAFKAFTGEGQKLGSTAPQVLSTSSPAQQAENEAKASSSILINESEPTTNIQIRLADGGRLVQKFNHSHRISDIRLFIVDARPAMAATSFILMTTFPNKELADESQTLKEANLFNAVIVQRLT

SEQ ID NO: 12 - protein, 218 aa, Homo sapiens
MANVELSSLARLPSLNKLRKLELSDNIISGGLEVLAEKCPNLTYLNLSGNKIKDLSTVEALQNLKNLKSLDLFNCEITNLEDYRESIFELLQQITYLDGFDQEDNEAPDSEEEDDEDGDEDDEEEEENEAGPPEGYEEEEEEEEDEDEDEDEDEAGSELGEGEEEVGLSYLMKEEIQDEEDDDDYVEEGEEEEEEEEGGLRGEKRKRDAEDDGEEEDD

SEQ ID NO: 13 - protein, 153 aa, Homo sapiens
MALVFVYGTLKRGQPNHRVLRDGAHGSAAFRARGRTLEPYPLVIAGEHNIPWLLHLPGSGRLVEGEVYAVDERMLRFLDDFESCPALYQRTVLRVQLLEDRAPGAEEPPAPTAVQCFVYSRATFPPEWAQLPHHDSYDSEGPHGLRYNPRENR

SEQ ID NO: 14 - protein, 220 aa, Homo sapiens
MMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 15 - protein, 374 aa, Homo sapiens
MGCSSSALNKAGDSSRFPSVTSNEHFSTAEESESCFAQPKPHALGRESTVDGNVQRESRPPLQKLKVSAEPTANGVKPLQEQPLAKDVAPGRDATDQSGSTEKTQPGEGLEESGPPQPGGKEDAPAAEGKKKDAGAGTEAESLKGNAEAQPLGPEAKGQPLQAAVEKDSLRAVEVTENPQTAAEMKPLGTTENVLTLQIAGELQPQGTVGKDEQAPLLETISKENESPEILEGSQFVETAEEQQLQATLGKEEQPQLLERIPKENVTPEVLDRSQLVEKPVMNDPFHKTPEGPGNMEQIQPEGIVGSMEHPARNVEAGAYVEMIRSIHTNEEDQRIEGETGEKVETDMENEKVSEGAETKEEETGEVVDLSAAT

SEQ ID NO: 16 - protein, 166 aa, Homo sapiens
MQKPSCGIVPLASPGTSAELQNNFIEYISFIHQYDARKTPNEPLQGKRHGAFVQREIKPGSRPTVPKGAEVLLNTPGSRSSEQSKKTEKGNSAESRMISPGLCQQNSQELLEPKTHLSETDVRQAAKACPSTLESREKTSGATQTTVGDALFTTHKPLNPPIKKSE

SEQ ID NO: 17 - protein, 426 aa, Homo sapiens [sequence omitted]

SEQ ID NO: 18 - protein, 215 aa, Homo sapiens
MRGLRWRYTRLPSQVEDTLSGEEGNEEEEEEEAAPDPAAAPEDPTVPQLTEASQVLSASEIRQLSFHFPPRVTGHPWSLVFCTSRDGFSLQSLYRRMEGCSGPVLLVLRDQDGQIFGAFSSSAIRLSKGFYGTGETFLFSFSPQLKVFKWTGSNSFFVKGDLDSLMMGSGSGRFGLWLDGDLFRGGSSPCPTFNNEVLARQEQFCIQELEAWLLS

SEQ ID NO: 19 - protein, 220 aa, Homo sapiens [sequence omitted]

SEQ ID NO: 20 - protein, 434 aa, Homo sapiens
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 21 - protein, 445 aa, Homo sapiens
MSELTKELMELVWGTKSSPGLSDTIFCRWTQGFVFSESEGSALEQFEGGPCAVIAPVQAFLLKKLLFSSEKSSWRDCSEEEQKELLCHTLCDILESACCDHSGSYCLVSWLRGKTTEETASISGSPAESSCQVEHSSALAVEELGFERFHALIQKRSFRSLPELKDAVLDQYSMWGNKFGVLLFLYSVLLTKGIENIKNEIEDASEPLIDPVYGHGSQSLINLLLTGHAVSNVWDGDRECSGMKLLGIHEQAAVGFLTLMEALRYCKVGSYLKSPKFPIWIVGSETHLTVFFAKDMALVAPEAPSEQARRVFQTYDPEDNGFIPDSLLEDVMKALDLVSDPEYINLMKNKLDPEGLGIILLGPFLQEFFPDQGSSGPESFTVYHYNGLKQSNYNEKVMYVEGTAVVMGFEDPMLQTDDTPIKRCLQTKWPYIELLWTTDRSPSLN

SEQ ID NO: 22 - protein, 140 aa, Homo sapiens
MTVEPSFNDAHVSKFSDQNSTELMNSVMVRTEEEISDTDLKQEESPSDLASAYVTEGLESPTIEEQVDQTIDDETILIVPSPHGFIQASDVIDTESVLPLTTLTDPILQHHQEESNIIGSSLGSPVSEDSKDVEDLVNCH

SEQ ID NO: 23 - protein, 329 aa, Homo sapiens
MEVAEPSSPTEEEEEEEEHSAEPRPRTRSNPEGAEDRAVGAQASVGSRSEGEGEAASADDGSLNTSGAGPKSWQVPPPAPEVQIRTPRVNCPEKVIICLDLSEEMSLPKLESFNGSKTNALNVSQKMIEMFVRTKHKIDKSHEFALVVVNDDTAWLSGLTSDPRELCSCLYDLETASCSTFNLEGLFSLIQQKTELPVTENVQTIPPPYVVRTILVYSRPPCQPQFSLTEPMKKMFQCPYFFFDVVYIHNGTEEKEEEMSWKDMFAFMGSLDTKGTSYKYEVALAGPALELHNCMAKLSAHPLQRPCQSHASYSLLEEEDEAIEVEATV

SEQ ID NO: 24 - protein, 268 aa, Homo sapiens
MEMKKKINLELRNRSPEEVTELVLDNCLCVNGEIEGLNDTFKELEFLSMANVELSSLARLPSLNKLRKLELSDNIISGGLEVLAEKCPNLTYLNLSGNKIKDLSTVEALQNLKNLKSLDLFNCEITNLEDYRESIFELLQQITYLDGFDQEDNEAPDSEEEDDEDGDEDDEEEEENEAGPPEGYEEEEEEEEEEDEDEDEDEDEAGSELGEGEEEVGLSYLMKEEIQDEEDDDDYVEEGEEEEEEEEGGLRGEKRKRDAEDDGEEEDD